(19) United States
(12) Patent Application Publication
(10) Pub. No.: US 2025/0197375 A1
(43) Pub. Date: Jun. 19, 2025

(54) PYRIMIDINES AND METHODS OF THEIR USE

(71) Applicant: Kineta, Inc., Seattle, WA (US)

(72) Inventors: Gnanasambandam Kumaravel, Lexington, MA (US); Madeline Macdonnell, Brighton, MA (US); Hairuo Peng, Needham, MA (US)

(21) Appl. No.: 18/717,141

(22) PCT Filed: Dec. 8, 2022
(86) PCT No.: PCT/US2022/052224

§ 371 Date: Jun. 6, 2024

Related U.S. Application Data
(60) Provisional application No. 63/287,479, filed on Dec. 8, 2021.

(51) Int. Cl. C07D 403/04; A61K 31/506; A61K 31/5377; A61K 31/5383; A61K 31/675; C07D 401/12; C07D 401/14; C07D 403/10; C07D 413/14; C07D 471/04; C07D 491/052; C07D 491/147; C07D 498/14; C07F 9/6558

(52) U.S. Cl. CPC C07D 403/04; A61K 31/506; A61K 31/5377; A61K 31/5383; A61K 31/675; C07D 401/12; C07D 401/14; C07D 403/10; C07D 413/14; C07D 471/04; C07D 491/052; C07D 491/147; C07D 498/14; C07F 9/65583

(57) ABSTRACT

Disclosed are compounds comprising pyrimidinyl core and pharmaceutical compositions useful in the treatment of neurological disorders. The compounds described herein, alone or in combination with other pharmaceutically active agents, can be used for treating or preventing neurological diseases, including FTLD-TDP, chronic traumatic encephalopathy, ALS, Alzheimer's disease, LATE, or frontotemporal lobar degeneration.

### FIELD OF THE INVENTION

The invention relates to heteroarenes and their use for therapeutic treatment of neurological disorders in patients, such as human patients.

### BACKGROUND

An incomplete understanding of the molecular perturbations that cause disease, as well as a limited arsenal of robust model systems, has contributed to a failure to generate successful disease-modifying therapies against common and progressive neurological disorders, such as ALS and FTD. Progress is being made on many fronts to find agents that can arrest the progress of these disorders. However, the present therapies for most, if not all, of these diseases provide very little relief. Accordingly, a need exists to develop therapies that can after the course of neurodegenerative diseases. More generally, a need exists for better methods and compositions for the treatment of neurodegenerative diseases in order to improve the quality of the lives of those afflicted by such diseases.

### SUMMARY

TDP-43 is a nuclear DNA/RNA binding protein involved in RNA splicing. Under pathological cell stress, TDP-43 translocates to the cytoplasm and aggregates into stress granules and related protein inclusions. These phenotypes are hallmarks of degenerating motor neurons and are found in 97% of all ALS cases. The highly penetrant nature of this pathology indicates that TDP-43 is broadly involved in both familial and sporadic ALS. Additionally, TDP-43 mutations that promote aggregation are linked to higher risk of developing ALS, suggesting protein misfolding and aggregation act as drivers of toxicity.

TDP-43 toxicity can be recapitulated in yeast models, where the protein induces a viability deficit and localizes to stress granules.

In an aspect, the disclosure provides a compound of Formula I:

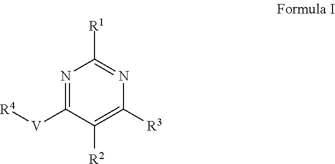

or a pharmaceutically acceptable sat thereof,
wherein
   V is —NH—, —NR⁵—, —CH₂NH—, —CH₂NR⁵—, —O—, —CO—, or —CHOH—;
   R¹ is optionally substituted morpholin-4-yl, pyridin-4-yl, pyridin-3-yl, optionally substituted 2-oxo-pyrrolidin-1-yl, optionally substituted piperidin-1-yl, or optionally substituted pyridiazin-4-yl;
   R² is halogen, —(CH₂)ₙOH, optionally substituted C₁₋₆ alkoxy, optionally substituted C₂-C₉ heteroaryl, optionally substituted 2-oxo-pyrrolidin-1-yl, —(CO)NR^{7a}R^{7b}, —P(O)R^{7c}R^{7d}, or —S(O)ₖR^{7e}; and R³ is optionally substituted pyridin-2-yl, optionally substituted pyridin-3-yl, optionally substituted pyridin-4-yl, optionally substituted pyrazol-1-yl, optionally substituted pyrazol-3-yl, optionally substituted pyridazin-3-yl, optionally substituted pyrimidin-4-yl, or optionally substituted C₆-C₁₀ aryl; or R² and R³, together with the ring to which they are attached, combine to form optionally substituted C₄-C₁₂ heteroaryl;
   n is 1, 2, 3, 4, 5, or 6;
   k is 0, 1, or 2;
   R⁴ is optionally substituted pyridin-4-yl, optionally substituted 1-methylpyridin-1-ium-4-yl, optionally substituted pyridin-3-yl, optionally substituted 1-methylpiperidin-3-yl, optionally substituted pyridazine-3-yl, or —NHR⁸;
   R⁵ is optionally substituted C₁-C₆ alkyl;
   R⁶ is H or optionally substituted C₁-C₆ alkyl;
   each of R^{7a} and R^{7b} is, independently, H or optionally substituted C₁-C₆ alkyl; or R^{7a} and R^{7b}, together with the nitrogen atom to which they are attached, combine to form optionally substituted C₂-C₉ heterocyclyl;
   each of R^{7c}, R^{7d}, and R^{7e} is independently optionally substituted C₁₋₆ alkyl, optionally substituted C₁₋₆ alkoxy, or hydroxyl; and
   R⁸ is optionally substituted phenyl or optionally substituted C₃-C₆ cycloalkyl.

In some embodiments, R¹ is optionally substituted morpholin-4-yl. In some embodiments, R¹ is optionally substituted pyridin-4-yl. In some embodiments, R¹ is optionally substituted pyridin-3-yl.

In some embodiments, R¹ is

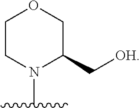 [SMILES: *N1CCOC[C@H]1CO]

In some embodiments, R¹ is

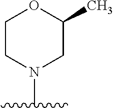 [SMILES: *N1CCO[C@@H](C)C1]

or

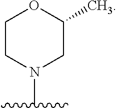 [SMILES: *N1CCO[C@H](C)C1]

In some embodiments, R¹ is morpholin-4-yl. In some embodiments, R¹ is optionally substituted piperidin-1-yl. In some embodiments, R¹ is

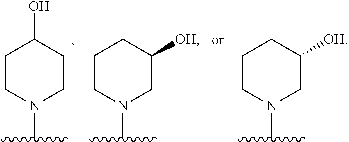

In some embodiments, V is —NH—. In some embodiments, V is —CH₂NH—. In some embodiments, V is —O—. In some embodiments, V is —CO—. In some embodiments, V is —CHOH—. In some embodiments, V is —NR⁵—. In some embodiments, V is —CH₂NR⁵—.

In some embodiments, R⁴ is optionally substituted pyridin-4-yl, optionally substituted 1-methylpyridin-1-ium-4-yl, optionally substituted pyridin-3-yl, optionally substituted 1-methylpiperidin-3-yl, optionally substituted pyridazine-3-yl. In some embodiments, R⁴ is optionally substituted pyridin-4-yl, optionally substituted pyridin-3-yl, optionally substituted 1-methylpiperidin-3-yl, optionally substituted pyridazine-3-yl. In some embodiments, R⁴ is optionally substituted pyridin-4-yl or optionally substituted pyridin-3-yl. In some embodiments, R⁴ is pyridin-4-yl or pyridin-3-yl.

In some embodiments, R² and R³, together with the ring to which they are attached, combine to form optionally substituted C₄-C₁₂ heteroaryl. In some embodiments, R² and R³, together with the ring to which they are attached, combine to form optionally substituted C₄-C₉ heteroaryl.

In some embodiments, the compound is of the following structure:

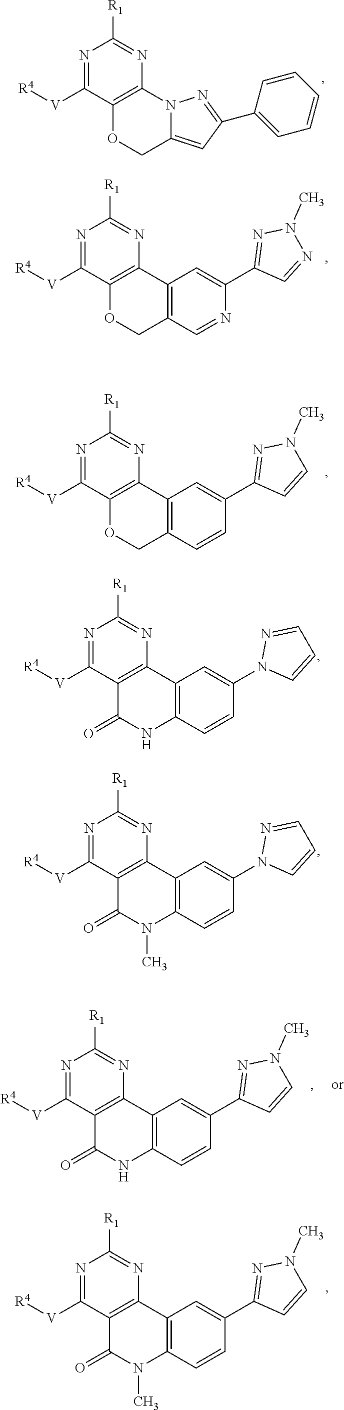

or a pharmaceutically acceptable salt thereof.

In some embodiments, R² is halogen, —(CH₂)ₙOH, optionally substituted C₁₋₆ alkoxy, optionally substituted C₂-C₉ heteroaryl, optionally substituted 2-oxo-pyrrolidin-1-yl, —(CO)NR^{7a}R^{7b}, —P(O)R^{7c}R^{7d}, or —S(O)ₖR^{7e}. In some embodiments, R² is optionally substituted C₁₋₆ alkoxy, optionally substituted C₂-C₉ heteroaryl, optionally substituted 2-oxo-pyrrolidin-1-yl, —(CO)NR^{7a}R^{7b}, —P(O)R^{7c}R^{7d}, or —S(O)ₖR^{7e}.

In some embodiments, R² is optionally substituted C₁₋₆ alkoxy. In some embodiments, R² is —OCH₃.

In some embodiments, R² is —(CO)NR^{7a}R^{7b}. In some embodiments, each of R^{7a} and R^{7b} is, independently, optionally substituted C₁-C₆ alkyl. In some embodiments, each of R^{7a} and R^{7b} is methyl. In some embodiments, R^{7a} and R^{7b}, together with the nitrogen atom to which they are attached, combine to form optionally substituted C₂-C₉ heterocyclyl. In some embodiments, —(CO)NR^{7a}R^{7b} is

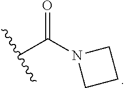 [SMILES: *C(=O)N1CCC1]

In some embodiments, R² is —P(O)R^{7c}R^{7d}, or —S(O)ₖR^{7e}. In some embodiments, R² is

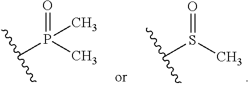

In some embodiments, R² is optionally substituted C₂-C₉ heteroaryl. In some embodiments, R² is

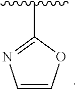 [SMILES: *c1ncco1]

In some embodiments, R² is halogen. In some embodiments, R² is Cl.

In some embodiments, R² is —(CH₂)ₙOH. In some embodiments, n is 1, 2, or 3. In some embodiments, n is 1.

In some embodiments, R³ is optionally substituted pyridin-2-yl, optionally substituted pyridin-3-yl, optionally substituted pyridin-4-yl, optionally substituted pyrazol-1-yl, optionally substituted pyrazol-3-yl, optionally substituted pyridazin-3-yl, optionally substituted pyrimidin-4-yl, or optionally substituted C₆-C₁₀ aryl.

In some embodiments, R³ is optionally substituted pyridin-3-yl, optionally substituted pyridin-4-yl, or optionally substituted C₆-C₁₀ aryl.

In some embodiments, R³ is optionally substituted Ce aryl. In some embodiments, R³ is

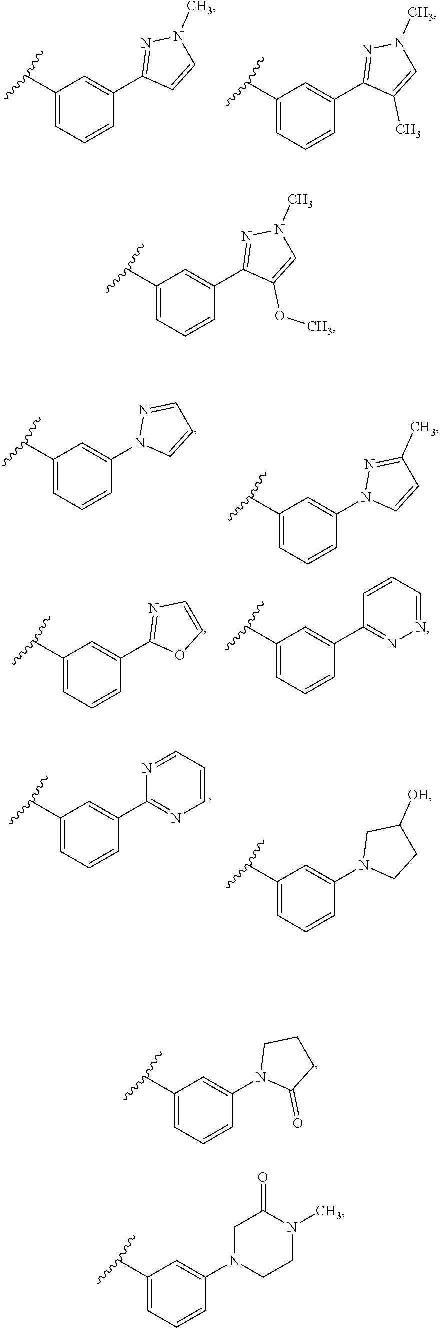

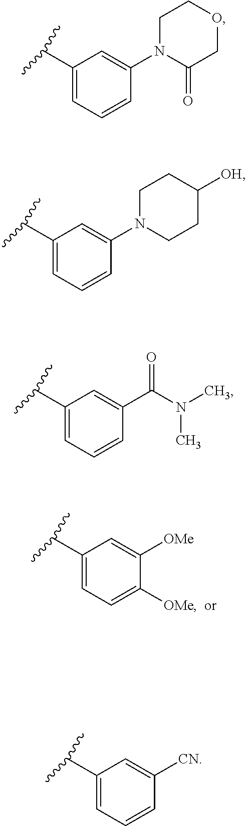 [SMILES: *c1cccc(N2CCOCC2=O)c1 ; *c1cccc(N2CCC(O)CC2)c1 ; *c1cccc(C(=O)N(C)C)c1 ; *c1ccc(OC)c(OC)c1 ; *c1cccc(C#N)c1]

In some embodiments, R³ is optionally substituted pyridin-3-yl or optionally substituted pyridin-4-yl. In some embodiments, R³ is

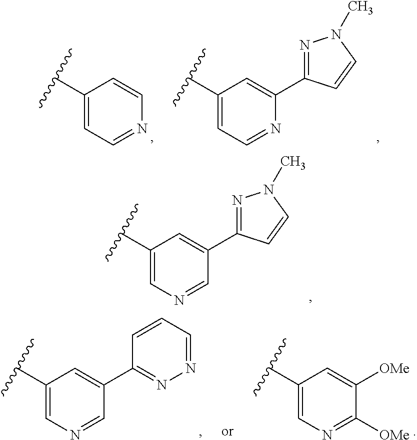

In some embodiments, R³ is optionally substituted pyridin-2-yl, optionally substituted pyrazol-1-yl, optionally substituted pyrazol-3-yl, optionally substituted pyridazin-3-yl, or optionally substituted pyrimidin-4-yl. In some embodiments, R³ is

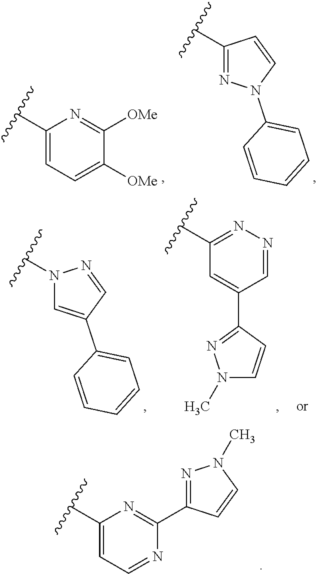

In an aspect, the disclosure provides a compound of Formula II:

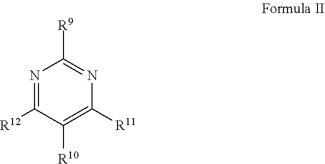

or a pharmaceutically acceptable salt thereof,
wherein
   R⁹ is optionally substituted morpholin-4-yl, optionally substituted morpholin-3-ylalkoxy, optionally substituted 2-(pyridin-2-yl)alkoxy, optionally substituted 1-methylpiperazin-2-yl, or optionally substituted C₆-C₁₀ aryl;
   R¹⁰ is optionally substituted C₁₋₆ alkoxy, —(CO)NR^{7a}R^{7b}, —P(O)R^{7c}R^{7d}, or —S(O)ₖR^{7e}; each of R^{7a} and R^{7b} is, independently, H or optionally substituted C₁-C₆ alkyl; or R^{7a} and R^{7b}, together with the nitrogen atom to which they are attached, combine to form optionally substituted C₂-C₉ heterocyclyl;
   each of R^{7c}, R^{7d}, and R^{7e} is independently optionally substituted C₁₋₆ alkyl, optionally substituted C₁₋₆ alkoxy, or hydroxyl;
   k is 0, 1, or 2;
   R¹¹ is optionally substituted morpholin-4-yl, optionally substituted pyridin-4-yl, optionally substituted pyrazol-4-yl, optionally substituted 1H-imidazol-2-yl, optionally substituted quinoline-4-yl, or optionally substituted C₆-C₁₀ aryl; and
   R¹² is optionally substituted 1-methylpiperazin-2-onyl, optionally substituted 2-(pyridin-2-yl)alkoxy, optionally substituted N-(pyridin-3-ylmethyl)amine, optionally substituted N-(pyridin-4-yl)amine, or optionally substituted C₆-C₁₀ aryl.

In some embodiments, R⁹ is morpholin-4-yl.

In some embodiments, R⁹ is optionally substituted morpholin-3-ylalkoxy. In some embodiments, R⁹ is

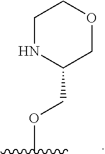 [SMILES: *OC[C@H]1COCCN1]

In some embodiments, R⁹ is optionally substituted 2-(pyridin-2-yl)alkoxy. In some embodiments, R⁹ is

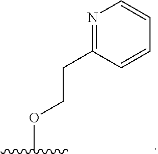 [SMILES: *OCCc1ccccn1]

In some embodiments, R⁹ is optionally substituted 1-methylpiperazin-2-yl. In some embodiments, R⁹ is

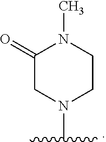 [SMILES: *N1CCN(C)C(=O)C1]

In some embodiments, R⁹ is optionally substituted C₆-C₁₀ aryl. In some embodiments, R⁹ is

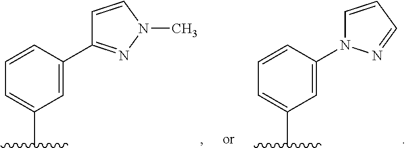 [SMILES: *c1cccc(-c2ccn(C)n2)c1 ; *c1cccc(-n2cccn2)c1]

In some embodiments, R¹⁰ is optionally substituted C₁₋₆ alkoxy. In some embodiments, R¹⁰ is methoxy.

In some embodiments, R¹¹ is optionally substituted morpholin-4-yl. In some embodiments, R¹¹ is morpholin-4-yl. In some embodiments, R¹¹ is optionally substituted pyridin-4-yl. In some embodiments, R¹¹ is pyridin-4-yl.

In some embodiments, R¹¹ is optionally substituted pyrazol-4-yl. In some embodiments, R¹¹ is

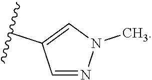 [SMILES: *c1cnn(C)c1]

In some embodiments, R¹¹ is optionally substituted 1H-imidazol-2-yl. In some embodiments, R¹¹ is

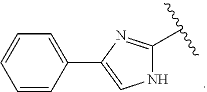 [SMILES: *c1nc(-c2ccccc2)c[nH]1]

In some embodiments, R¹¹ is optionally substituted quinolin-6-yl. In some embodiments, R¹¹ is

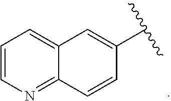 [SMILES: *c1ccc2ncccc2c1]

In some embodiments, R¹¹ is optionally substituted C₆-C₁₀ aryl. In some embodiments, R¹¹ is

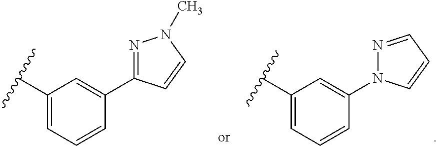 [SMILES: *c1cccc(-c2ccn(C)n2)c1 ; *c1cccc(-n2cccn2)c1]

In some embodiments, R¹² is optionally substituted 1-methylpiperazin-2-only. In some embodiments, R¹² is

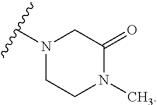 [SMILES: *N1CCN(C)C(=O)C1]

In some embodiments, R¹² is optionally substituted 2-(pyridin-2-yl)alkoxy. In some embodiments, R¹² is

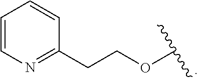 [SMILES: *OCCc1ccccn1]

In some embodiments, R¹² is optionally substituted N-(pyridin-3-ylmethyl)amine. In some embodiments, R¹² is

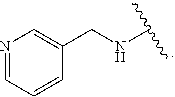 [SMILES: *NCc1cccnc1]

In some embodiments, R¹² is optionally substituted N-(pyridin-4-yl)amine. In some embodiments, R¹² is

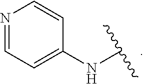 [SMILES: *Nc1ccncc1]

In some embodiments, R¹² is optionally substituted C₆-C₁₀ aryl. In some embodiments, R¹² is

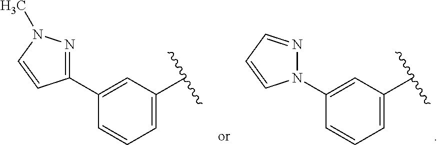 [SMILES: *c1cccc(-c2ccn(C)n2)c1 ; *c1cccc(-n2cccn2)c1]

In some embodiments, the compound has the structure of any one of compounds 1-78 in Table 1, or a pharmaceutically acceptable salt thereof. In some embodiments, the compound has the structure of any one of compounds 1-3, 6-29, 31-36, 38-41, 43-51, 53-57, 59, 62, and 64-78 in Table 1, or a pharmaceutically acceptable salt thereof. In some embodiments, the compound has the structure of any one of compounds 4, 5, 30, 37, 42, 52, 58, 60, 61, and 63 in Table 1, or a pharmaceutically acceptable salt thereof.

In an aspect, the compound has the structure of any one of compounds 1-78 in Table 1, or a pharmaceutically acceptable salt thereof.

In an aspect, the compound has the structure of any one of compounds 1-3, 6-29, 31-36, 38-41, 43-51, 53-57, 59, 62, and 64-78 in Table 1, or a pharmaceutically acceptable salt thereof.

In an aspect, the compound has the structure of any one of compounds 4, 5, 30, 37, 42, 52, 58, 60, 61, and 63 in Table 1, or a pharmaceutically acceptable salt thereof.

In an aspect, the compound has the structure of any one of compounds 66 and 34 in Table 1, or a pharmaceutically acceptable salt thereof.

TABLE 1
Compounds of the Invention

| No. | Structure |
|---|---|
| 1 | 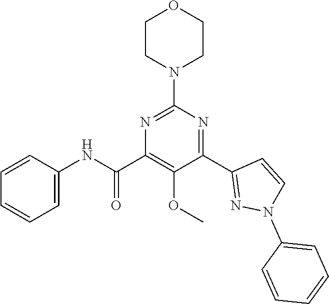 [SMILES: COc1c(C(=O)Nc2ccccc2)nc(N2CCOCC2)nc1-c1ccn(-c2ccccc2)n1] |
| 2 | 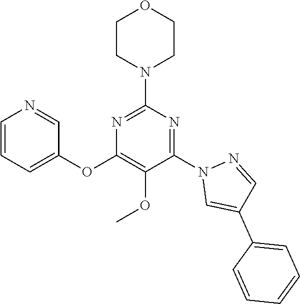 [SMILES: COc1c(Oc2cccnc2)nc(N2CCOCC2)nc1-n1cc(-c2ccccc2)cn1] |
| 3 | 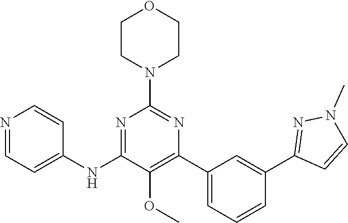 [SMILES: COc1c(Nc2ccncc2)nc(N2CCOCC2)nc1-c1cccc(-c2ccn(C)n2)c1] |
| 4 | 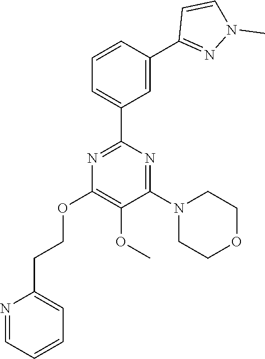 [SMILES: COc1c(OCCc2ccccn2)nc(-c2cccc(-c3ccn(C)n3)c2)nc1N1CCOCC1] |
| 5 | 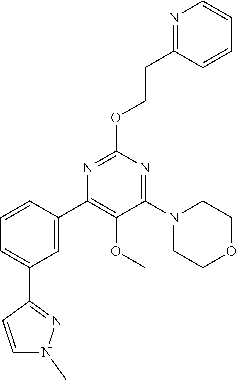 [SMILES: COc1c(-c2cccc(-c3ccn(C)n3)c2)nc(OCCc2ccccn2)nc1N1CCOCC1] |
| 6 | 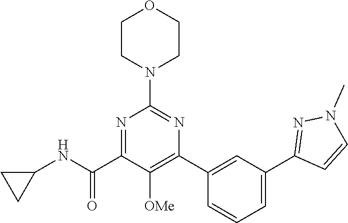 [SMILES: COc1c(C(=O)NC2CC2)nc(N2CCOCC2)nc1-c1cccc(-c2ccn(C)n2)c1] |
| 7 | 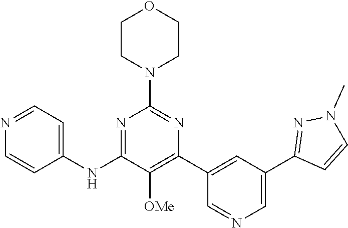 [SMILES: COc1c(Nc2ccncc2)nc(N2CCOCC2)nc1-c1cncc(-c2ccn(C)n2)c1] |
| 8 | 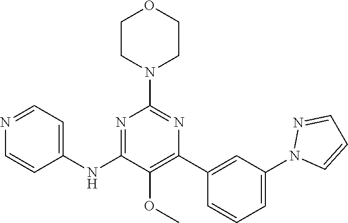 [SMILES: COc1c(Nc2ccncc2)nc(N2CCOCC2)nc1-c1cccc(-n2cccn2)c1] |
| 9 | 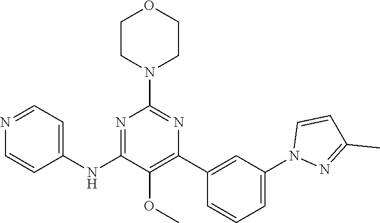 [SMILES: COc1c(Nc2ccncc2)nc(N2CCOCC2)nc1-c1cccc(-n2ccc(C)n2)c1] |
| 10 | 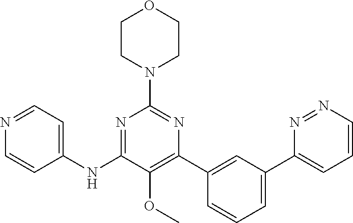 [SMILES: COc1c(Nc2ccncc2)nc(N2CCOCC2)nc1-c1cccc(-c2cccnn2)c1] |
| 11 | 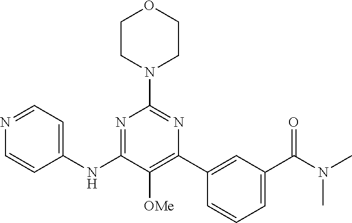 [SMILES: COc1c(Nc2ccncc2)nc(N2CCOCC2)nc1-c1cccc(C(=O)N(C)C)c1] |
| 12 | 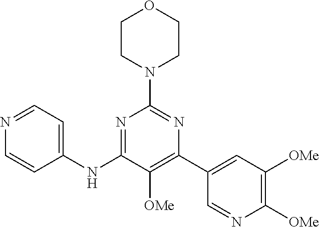 [SMILES: COc1cc(-c2nc(N3CCOCC3)nc(Nc3ccncc3)c2OC)cnc1OC] |
| 13 | 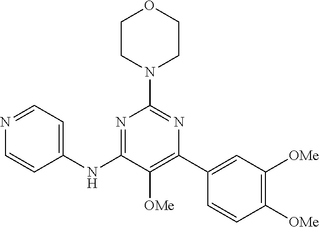 [SMILES: COc1ccc(-c2nc(N3CCOCC3)nc(Nc3ccncc3)c2OC)cc1OC] |
| 14 | 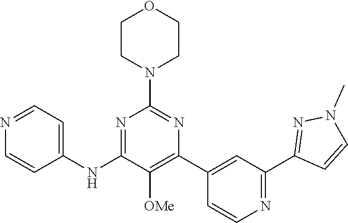 [SMILES: COc1c(Nc2ccncc2)nc(N2CCOCC2)nc1-c1ccnc(-c2ccn(C)n2)c1] |
| 15 | 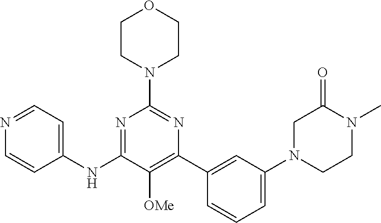 [SMILES: COc1c(Nc2ccncc2)nc(N2CCOCC2)nc1-c1cccc(N2CCN(C)C(=O)C2)c1] |
| 16 | 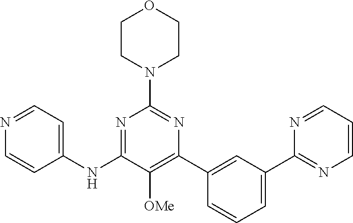 [SMILES: COc1c(Nc2ccncc2)nc(N2CCOCC2)nc1-c1cccc(-c2ncccn2)c1] |
| 17 | 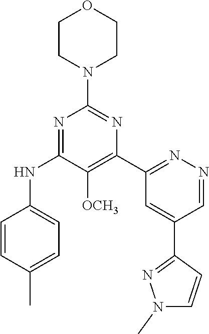 [SMILES: COc1c(Nc2ccc(C)cc2)nc(N2CCOCC2)nc1-c1cc(-c2ccn(C)n2)cnn1] |
| 18 | 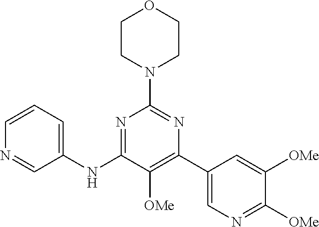 [SMILES: COc1cc(-c2nc(N3CCOCC3)nc(Nc3cccnc3)c2OC)cnc1OC] |
| 19 | 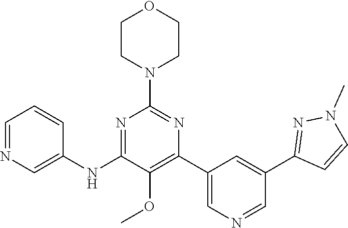 [SMILES: COc1c(Nc2cccnc2)nc(N2CCOCC2)nc1-c1cncc(-c2ccn(C)n2)c1] |
| 20 | 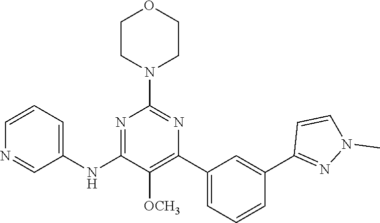 [SMILES: COc1c(Nc2cccnc2)nc(N2CCOCC2)nc1-c1cccc(-c2ccn(C)n2)c1] |
| 21 | 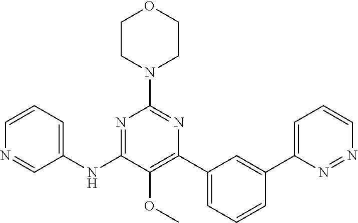 [SMILES: COc1c(Nc2cccnc2)nc(N2CCOCC2)nc1-c1cccc(-c2cccnn2)c1] |
| 22 | 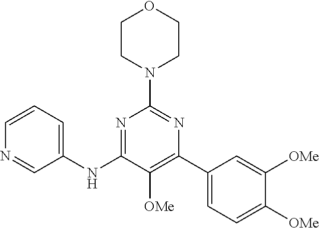 [SMILES: COc1ccc(-c2nc(N3CCOCC3)nc(Nc3cccnc3)c2OC)cc1OC] |
| 23 | 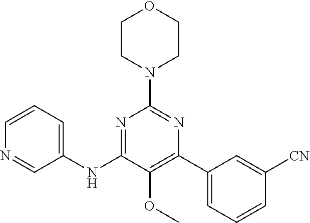 [SMILES: COc1c(Nc2cccnc2)nc(N2CCOCC2)nc1-c1cccc(C#N)c1] |
| 24 | 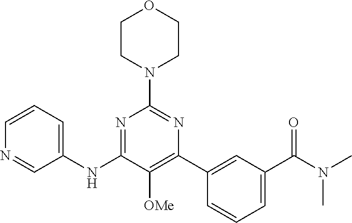 [SMILES: COc1c(Nc2cccnc2)nc(N2CCOCC2)nc1-c1cccc(C(=O)N(C)C)c1] |
| 25 | 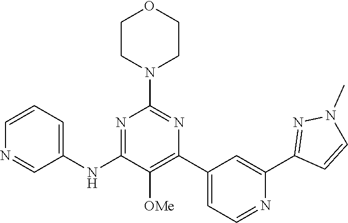 [SMILES: COc1c(Nc2cccnc2)nc(N2CCOCC2)nc1-c1ccnc(-c2ccn(C)n2)c1] |
| 26 | 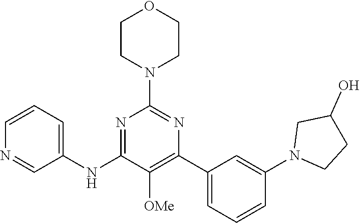 [SMILES: COc1c(Nc2cccnc2)nc(N2CCOCC2)nc1-c1cccc(N2CCC(O)C2)c1] |
| 27 | 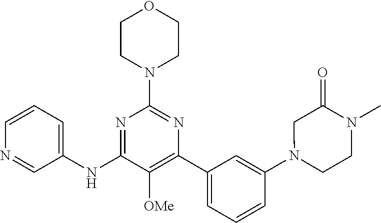 [SMILES: COc1c(Nc2cccnc2)nc(N2CCOCC2)nc1-c1cccc(N2CCN(C)C(=O)C2)c1] |
| 28 | 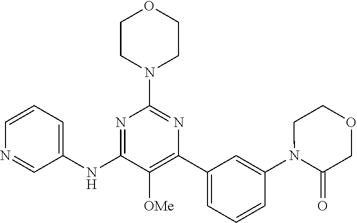 [SMILES: COc1c(Nc2cccnc2)nc(N2CCOCC2)nc1-c1cccc(N2CCOCC2=O)c1] |
| 29 | 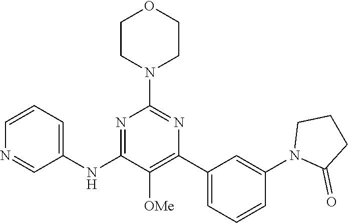 [SMILES: COc1c(Nc2cccnc2)nc(N2CCOCC2)nc1-c1cccc(N2CCCC2=O)c1] |
| 30 | 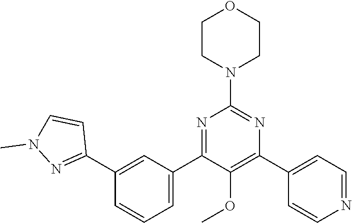 [SMILES: COc1c(-c2ccncc2)nc(N2CCOCC2)nc1-c1cccc(-c2ccn(C)n2)c1] |
| 31 | 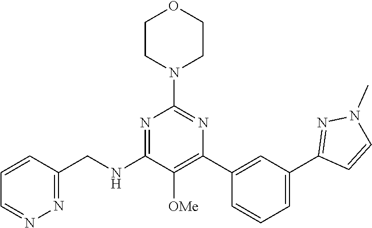 [SMILES: COc1c(NCc2cccnn2)nc(N2CCOCC2)nc1-c1cccc(-c2ccn(C)n2)c1] |
| 32 | 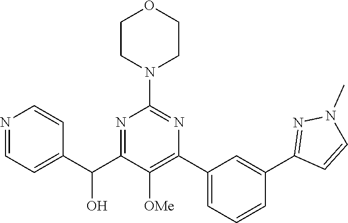 [SMILES: COc1c(-c2cccc(-c3ccn(C)n3)c2)nc(N2CCOCC2)nc1C(O)c1ccncc1] |
| 33 | 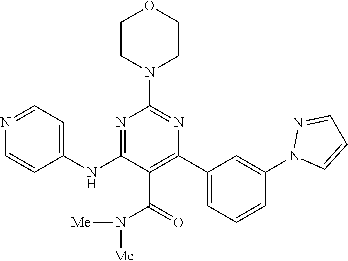 [SMILES: CN(C)C(=O)c1c(Nc2ccncc2)nc(N2CCOCC2)nc1-c1cccc(-n2cccn2)c1] |
| 34 | 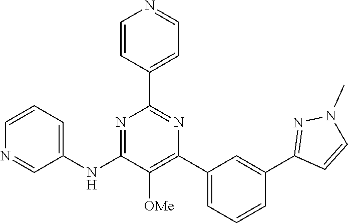 [SMILES: COc1c(Nc2cccnc2)nc(-c2ccncc2)nc1-c1cccc(-c2ccn(C)n2)c1] |
| 35 | 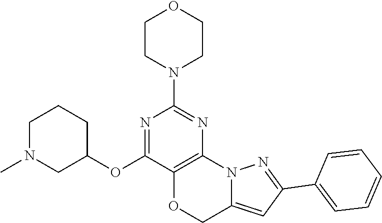 [SMILES: CN1CCCC(Oc2nc(N3CCOCC3)nc3c2OCc2cc(-c4ccccc4)nn2-3)C1] |
| 36 | 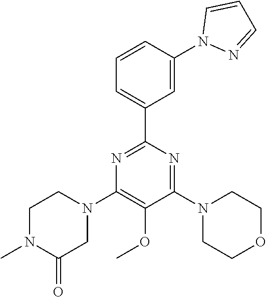 [SMILES: COc1c(N2CCOCC2)nc(-c2cccc(-n3cccn3)c2)nc1N1CCN(C)C(=O)C1] |
| 37 | 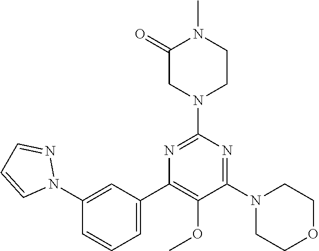 [SMILES: COc1c(-c2cccc(-n3cccn3)c2)nc(N2CCN(C)C(=O)C2)nc1N1CCOCC1] |
| 38 | 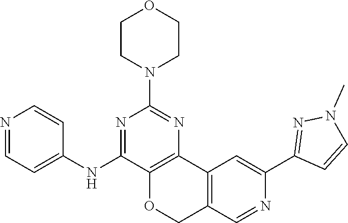 [SMILES: Cn1ccc(-c2cc3c(cn2)COc2c(Nc4ccncc4)nc(N4CCOCC4)nc2-3)n1] |
| 39 | 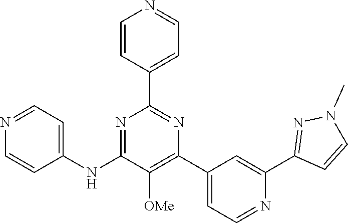 [SMILES: COc1c(Nc2ccncc2)nc(-c2ccncc2)nc1-c1ccnc(-c2ccn(C)n2)c1] |
| 40 | 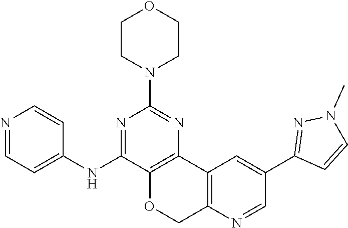 [SMILES: Cn1ccc(-c2cnc3c(c2)-c2nc(N4CCOCC4)nc(Nc4ccncc4)c2OC3)n1] |
| 41 | 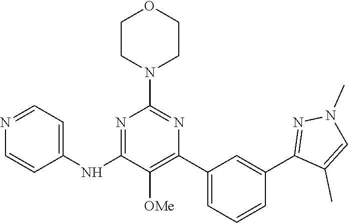 [SMILES: COc1c(Nc2ccncc2)nc(N2CCOCC2)nc1-c1cccc(-c2nn(C)cc2C)c1] |
| 42 | 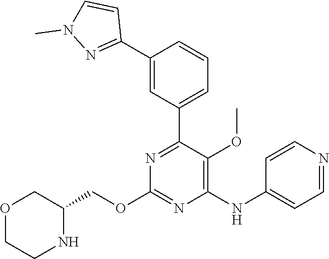 [SMILES: COc1c(Nc2ccncc2)nc(OC[C@H]2COCCN2)nc1-c1cccc(-c2ccn(C)n2)c1] |
| 43 | 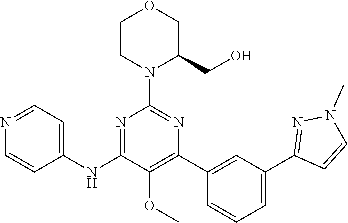 [SMILES: COc1c(Nc2ccncc2)nc(N2CCOC[C@H]2CO)nc1-c1cccc(-c2ccn(C)n2)c1] |
| 44 | 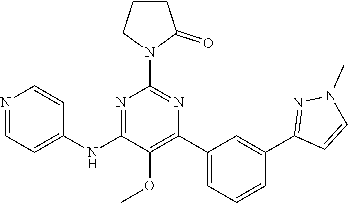 [SMILES: COc1c(Nc2ccncc2)nc(N2CCCC2=O)nc1-c1cccc(-c2ccn(C)n2)c1] |
| 45 | 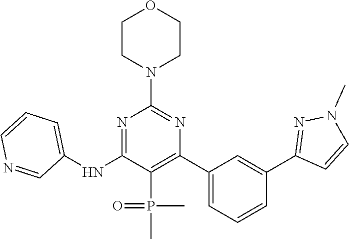 [SMILES: Cn1ccc(-c2cccc(-c3nc(N4CCOCC4)nc(Nc4cccnc4)c3P(C)(C)=O)c2)n1] |
| 46 | 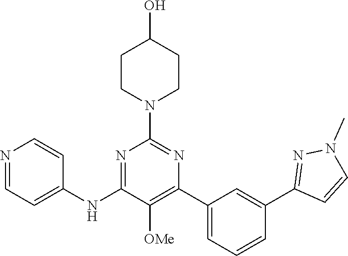 [SMILES: COc1c(Nc2ccncc2)nc(N2CCC(O)CC2)nc1-c1cccc(-c2ccn(C)n2)c1] |
| 47 | 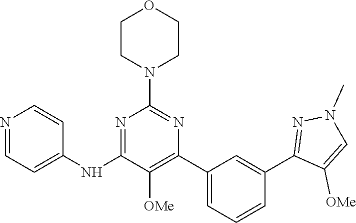 [SMILES: COc1cn(C)nc1-c1cccc(-c2nc(N3CCOCC3)nc(Nc3ccncc3)c2OC)c1] |
| 48 | 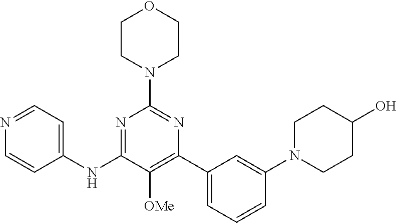 [SMILES: COc1c(Nc2ccncc2)nc(N2CCOCC2)nc1-c1cccc(N2CCC(O)CC2)c1] |
| 49 | 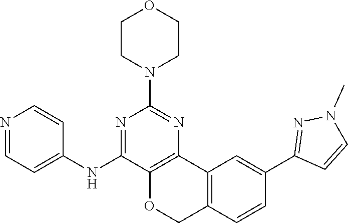 [SMILES: Cn1ccc(-c2ccc3c(c2)-c2nc(N4CCOCC4)nc(Nc4ccncc4)c2OC3)n1] |
| 50 | 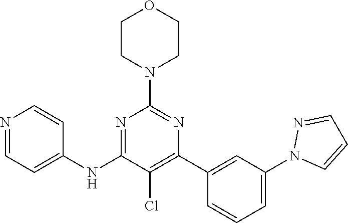 [SMILES: Clc1c(Nc2ccncc2)nc(N2CCOCC2)nc1-c1cccc(-n2cccn2)c1] |
| 51 | 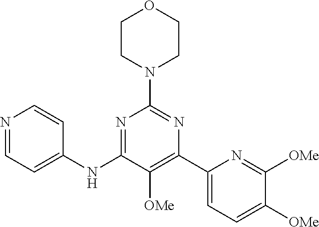 [SMILES: COc1ccc(-c2nc(N3CCOCC3)nc(Nc3ccncc3)c2OC)nc1OC] |
| 52 | 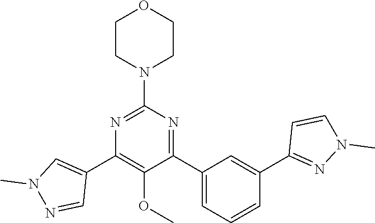 |
| 53 | 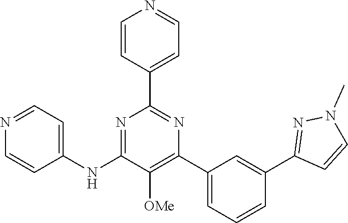 [SMILES: COc1c(Nc2ccncc2)nc(-c2ccncc2)nc1-c1cccc(-c2ccn(C)n2)c1] |
| 54 | 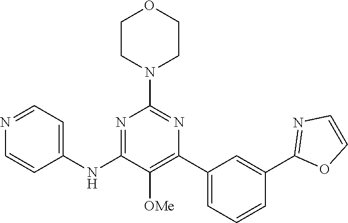 [SMILES: COc1c(Nc2ccncc2)nc(N2CCOCC2)nc1-c1cccc(-c2ncco2)c1] |
| 55 | 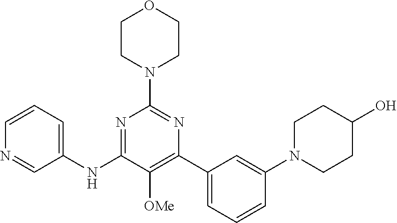 [SMILES: COc1c(Nc2cccnc2)nc(N2CCOCC2)nc1-c1cccc(N2CCC(O)CC2)c1] |
| 56 | 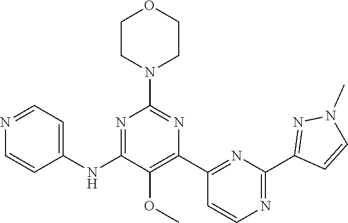 [SMILES: COc1c(Nc2ccncc2)nc(N2CCOCC2)nc1-c1ccnc(-c2ccn(C)n2)n1] |
| 57 | 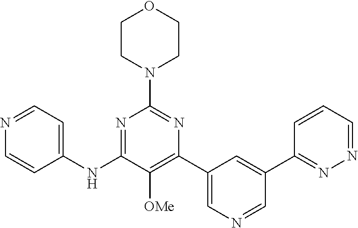 [SMILES: COc1c(Nc2ccncc2)nc(N2CCOCC2)nc1-c1cncc(-c2cccnn2)c1] |
| 58 | 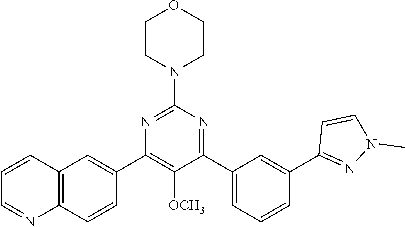 [SMILES: COc1c(-c2cccc(-c3ccn(C)n3)c2)nc(N2CCOCC2)nc1-c1ccc2ncccc2c1] |
| 59 | 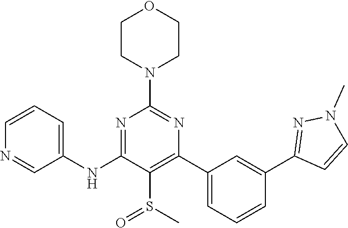 [SMILES: Cn1ccc(-c2cccc(-c3nc(N4CCOCC4)nc(Nc4cccnc4)c3S(C)=O)c2)n1] |
| 60 | 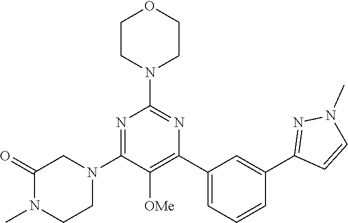 [SMILES: COc1c(-c2cccc(-c3ccn(C)n3)c2)nc(N2CCOCC2)nc1N1CCN(C)C(=O)C1] |
| 61 | 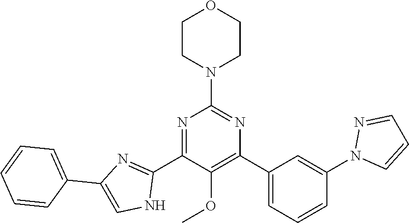 [SMILES: COc1c(-c2cccc(-n3cccn3)c2)nc(N2CCOCC2)nc1-c1nc(-c2ccccc2)c[nH]1] |
| 62 | 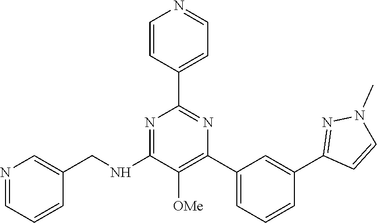 [SMILES: COc1c(NCc2cccnc2)nc(-c2ccncc2)nc1-c1cccc(-c2ccn(C)n2)c1] |
| 63 | 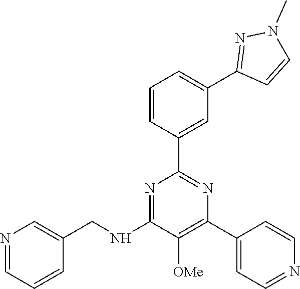 [SMILES: COc1c(NCc2cccnc2)nc(-c2cccc(-c3ccn(C)n3)c2)nc1-c1ccncc1] |
| 64 | 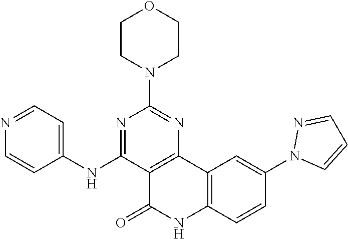 [SMILES: O=c1[nH]c2ccc(-n3cccn3)cc2c2nc(N3CCOCC3)nc(Nc3ccncc3)c12] |
| 65 | 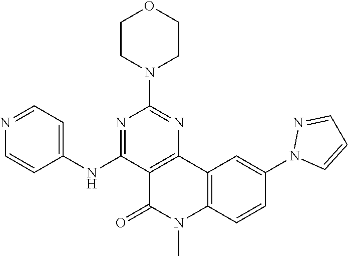 [SMILES: Cn1c(=O)c2c(Nc3ccncc3)nc(N3CCOCC3)nc2c2cc(-n3cccn3)ccc21] |
| 66 | 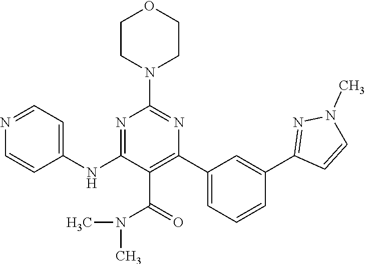 [SMILES: CN(C)C(=O)c1c(Nc2ccncc2)nc(N2CCOCC2)nc1-c1cccc(-c2ccn(C)n2)c1] |
| 67 | 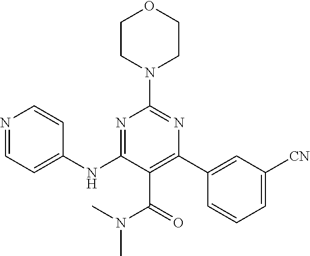 [SMILES: CN(C)C(=O)c1c(Nc2ccncc2)nc(N2CCOCC2)nc1-c1cccc(C#N)c1] |
| 68 | 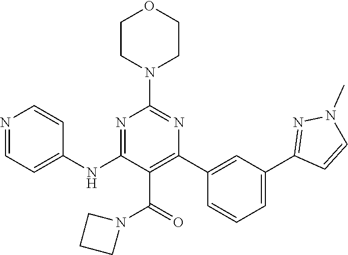 [SMILES: Cn1ccc(-c2cccc(-c3nc(N4CCOCC4)nc(Nc4ccncc4)c3C(=O)N3CCC3)c2)n1] |
| 69 | 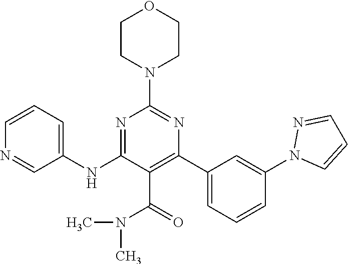 [SMILES: CN(C)C(=O)c1c(Nc2cccnc2)nc(N2CCOCC2)nc1-c1cccc(-n2cccn2)c1] |
| 70 | 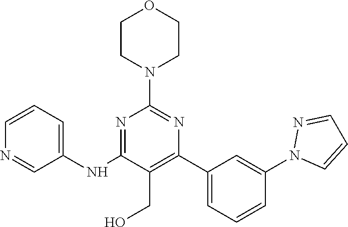 [SMILES: OCc1c(Nc2cccnc2)nc(N2CCOCC2)nc1-c1cccc(-n2cccn2)c1] |
| 71 | 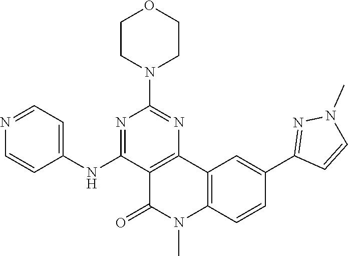 [SMILES: Cn1ccc(-c2ccc3c(c2)c2nc(N4CCOCC4)nc(Nc4ccncc4)c2c(=O)n3C)n1] |
| 72 | 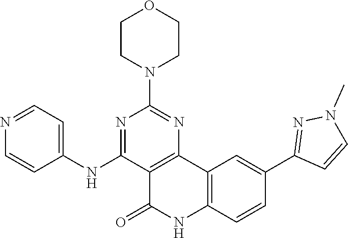 [SMILES: Cn1ccc(-c2ccc3[nH]c(=O)c4c(Nc5ccncc5)nc(N5CCOCC5)nc4c3c2)n1] |
| 73 | 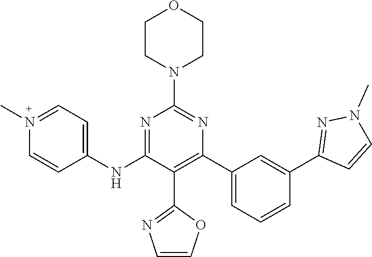 |
| 74 | 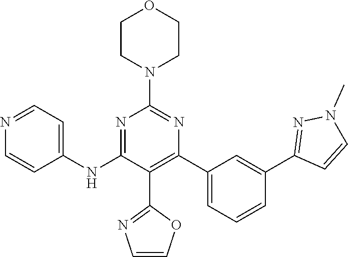 [SMILES: Cn1ccc(-c2cccc(-c3nc(N4CCOCC4)nc(Nc4ccncc4)c3-c3ncco3)c2)n1] |
| 75 | 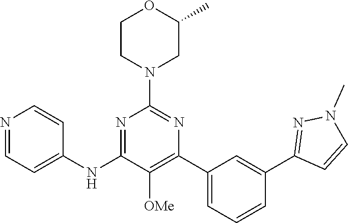 [SMILES: COc1c(Nc2ccncc2)nc(N2CCO[C@H](C)C2)nc1-c1cccc(-c2ccn(C)n2)c1] |
| 76 | 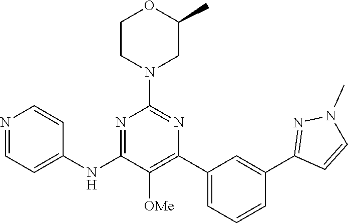 [SMILES: COc1c(Nc2ccncc2)nc(N2CCO[C@@H](C)C2)nc1-c1cccc(-c2ccn(C)n2)c1] |
| 77 | 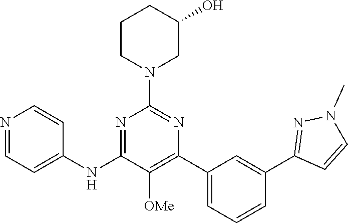 [SMILES: COc1c(Nc2ccncc2)nc(N2CCC[C@H](O)C2)nc1-c1cccc(-c2ccn(C)n2)c1] |
| 78 | 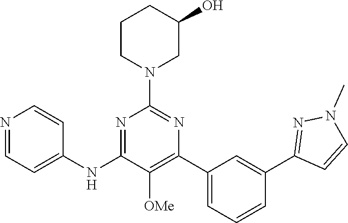 [SMILES: COc1c(Nc2ccncc2)nc(N2CCC[C@@H](O)C2)nc1-c1cccc(-c2ccn(C)n2)c1] |

In an aspect, the invention provides an intermediate in the synthesis of some compounds of the invention. Non-limiting examples of the intermediates include compounds i-1 to i-4 in Table 2.

TABLE 2

| No. | Structure |
|---|---|
| i-1 | 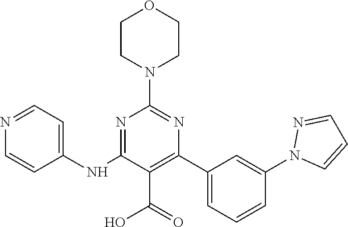 [SMILES: O=C(O)c1c(Nc2ccncc2)nc(N2CCOCC2)nc1-c1cccc(-n2cccn2)c1] |
| i-2 | 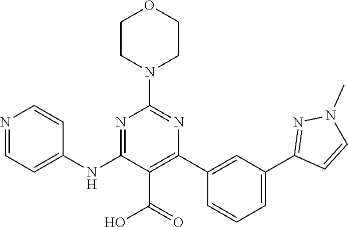 [SMILES: Cn1ccc(-c2cccc(-c3nc(N4CCOCC4)nc(Nc4ccncc4)c3C(=O)O)c2)n1] |
| i-3 | 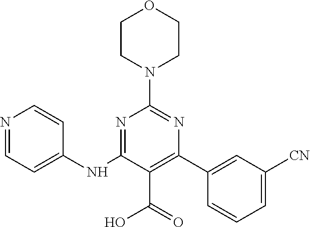 [SMILES: N#Cc1cccc(-c2nc(N3CCOCC3)nc(Nc3ccncc3)c2C(=O)O)c1] |
| i-4 | 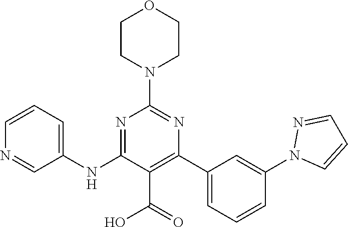 [SMILES: O=C(O)c1c(Nc2cccnc2)nc(N2CCOCC2)nc1-c1cccc(-n2cccn2)c1] |

In an aspect, the invention features a pharmaceutical composition comprising any of the foregoing compounds and a pharmaceutically acceptable excipient.

In an aspect, the invention features a method of treating a neurological disorder (e.g., frontotemporal dementia (FTLD-TDP), chronic traumatic encephalopathy, ALS, Alzheimer's disease, limbic-predominant age-related TDP-43 encephalopathy (LATE), or frontotemporal lobar degeneration) in a subject in need thereof. This method includes administering an effective amount of any of the foregoing compounds or pharmaceutical compositions.

In an aspect, the invention features a method of inhibiting toxicity in a cell (e.g., mammalian neural cell) related to a protein (e.g., TDP-43 or C9orf72). This method includes administering an effective amount of any of the foregoing compounds or pharmaceutical compositions.

In an aspect, the invention features a method of treating a TDP-43-associated disorder or C9orf72-associated disorder (e.g., FTLD-TDP, chronic traumatic encephalopathy, ALS, Alzheimer's disease, LATE, or frontotemporal lobar degeneration) in a subject in need thereof. This method includes administering to the subject an effective amount of a compounds described herein or a pharmaceutical composition containing one or more compounds described herein. In some embodiments, the method includes administering to the subject in need thereof an effective amount of the compound of Formula I or a pharmaceutically acceptable sat thereof (e.g., a compound having the structure of any one of compounds 1-3, 6-29, 31-36, 38-41, 43-51, 53-57, 59, 62, and 64-78 in Table 1 or a pharmaceutically acceptable sat thereof). In some embodiments, the method includes administering to the subject in need thereof an effective amount of the compound of Formula II or a pharmaceutically acceptable sat thereof (e.g., a compound having the structure of any one of compounds 4, 5, 30, 37, 42, 52, 58, 60, 61, and 63 in Table 1 or a pharmaceutically acceptable sat thereof). In some embodiments, the method includes administering to the subject in need thereof an effective amount of the compound having the structure of any one of compounds 1-78 in Table 1 or a pharmaceutically acceptable sat thereof.

In an aspect, the invention features a method of inhibiting PIKfyve. This method includes contacting a cell with an effective amount of any of the foregoing compounds or pharmaceutical compositions. In some embodiments, the method includes administering to the subject in need thereof an effective amount of the compound of Formula I or a pharmaceutically acceptable salt thereof (e.g., a compound having the structure of any one of compounds 1-3, 6-29, 31-36, 38-41, 43-51, 53-57, 59, 62, and 64-78 in Table 1 or a pharmaceutically acceptable salt thereof). In some embodiments, the method includes administering to the subject in need thereof an effective amount of the compound of Formula II or a pharmaceutically acceptable salt thereof (e.g., a compound having the structure of any one of compounds 4, 5, 30, 37, 42, 52, 58, 60, 61, and 63 in Table 1 or a pharmaceutically acceptable salt thereof). In some embodiments, the method includes administering to the subject in need thereof an effective amount of the compound having the structure of any one of compounds 1-78 in Table 1 or a pharmaceutically acceptable salt thereof.

In another aspect, the invention features a method of treating a neurological disorder in a patient, such as a human patient, identified as likely to benefit from treatment with a compound of the invention on the basis of TDP-43 toxicity. In this aspect, the method may include (i) determining that the patient exhibits, or is prone to develop, TDP-43 toxicity, and (ii) providing to the patient a therapeutically effective amount of a compound of the invention. In some embodiments, the patient has previously been determined to exhibit, or to be prone to developing, TDP-43 toxicity, and the method includes providing to the patient a therapeutically effective amount of a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In an additional aspect, the invention features a method of treating a neurological disorder in a patient, such as a human patient, identified as likely to benefit from treatment with a compound of the invention on the basis of TDP-43 expression. In this aspect, the method includes (i) determining that the patient expresses a mutant form of TDP-43 having a mutation associated with TDP-43 aggregation (e.g., a mutation selected from Q331K, M337V, Q343R, N345K, R361S, and N390D), and (ii) providing to the patient a therapeutically effective amount of a compound of the invention. In some embodiments, the patient has previously been determined to express a mutant form of TDP-43 having a mutation associated with TDP-43 aggregation, such as a Q331K, M337V, Q343R, N345K, R361S, or N390D mutation, and the method includes providing to the patient a therapeutically effective amount of a compound of the invention.

In another aspect, the invention features a method of determining whether a patient (e.g., a human patient) having a neurological disorder is likely to benefit from treatment with a compound of the invention by (i) determining whether the patient exhibits, or is prone to develop, TDP-43 aggregation and (ii) identifying the patient as likely to benefit from treatment with a compound of the invention if the patient exhibits, or is prone to develop, TDP-43 aggregation. In some embodiments, the method further includes the step of (iii) informing the patient whether he or she is likely to benefit from treatment with a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In another aspect, the invention features a method of determining whether a patient (e.g., a human patient) having a neurological disorder is likely to benefit from treatment with a compound of the invention by (i) determining whether the patient expresses a TDP-43 mutant having a mutation associated with TDP-43 aggregation (e.g., a mutation selected from Q331K, M337V, Q343R, N345K, R361S, and N390D) and (ii) identifying the patient as likely to benefit from treatment with a compound of the invention if the patient expresses a TDP-43 mutant. In some embodiments, the method further includes the step of (iii) informing the patient whether he or she is likely to benefit from treatment with a compound of the invention. The TDP-43 isoform expressed by the patient may be assessed, for example, by isolated TDP-43 protein from a sample obtained from the patient and sequencing the protein using molecular biology techniques described herein or known in the art. In some embodiments, the TDP-43 isoform expressed by the patient is determined by analyzing the patient's genotype at the TDP-43 locus, for example, by sequencing the TDP-43 gene in a sample obtained from the patient. In some embodiments, the method includes the step of obtaining the sample from the patient.

In some embodiments of any of the above aspects, the compound of the invention is provided to the patient by administration of the compound of the invention to the patient. In some embodiments, the compound of the invention is provided to the patient by administration of a prodrug that is converted in vivo to the compound of the invention.

In some embodiments of any of the above aspects, the neurological disorder is a neuromuscular disorder, such as a neuromuscular disorder selected from amyotrophic lateral sclerosis, congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome. In some embodiments, the neurological disorder is amyotrophic lateral sclerosis.

In some embodiments of any of the above aspects, the neurological disorder is selected from frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy.

In some embodiments, the neurological disorder is amyotrophic lateral sclerosis, and following administration of the compound of the invention to the patient, the patient exhibits one or more, or all, of the following responses:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the compound of the invention (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, weeks, 46 weeks, 47 weeks, 48 weeks, or more, following the initial administration of the compound of the invention to the patient);
(ii) an increase in slow vital capacity, such as an increase in the patient's slow vital capacity within one or more days, weeks, or months following administration of the compound of the invention (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iii) a reduction in decremental responses exhibited by the patient upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(vi) a decrease in the frequency and/or severity of muscle cramps, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient); and/or
(vii) a decrease in TDP-43 aggregation, such as a decrease in TDP-43 aggregation within one or more days, weeks, or months following administration of the compound of the invention (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient.

### Chemical Terms

it is to be understood that the terminology employed herein is for the purpose of describing particular embodiments and is not intended to be limiting.

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion, e.g., the interconversion illustrated in the scheme below:

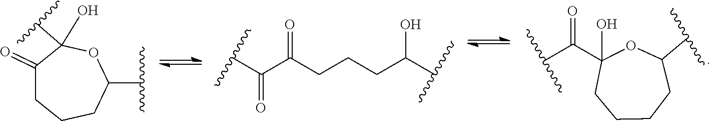 [SMILES: *C1CCCC(=O)C(*)(O)O1 ; *C(=O)C(=O)CCCC(*)O ; *C(=O)C1(O)CCCCC(*)O1]

Those skilled in the art will appreciate that, in some embodiments, isotopes of compounds described herein may be prepared and/or utilized in accordance with the present invention. “Isotopes” refers to atoms having the same atomic number but different mass numbers resulting from a different number of neutrons in the nuclei. For example, isotopes of hydrogen include tritium and deuterium. In some embodiments, an isotopic substitution (e.g., substitution of hydrogen with deuterium) may alter the physiciochemical properties of the molecules, such as metabolism and/or the rate of racemization of a chiral center.

As is known in the art, many chemical entities (in particular, many organic molecules and/or many small molecules) can adopt a variety of different solid forms such as, for example, amorphous forms and/or crystalline forms (e.g., polymorphs, hydrates, solvates, etc). In some embodiments, such entities may be utilized in any form, including in any solid form. In some embodiments, such entities are utilized in a particular form, for example in a particular solid form.

In some embodiments, compounds described and/or depicted herein may be provided and/or utilized in salt form.

In certain embodiments, compounds described and/or depicted herein may be provided and/or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional.

The term “acyl,” as used herein, represents a hydrogen or an alkyl group, as defined herein that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆₋₁₀ aryl, C₁-C₁₀ alkyl C₆₋₁₀ aryl, or C₁-C₂₀ alkyl C₆₋₁₀ aryl), such as, benzyl and phenethyl. In some embodiments, the akyl and the aryl each can be further substituted with 1, 2, 3, or 4 substituent groups as defined herein for the respective groups.

The term “azido,” as used herein, represents a —N₃ group.

The term “cyano,” as used herein, represents a CN group.

The terms “carbocyclyl,” as used herein, refer to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroakynylene is a divalent heteroalkynyl group.

The term “heteroaryl,” as used herein, refers to a mono- or polycyclic radical of 5 to 12 atoms having at least one aromatic ring and containing one, two, three, or four ring heteroatoms selected from N, O, and S, with the remaining ring atoms being C. One or two ring carbon atoms of the heteroaryl group may be replaced with a carbonyl group. Examples of heteroaryl groups are pyridyl (e.g., pyridin-2-yl, pyridin-3-yl, and pyridin-4-yl), pyrazolyl (e.g., pyrazol-1-yl and pyrazol-3-yl), pyrimidinyl (e.g., pyrimidin-4-yl), pyridazinyl (e.g., pyridazin-3-yl), benzooxazolyl, benzoimidazolyl, benzothiazolyl, imidazolyl, oxazolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl. In some embodiments, the akyl and the heteroaryl each can be further substituted with 1, 2, 3, or 4 substituent groups as defined herein for the respective groups.

The term “heterocyclyl,” as used herein, denotes a mono- or polycyclic radical having 3 to 12 atoms having at least one ring containing one, two, three, or four ring heteroatoms selected from N, O or S and no aromatic ring. Examples of heterocyclyl groups include, but are not limited to, morpholinyl (e.g., morpholin-4-yl), thiomorpholinyl, furyl, piperazinyl, piperidinyl (e.g., piperidin-1-yl), pyranyl, pyrrolidinyl (e.g., pyrrolidin-1-yl), tetrahydropyranyl, tetrahydrofuranyl, and 1,3-dioxanyl. A heterocyclyl group may be aromatic or non-aromatic. An aromatic heterocyclyl is also referred to as heteroaryl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the akyl and the heterocyclyl each can be further substituted with 1, 2, 3, or 4 substituent groups as defined herein for the respective groups.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999). N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an NO₂ group.

The term “oxyheteroaryl,” as used herein, represents a heteroaryl group having at least one endocyclic oxygen atom.

The term “oxyheterocyclyl,” as used herein, represents a heterocyclyl group having at least one endocyclic oxygen atom.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), halo (e.g., fluoro), hydroxyl, oxo, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers.

When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer.

Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

As used herein, the term “administration” refers to the administration of a composition (e.g., a compound, a complex or a preparation that includes a compound or complex as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

As used herein, the terms “approximately” and “about” are each intended to encompass normal statistical variation as would be understood by those of ordinary skill in the art as appropriate to the relevant context. In certain embodiments, the terms “approximately” or “about” each refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population).

As used herein, the terms “benefit” and “response” are used interchangeably in the context of a subject, such as a human subject undergoing therapy for the treatment of a neurological disorder, for example, amyotrophic lateral sclerosis, frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy. The terms “benefit” and “response” refer to any clinical improvement in the subject's condition.

Exemplary benefits in the context of a subject undergoing treatment for a neurological disorder using the compositions and methods described herein (e.g., in the context of a human subject undergoing treatment for a neurological disorder described herein, such as amyotrophic lateral sclerosis, with a FYVE-type zinc finger containing phosphoinositide kinase (PIKfyve) inhibitor described herein, such as an inhibitory small molecule, antibody, antigen-binding fragment thereof, or interfering RNA molecule) include the slowing and halting of disease progression, as well as suppression of one or more symptoms associated with the disease. Particularly, in the context of a patient (e.g., a human patient) undergoing treatment for amyotrophic lateral sclerosis with a compound of the invention, examples of clinical “benefits” and “responses” are (i) an improvement in the subject's condition as assessed using the amyotrophic lateral sclerosis functional rating scale (ALSFRS) or the revised ALSFRS (ALSFRS-R) following administration of the compound of the invention, such as an improvement in the subject's ALSFRS or ALSFRS-R score within one or more days, weeks, or months following administration of the compound of the invention (e.g., an improvement in the subject's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, weeks, 46 weeks, 47 weeks, 48 weeks, or more, following the initial administration of the compound of the invention to the subject); (ii) an increase in the subject's slow vital capacity following administration of the compound of the invention, such as an increase in the subject's slow vital capacity within one or more days, weeks, or months following administration of the compound of the invention (e.g., an increase in the subject's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iii) a reduction in decremental responses exhibited by the subject upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iv) an improvement in the subject's muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, weeks, 46 weeks, 47 weeks, 48 weeks, or more, following the initial administration of the compound of the invention to the subject); (v) an improvement in the subject's quality of life, as assessed, for example, using the amyotrophic lateral sclerosis-specific quality of life (ALS-specific QOL) questionnaire, such as an improvement in the subject's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); and (vi) a decrease in the frequency and/or severity of muscle cramps exhibited by the subject, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject).

As used herein, the term “dosage form” refers to a physically discrete unit of an active compound (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

In the practice of the methods of the present invention, an “effective amount” of any one of the compounds of the invention or a combination of any of the compounds of the invention or a pharmaceutically acceptable salt thereof, is administered via any of the usual and acceptable methods known in the art, either singly or in combination.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of formula (I). For example pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 88:1-19, 1977 and in Pharmaceutical Salts: Properties, Selection, and Use, (Eds. P. H. Stahl and C. G. Wermuth), Wley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

The terms “PIKfyve” and “FYVE-type zinc finger containing phosphoinositide kinase” are used interchangeably herein and refer to the enzyme that catalyzes phosphorylation of phosphatidylinositol 3-phosphate to produce phosphatidylinositol 3,5-bisphosphate, for example, in human subjects. The terms “PIKfyve” and “FYVE-type zinc finger containing phosphoinositide kinase” refer not only to wild-type forms of PIKfyve, but also to variants of wild-type PIKfyve proteins and nucleic acids encoding the same. The gene encoding PIKfyve can be accessed under NCBI Reference Sequence No. NG_021188.1.

Exemplary transcript sequences of wild-type form of human PIKfyve can be accessed under NCBI Reference Sequence Nos. NM_015040.4, NM_152671.3, and NM_001178000.1. Exemplary protein sequences of wild-type form of human PIKfyve can be accessed under NCBI Reference Sequence Nos. NP_055855.2, NP_889884.1, and NP_001171471.1.

As used herein, the term “PIKfyve inhibitor” refers to substances, such as compounds of Formula I. Inhibitors of this type may, for example, competitively inhibit PIKfyve activity by specifically binding the PIKfyve enzyme (e.g., by virtue of the affinity of the inhibitor for the PIKfyve active site), thereby precluding, hindering, or halting the entry of one or more endogenous substrates of PIKfyve into the enzyme's active site. Additional examples of PIKfyve inhibitors that suppress the activity of the PIKfyve enzyme include substances that may bind PIKfyve at a site distal from the active site and attenuate the binding of endogenous substrates to the PIKfyve active site by way of a change in the enzyme's spatial conformation upon binding of the inhibitor. In addition to encompassing substances that modulate PIKfyve activity, the term “PIKfyve inhibitor” refers to substances that reduce the concentration and/or stability of PIKfyve mRNA transcripts in vivo, as well as those that suppress the translation of functional PIKfyve enzyme.

The term “pure” means substantially pure or free of unwanted components (e.g., other compounds and/or other components of a cell lysate), material defilement, admixture or imperfection.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

A variety of clinical indicators can be used to identify a patient as “at risk” of developing a particular neurological disease. Examples of patients (e.g., human patients) that are “at risk” of developing a neurological disease, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include (i) subjects exhibiting or prone to exhibit aggregation of TAR-DNA binding protein (TDP)-43, and (ii) subjects expressing a mutant form of TDP-43 containing a mutation associated with TDP-43 aggregation and toxicity, such as a mutation selected from Q331K, M337V, Q343R, N345K, R361S, and N390D. Subjects that are “at risk” of developing amyotrophic lateral sclerosis may exhibit one or both of these characteristics, for example, prior to the first administration of a PIKfyve inhibitor in accordance with the compositions and methods described herein.

As used herein, the terms “TAR-DNA binding protein-43” and “TDP-43” are used interchangeably and refer to the transcription repressor protein involved in modulating HIV-1 transcription and alternative splicing of the cystic fibrosis transmembrane conductance regulator (CFTR) pre-mRNA transcript, for example, in human subjects. The terms “TAR-DNA binding protein-43” and “TDP-43” refer not only to wild-type forms of TDP-43, but also to variants of wild-type TDP-43 proteins and nucleic acids encoding the same. The amino acid sequence and corresponding mRNA sequence of a wild-type form of human TDP-43 are provided under NCBI Reference Sequence Nos. NM_007375.3 and NP_031401.1, respectively.

The terms “TAR-DNA binding protein-43” and “TDP-43” as used herein include, for example, forms of the human TDP-43 protein that have an amino acid sequence that is at least 85% identical to the amino acid sequence of NCBI Reference Sequence No. NP_031401.1 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NP_031401.1) and/or forms of the human TDP-43 protein that contain one or more substitutions, insertions, and/or deletions (e.g., one or more conservative and/or nonconservative amino acid substitutions, such as up to 5, 10, 15, 20, 25, or more, conservative or nonconservative amino acid substitutions) relative to a wild-type TDP-43 protein. For instance, patients that may be treated for a neurological disorder as described herein, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include human patients that express a form of TDP-43 having a mutation associated with elevated TDP-43 aggregation and toxicity, such as a mutation selected from Q331K, M337V, Q343R, N345K, R361S, and N390D. Similarly, the terms “TAR-DNA binding protein-43” and “TDP-43” as used herein include, for example, forms of the human TDP-43 gene that encode an mRNA transcript having a nucleic acid sequence that is at least 85% identical to the nucleic acid sequence of NCBI Reference Sequence No. NM_007375.3 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NM_007375.3).

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

A “therapeutic regimen” refers to a dosing regimen whose administration across a relevant population is correlated with a desired or beneficial therapeutic outcome.

The term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### BRIEF DESCRIPTION OF THE DRAWINGS

FIG. 1 is a scheme showing an approach to generation of a control TDP-43 yeast model (FAB1 TDP-43). A control yeast TDP-43 model was generated by integrating the human TDP-43 gene and the GAL1 promoter into the yeast genome. The yeast ortholog of human PIKFYVE is FAB1.

FIG. 2 is a scheme showing an approach to generation of a humanized PIKFYVE TDP-43 yeast model (PIKFYVE TDP-43). FAB1 gene through homologous recombination with a G418 resistance cassette (fab1::G418^{R}) (FIG. 2). PIKFYVE was cloned downstream of the GPD promoter harbored on a URA3-containing plasmid and introduced into the fab1::G418R ura3 strain. The pGAL1-TDP-43 construct was then introduced into the “humanized” yeast strain and assessed for cytotoxicity.

FIG. 3 is a histogram generated from the flow cytometry-based viability assay of FAB1 TDP-43.

FIG. 4 is a histogram generated from the flow cytometry-based viability assay of PIKFYVE TDP-43. Upon induction of TDP-43, there was a marked increase in inviable cells (rightmost population), with a more pronounced effect in PIKFYVE TDP-43 than in FAB1 TDP-43 strain (see FIG. 3).

FIG. 5 is an overlay of histograms generated from the flow cytometry-based viability assay of FAB1 TDP-43 in the presence of APY0201.

FIG. 6 is an overlay of histograms generated from the flow cytometry-based viability assay of PIKFYVE TDP-43 in the presence of APY0201.

FIG. 7 is a scatter plot comparing cytoprotection efficacy in PIKFYVE TDP-43 to PIKfyve inhibitory activity of test compounds.

### DETAILED DESCRIPTION

The present invention features compositions and methods for treating neurological disorders, such as amyotrophic lateral sclerosis and other neuromuscular disorders, as well as frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy among others. Particularly, the invention provides inhibitors of FYVE-type zinc finger containing phosphoinositide kinase (PIKfyve), that may be administered to a patient (e.g., a human patient) so as to treat or prevent a neurological disorder, such as one or more of the foregoing conditions. In the context of therapeutic treatment, the PIKfyve inhibitor may be administered to the patient to alleviate one or more symptoms of the disorder and/or to remedy an underlying molecular pathology associated with the disease, such as to suppress or prevent aggregation of TAR-DNA binding protein (TDP)-43.

The disclosure herein is based, in part, on the discovery that PIKfyve inhibition modulates TDP-43 aggregation in cells. Suppression of TDP-43 aggregation exerts beneficial effects in patients suffering from a neurological disorder. Many pathological conditions have been correlated with TDP-43-promoted aggregation and toxicity, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy. Without being limited by mechanism, by administering an inhibitor of PIKfyve, patients suffering from diseases associated with TDP-43 aggregation and toxicity may be treated, for example, due to the suppression of TDP-43 aggregation induced by the PIKfyve inhibitor.

Patients that are likely to respond to PIKfyve inhibition as described herein include those that have or are at risk of developing TDP-43 aggregation, such as those that express a mutant form of TDP-43 associated with TDP-43 aggregation and toxicity in vivo. Examples of such mutations in TDP-43 that have been correlated with elevated TDP-43 aggregation and toxicity include Q331K, M337V, Q343R, N345K, R361S, and N390D, among others. The compositions and methods described herein thus provide the additional clinical benefit of enabling the identification of patients that are likely to respond to PIKfyve inhibitor therapy, as well as processes for treating these patients accordingly.

The sections that follow provide a description of exemplary PIKfyve inhibitors that may be used in conjunction with the compositions and methods disclosed herein. The sections below additionally provide a description of various exemplary mutes of administration and pharmaceutical compositions that may be used for delivery of these substances for the treatment of a neurological disorder.

### PIKfyve Inhibitors

Exemplary PIKfyve inhibitors described herein include compounds of Formula I:

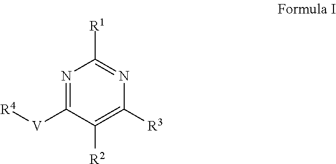

or a pharmaceutically acceptable salt thereof,
wherein
   V is —NH—, —NR⁵—, —CH₂NH—, —CH₂NR⁵—, —O—, —CO—, or —CHOH—;
   R¹ is optionally substituted morpholin-4-yl, pyridin-4-yl, pyridin-3-yl, optionally substituted 2-oxo-pyrrolidin-1-yl, optionally substituted piperidin-1-yl, or optionally substituted pyridiazin-4-yl;
   R² is halogen, —(CH₂)ₙOH, optionally substituted C₁₋₆ alkoxy, optionally substituted C₂-C₉ heteroaryl, optionally substituted 2-oxo-pyrrolidin-1-yl, —(CO)NR^{7a}R^{7b}, —P(O)R^{7c}R^{7d}, or —S(O)ₖR^{7e}; and R³ is optionally substituted pyridin-2-yl, optionally substituted pyridin-3-yl, optionally substituted pyridin-4-yl, optionally substituted pyrazol-1-yl, optionally substituted pyrazol-3-yl, optionally substituted pyridazin-3-yl, optionally substituted pyrimidin-4-yl, or optionally substituted C₆-C₁₀ aryl; or R² and R³, together with the ring to which they are attached, combine to form optionally substituted C₄-C₁₂ heteroaryl;
   n is 1, 2, 3, 4, 5, or 6;
   k is 0, 1, or 2;
   R⁴ is optionally substituted pyridin-4-yl, optionally substituted 1-methylpyridin-1-ium-4-yl, optionally substituted pyridin-3-yl, optionally substituted 1-methylpiperidin-3-yl, optionally substituted pyridazine-3-yl, or —NHR⁸;
   R⁵ is optionally substituted C₁-C₆ alkyl;
   R⁶ is H or optionally substituted C₁-C₆ alkyl;
   each of R^{7a} and R^{7b} is, independently, H or optionally substituted C₁-C₆ alkyl; or R^{7a} and R^{7b}, together with the nitrogen atom to which they are attached, combine to form optionally substituted C₂-C₉ heterocyclyl;
   each of R^{7c}, R^{7d}, and R^{7e} is independently optionally substituted C₁₋₆ alkyl, optionally substituted C₁₋₆ alkoxy, or hydroxyl; and
   R⁸ is optionally substituted phenyl or optionally substituted C₁-C₆ cycloalkyl.

Exemplary PIKfyve inhibitors described herein also include compounds of Formula II:

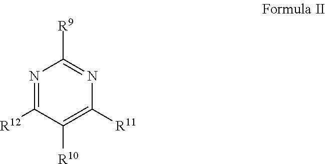

or a pharmaceutically acceptable salt thereof,
wherein
   R⁹ is optionally substituted morpholin-4-yl, optionally substituted morpholin-3-ylalkoxy, optionally substituted 2-(pyridin-2-yl)alkoxy, optionally substituted 1-methylpiperazin-2-yl, or optionally substituted C₆-C₁₀ aryl;
   R¹⁰ is optionally substituted C₁₋₆ alkoxy, —(CO)NR^{7a}R^{7b}, —P(O)R^{7c}R^{7d}, or —S(O)ₖR^{7e};
   each of R^{7a} and R^{7b} is, independently, H or optionally substituted C₁-C₆ alkyl; or R^{7a} and R^{7b}, together with the nitrogen atom to which they are attached, combine to form optionally substituted C₂-C₉ heterocyclyl;
   each of R^{7c}, R^{7d}, and R^{7e} is independently optionally substituted C₁₋₆ alkyl, optionally substituted C₁₋₆ alkoxy, or hydroxyl;
   k is 0, 1, or 2;
   R¹¹ is optionally substituted morpholin-4-yl, optionally substituted pyridin-4-yl, optionally substituted pyrazol-4-yl, optionally substituted 1H-imidazol-2-yl, optionally substituted quinoline-6-yl, or optionally substituted C₆-C₁₀ aryl; and
   R¹² is optionally substituted 1-methylpiperazin-2-onyl, optionally substituted 2-(pyridin-2-yl)alkoxy, optionally substituted N-(pyridin-3-ylmethyl)amine, optionally substituted N-(pyridin-4-yl)amine, or optionally substituted C₆-C₁₀ aryl.

### Methods of Treatment

Suppression of PIKfyve Activity and TDP-43 Aggregation to Treat Neurological Disorders Using the compositions and methods described herein, a patient suffering from a neurological disorder may be administered a PIKfyve inhibitor, such as a small molecule described herein, so as to treat the disorder and/or to suppress one or more symptoms associated with the disorder. Exemplary neurological disorders that may be treated using the compositions and methods described herein are, without limitation, amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy, as well as neuromuscular diseases such as congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome.

The present disclosure is based, in part, on the discovery that PIKfyve inhibitors, such as the agents described herein, are capable of attenuating TDP-43 toxicity. TDP-43-promoted toxicity has been associated with various neurological diseases. The discovery that PIKfyve inhibitors modulate TDP-43 aggregation provides an important therapeutic benefit. Using a PIKfyve inhibitor, such as a PIKfyve inhibitor described herein, a patient suffering from a neurological disorder or at risk of developing such a condition may be treated in a manner that remedies an underlying molecular etiology of the disease. Without being limited by mechanism, the compositions and methods described herein can be used to treat or prevent such neurological conditions, for example, by suppressing the TDP-43 aggregation that promotes pathology.

Additionally, the compositions and methods described herein provide the beneficial feature of enabling the identification and treatment of patients that are likely to respond to PIKfyve inhibitor therapy. For example, in some embodiments, a patient (e.g., a human patient suffering from or at risk of developing a neurological disease described herein, such as amyotrophic lateral sclerosis) is administered a PIKfyve inhibitor if the patient is identified as likely to respond to this form of treatment. Patients may be identified as such on the basis, for example, of susceptibility to TDP-43 aggregation. In some embodiments, the patient is identified is likely to respond to PIKfyve inhibitor treatment based on the isoform of TDP-43 expressed by the patient. For example, patients expressing TDP-43 isoforms having a mutation selected from Q331K, M337V, Q343R, N345K, R361S, and N390D, among others, are more likely to develop TDP-43-promoted aggregation and toxicity relative to patients that do not express such isoforms of TDP-43. Using the compositions and methods described herein, a patient may be identified as likely to respond to PIKfyve inhibitor therapy on the basis of expressing such an isoform of TDP-43, and may subsequently be administered a PIKfyve inhibitor so as to treat or prevent one or more neurological disorders, such as one or more of the neurological disorders described herein.

### Assessing Patient Response

A variety of methods known in the art and described herein can be used to determine whether a patient having a neurological disorder (e.g., a patient at risk of developing TDP-43 aggregation, such as a patient expressing a mutant form of TDP-43 having a mutation associated with elevated TDP-43 aggregation and toxicity, for example, a mutation selected from Q331K, M337V, Q343R, N345K, R361S, and N390D) is responding favorably to PIKfyve inhibition. For example, successful treatment of a patient having a neurological disease, such as amyotrophic lateral sclerosis, with a PIKfyve inhibitor described herein may be signaled by:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the PIKfyve inhibitor (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(ii) an increase in slow vital capacity, such as an increase in the patient's slow vital capacity within one or more days, weeks, or months following administration of the PIKfyve inhibitor (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iii) a reduction in decremental responses exhibited by the patient upon repetitive nerve stimulation, such as a reduction that is observed within one or more days, weeks, or months following administration of the PIKfyve inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the PIKfyve inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the PIKfyve inhibitor (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(vi) a decrease in the frequency and/or severity of muscle cramps, such as a decrease in cramp frequency and/or severity within one or more days, weeks, or months following administration of the PIKfyve inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient); and/or
(vii) a decrease in TDP-43 aggregation, such as a decrease in TDP-43 aggregation within one or more days, weeks, or months following administration of the PIKfyve inhibitor (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, weeks, 46 weeks, 47 weeks, 48 weeks, or more, following the initial administration of the PIKfyve inhibitor to the patient.

### Combination Formulations and Uses Thereof

The compounds of the invention can be combined with one or more therapeutic agents. In particular, the therapeutic agent can be one that treats or prophylactically treats any neurological disorder described herein.

### Combination Therapies

A compound of the invention can be used alone or in combination with other agents that treat neurological disorders or symptoms associated therewith, or in combination with other types of treatment to treat, prevent, and/or reduce the risk of any neurological disorders. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

### Pharmaceutical Compositions

The compounds of the invention are preferably formulated into pharmaceutical compositions for administration to human subjects in a biologically compatible form suitable for administration in vivo. Accordingly, in another aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers.

A compound of the invention may also be administered parenterally. Solutions of a compound of the invention can be prepared in water suitably mixed with a surfactant. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20^{th} ed.) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg.

Alternatively, the dosage amount can be calculated using the body weight of the patient. For example, the dose of a compound, or pharmaceutical composition thereof, administered to a patient may range from 0.1-50 mg/kg.

The following examples are meant to illustrate the invention. They are not meant to limit the invention in anyway.

Abbreviations

| | |
|---|---|
| BINAP | (2,2′-bis(diphenylphosphino)-1,1′-binaphthyl) |
| CO | Carbon monoxide |
| DCM | Dichloromethane |
| DIPEA | Diisopropyl ethylamine |
| DMAC | N,N-Dimethylacetamide |
| DMF | N,N-Dimethylformamide |
| DMSO | Dimethylsulfoxide |
| DPPF | 1,1′-Bis(diphenylphosphino)ferrocene |
| EDCI | 1-Ethyl-3-(3-dimethylaminopropyl)carbodiimide |
| HATU | (1-[Bis(dimethylamino)methylene]-1H-1,2,3-triazolo[4,5-b]pyridinium 3-oxide |
| | hexafluorophosphate |
| HOBt | Hydroxybenzotriazole |
| LAH | Lithium aluminum hydride |
| LDA | Lithium diisopropylamide |
| mW | Microwave |
| NCS | N-chlorosuccinimide |
| NIS | N-iodosuccinimide |
| NMP | N-Methyl-2-pyrrolidone |
| NMR | s-singlet, d-doublet, t-triplet, dd-doublet of doublet, td-triplet of doublet, bs- |
| | broad singlet, m-multiplet, q-quartet. |
| PCy3 | Tricyclohexylphosphine |
| Pd(dppf)Cl₂ | [1,1′-Bis(diphenylphosphino)ferrocene]dichloropalladium(II) |
| Pd(dppf)Cl₂:DCM | [1,1′-Bis(diphenylphosphino) ferrocene]dichloropalladium(II), complex with |
| | dichloromethane |
| Pd(Pt-bu3)₂ | Bis(tri-tert-butylphosphine)palladium(0) |
| Pd₂dba₃ | Tris(dibenzylideneacetone)dipalladium(0) |
| Pin₂B₂/B₂Pin₂ | Bis (pinacolato)diboron |
| prep-HPLC | preparative-scale high performance liquid chromatography |
| prep-TLC | Preparative thin layer chromatography |
| RT | Room temperature |
| Abbreviations | |
| Ru-Phos | 2-Dicyclohexylphosphino-2′,6′-diisopropoxybiphenyl |
| TEA | Triethylamine |
| THF | Tetrahydrofuran |
| TrixiePhos | rac-2-(Di-tert-butylphosphino)-1,1′-binaphthyl |
| XantPhos | 4,5-Bis(diphenylphosphino)-9,9-dimethylxanthene |
| X-Phos | 2-Dicyclohexylphosphino-2′,4′,6′-triisopropylbiphenyl |
| XPhos-Pd G2 | Chloro(2-dicyclohexylphosphino-2′,4′,6′-triisopropyl-1,1′-biphenyl)[2-(2′-amino- |
| | 1,1′-biphenyl)]palladium(II) |

### Example 1. Synthesis of 5-methoxy-2-morpholino-N-phenyl-6-(1-phenyl-1H-pyrazol-3-yl)pyrimidine-4-carboxamide (Compound 1)

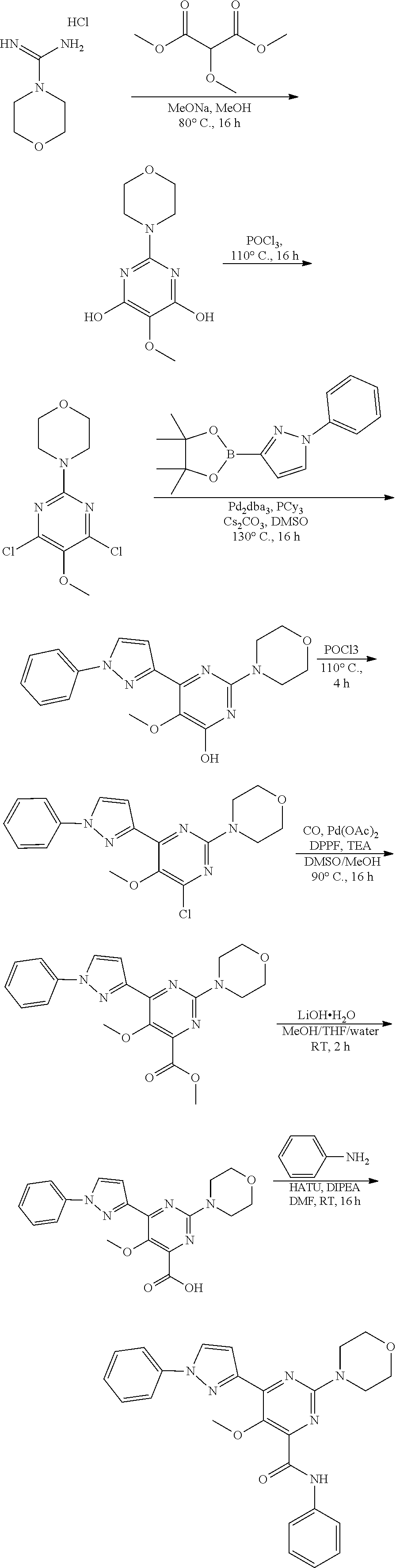

### Step 1: Synthesis of 5-methoxy-2-morpholinopyrimidine-4,6-diol

To a solution of morpholine-4-carboximidamide hydrochloride (2 g, 12.1 mmol) and dimethyl 2-methoxymalonate (1.95 g, 12.1 mmol) in MeOH (35 mL) was added MeONa (30% solution in MeOH, 6.8 mL, 36.3 mmol). After the addition, the reaction mixture was stirred at 80° C. for 17 h and then concentrated to afford 5-methoxy-2-morpholinopyrimidine-4,6-diol (2.5 g, 91%) as brown solid, which was used directly in next step without further purification.

### Step 2: Synthesis of 4-(4,6-dichloro-5-methoxypyrimidin-2-yl)morpholine

A mixture of 5-methoxy-2-morpholinopyrimidine-4,6-diol (2.5 g, 11 mmol) and phosphorus oxychloride (25 mL) was stirred at 110° C. for 16 h and then concentrated. The resultant residue was diluted with ethyl acetate/water (20 mL/20 mL) and extracted with ethyl acetate (20 mL) twice. The combined organic phase was washed with brine (30 mL), dried over sodium sulfate, filtered and concentrated. The crude product obtained was purified by flash column chromatography (Biotage, 40 g silica gel, eluted with ethyl acetate in petroleum ether from 20% to 40%) to afford 4-(4,6-dichloro-5-methoxypyrimidin-2-yl)morpholine (1.5 g, 51.6%) as pale yellow solid. LCMS (ESI) m/z: 264.1 [M+H]⁺.

### Step 3: Synthesis of 5-methoxy-2-morpholino-6-(1-phenyl-1H-pyrazol-3-yl)pyrimidin-4-ol

A mixture of 4-(4,6-dichloro-5-methoxypyrimidin-2-yl)morpholine (0.14 g, 0.53 mmol), 1-phenyl-3-(4,4,5,5-tetramethyl-1,3,2-dioxaborolan-2-yl)-1H-pyrazole (0.16 g, 0.58 mmol), tris(dibenzylidene acetone)dipalladium (49 mg, 0.053 mmol), tricyclohexyl phosphine (30 mg, 0.1 mmol) and cesium carbonate (0.34 g, 1.06 mmol) in dimethyl sulfoxide (20 mL) was stirred under nitrogen atmosphere at 130° C. for 16 h. The reaction was cooled down and the mixture was diluted with ethyl acetate (20 mL) and filtered through a pad of celite. The filtrate was extracted with water (40 mL) and the aqueous phase was lyophilized to afford 5-methoxy-2-morpholino-6-(1-phenyl-1H-pyrazol-3-yl)pyrimidin-4-ol (0.15 g, 80%) as white solid. LCMS (ESI) m/z: 354.1 [M+H]⁺.

### Step 4: Synthesis of 4-(4-chloro-5-methoxy-6-(1-phenyl-1H-pyrazol-3-yl)pyrimidin-2-yl)morpholine

A mixture of 5-methoxy-2-morpholino-6-(1-phenyl-1H-pyrazol-3-yl)pyrimidin-4-ol (0.15 g, 0.42 mmol) and phosphorus oxychloride (8 mL) was stirred at 110° C. for 16 h. The mixture was concentrated, the residue was diluted with ethyl acetate/water (20 mL/20 mL) and extracted with ethyl acetate (20 mL) twice. The combined organic phase was washed with brine (30 mL), dried over sodium sulfate, filtered and concentrated. The residue was purified by flash chromatography (Biotage, 40 g silica gel, eluted with ethyl acetate in petroleum ether from 20% to 40%) to afford 4-(4-chloro-5-methoxy-6-(1-phenyl-1H-pyrazol-3-yl)pyrimidin-2-yl)morpholine (0.11 g, 70.6%) as white solid. LCMS (ESI) m/z: 372.1 [M+H]⁺.

### Step 5: Synthesis of methyl 5-methoxy-2-morpholino-6-(1-phenyl-1H-pyrazol-3-yl)pyrimidine-4-carboxylate

A solution of 4-(4-chloro-5-methoxy-6-(1-phenyl-1H-pyrazol-3-yl)pyrimidin-2-yl)morpholine (100 mg, 0.27 mmol), triethylamine (82 mg, 0.81 mmol), palladium(II) acetate (6 mg, 0.03 mmol) and 1,1′-bis(diphenylphosphino)ferrocene (30 mg, 0.06 mmol) in methanol (4 mL) and dimethyl sulfoxide (6 mL) was stirred at 80° C. for 16 h under carbon monoxide atmosphere. After cooling to room temperature, the reaction mixture was filtered through celite. The filtrate was diluted with ethyl acetate (30 mL), washed with water (20 mL×3) and brine (30 mL), dried over sodium sulfate, filtered and concentrated. The residue was purified by silica gel column chromatography, eluting with ethyl acetate to give methyl 5-methoxy-2-morpholino-6-(1-phenyl-1H-pyrazol-3-yl)pyrimidine-4-carboxylate (25 mg, 23.4%) as white solid. LCMS (ESI) m/z: 396.1 [M+H]⁺.

### Step 6: Synthesis of 5-methoxy-2-morpholino-6-(1-phenyl-1H-pyrazol-3-yl)pyrimidine-4-carboxylic acid

To a solution of methyl 5-methoxy-2-morpholino-6-(1-phenyl-1H-pyrazol-3-yl)pyrimidine-4-carboxylate (20 mg, 0.05 mmol) in tetrahydrofuran/methanol/water (4 mL/1 mL/1 mL) was added lithium hydroxide monohydrate (4 mg, 0.1 mmol) and the reaction mixture was stirred at room temperature for 2 h. It was concentrated and the residue was diluted with water (5 mL), adjusted pH to 3-4 with aqueous 2N HCl and extracted with ethyl acetate (10 mL) twice. The combined organic phase was dried over sodium sulfate, filtered and concentrated to afford 5-methoxy-2-morpholino-6-(1-phenyl-1H-pyrazol-3-yl)pyrimidine-4-carboxylic acid (15 mg, 78.7%) as white solid. LCMS (ESI) m/z: 382.2 [M+H]⁺.

### Step 7: Synthesis of 5-methoxy-2-morpholino-N-phenyl-6-(1-phenyl-1H-pyrazol-3-yl)pyrimidine-4-carboxamide

To a solution of 5-methoxy-2-morpholino-6-(1-phenyl-1H-pyrazol-3-yl)pyrimidine-4-carboxylic acid (15 mg, 0.039 mmol) and aniline (4 mg, 0.039 mmol) in N,N-dimethylformamide (5 mL) were added HATU (15 mg, 0.047 mmol) and DIPEA (10 mg, 0.08 mmol). The resultant reaction mixture was stirred at room temperature for 16 h. The mixture was filtered and the filtrate was purified by prep-HPLC to afford 5-methoxy-2-morpholino-N-phenyl-6-(1-phenyl-1H-pyrazol-3-ylpyrimidine-4-carboxamide (4.3 mg, 24.2%) as white solid. ¹H NMR (400 MHz, DMSO-d₆) δ 10.62 (s, 1H), 8.68 (d, J=2.6 Hz, 1H), 7.97 (d, J=7.7 Hz, 2H), 7.73 (d, J=7.6 Hz, 2H), 7.56 (t, J=8.0 Hz, 2H), 7.38 (t, J=7.8 Hz, 3H), 7.22 (d, J=2.5 Hz, 1H), 7.14 (t, J=7.4 Hz, 1H), 3.84 (s, 3H), 3.76 (d, J=5 Hz, 4H), 3.71 (d, J=4.9 Hz, 4H); LCMS (ESI) m/z: 457.1 [M+H]⁺.

### Example 2. Synthesis of 4-(5-methoxy-4-(4-phenyl-1H-pyrazol-1-yl)-6-(pyridin-3-yloxy)pyrimidin-2-yl) morpholine (Compound 2)

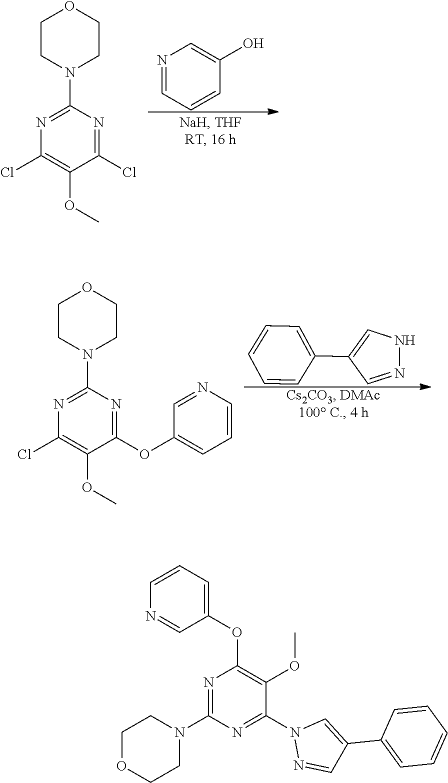

### Step 1: Synthesis of 4-(4-chloro-5-methoxy-6-(pyridin-3-yloxy)pyrimidin-2-yl)morpholine

To a solution of pyridin-3-ol (0.18 g, 1.9 mmol) in tetrahydrofuran (20 mL) at 0° C. was added sodium hydride (60% in mineral oil, 0.11 g, 2.85 mmol) in portions. After the addition, the mixture was warmed up and stirred at room temperature for 20 min. Then a solution of 4-(4,6-dichloro-5-methoxypyrimidin-2-yl)morpholine (0.5 g, 1.9 mmol) in tetrahydrofuran (10 mL) was added. The resultant mixture was heated to reflux with stirring for 16 h. It was cooled down, dilute with ethyl acetate/water (20 mL/20 mL) and extracted with ethyl acetate (20 mL*2). The combined organic phase was washed with brine (20 mL), dried over sodium sulfate, filtered and concentrated. The crude product obtained was purified by flash chromatography (Biotage, 40 g silica gel, eluted with ethyl acetate in petroleum ether form 50% to 70%) to afford 4-(4-chloro-5-methoxy-6-(pyridin-3-yloxy)pyrimidin-2-yl)morpholine (0.46 g, 75.4%) as white solid. LCMS (ESI) m/z: 323.1 [M+H]⁺.

### Step 2: Synthesis of 4-(5-methoxy-4-(4-phenyl-1H-pyrazol-1-yl)-6-(pyridin-3-yloxy)pyrimidin-2-yl)morpholine

A mixture of 4-(4-chloro-5-methoxy-6-(pyridin-3-yloxy)pyrimidin-2-yl)morpholine (40 mg, 0.12 mmol), 4-phenyl-1H-pyrazole (18 mg, 0.12 mmol) and cesium carbonate (80 mg, 0.24 mmol) in N,N-dimethylacetamide (6 mL) was stirred at 100° C. for 4 h. The resultant mixture was filtered through a pad of celite and concentrated. The residue was subjected to prep-HPLC to obtain 4-(5-methoxy-4-(4-phenyl-1H-pyrazol-1-yl)-6-(pyridin-3-yloxy)pyrimidin-2-yl)morpholine (12.5 mg, 23.6%) as white solid. ¹H NMR (400 MHz, DMSO-d₆) δ 8.94 (s, 1H), 8.60 (d, J=2.7 Hz, 1H), 8.50 (dd, J=4.7, 1.3 Hz, 1H), 8.39 (s, 1H), 7.85-7.73 (m, 3H), 7.54 (d, J=4.7 Hz, 1H), 7.43 (t, J=7.7 Hz, 2H), 7.29 (s, 1H), 3.86 (s, 3H), 3.58 (d, J=4 Hz, 4H), 3.49 (bs, 4H), LCMS (ESI) m/z: 431.1 [M+H]⁺.

### Example 3. Synthesis of 5-methoxy-6-(3-(1-methyl-1H-pyrazol-3-yl)phenyl)-2-morpholino-N-(pyridin-4-yl)pyrimidin-4-amine (Compound 3)

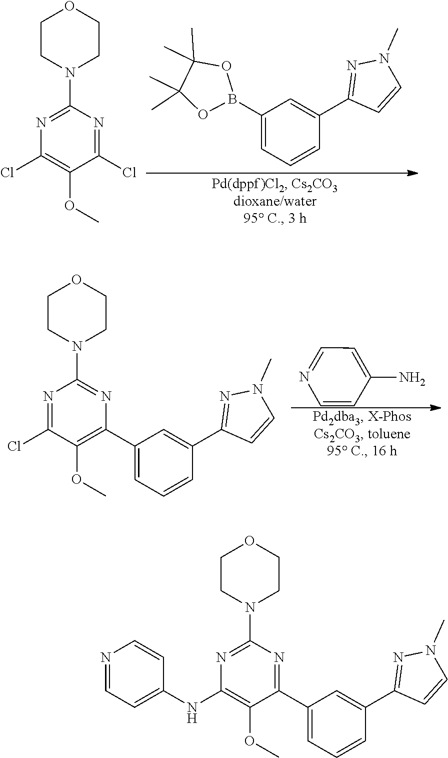

### Step 1: Preparation of 4-(4-chloro-5-methoxy-6-(3-(1-methyl-1H-pyrazol-3-yl)phenyl)pyrimidin-2-yl)morpholine

A mixture of 4-(4,6-dichloro-5-methoxypyrimidin-2-yl)morpholine (0.2 g, 0.76 mmol), 1-methyl-3-(3-(4,4,5,5-tetramethyl-1,3,2-dioxaborolan-2-yl)phenyl)-1H-pyrazole (0.21 g, 0.76 mmol), 1,1′-bis(diphenylphosphino)ferrocene-palladium(II)dichloride dichloromethane complex (62 mg, 0.075 mmol) and cesium carbonate (0.62 g, 1.89 mmol) in 1,4-dioxane/water (20 mL/4 mL) was stirred at 95° C. for 3 h under argon atmosphere. It was concentrated, the residue was diluted with water (20 mL) and extracted with ethyl acetate (20 mL×2). The combined organic phase was washed with brine (20 mL), dried over sodium sulfate, filtered and concentrated. The crude product obtained was purified by flash chromatography (Biotage, 40 g silica gel, eluted with petroleum ether in ethyl acetate from 20% to 40%) to afford 4-(4-chloro-5-methoxy-6-(3-(1-methyl-1H-pyrazol-3-yl)phenyl)pyrimidin-2-yl)morpholine (0.15 g, 51.3%) as white solid. LCMS (ESI) m/z: 385.8/387.8 [M+H]⁺.

### Step 2: Synthesis of 5-methoxy-6-(3-(1-methyl-1H-pyrazol-3-yl)phenyl)-2-morpholino-N-(pyridin-4-yl)pyrimidin-4-amine

A mixture of 4-(4-chloro-5-methoxy-6-(3-(1-methyl-1H-pyrazol-3-yl)phenyl)pyrimidin-2-yl)morpholine (80 mg, 0.21 mmol), pyridin-4-amine (20 mg, 0.21 mmol), tris(dibenzylidene acetone)dipalladium (19 mg, 0.02 mmol), XPhos (20 mg, 0.04 mmol) and cesium carbonate (0.17 g, 0.52 mmol) in toluene (15 mL) was stirred at 95° C. for 16 h under argon atmosphere. It was concentrated, the residue was diluted with water (20 mL) and extracted with dichloromethane (20 mL×2). The combined organic phase was washed with brine (20 mL), dried over sodium sulfate, filtered and concentrated. The residue was subjected to prep-HPLC to afford 5-methoxy-6-(3-(1-methyl-1H-pyrazol-3-yl)phenyl)-2-morpholino-N-(pyridin-4-ylpyrimidin-4-amine (17.8 mg, 19%) as white solid. ¹H NMR (400 MHz, DMSO-d₆) δ 9.36 (s, 1H), 8.44-8.42 (m, 3H), 7.91-7.86 (m, 4H), 7.77 (d, J=2.2 Hz, 1H), 7.52 (t, J=7.6 Hz, 1H), 6.71 (d, J=2.2 Hz, 1H), 3.90 (s, 3H), 3.70 (s, 8H), 3.45 (s, 3H); LCMS (ESI) m/z: 443.9 [M]⁺.

### Example 4. Synthesis of 4-(6-methoxy-2-(3-(1-methyl-1H-pyrazol-3-yl)phenyl)-6-(2-(pyridin-2-yl)ethoxy) pyrimidin-4-yl)morpholine (Compound 4)

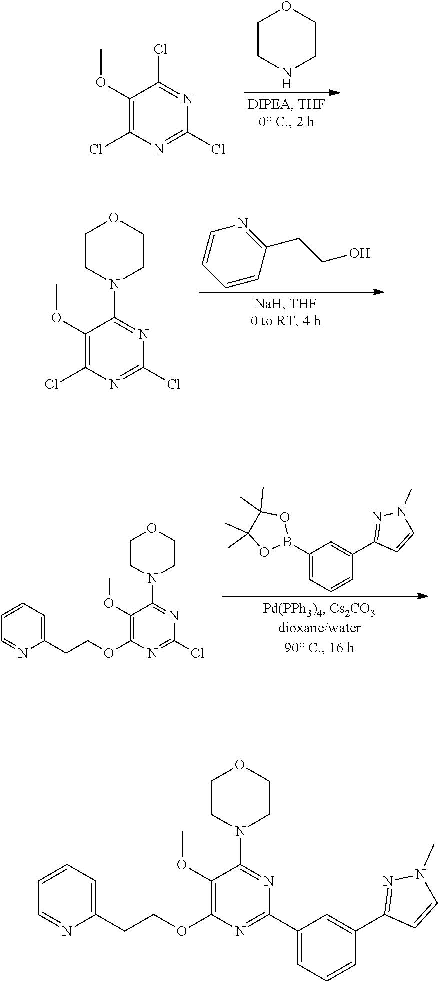

### Step 1: Synthesis of 4-(2,6-dichloro-5-methoxypyrimidin-4-yl)morpholine

To a solution of 2,4,6-trichloro-5-methoxypyrimidine (0.4 g, 1.88 mmol) and morpholine (0.16 g, 1.88 mmol) in tetrahydrofuran (20 mL) was added N,N-diisopropylethylamine (0.48 g, 3.77 mmol). And the reaction mixture was stirred at room temperature for 2 h. It was concentrated and the crude product obtained was purified by column chromatography (on silica gel) to obtain 4-(2,6-dichloro-5-methoxypyrimidin-4-yl)morpholine (0.49 g, 98%) as white solid. LCMS (ESI) m/z: 264.0 [M+H]⁺.

### Step 2: Synthesis of 4-(2-chloro-6-methoxy-6-(2-(pyridin-2-yl)ethoxy)pyrimidin-4-yl)morpholine

To a solution of 2-(pyridin-2-yl)ethan-1-ol (0.19 g, 1.52 mmol) in tetrahydrofuran (20 mL) at 0° C. was added sodium hydride (60%, 91 mg, 2.28 mmol) in portions. After the addition, the mixture was stirred at 0° C. for 30 min followed by the drop-wise addition of 4-(2,6-dichloro-5-methoxypyrimidin-4-yl)morpholine (0.4 g, 1.52 mmol) in tetrahydrofuran (4 mL) at the same temperature. The resultant mixture was warmed up and stirred at room temperature for 4 h, then diluted with ethyl acetate/water (20 mL/20 mL) and extracted with ethyl acetate (20 mL×2). The combined organic phase was washed with brine (30 mL), dried over sodium sulfate, filtered and concentrated. The crude product obtained was purified by flash chromatography (Biotage, 40 g silica gel, eluted 7N ammonia methanol in DCM from 20% to 40%) to afford 4-(2-chloro-5-methoxy-6-(2-(pyridin-2-yl)ethoxy) pyrimidin-4-yl)morpholine (0.26 g, 48.9%) as white solid. LCMS (ESI) m/z: 351.1[M+H]⁺. (Rt: 1.81 min.)

The regioisomer 4-(6-chloro-5-methoxy-2-(2-(pyridin-2-yl)ethoxy)pyrimidin-4-yl)morpholine (0.14 g, 26.3%) was also obtained as white solid. LCMS (ESI) m/z: 351.1[M+H]⁺; (Rt=1.68 min). The two isomers were confirmed by NOESY.

### Step 3: Synthesis of 4-(5-methoxy-2-(3-(1-methyl-1H-pyrazol-3-yl)phenyl)-6-(2-(pyridin-2-yl)ethoxy)pyrimidin-4-yl)morpholine

A mixture of 4-(2-chloro-5-methoxy-6-(2-(pyridin-2-yl)ethoxy) pyrimidin-4-yl)morpholine (0.1 g, 0.28 mmol), 1-methyl-3-(3-(4,4,5,5-tetramethyl-1,3,2-dioxaborolan-2-yl)phenyl)-1H-pyrazole (81 mg, 0.28 mmol), tetrakis(triphenylphosphine)palladium (33 mg, 0.03 mmol) and cesium carbonate (0.23 g, 0.71 mmol) in dioxane/water (20 mL/4 mL) was stirred at 90° C. for 16 h. The resultant mixture was filtered and concentrated. The residue was subjected to prep-HPLC to obtain 4-(5-methoxy-2-(3-(1-methyl-1H-pyrazol-3-yl)phenyl)-6-(2-(pyridin-2-yl)ethoxy) pyrimidin-4-yl)morpholine (28.7 mg, 21.7%) as white solid. ¹H NMR (400 MHz, CDCl₃) δ 8.69 (t, J=1.6 Hz, 1H), 8.57 (d, J=4.0 Hz, 1H), 8.25 (dt, J=4.0, 1.6 Hz, 1H), 7.88 (dt, J=4.0, 1.6 Hz, 1H), 7.61 (td, J=7.7, 1.8 Hz, 1H), 7.45 (t, J=8 Hz, 1H), 7.41 (d, J=1.6 Hz, 1H), 7.32 (d, J=7.8 Hz, 1H), 7.14 (dd, J=7.0, 5.4 Hz, 1H), 6.62 (d, J=2.2 Hz, 1H), 4.93 (t, J=6.6 Hz, 2H), 3.98 (s, 3H), 3.83 (s, 8H), 3.59 (s, 3H), 3.37 (t, J=6.6 Hz, 2H); LCMS (ESI) m/z: 473.3 [M+H]⁺.

### Example 5. Synthesis of 4-(5-methoxy-6-(3-(1-methyl-H-pyrazol-3-yl)phenyl)-2-(2-(pyridin-2-yl)ethoxy) pyrimidin-4-yl)morpholine (Compound 5)

The following compound was synthesized according to the protocol described above in Example 4:

| No. | Structure | NMR, MS |
|---|---|---|
| 5 | 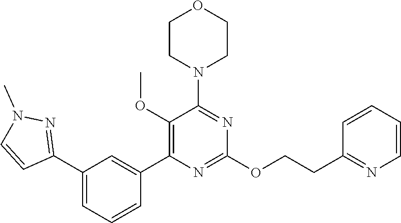 [SMILES: COc1c(-c2cccc(-c3ccn(C)n3)c2)nc(OCCc2ccccn2)nc1N1CCOCC1] | ¹H NMR (400 MHz, CDCl₃) δ 8.55 (d, J = 4.0 Hz, 1H), 8.37 (t, J = 1.6 Hz, 1H), 7.88 (td, J = 12.0, 4.0 Hz, 2H), 7.59 (td, J = 7.7, 1.8 Hz, 1H), 7.45 (t, J = 7.8 Hz, 1H), 7.39 (d, J = 2.2 Hz, 1H), 7.31 (d, J = 7.8 Hz, 1H), 7.13 (dd, J = 6.6, 5.0 Hz, 1H), 6.59 (d, J = 2.2 Hz, 1H), 4.73 (t, J = 6.9 Hz, 2H), 3.96 (s, 3H), 3.83 (d, J = 3.6 Hz, 8H), 3.41 (s, 3H), 3.33 (t, J = 6.9 Hz, 2H); LCMS (ESI) m/z: 472.8 [M]⁺. |

### Example 6. Synthesis of N-cyclopropyl-4-methoxy-6-(3-(1-methyl-H-pyrazol-3-yl)phenyl)-2-morpholino pyrimidine-4-carboxamide (Compound 6)

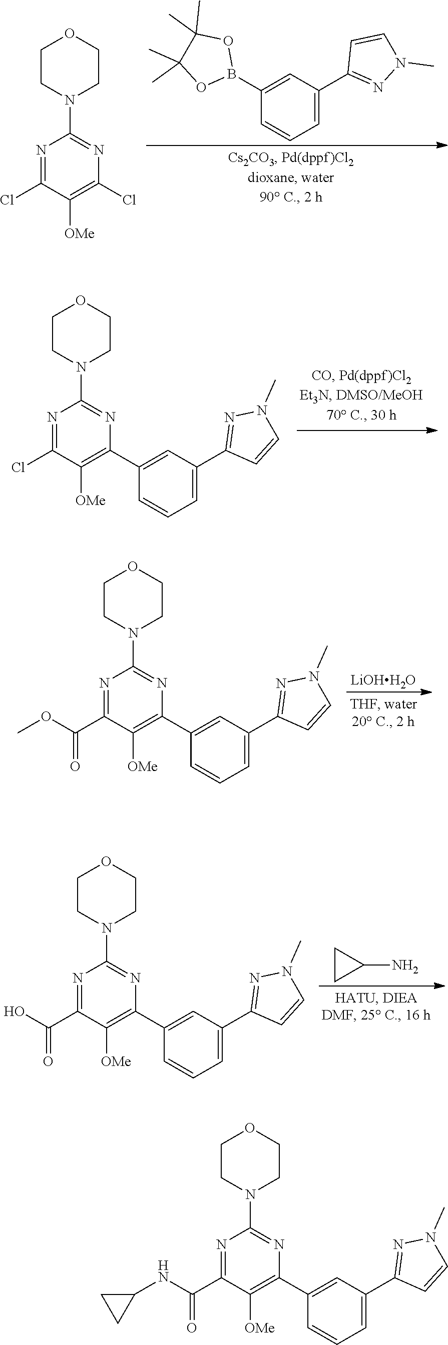

### Step 1: Synthesis of 4-(4-chloro-6-methoxy-6-(3-(1-methyl-1H-pyrazol-3-yl)phenyl)pyrimidin-2-yl)morpholine

To a solution of 4-(4,6-dichloro-5-methoxypyrimidin-2-yl)morpholine (0.26 g, 0.984 mmol) and 1-methyl-3-(3-(4,4,5,5-tetramethyl-1,3,2-dioxaborolan-2-yl)phenyl)-1H-pyrazole (0.28 g, 0.984 mmol) in dioxane/water (10 mL/2 mL) were added cesium carbonate (0.64 g, 1.97 mmol) and [1,1′-bis(diphenylphosphino)ferrocene]dichloropalladium(II) (0.073 g, 0.1 mmol). The resultant mixture was stirred at 90° C. for 2 h, then poured into ice-water and extracted with ethyl acetate (15 mL×3). The combined organic layer was washed with brine, dried and evaporated to dryness. The crude product obtained was chromatographed on silica gel (petroleum ether/ethyl acetate=20:1) to obtain the target product (200 mg, 53%) as yellow solid. LCMS (ESI) m/z: 385.8/387.8 [M]⁺.

### Step 2: Synthesis of methyl 5-methoxy-6-(3-(1-methyl-1H-pyrazol-3-yl)phenyl)-2-morpholinopyrimidine-4-carboxylate

A mixture of 4-(4-chloro-5-methoxy-6-(3-(1-methyl-1H-pyrazol-3-yl)phenyl)pyrimidin-2-yl)morpholine (0.2 g, 0.52 mmol), triethylamine (0.16 g, 1.56 mmol) and [1,1′-bis(diphenyl phosphino)ferrocene]dichloropalladium(II) (0.04 g, 0.05 mmol) in methanol/dimethyl sulfoxide (10 mL/5 mL) was stirred at 75° C. under carbon monoxide for 16 h. The reaction mixture was concentrated and the crude product obtained was purified by column chromatography (on silica gel using petroleum ether/ethyl acetate=2:1) to obtain the target product as yellow solid (0.12 g, 56%). LCMS (ESI) m/z: 410.1 [M+H]⁺.

### Step 3: Synthesis of 5-methoxy-6-(3-(1-methyl-1H-pyrazol-3-yl)phenyl)-2-morpholinopyrimidine-4-carboxylic acid

A mixture of methyl 5-methoxy-6-(3-(1-methyl-1H-pyrazol-3-yl)phenyl)-2-morpholinopyrimidine-4-carboxylate (0.12 g, 0.29 mmol) and lithium hydroxide hydrate (0.025 g, 0.58 mmol) in water (1 mL)/tetrahydrofuran (4 mL) was stirred at 25° C. for 5 h. It was concentrated to give the target product (0.1 g, 86%) as yellow solid. LCMS (ESI) m/z: 396.1 [M+H]⁺.

### Step 4: Synthesis of N-cyclopropyl-6-methoxy-6-(3-(1-methyl-1H-pyrazol-3-yl)phenyl)-2-morpholinopyrimidine-4-carboxamide

A solution of 5-methoxy-6-(3-(1-methyl-1H-pyrazol-3-yl)phenyl)-2-morpholinopyrimidine-4-carboxylic acid (120 mg, 0.3 mmol), cyclopropanamine (52 mg, 0.91 mmol), N,N-diisopropylethylamine (120 mg, 0.91 mmol) and 1-[bis(dimethylamino)methylene]-1H-1,2,3-triazolo[4,5-b]pyridinium 3-oxid hexafluorophosphate (171 mg, 0.45 mmol) in N,N-dimethylformamide (5 mL) was stirred at 25° C. for 2 h. The resultant mixture was filtered and concentrated. The residue was subjected to prep-HPLC (SunFire C18, 4.6*50 mm, 3.5 um column Xbridge C18 3.5 μm 4.6×50 mm column. The mobile phase was acetonitrile/0.1% Ammonium bicarbonate aqueous solution) to obtain the target product as white solid (42.6 mg, 32%). ¹H NMR (400 MHz, DMSO-d₆) δ 8.65 (d, J=4.5 Hz, 1H), 8.41 (t, J=1.5 Hz, 1H), 7.97-7.86 (m, 2H), 7.77 (d, J=2.2 Hz, 1H), 7.53 (t, J=7.8 Hz, 1H), 6.73 (d, J=2.2 Hz, 1H), 3.90 (s, 3H), 3.70 (d, J=3.6 Hz, 8H), 3.48 (d, J=6.2 Hz, 3H), 2.83 (pent, J=4.1 Hz, 1H), 0.76-0.69 (m, 2H), 0.59-0.49 (m, 2H); LCMS (ESI) m/z: 435.1 [M+H]⁺.

### Example 7. Synthesis of 5-methoxy-6-(5-(1-methyl-1H-pyrazol-3-yl)pyridin-3-yl)-2-morpholino-N-(pyridin-4-yl)pyrimidin-4-amine (Compound 7)

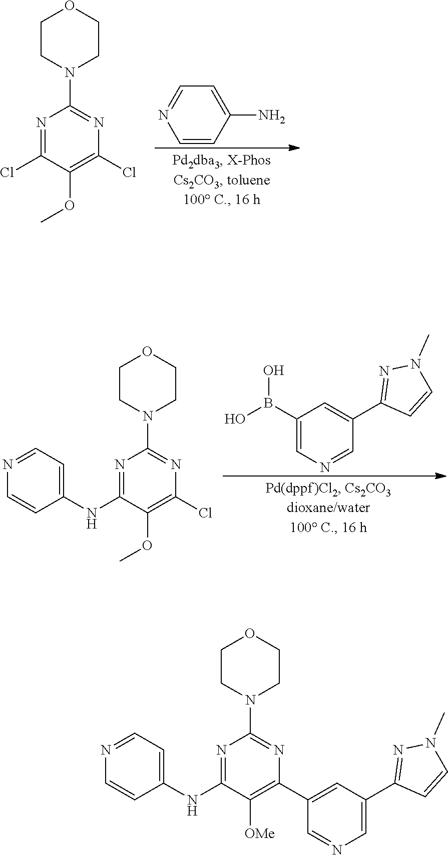 [SMILES: COc1c(Cl)nc(N2CCOCC2)nc1Cl ; Nc1ccncc1 ; COc1c(Cl)nc(N2CCOCC2)nc1Nc1ccncc1 ; Cn1ccc(-c2cncc(B(O)O)c2)n1 ; COc1c(Nc2ccncc2)nc(N2CCOCC2)nc1-c1cncc(-c2ccn(C)n2)c1]

### Step 1: Synthesis of 6-chloro-5-methoxy-2-morpholino-N-(pyridin-4-yl)pyrimidin-4-amine

A mixture of 4-(4,6-dichloro-5-methoxypyrimidin-2-yl)morpholine (0.5 g, 1.89 mmol), pyridin-4-amine (0.16 g, 1.7 mmol), tris(dibenzylideneacetone)dipalladium (0.17 g, 0.19 mmol), X-Phos (0.18 g, 0.38 mmol) and cesium carbonate (1.54 g, 4.73 mmol) in toluene (25 mL) was stirred at 100° C. for 16 h under nitrogen atmosphere. The mixture was concentrated, the residue was diluted with water (20 mL) and extracted with dichloromethane (20 mL×2). The combined organic phase was washed with brine (20 mL), dried over sodium sulfate, filtered and concentrated. The resultant crude product was purified by flash chromatography (eluted with petroleum ether in ethyl acetate from 30% to 60%) to afford 6-chloro-5-methoxy-2-morpholino-N-(pyridin-4-yl)pyrimidin-4-amine (0.2 g, 36.7%) as white solid. LCMS (ESI) m/z: 321.8 [M]⁺.

### Step 2: Synthesis of 5-methoxy-6-(5-(1-methyl-H-pyrazol-3-yl)pyridin-3-yl)-2-morpholino-N-(pyridin-4-yl)pyrimidin-4-amine

A mixture of 6-chloro-5-methoxy-2-morpholino-N-(pyridin-4-yl)pyrimidin-4-amine (80 mg, 0.25 mmol), (5-(1-methyl-1H-pyrazol-3-yl)pyridin-3-yl)boronic acid (60.7 mg, 0.3 mmol), 1,1′-bis(diphenylphosphino)ferrocene-palladium(II)dichloride dichloromethane complex (20 mg, 0.025 mmol) and cesium carbonate (0.2 g, 0.62 mmol) in 1,4-dioxane/water (15 mL/3 mL) was stirred at 100° C. for 16 h under nitrogen atmosphere. The reaction mixture was concentrated, followed by the addition of water (20 mL) and the mixture was extracted with ethyl acetate (20 mL×2). The combined organic phase was washed with brine (20 mL), dried over sodium sulfate, filtered and concentrated. The residue was subjected to prep-HPLC to obtain 5-methoxy-6-(5-(1-methyl-1H-pyrazol-3-yl)pyridin-3-yl)-2-morpholino-N-(pyridin-4-ylpyrimidin-4-amine (45 mg, 40.5%) as white solid. ¹H NMR (400 MHz, DMSO-d₆) δ 9.44 (s, 1H), 9.07-9.06 (m, 2H), 8.69 (s, 1H), 8.43 (d, J=6.1 Hz, 2H), 7.89 (d, J=6.2 Hz, 2H), 7.83 (d, J=2.0 Hz, 1H), 6.87 (d, J=2.1 Hz, 1H), 3.93 (s, 3H), 3.71 (s, 8H), 3.49 (s, 3H); LCMS (ESI) m/z: 444.8 [M]⁺.

### Example 8. Synthesis of 6-(3-(1H-pyrazol-1-yl)phenyl)-5-methoxy-2-morpholino-N-(pyridin-4-yl)pyrimidin-4-amine (Compound 8)

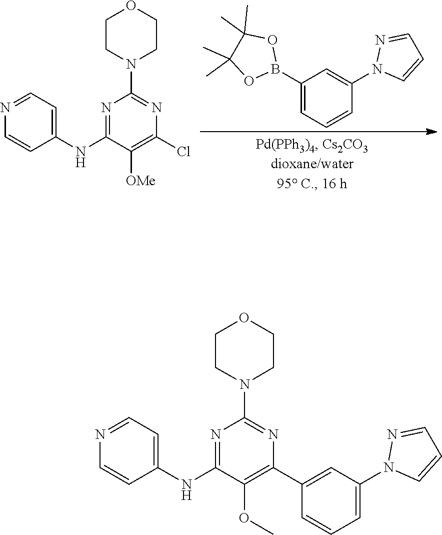 [SMILES: COc1c(Cl)nc(N2CCOCC2)nc1Nc1ccncc1 ; CC1(C)OB(c2cccc(-n3cccn3)c2)OC1(C)C ; COc1c(Nc2ccncc2)nc(N2CCOCC2)nc1-c1cccc(-n2cccn2)c1]

A mixture of 6-chloro-5-methoxy-2-morpholino-N-(pyridin-4-yl)pyrimidin-4-amine (40 mg, 0.12 mmol), 1-(3-(4,4,5,5-tetramethyl-1,3,2-dioxaborolan-2-yl)phenyl)-1H-pyrazole (33.6 mg, 0.12 mmol), tetrakis(triphenylphosphine)palladium (14 mg, 0.012 mmol) and cesium carbonate (0.1 g, 0.31 mmol) in 1,4-dioxane/water (5 mL/1 mL) was stirred at 95° C. for 16 h under nitrogen atmosphere. The reaction mixture was concentrated, then diluted with water (10 mL) and extracted with ethyl acetate (10 mL×2). The combined organic phase was washed with brine (10 mL), dried over sodium sulfate, filtered and concentrated. The residue was purified by prep-HPLC to afford 6-(3-(1H-pyrazol-1-yl)phenyl)-5-methoxy-2-morpholino-N-(pyridin-4-yl)pyrimidin-4-amine (17.8 mg, 33.3%) as white solid. ¹H NMR (400 MHz, DMSO-d₆) δ 9.42 (s, 1H), 8.56 (d, J=2.4 Hz, 1H), 8.48 (d, J=1.8 Hz, 1H), 8.43 (d, J=6.1 Hz, 2H), 7.99-7.86 (m, 4H), 7.79 (d, J=1.6 Hz, 1H), 7.63 (t, J=7.9 Hz, 1H), 6.62-6.55 (m, 1H), 3.71 (s, 8H), 3.48 (s, 3H); LCMS (ESI) m/z: 430.2 [M+H]⁺.

### Example 9. Synthesis of 6-methoxy-6-(3-(3-methyl-1H-pyrazol-1-yl)phenyl)-2-morpholino-N-(pyridin-4-yl)pyrimidin-4-amine (Compound 9)

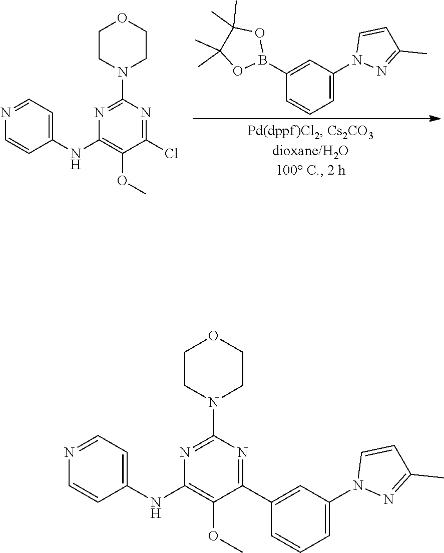

A solution of 6-chloro-5-methoxy-2-morpholino-N-(pyridin-4-yl)pyrimidin-4-amine (100 mg, 0.28 mmol), 3-methyl-1-(3-(4,4,5,5-tetramethyl-1,3,2-dioxaborolan-2-yl)phenyl)-1H-pyrazole (48 mg, 0.32 mmol), [1,1′-bis(diphenylphosphino)ferrocene]dichloropalladium(II) (20 mg, 0.0028 mmol) and cesium carbonate (209 mg, 0.84 mmol) in dioxane (5 mL) and water (0.5 mL) was stirred at 100° C. for 2 h under nitrogen atmosphere. Then water (20 mL) was added and the mixture was extracted with ethyl acetate (50 mL×3). The organic layer was dried and concentrated to obtain the crude product. It was then purified by prep-TLC (petroleum ether ethyl acetate from 50:1 to 10:1) to obtain 4-(3-(3-(1H-pyrazol-1-yl)phenyl)-7-(pyridin-4-yl)-1H-pyrazolo[4,3-d]pyrimidin-5-yl)morpholine (23.4 mg, 34%) as white solid. ¹H NMR (400 MHz, DMSO-d₆) δ 9.45 (s, 1H), 8.42 (dd, J=4.6, 2.8 Hz, 4H), 7.94-7.85 (m, 4H), 7.59 (t, J=7.9 Hz, 1H), 3.71 (s, 8H), 3.47 (s, 3H), 2.29 (s, 3H); LCMS (ESI) m/z: 443.9 [M]⁺.

### Example 10. Synthesis of 5-methoxy-2-morpholino-6-(3-(pyridazin-3-yl)phenyl)-N-(pyridin-4-yl)pyrimidin-4-amine (Compound 10), 3-(5-methoxy-2-morpholino-6-(pyridin-4-ylamino) pyrimidin-4-yl)-N,N-dimethylbenzamide (Compound 11), and 6-(5,6-dimethoxypyridin-3-yl)-5-methoxy-2-morpholino-N-(pyridin-4-yl)pyrimidin-4-amine (Compound 12)

The following compounds were synthesized according to the above protocol in Example 9.

| No. | Structure | NMR, MS |
|---|---|---|
| 10 | 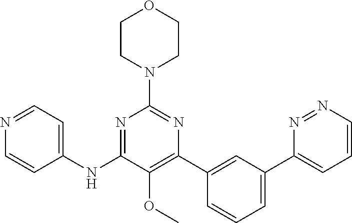 [SMILES: COc1c(Nc2ccncc2)nc(N2CCOCC2)nc1-c1cccc(-c2cccnn2)c1] | ¹H NMR (400 MHz, DMSO-d₆) δ 9.49 (s, 1H), 9.26 (d, J = 5.0 Hz, 1H), 8.81 (s, 1H), 8.44 (d, J = 6.4 Hz, 2H), 8.26 (dd, J = 16.9, 8.5 Hz, 2H), 8.18 (d, J = 8.3 Hz, 1H), 7.93 (d, J = 5.9 Hz, 2H), 7.84 (dd, J = 8.6, 4.7 Hz, 1H), 7.72 (t, J = 7.9 Hz, 1H), 3.72 (s, 8H), 3.49 (s, 3H); LCMS (ESI) m/z: 441.8 [M]⁺. |
| 11 | 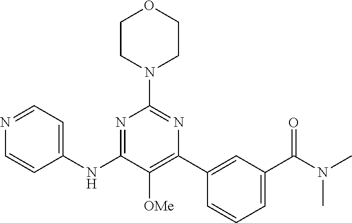 [SMILES: COc1c(Nc2ccncc2)nc(N2CCOCC2)nc1-c1cccc(C(=O)N(C)C)c1] | ¹H NMR (400 MHz, DMSO-d₆) δ 9.36 (s, 1H), 8.42 (d, J = 6.1 Hz, 2H), 8.09 (d, J = 7.6 Hz, 1H), 8.01 (s, 1H), 7.88 (d, J = 6.2 Hz, 2H), 7.63-7.46 (m, 2H), 3.69 (s, 8H), 3.45 (s, 3H), 2.99 (d, J = 20.3 Hz, 6H). LCMS (ESI) m/z: 435.4 [M + H]⁺. |
| 12 | 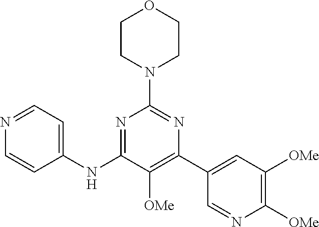 [SMILES: COc1cc(-c2nc(N3CCOCC3)nc(Nc3ccncc3)c2OC)cnc1OC] | ¹H NMR (400 MHz, DMSO-d₆) δ 9.37 (s, 1H), 8.48 (d, J = 1.8 Hz, 1H), 8.41 (d, J = 6.2 Hz, 2H), 7.88 (dd, J = 5.9, 4.2 Hz, 3H), 3.95 (s, 3H), 3.86 (s, 3H), 3.70 (s, 8H), 3.52 (s, 3H). LCMS (ESI) m/z: 424.8[M + H]⁺. |

### Example 11. Synthesis of 6-(3,4-dimethoxyphenyl)-5-methoxy-2-morpholino-N-(pyridin-4-yl)pyrimidin-4-amine (Compound 13)

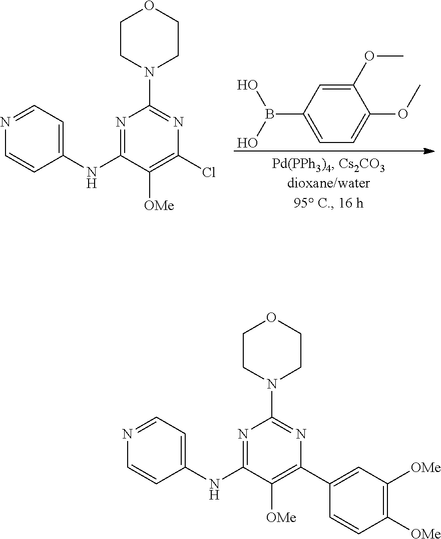 [SMILES: COc1c(Cl)nc(N2CCOCC2)nc1Nc1ccncc1 ; COc1ccc(B(O)O)cc1OC ; COc1ccc(-c2nc(N3CCOCC3)nc(Nc3ccncc3)c2OC)cc1OC]

A mixture of 6-chloro-5-methoxy-2-morpholino-N-(pyridin-4-yl)pyrimidin-4-amine (55 mg, 0.17 mmol), (3,4-dimethoxyphenyl) boronic acid (37.6 mg, 0.20 mmol), tetrakis(triphenyl phosphine) palladium (20 mg, 0.017 mmol) and cesium carbonate (0.14 g, 0.43 mmol) in 1,4-dioxane/water (10 mL/2 mL) was stirred at 95° C. for 16 h under argon atmosphere. The reaction mixture was concentrated, diluted with water (15 mL) and extracted with ethyl acetate (15 mL×2). The combined organic phase was washed with brine (20 mL), dried over sodium sulfate, filtered and concentrated. The residue was purified by prep-HPLC to afford 6-(3,4-dimethoxyphenyl)-5-methoxy-2-morpholino-N-(pyridin-4-ylpyrimidin-4-amine (63.9 mg, 88.9%) as white solid. ¹H NMR (400 MHz, DMSO-d₆) δ 9.29 (s, 1H), 8.40 (d, J=6.3 Hz, 2H), 7.87 (dd, J=4.9, 1.4 Hz, 2H), 7.78 (s, 2H), 7.09 (d, J=9.1 Hz, 1H), 3.82 (d, J=6.7 Hz, 6H), 3.70 (d, J=3.0 Hz, 8H), 3.49 (s, 3H); LCMS (ESI) m/z: 424.6 [M+H]⁺.

### Example 12. Synthesis of 5-methoxy-6-(2-(1-methyl-H-pyrazol-3-yl)pyridin-4-yl)-2-morpholino-N-(pyridin-4-yl)pyrimidin-4-amine (Compound 14)

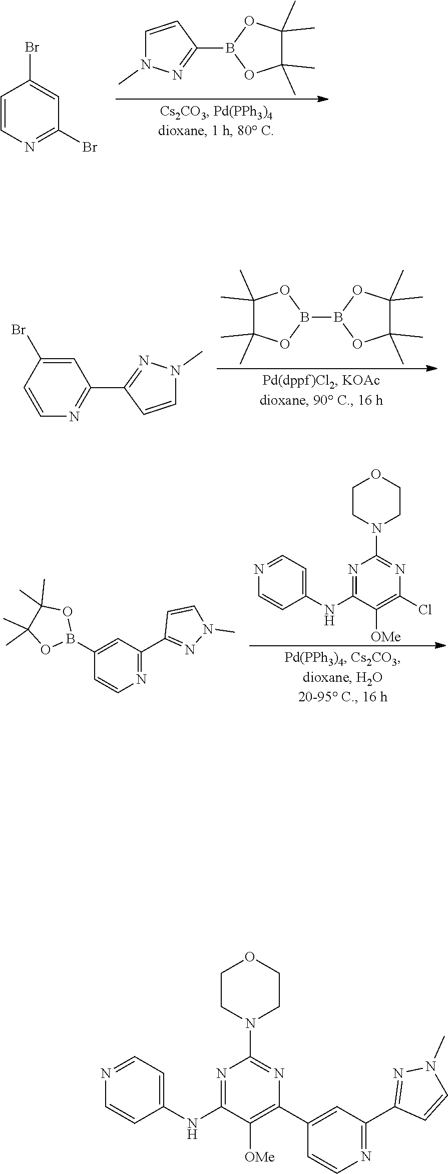

### Step 1: Synthesis of 4-bromo-2-(1-methyl-1H-pyrazol-3-yl)pyridine

A mixture of 2,4-dibromopyridine (472 mg, 2 mmol), 1-methyl-3-(4,4,5,5-tetramethyl-1,3,2-dioxaborolan-2-yl)-1H-pyrazole (416 mg, 2 mmol), tetrakis(triphenylphosphin)palladium (347 mg, 0.3 mmol) and cesium carbonate (1.956 g, 6 mmol) in water (3 mL) and dioxane (30 mL) was stirred at 80° C. for 16 h under argon atmosphere. The reaction mixture was cooled and concentrated. The crude product obtained was purified by silica gel column chromatography (methanol/dichloromethane=0%-3%) to obtain 4-bromo-2-(1-methyl-1H-pyrazol-3-yl)pyridine (450 mg, 61%) as yellow oil. LCMS (ESI) m/z: 238.0 [M+H]⁺.

### Step 2: Synthesis of 2-(1-methyl-1H-pyrazol-3-yl)-4-(4,4,5,5-tetramethyl-1,3,2-dioxaborolan-2-yl)pyridine

A mixture of 4-bromo-2-(1-methyl-1H-pyrazol-3-yl)pyridine (95 mg, 0.4 mmol), bis(pinacolato)diboron (122 mg, 0.48 mmol), [1,1′-bis(diphenylphosphino)ferrocene]dichloro palladium(II) dichloromethane complex (33 mg, 0.04 mmol) and potassium acetate (118 mg, 1.2 mmol) in dioxane (10 mL) was stirred at 90° C. for 16 h. The resultant mixture was used directly in the next step. LCMS (ESI) m/z: 286.2 [M+H]⁺.

### Step 3: Synthesis of 5-methoxy-6-(2-(1-methyl-1H-pyrazol-3-yl)pyridin-4-yl)-2-morpholino-N-(pyridin-4-yl)pyrimidin-4-amine

A mixture of 6-chloro-5-methoxy-2-morpholino-N-(pyridin-4-yl)pyrimidin-4-amine (128 mg, 0.4 mmol), tetrakis(triphenylphosphin)palladium (46 mg, 0.04 mmol), cesium carbonate (391 mg, 1.2 mmol) and water (0.5 mL) was added to the reaction mixture from above step and the resultant mixture was stirred at 95° C. for 16 h under argon. It was concentrated and purified by prep-HPLC (BOSTON pHlex ODS 10 um 21.2×250 mm 120 A. The mobile phase was acetonitrile/0.1% ammonium bicarbonate) to obtain 5-methoxy-6-(2-(1-methyl-1H-pyrazol-3-yl)pyridin-4-yl)-2-morpholino-N-(pyridin-4-yl)pyrimidin-4-amine (76.1 mg, 34%) as yellow solid. ¹H NMR (400 MHz, DMSO-d₆) δ 9.43 (bs, 1H), 8.70 (d, J=5.2 Hz, 1H), 8.52 (s, 1H), 8.43 (d, J=6.3 Hz, 2H), 7.87 (d, J=6.4 Hz, 1H) 7.80 (d, J=2.4 Hz, 1H), 7.83-7.79 (m, 2H), 6.85 (d, J=2.2 Hz, 1H), 3.94 (s, 3H), 3.71 (s, 8H), 3.50 (s, 3H). LCMS (ESI) m/z: 445.2 [M+H]⁺.

### Example 13. Synthesis of 4-(3-(5-methoxy-2-morpholino-6-(pyridin-4-ylamino)pyrimidin-4-yl)phenyl)-1-methylpiperazin-2-one (Compound 15)

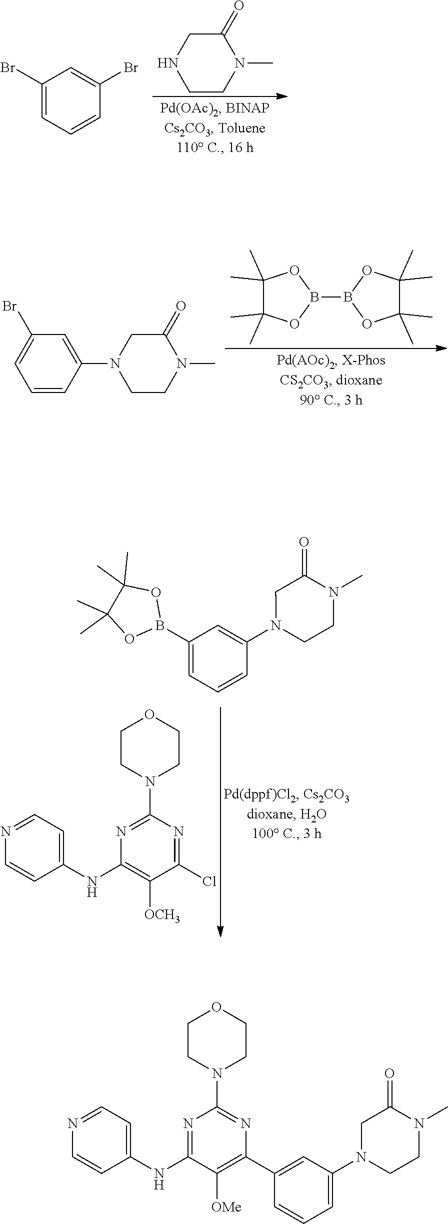

### Step 1: Synthesis of 4-(3-bromophenyl)-1-methylpiperazin-2-one

A mixture of 1-methylpiperazin-2-one (1 g, 8.76 mmol), 1,3-dibromobenzene (6.2 g, 26.28 mmol), cesium carbonate (11.42 g, 35.04 mmol), palladium (II) acetate (0.39 g, 1.75 mmol), and 1.1′-binaphthyl-2.2′-diphenyl phosphine (1.64 g, 2.63 mmol) in toluene (25 mL) was stirred at 100° C. for 16 h. The reaction mixture was concentrated, and the residue was subjected to silica gel column chromatography (petroleum ether:ethyl acetate=2:1) to obtain 4-(3-bromophenyl)-1-methylpiperazin-2-one (1.5 g, 64%) as yellow solid. LCMS (ESI) m/z: 269.1 [M+H]⁺.

### Step 2: Synthesis of 1-methyl-4-(3-(4,4,5,5-tetramethyl-1,3,2-dioxaborolan-2-yl)phenyl)piperazin-2-one

A mixture of 4-(3-bromophenyl)-1-methylpiperazin-2-one (0.4 g, 1.49 mmol), 4,4,4′,4′,5,5,5′,5′-octamethyl-2,2′-bi(1,3,2-dioxaborolane) (0.57 g, 2.23 mmol), potassium acetate (0.37 g, 3.72 mmol), tris(dibenzylideneacetone)dipalladium(0) (0.14 g, 0.15 mmol), and 2-(dicyclohexylphosphino)-2′,4′,6′-triisopropylbiphenyl (0.11 g, 0.23 mmol) in 1,4-dioxane (15 mL) was stirred at 85° C. for 4 h. The reaction mixture was then filtered, the filtrate was concentrated and the crude product thus obtained was purified by silica gel column chromatography (petroleum ether ethyl acetate=10:1) to obtain 1-methyl-4-(3-(4,4,5,5-tetramethyl-1,3,2-dioxaborolan-2-yl)phenyl)piperazin-2-one (0.3 g, 64%) as yellow solid. LCMS (ESI) m/z: 317.1 [M+H]⁺.

### Step 3: Synthesis of 4-(3-(5-methoxy-2-morpholino-6-(pyridin-4-ylamino)pyrimidin-4-yl)phenyl)-1-methylpiperazin-2-one

To a solution of 6-chloro-5-methoxy-2-morpholino-N-(pyridin-4-yl)pyrimidin-4-amine (0.1 g, 0.31 mmol) and 1-methyl-4-(3-(4,4,5,5-tetramethyl-1,3,2-dioxaborolan-2-yl)phenyl)piperazin-2-one (0.14 g, 0.44 mmol) in dioxane/water (5 mL/1 mL) were added cesium carbonate (0.2 g, 0.62 mmol) and [1,1′-bis(diphenylphosphino)ferrocene]dichloropalladium(II) (0.022 g, 0.03 mmol). The resultant mixture was stirred at 90° C. for 2 h and poured into ice-water. The aqueous medium was extracted with ethyl acetate (15 mL*3), the combined organic layer was washed with brine, dried and concentrated. The obtained crude product was purified by prep-HPLC (SunFire C18, 4.6*50 mm, 3.5 um column. The elution system used was a gradient of 5%-95% over 1.5 min at 2 ml/min and the solvent was acetonitrile/0.01% ammonium bicarbonate aqueous solution) to obtain 4-(3-(5-methoxy-2-morpholino-6-(pyridin-4-ylamino)pyrimidin-4-ylphenyl-1-methylpiperazin-2-one (51.3 mg, 35%) as white solid. ¹H NMR (400 MHz, DMSO-d₆) δ 9.34 (s, 1H), 8.41 (d, J=6.2 Hz, 2H), 7.88 (d, J=6.4 Hz, 2H), 7.56 (s, 1H), 7.47 (d, J=7.6 Hz, 1H), 7.37 (t, J=7.9 Hz, 1H), 7.09 (d, J=8.2 Hz, 1H), 3.80 (s, 2H), 3.69 (d, J=3.3 Hz, 8H), 3.54 (t, J=8 Hz, 2H), 3.46 (s, 2H), 3.45 (s, 3H), 2.91 (s, 3H). LCMS (ESI) m/z: 475.8 [M]⁺.

### Example 14. Synthesis of 5-methoxy-2-morpholino-N-(pyridin-4-yl)-6-(3-(pyrimidin-2-yl)phenyl)pyrimidin-4-amine (Compound 16)

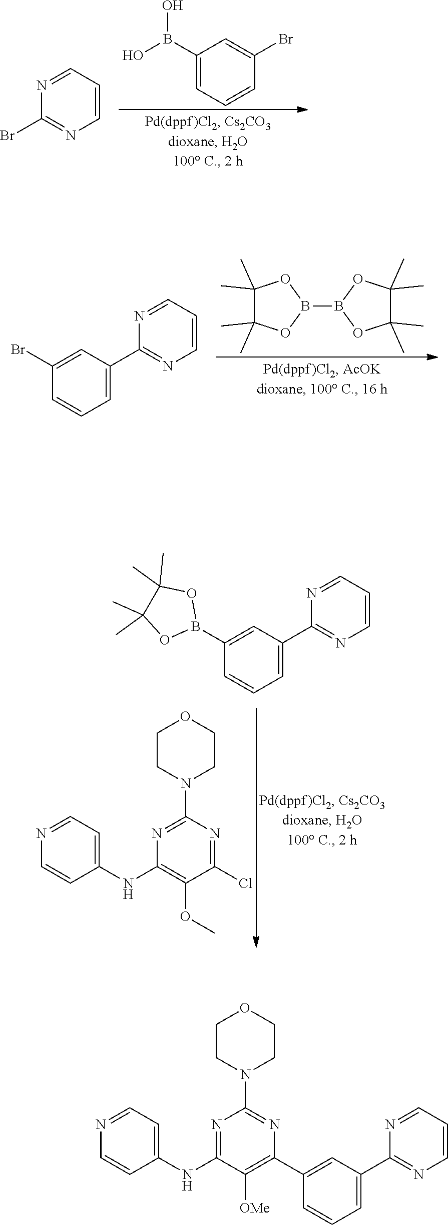

### Step 1: Synthesis of 2-(3-bromophenyl)pyrimidine

A solution of 2-bromopyrimidine (300 mg, 2.0 mmol), (3-bromophenyl)boronic acid (400 mg, 2.1 mmol), [1,1′-bis(diphenylphosphino)ferrocene]dichloropalladium(II) (240 mg, 0.28 mmol) and cesium carbonate (1960 mg, 6.0 mmol) in 1,4-dioxane (20 mL) and water (2 mL) was stirred at 100° C. for 2 h under nitrogen atmosphere. Then water (20 mL) was added and mixture was extracted with ethyl acetate (50 mL×3). The combined organic layer was dried and concentrated. The crude product thus obtained was purified by prep-TLC (petroleum ether ethyl acetate from 50:1 to 10:1) to obtain 2-(3-bromophenyl)pyrimidine (210 mg, 47%) as yellow oil. LCMS (ESI) m/z: 234.8 [M+H]⁺

### Step 2: Synthesis of 2-(3-(4,4,5,5-tetramethyl-1,3,2-dioxaborolan-2-yl)phenyl)pyrimidine

To a solution of 2-(3-bromophenyl)pyrimidine (200 mg, 0.9 mmol) in dioxane (20 mL) were added bis (pinacolato) diboron (300 mg, 1.2 mmol), [1′1-bis(diphenylphosphino)ferrocene]dichloro palladium(II) (82 mg, 0.09 mmol) and potassium acetate (877 mg, 2.7 mmol) at 25° C. The reaction mixture was stirred at 100° C. for 16 h under nitrogen atmosphere and concentrated. The resultant residue was subjected to silica gel column chromatography (2% methanol in dichloromethane) to obtain 2-(3-(4,4,5,5-tetramethyl-1,3,2-dioxaborolan-2-yl)phenyl)pyrimidine (180 mg, 71.0%) as white solid. LCMS (ESI) m/z: 283.2 [M+H]⁺.

### Step 3: Synthesis of 5-methoxy-2-morpholino-N-(pyridin-4-yl)-6-(3-(pyrimidin-2-yl)phenyl)pyrimidin-4-amine

A solution of 6-chloro-5-methoxy-2-morpholino-N-(pyridin-4-yl)pyrimidin-4-amine (100 mg, 0.28 mmol), 2-(3-(4,4,5,5-tetramethyl-1,3,2-dioxaborolan-2-yl)phenyl)pyrimidine (48 mg, 0.32 mmol), [1,1′-bis(diphenylphosphino)ferrocene]dichloropalladium(II) (20 mg, 0.0028 mmol) and cesium carbonate (209 mg, 0.84 mmol) in 1,4-dioxane (5 mL) and water (0.5 mL) was stirred at 100° C. for 2 h under nitrogen atmosphere. Water (20 mL) was added to the mixture and it was extracted with ethyl acetate (50 mL×3). The organic layer was dried, concentrated and the resultant crude product was purified by prep-TLC (petroleum ether ethyl acetate from 50:1 to 10:1) to obtain 5-methoxy-2-morpholino-N-(pyridin-4-yl)-6-(3-(pyrimidin-2-ylphenyl)pyrimidin-4-amine (13.4 mg, 11%) as white solid. ¹H NMR (400 MHz, DMSO-d₆) δ 9.40 (s, 1H), 9.13 (s, 1H), 8.95 (d, J=4.8 Hz, 2H), 8.52-8.42 (m, 3H), 8.16 (d, J=7.9 Hz, 1H), 7.90 (d, J=6.1 Hz, 2H), 7.68 (t, J=7.8 Hz, 1H), 7.49 (t, J=4.8 Hz, 1H), 3.72 (s, 8H), 3.47 (s, 3H). LCMS (ESI) m/z: 441.8 [M]⁺.

### Example 15. Synthesis of 5-methoxy-6-(5-(1-methyl-1H-pyrazol-3-yl)pyridazin-3-yl)-2-morpholino-N-(p-tolyl)pyrimidin-4-amine (Compound 17)

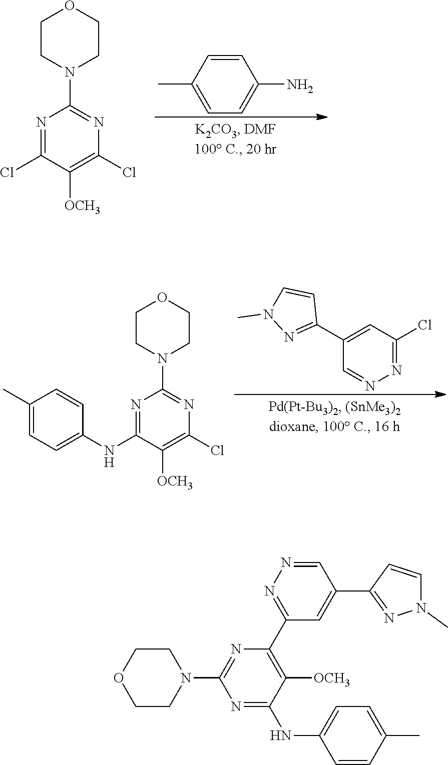

### Step 1: Synthesis of 6-chloro-5-methoxy-2-morpholino-N-(p-tolyl)pyrimidin-4-amine

To a solution of 4-(4,6-dichloro-5-methoxypyrimidin-2-yl)morpholine (530 mg, 2.0 mmol) in N,N-dimethylformamide (10 mL) were added p-toluidine (214 mg, 2.0 mmol) and potassium carbonate (556 mg, 4.0 mmol). The mixture was stirred at 100° C. for 20 h, then quenched with water (15 mL) and extracted with ethyl acetate (20 mL*3). The organic layer was combined, washed with brine (30 mL), dried over sodium sulfate, filtered and concentrated. The residue was purified by flash chromatography on silica gel (petroleum ether-ethyl acetate=75:25) to obtain 6-chloro-5-methoxy-2-morpholino-N-(p-tolyl)pyrimidin-4-amine as yellow solid. (340 mg, 50.5%). LCMS (ESI) m/z: 335.1 [M+H]⁺.

### Step 2: Synthesis of 5-methoxy-6(6-(1-methyl-H-pyrazol-3-yl)pyridazin-3-yl)-2-morpholino-N-(p-tolyl)pyrimidin-4-amine

A mixture of 6-chloro-5-methoxy-2-morpholino-N-(p-tolyl)pyrimidin-4-amine (0.1 g, 0.3 mmol), 3-chloro-5-(1-methyl-1H-pyrazol-3-yl)pyridazine (0.1 g, 0.51 mmol), hexamethyldistannane (0.17 g, 0.51 mmol) and bis(tri-tert-butylphosphine) palladium(0) (0.03 g, 0.06 mmol) in 1,4-dioxane (4 mL) was stirred at 100° C. under nitrogen atmosphere for 16 h. The mixture was concentrated and the residue was subjected to prep-HPLC (SunFire C18, 4.6*50 mm, 3.5 um column. The elution system used was a gradient of 5%-95% over 1.5 min at 2 ml/min and the solvent was acetonitrile/0.01% ammonium bicarbonate aqueous solution) to obtain 5-methoxy-6-(5-(1-methyl-1H-pyrazol-3-yl)pyridazin-3-yl)-2-morpholino-N-(p-tolyl)pyrimidin-4-amine as yellow solid (3.4 mg, 2.5%). ¹H NMR (400 MHz, DMSO-d₆) δ 9.64 (d, J=2.1 Hz, 1H), 8.45 (d, J=2.1 Hz, 1H), 7.76 (d, J=2.4 Hz, 1H), 7.60 (d, J=8.4 Hz, 2H), 7.16 (d, J=8.3 Hz, 2H), 7.01 (d, J=2.4 Hz, 1H), 4.02 (s, 3H), 3.75 (s, 8H), 3.63 (s, 3H), 2.33 (s, 3H). LCMS (ESI) m/z: 459.1 [M+H]⁺.

### Example 16. Synthesis of 6-(5,6-dimethoxypyridin-3-yl)-5-methoxy-2-morpholino-N-(pyridin-3-yl)pyrimidin-4-amine (Compound 18)

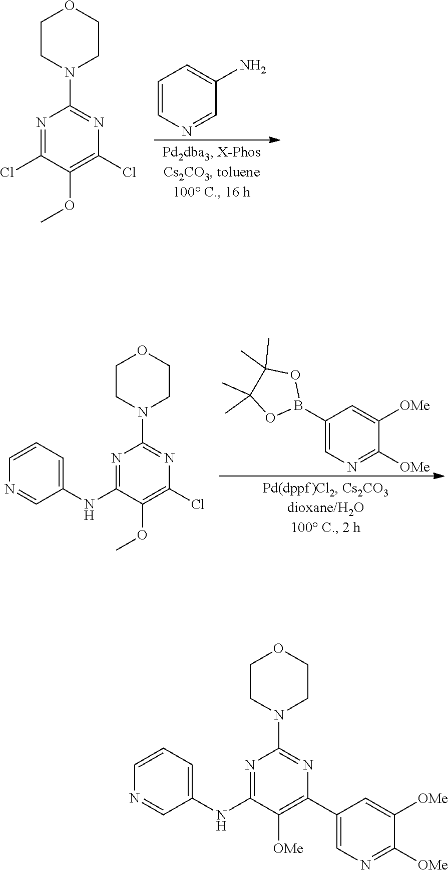 [SMILES: COc1c(Cl)nc(N2CCOCC2)nc1Cl ; Nc1cccnc1 ; COc1c(Cl)nc(N2CCOCC2)nc1Nc1cccnc1 ; COc1cc(B2OC(C)(C)C(C)(C)O2)cnc1OC ; COc1cc(-c2nc(N3CCOCC3)nc(Nc3cccnc3)c2OC)cnc1OC]

### Step 1: Synthesis of 6-chloro-5-methoxy-2-morpholino-N-(pyridin-3-yl)pyrimidin-4-amine

A mixture of 4-(4,6-dichloro-5-methoxypyrimidin-2-yl)morpholine (0.5 g, 1.89 mmol), pyridin-3-amine (0.16 g, 1.7 mmol), tris(dibenzylideneacetone)dipalladium (0.17 g, 0.19 mmol), 2-(dicyclohexylphosphino)-2′,4′,6′-triisopropylbiphenyl (0.18 g, 0.38 mmol) and cesium carbonate (1.54 g, 4.73 mmol) in toluene (25 mL) was stirred at 100° C. for 16 h under nitrogen atmosphere. The reaction mixture was concentrated, the residue was diluted with water (20 mL) and extracted with dichloromethane (20 mL×2). The combined organic phase was washed with brine (20 mL), dried over sodium sulfate, filtered and concentrated. The obtained residue was subjected to flash chromatography (eluted with petroleum ether in ethyl acetate from 30% to 60%) to afford 6-chloro-5-methoxy-2-morpholino-N-(pyridin-3-yl)pyrimidin-4-amine (0.2 g, 36.7%) as white solid. LCMS (ESI) m/z: 321.8 [M]⁺.

### Step 2: Synthesis of 6-(5,6-dimethoxypyridin-3-yl)-5-methoxy-2-morpholino-N-(pyridin-3-yl)pyrimidin-4-amine

A mixture of 6-chloro-5-methoxy-2-morpholino-N-(pyridin-3-yl)pyrimidin-4-amine (80 mg, 0.25 mmol), 2,3-dimethoxy-5-(4,4,5,5-tetramethyl-1,3,2-dioxaborolan-2-yl)pyridine (79.5 mg, 0.3 mmol), 1,1′-bis(diphenylphosphino)ferrocene-palladium(II)dichloride dichloromethane complex (20 mg, 0.025 mmol) and cesium carbonate (0.2 g, 0.62 mmol) in 1,4-dioxane/water (15 mL/3 mL) was stirred at 100° C. for 16 h under nitrogen atmosphere. The reaction mixture was concentrated, the residue was diluted with water (20 mL) and the mixture was extracted with ethyl acetate (20 mL×2). The combined organic phase was washed with brine (20 mL), dried over sodium sulfate, filtered and concentrated. The resultant residue was subjected to prep-HPLC to afford 6-(5,6-dimethoxypyridin-3-yl-5-methoxy-2-morpholino-N-(pyridin-3-ylpyrimidin-4-amine (27.6 mg, 26%) as white solid. ¹H NMR (400 MHz, DMSO-d₆) δ 9.17 (s, 1H), 8.98 (d, J=2.6 Hz, 1H), 8.47 (d, J=1.8 Hz, 1H), 8.22 (d, J=4.7 Hz, 1H), 8.18 (d, J=9.2 Hz, 1H), 7.89 (d, J=1.8 Hz, 1H), 7.36 (dd, J=8.3, 4.6 Hz, 1H), 3.94 (s, 3H), 3.86 (s, 3H), 3.66 (d, J=5.8 Hz, 8H), 3.54 (s, 3H). LCMS (ESI) m/z: 424.8 [M]⁺.

### Example 17. Synthesis of Compounds 19-24

The following compounds were synthesized according to the protocol described above in Example 16: 5-methoxy-6-(5-(1-methyl-1H-pyrazol-3-yl)pyridin-3-yl)-2-morpholino-N-(pyridin-3-yl)pyrimidin-4-amine (Compound 19), 5-methoxy-6-(3-(1-methyl-1H-pyrazol-3-yl)phenyl)-2-morpholino-N-(pyridin-3-yl)pyrimidin-4-amine (Compound 20), 5-methoxy-2-morpholino-6-(3-(pyridazin-3-yl)phenyl)-N-(pyridin-3-ylpyrimidin-4-amine (Compound 21), 6-(3,4-dimethoxyphenyl)-5-methoxy-2-morpholino-N-(pyridin-3-ylpyrimidin-4-amine (Compound 22), 3-(5-methoxy-2-morpholino-6-(pyridin-3-ylamino)pyrimidin-4-yl)benzonitrile (Compound 23), and 3-(5-methoxy-2-morpholino-6-(pyridin-3-ylamino)pyrimidin-4-yl-N,N-dimethylbenzamide (Compound 24).

| No. | Structure | NMR, MS |
|---|---|---|
| 19 | 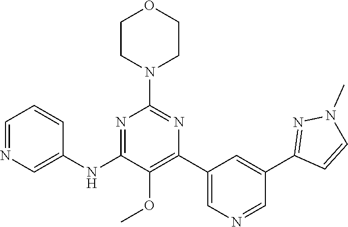 [SMILES: COc1c(Nc2cccnc2)nc(N2CCOCC2)nc1-c1cncc(-c2ccn(C)n2)c1] | ¹H NMR (400 MHz, DMSO-d₆) δ 9.25 (s, 1H), 9.06 (d, J = 1.6 Hz, 2H), 8.99 (d, J = 2.3 Hz, 1H), 8.69 (t, J = 2.1 Hz, 1H), 8.30-8.22 (m, 1H), 8.21-8.16 (m, 1H), 7.83 (d, J = 2.2 Hz, 1H), 7.38 (dd, J = 8.3, 4.7 Hz, 1H), 6.87 (d, J = 2.3 Hz, 1H), 3.93 (s, 3H), 3.67 (d, J = 4 Hz, 8H), 3.51 (s, 3H). LCMS (ESI) m/z: 445.2 [M + H]⁺. |
| 20 | 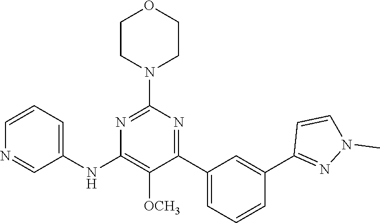 [SMILES: COc1c(Nc2cccnc2)nc(N2CCOCC2)nc1-c1cccc(-c2ccn(C)n2)c1] | ¹H NMR (400 MHz, DMSO-d₆) δ 9.18 (s, 1H), 9.00 (d, J = 2.3 Hz, 1H), 8.44 (s, 1H), 8.22 (t, J = 6.3 Hz, 2H), 7.94-7.82 (m, 2H), 7.77 (d, J = 2.2 Hz, 1H), 7.51 (t, J = 7.7 Hz, 1H), 7.38 (dd, J = 8.2, 4.6 Hz, 1H), 6.71 (d, J = 2.2 Hz, 1H), 3.90 (s, 3H), 3.67 (d, J = 6.1 Hz, 8H), 3.47 (s, 3H).). LCMS (ESI) m/z: 443.8 [M]⁺. |
| 21 | 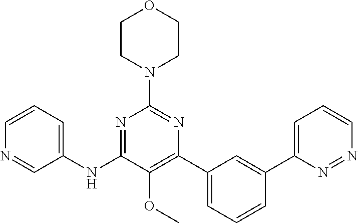 [SMILES: COc1c(Nc2cccnc2)nc(N2CCOCC2)nc1-c1cccc(-c2cccnn2)c1] | ¹H NMR (400 MHz, DMSO-d₆) δ 9.26 (dd, J = 4.9, 1.4 Hz, 1H), 9.19 (s, 1H), 9.00 (d, J = 2.4 Hz, 1H), 8.80 (s, 1H), 8.27 (dd, J = 8.7, 1.4 Hz, 1H), 8.25-8.15 (m, 4H), 7.83 (dd, J = 8.6, 4.9 Hz, 1H), 7.70 (t, J = 7.8 Hz, 1H), 7.38 (dd, J = 8.3, 4.7 Hz, 1H), 3.67 (d, J = 2.7 Hz, 8H), 3.51 (s, 3H).LCMS (ESI) m/z: 441.8 [M]⁺. |
| 22 | 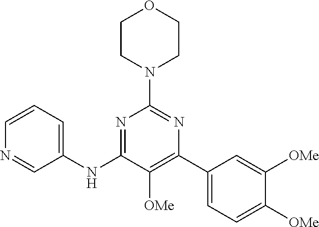 [SMILES: COc1ccc(-c2nc(N3CCOCC3)nc(Nc3cccnc3)c2OC)cc1OC] | ¹H NMR (400 MHz, DMSO-d₆) δ 9.11 (s, 1H), 8.99 (d, J = 2.2 Hz, 1H), 8.26-8.17 (m, 2H), 7.73 (dd, J = 4.3, 2.5 Hz, 2H), 7.37 (dd, J = 8.2, 4.7 Hz, 1H), 7.08 (d, J = 9.1 Hz, 1H), 3.83 (s, 3H), 3.81 (s, 3H), 3.66 (d, J = 8.1 Hz, 8H), 3.50 (s, 3H). LCMS (ESI) m/z: 423.8 [M]⁺. |
| 23 | 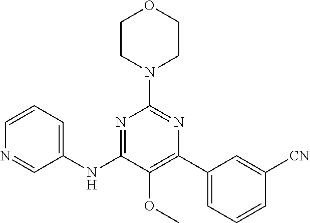 [SMILES: COc1c(Nc2cccnc2)nc(N2CCOCC2)nc1-c1cccc(C#N)c1] | ¹H NMR (400 MHz, DMSO-d₆) δ 9.24 (s, 1H), 8.98 (d, J = 2.4 Hz, 1H), 8.37 (d, J = 11.1 Hz, 2H), 8.24 (d, J = 3.5 Hz, 1H), 8.18 (d, J = 8.1 Hz, 1H), 7.96 (d, J = 7.7 Hz, 1H), 7.73 (t, J = 7.8 Hz, 1H), 7.37 (dd, J = 8.3, 4.7 Hz, 1H), 3.66 (d, J = 4.3 Hz, 8H), 3.49 (s, 3H). LCMS (ESI) m/z: 389.1 [M + H]⁺. |
| 24 | 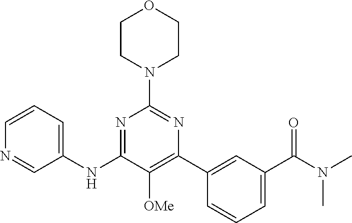 [SMILES: COc1c(Nc2cccnc2)nc(N2CCOCC2)nc1-c1cccc(C(=O)N(C)C)c1] | ¹H NMR (400 MHz, DMSO-d₆) δ 9.18 (s, 1H), 8.98 (d, J = 2.2 Hz, 1H), 8.23 (d, J = 4.5 Hz, 1H), 8.21-8.14 (m, 1H), 8.09 (dd, J = 8.0, 4.0Hz, 1H), 8.02 (d, J = 1.5 Hz, 1H), 7.57 (t, J = 7.7 Hz, 1H), 7.50 (dt, J = 7.6, 1.4 Hz, 1H), 7.37 (dd, J = 8.3, 4.7 Hz, 1H), 3.65 (d, J = 7.5 Hz, 8H), 3.47 (s, 3H), 3.02 (s, 3H), 2.97 (s, 3H). LCMS (ESI) m/z: 434.9 [M]⁺ |

### Example 18. Synthesis of 5-methoxy-6-(2-(1-methyl-1H-pyrazol-3-yl)pyridin-4-yl)-2-morpholino-N-(pyridin-3-yl)pyrimidin-4-amine (Compound 25)

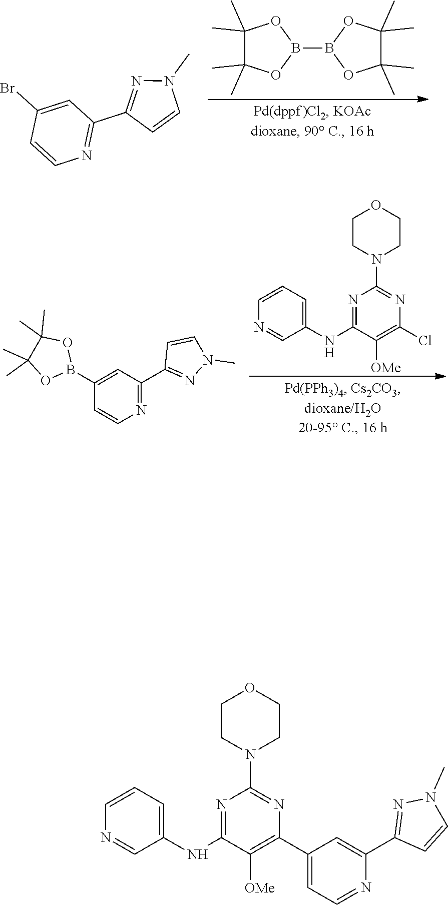

### Step 1: Synthesis of 2-(1-methyl-1H-pyrazol-3-yl)-4-(4,4,5,5-tetramethyl-1,3,2-dioxaborolan-2-yl)pyridine

A mixture of 4-bromo-2-(1-methyl-1H-pyrazol-3-yl)pyridine (71 mg, 0.3 mmol), 4,4,4′,4′,5,5,5′,5′-octamethyl-2,2′-bi(1,3,2-dioxaborolane (91 mg, 0.36 mmol), [1,1′-bis(diphenylphosphino) ferrocene]dichloropalladium(II) dichloromethane complex (24 mg, 0.03 mmol) and potassium acetate (88 mg, 0.9 mmol) in 1,4-dioxane (8 mL) was stirred at 90° C. for 16 h. The reaction mixture was cooled down and directly used in the next step. LCMS (ESI) m/z: 279.1 [M−4]⁺.

### Step 2: Synthesis of 5-methoxy-6-(2-(1-methyl-1H-pyrazol-3-yl)pyridin-4-yl)-2-morpholino-N-(pyridin-3-yl)pyrimidin-4-amine

To the mixture from step 1, were added 6-chloro-5-methoxy-2-morpholino-N-(pyridin-3-yl)pyrimidin-4-amine (96 mg, 0.3 mmol), tetrakis(triphenylphosphin)palladium (35 mg, 0.03 mmol), cesium carbonate (293 mg, 0.9 mmol) and water (1 mL) and the resultant mixture was stirred at 95° C. for 16 h under argon atmosphere. It was then concentrated and subjected to prep-HPLC (BOSTON pHlex ODS 10 um 21.2×250 mm 120 A. The mobile phase was acetonitrile/0.1% ammonium bicarbonate) to obtain 5-methoxy-6-(2-(1-methyl-1H-pyrazol-3-yl)pyridin-4-yl)-2-morpholino-N-(pyridin-3-yl)pyrimidin-4-amine (34.6 mg, 19%) as white solid. ¹H NMR (400 MHz, DMSO-d₆) δ 9.27 (s, 1H), 8.99 (d, J=2.4 Hz, 1H), 8.69 (d, J=5.2 Hz, 1H), 8.52 (s, 1H), 8.25 (dd, J=4.6, 1.3 Hz, 1H), 8.20 (ddd, J=8.4, 2.3, 1.5 Hz, 1H), 7.82 (dd, J=5.2, 1.6 Hz, 1H), 7.80 (d, J=2.2 Hz, 1H), 7.39 (dd, J=8.3, 4.7 Hz, 1H), 6.85 (d, J=2.2 Hz, 1H), 3.94 (s, 3H), 3.67 (d, J=4.5 Hz, 8H), 3.52 (s, 3H). LCMS (ESI) m/z: 444.8 [M]⁺.

### Example 19. Synthesis of 1-(3-(5-methoxy-2-morpholino-6-(pyridin-3-ylamino)pyrimidin-4-yl)phenyl)pyrrolidin-3-ol (Compound 26)

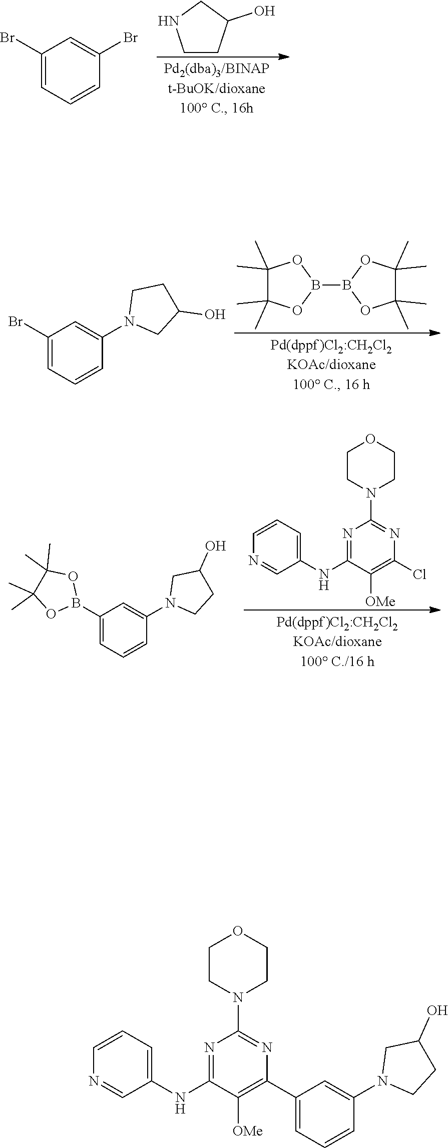

### Step 1: Synthesis of 1-(3-bromophenyl)pyrrolidin-3-ol

A mixture of 1,3-dibromobenzene (2.34 g, 10.0 mmol), pyrrolidin-3-ol (870 mg, 10.0 mmol), tris(dibenzylideneacetone)-dipalladium(0) (458 mg, 0.5 mmol), 1.1′-binaphthyl-2.2′-diphemyl phosphine (622 mg, 1.0 mmol) and potassium tert-butoxide (2.24 g, 20.0 mmol) in 1.4-dioxane (40 mL) was stirred at 100° C. under nitrogen atmosphere for 16 h. The mixture was then poured into water, extracted with dichloromethane (200 mL*2). The combined organic phase was concentrated and the residue obtained was subjected to silica gel column chromatography (70% of ethyl acetate in petroleum ether) to afford 1-(3-bromophenyl)pyrrolidin-3-ol (1.3 g, 46%) as grey solid. LCMS (ESI) m/z: 241.6/243.8 [M+H]⁺.

### Step 2: Synthesis of 1-(3-(4,4,5,5-tetramethyl-1,3,2-dioxaborolan-2-yl)phenyl)pyrrolidin-3-ol

A mixture of 1-(3-bromophenyl)pyrrolidin-3-ol (1.2 g, 5.0 mmol), 4,4,4′,4′,5,5,5′,5′-octamethyl-2,2′-bi(1,3,2-dioxaborolane) (2.54 g, 10.0 mmol), 1,1′-bis(diphenylphosphino)ferrocene-palladium(II)dichloride dichloromethane complex (408 mg, 0.5 mmol) and potassium acetate (980 mg, 10.0 mmol) in 1,4-dioxane (40 mL) was stirred at 100° C. under nitrogen atmosphere for 16 h. The resultant mixture was poured into water and extracted with ethyl acetate (150 mL*2). The combined organic phase was concentrated and subjected to silica gel column chromatography (60% ethyl acetate in petroleum ether) to obtain 1-(3-(4,4,5,5-tetramethyl-1,3,2-dioxaborolan-2-yl)phenyl)pyrrolidin-3-ol (1.4 g, 97%) as brown oil. LCMS (ESI) m/z: 289.9 [M]⁺.

### Step 3: Synthesis of 1-(3-(5-methoxy-2-morpholino-6-(pyridin-3-ylamino)pyrimidin-4-yl)phenyl)pyrrolidin-3-ol

A mixture of 6-chloro-5-methoxy-2-morpholino-N-(pyridin-3-yl)pyrimidin-4-amine (100 mg, 0.38 mmol), 1-(3-(4,4,5,5-tetramethyl-1,3,2-dioxaborolan-2-yl)phenyl)pyrrolidin-3-ol (136 mg, 0.47 mmol), 1,1′-bis(diphenylphosphino)ferrocene-palladium(II)dichloride dichloromethane complex (25 mg, 0.03 mmol) and cesium carbonate (201 mg, 0.62 mmol) in dioxane (5 mL) and water (0.5 mL) was stirred at 100° C. under nitrogen atmosphere for 16 h. The mixture was then poured into water and extracted with ethyl acetate (100 mL*2). The combined organic phase was concentrated and subjected to silica gel column chromatography (15% methanol in dichloromethane) and then to prep-HPLC (Column Xbridge 21.2*250 mm C18, 10 um, Mobile Phase A: water (10 mmol/L ammonium bicarbonate) B: acetonitrile) to obtain 1-(3-(5-methoxy-2-morpholino-6-(pyridin-3-ylamino)pyrimidin-4-yl)phenyl)pyrrolidin-3-ol (60.3 mg, 42%) as off-white solid. ¹H NMR (400 MHz, DMSO-d₆) δ 9.11 (s, 1H), 8.99 (d, J=1.9 Hz, 1H), 8.20 (t, J=5.9 Hz, 2H), 7.36 (dd, J=8.2, 4.7 Hz, 1H), 7.26 (d, J=5.5 Hz, 2H), 7.19 (s, 1H), 6.60 (d, J=3.4 Hz, 1H), 4.97 (d, J=3.6 Hz, 1H), 4.43 (s, 1H), 3.65 (d, J=9.8 Hz, 8H), 3.49-3.42 (m, 4H), 3.37 (d, J=7.5 Hz, 2H), 3.11 (d, J=9.1 Hz, 1H), 2.10-2.03 (m, 1H), 1.93 (d, J=3.5 Hz, 1H); LCMS (ESI) m/z: 448.8 [M]⁺.

### Example 20. Synthesis of 4-(3-(5-methoxy-2-morpholino-6-(pyridin-3-ylamino)pyrimidin-4-yl)phenyl)-1-methylpiperazin-2-one (Compound 27)

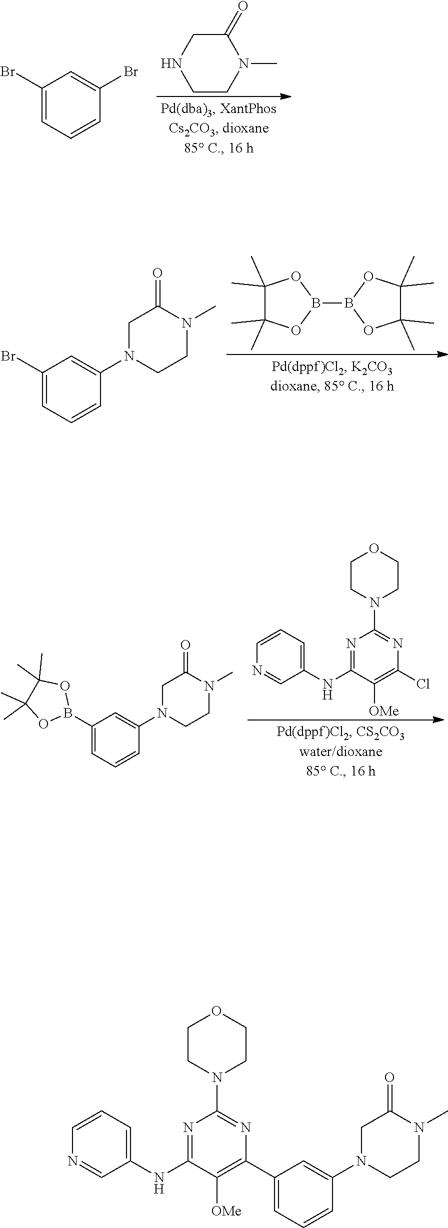

### Step 1: Synthesis of 4-(3-bromophenyl)-1-methylpiperazin-2-one

A mixture of 1,3-dibromobenzene (1000 mg, 4.24 mmol), 1-methylpiperazin-2-one (320.2 mg, 2.82 mmol), tris(dibenzylideneacetone)dipalladium (250 mg, 0.28 mmol), 2-dicyclohexyl phosphino-2′,6′-diisopropoxybiphenyl (260 mg, 0.56 mmol) and cesium carbonate (2700 mg, 8.46 mmol) in 1,4-dioxane (10 mL) was stirred at 85° C. under argon atmosphere for 16 h. The mixture was then filtered and the filtrate was concentrated. The residue was subjected to prep-TLC (dichloromethane/methanol=10/1) to obtain 4-(3-bromophenyl)-1-methylpiperazin-2-one (550 mg, 50%) as white solid. LCMS (ESI) m/z: 271.0 [M+H]⁺.

### Step 2: Synthesis of 1-methyl-4-(3-(4,4,5,5-tetramethyl-1,3,2-dioxaborolan-2-yl)phenyl)piperazin-2-one

A mixture of 4-(3-bromophenyl)-1-methylpiperazin-2-one (230 mg, 0.85 mmol), 4,4,4′,4′,5,5,5′,5′-octamethyl-2,2′-bi(1,3,2-dioxaborolane) (324 mg, 1.28 mmol), [1,1′-bis (diphenylphosphino)ferrocene]dichloropalladium(II) (58 mg, 0.08 mmol), and potassium carbonate (351 mg, 2.55 mmol) in 1,4-dioxane (5 mL) was stirred at 85° C. under argon atmosphere for 16 h. The resultant mixture was filtered, and the filtrate was concentrated. The residue was subjected to prep-TLC (dichloromethane/methanol=10/1) to obtain 1-methyl-4-(3-(4,4,5,5-tetramethyl-1,3,2-dioxaborolan-2-yl)phenyl)piperazin-2-one (150 mg, 55%) as white solid. LCMS (ESI) m/z: 317.2 [M+H]⁺.

### Step 3: Synthesis of 4-(3-(5-methoxy-2-morpholino-6-(pyridin-3-ylamino)pyrimidin-4-yl)phenyl)-1-methylpiperazin-2-one

A mixture of 1-methyl-4-(3-(4,4,5,5-tetramethyl-1,3,2-dioxaborolan-2-yl)phenyl)piperazin-2-one (95 mg, 0.3 mmol), 6-chloro-5-methoxy-2-morpholino-N-(pyridin-3-yl)pyrimidin-4-amine (100 mg, 0.3 mmol), [1,1′-bis(diphenylphosphino)ferrocene]dichloropalladium(II) (21 mg, 0.03 mmol) and cesium carbonate (195 mg, 0.6 mmol) in 1,4-dioxane (5 mL) and water (0.5 mL) was stirred at 85° C. for 16 h under argon atmosphere. Water (50 mL) was added to the reaction mixture and it was extracted with ethyl acetate (50 mL×3). The combined organic layer was dried and concentrated. The residue obtained was subjected to prep-HPLC to obtain 4-(3-(5-methoxy-2-morpholino-6-(pyridin-3-ylamino)pyrimidin-4-yl)phenyl)-1-methylpiperazin-2-one (18 mg, 13%) as white solid. ¹H NMR (400 MHz, DMSO-d₆) δ 9.14 (s, 1H), 9.00 (s, 1H), 8.20 (d, J=9.4 Hz, 2H), 7.56 (s, 1H), 7.47 (d, J=7.2 Hz, 1H), 7.36 (t, J=7.8 Hz, 2H), 7.08 (d, J=7.8 Hz, 1H), 3.80 (s, 2H), 3.65 (d, J=8.9 Hz, 8H), 3.53 (d, J=5.7 Hz, 2H), 3.46 (s, 5H), 2.91 (s, 3H). LCMS (ESI) m/z: 475.8 [M]⁺.

### Example 21. Synthesis of 4-(3-(5-methoxy-2-morpholino-6-(pyridin-3-ylamino)pyrimidin-4-yl)phenyl)morpholin-3-one (Compound 28)

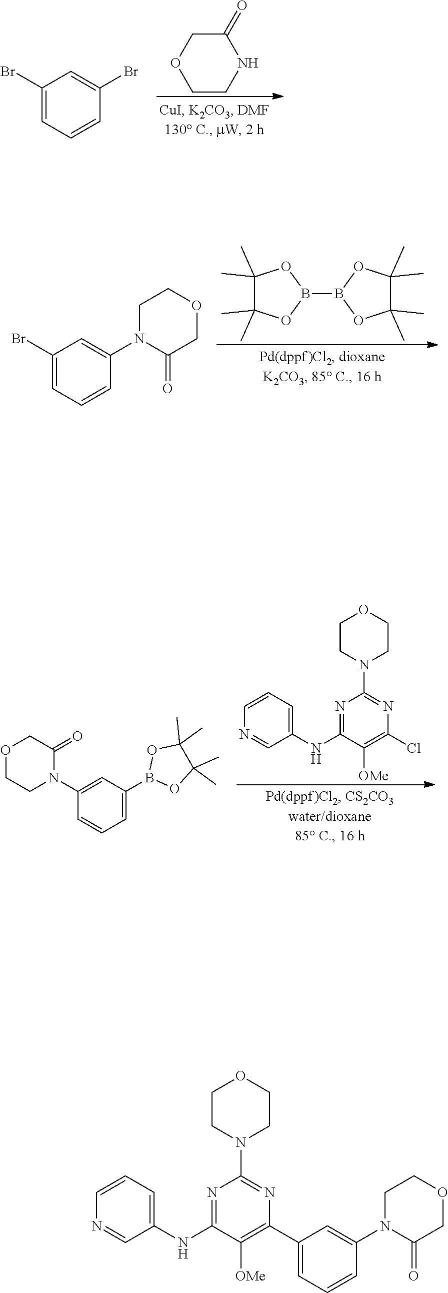

### Step 1: Synthesis of 4-(3-bromophenyl)morpholin-3-one

A mixture of 1,3-dibromobenzene (1.4 g, 5.94 mmol), morpholin-3-one (500 mg, 4.95 mmol), cuprous iodide (95 mg, 0.5 mmol) and potassium carbonate (1.4 g, 9.9 mmol) in DMF (8 mL) was stirred at 130° C. under microwave irradiation for 2 h. The resultant mixture was filtered, and the filtrate was concentrated. The residue was then subjected to prep-TLC (dichloromethane/methanol=10/1) to obtain 4-(3-bromophenyl)morpholin-3-one (800 mg, 53%) as colorless oil. LCMS (ESI) m/z: 258.0 [M+H]⁺.

### Step 2: Synthesis of 4-(3-(4,4,5,5-tetramethyl-1,3,2-dioxaborolan-2-yl)phenyl)morpholin-3-one

A mixture of 4-(3-bromophenyl)morpholin-3-one (330 mg, 1.28 mmol), 4,4,4′,4′,5,5,5′,5′-octamethyl-2,2′-bi(1,3,2-dioxaborolane) (489 mg, 1.93 mmol), [1,1′-bis(diphenylphosphino) ferrocene]dichloropalladium(II) (95 mg, 0.13 mmol), and potassium carbonate (529 mg, 3.84 mmol) in dioxane (5 mL) was stirred at 85° C. under argon atmosphere for 16 h. The mixture was filtered, and the filtrate was concentrated. The residue was subjected to prep-TLC (dichloromethane/methanol=10/1) to obtain 4-(3-(4,4,5,5-tetramethyl-1,3,2-dioxaborolan-2-yl)phenyl)morpholin-3-one (250 mg, 64%) as white solid. LCMS (ESI) m/z: 304.3 [M+H]⁺.

### Step 3: Synthesis of 4-(3-(5-methoxy-2-morpholino-6-(pyridin-3-ylamino)pyrimidin-4-yl)phenyl)morpholin-3-one

A mixture of 4-(3-(4,4,5,5-tetramethyl-1,3,2-dioxaborolan-2-yl)phenyl)morpholin-3-one (91 mg, 0.3 mmol), 8-chloro-5-methoxy-2-morpholino-N-(pyridin-3-yl)pyrimidin-4-amine (100 mg, 0.3 mmol), [1,1′-bis(diphenylphosphino)ferrocene]dichloropalladium(II) (21 mg, 0.03 mmol) and cesium carbonate (195 mg, 0.6 mmol) in dioxane (5 mL) and water (0.5 mL) was stirred at 85° C. for 16 h under argon atmosphere. Water (50 mL) was added to the reaction mixture and it was extracted with ethyl acetate (50 mL×3). The combined organic layer was dried and concentrated. The crude product thus obtained was purified by prep-HPLC to obtain 4-(3-(5-methoxy-2-morpholino-6-(pyridin-3-ylamino)pyrimidin-4-yl)phenyl)morpholin-3-one (32 mg, 23%) as white solid. ¹H NMR (400 MHz, DMSO-d₆) δ 9.15 (s, 1H), 8.98 (d, J=2.3 Hz, 1H), 8.20 (dd, J=14.7, 6.9 Hz, 2H), 8.05 (s, 1H), 7.94 (d, J=7.4 Hz, 1H), 7.57-7.47 (m, 2H), 7.37 (dd, J=8.3, 4.6 Hz, 1H), 4.24 (s, 2H), 4.05-3.98 (m, 2H), 3.83-3.78 (m, 2H), 3.65 (d, J=7.6 Hz, 8H), 3.50 (s, 3H). LCMS (ESI) m/z: 462.8 [M]⁺.

### Example 22. Synthesis of 1-(3-(5-methoxy-2-morpholino-6-(pyridin-3-ylamino)pyrimidin-4-yl)phenyl)pyrrolidin-2-one (Compound 29)

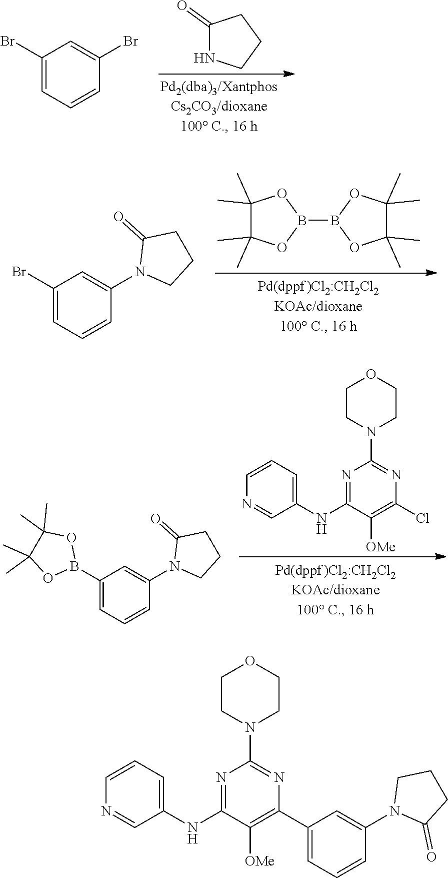

### Step 1: Synthesis of 1-(3-bromophenyl)pyrrolidin-2-one

A mixture of 1,3-dibromobenzene (2.34 g, 10.0 mmol), pyrrolidin-2-one (850 mg, 10.0 mmol), palladium acetate (112 mg, 0.5 mmol), 4,5-bis(diphenylphosphino)-9,9-dimethylxanthene (578 mg, 1.0 mmol) and cesium carbonate (6.5 g, 20.0 mmol) in dioxane (40 mL) was stirred at 100° C. under nitrogen atmosphere for 16 h. The resultant mixture was poured into water and extracted with dichloromethane (200 mL*2). The combined organic phase was concentrated and the residue was subjected to silica gel column chromatography (70% of ethyl acetate in petroleum ether) to obtain 1-(3-bromophenyl)pyrrolidin-2-one (1.1 g, 46%) as grey solid. LCMS (ESI) m/z: 240.8/242.8 [M+H]⁺.

### Step 2: Synthesis of 1-(3-(4,4,5,5-tetramethyl-1,3,2-dioxaborolan-2-yl)phenyl)pyrrolidin-2-one

A mixture of 1-(3-bromophenyl)pyrrolidin-2-one (1.0 g, 4.1 mmol), 4,4,4′,4′,5,5,5′,5′-octamethyl-2,2′-bi(1,3,2-dioxaborolane) (2.0 g, 8.2 mmol), 1,1′-bis(diphenylphosphino)ferrocene-palladium (II)dichloride dichloromethane complex (334 mg, 0.41 mmol) and potassium acetate (804 mg, 8.2 mmol) in dioxane (40 mL) was stirred at 100° C. under nitrogen atmosphere for 16 h. The resultant mixture was poured into water and extracted with ethyl acetate (150 mL*2). The combined organic phase was concentrated and the residue was subjected to silica gel column chromatography (50% ethyl acetate in petroleum ether) to obtain 1-(3-(4,4,5,5-tetramethyl-1,3,2-dioxaborolan-2-yl)phenyl)pyrrolidin-2-one (1.2 g) as light yellow solid. LCMS (ESI) m/z: 287.9 [M+H]⁺.

### Step 3: Synthesis of 1-(3-(5-methoxy-2-morpholino-6-(pyridin-3-ylamino)pyrimidin-4-yl)phenyl)pyrrolidin-2-one

A mixture of 6-chloro-5-methoxy-2-morpholino-N-(pyridin-3-yl)pyrimidin-4-amine (100 mg, 0.38 mmol), 1-(3-(4,4,5,5-tetramethyl-1,3,2-dioxaborolan-2-yl)phenyl)pyrrolidin-2-one (135 mg, 0.47 mmol), 1,1′-bis(diphenylphosphino)ferrocene-palladium(II)dichloride dichloromethane complex (25 mg, 0.03 mmol) and cesium carbonate (201 mg, 0.62 mmol), dioxane (5 mL) and water (0.5 mL) was stirred at 100° C. under nitrogen atmosphere for 16 h. The resultant mixture was poured into water and extracted with ethyl acetate (100 mL*2). The combined organic phase was concentrated and residue was subjected first to silica gel column chromatography (15% methanol in dichloromethane) and then to prep-HPLC (Column Xbridge 21.2*250 mm C18, 10 um, Mobile Phase A: water (10 mmol/L ammonium bicarbonate) B: acetonitrile) to obtain 1-(3-(5-methoxy-2-morpholino-6-(pyridin-3-ylamino)pyrimidin-4-yl)phenyl)pyrrolidin-2-one (36.4 mg, 26.1%) as grey solid. ¹H NMR (400 MHz, DMSO-d₆) δ 9.17 (s, 1H), 8.99 (d, J=2.1 Hz, 1H), 8.40 (s, 1H), 8.25-8.17 (m, 2H), 7.80 (d, J=7.7 Hz, 1H), 7.72 (d, J=7.6 Hz, 1H), 7.49 (t, J=8.0 Hz, 1H), 7.37 (dd, J=8.3, 4.7 Hz, 1H), 3.89 (t, J=7.0 Hz, 2H), 3.65 (d, J=7.8 Hz, 8H), 3.49 (s, 3H), 2.54 (d, J=8.0 Hz, 2H), 2.13-2.05 (m, 2H); LCMS (ESI) m/z: 446.9 [M+H]⁺.

### Example 23. Synthesis of 4-(5-methoxy-4-(3-(1-methyl-1H-pyrazol-3-yl)phenyl)-6-(pyridin-4-yl)pyrimidin-2-yl)morpholine (Compound 30)

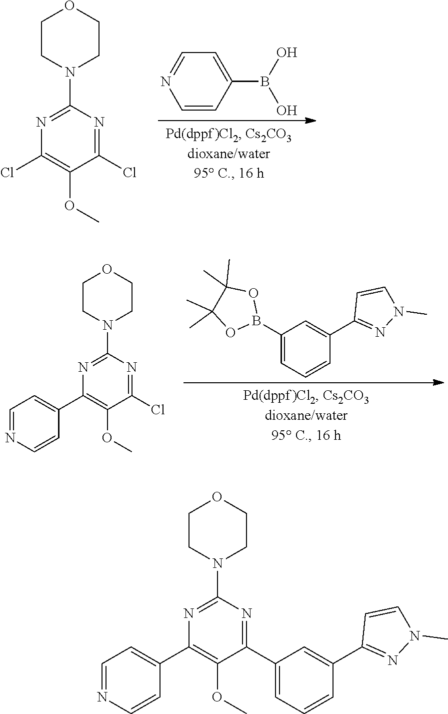

### Step 1: Synthesis of 4-(4-chloro-5-methoxy-6-(pyridin-4-yl)pyrimidin-2-yl)morpholine

A mixture of 4-(4,6-dichloro-5-methoxypyrimidin-2-yl)morpholine (0.45 g, 1.70 mmol), pyridin-4-ylboronic acid (0.21 g, 1.70 mmol), 1,1′-bis(diphenylphosphino)ferrocene-palladium(II) dichloride dichloromethane complex (0.14 g, 0.17 mmol), and cesium carbonate (1.39 g, 4.26 mmol) in 1,4-dioxane/water (20 mL/3 mL) was stirred at 95° C. for 16 h under nitrogen atmosphere. It was concentrated and the residue was diluted with water (20 mL) and extracted with dichloromethane (20 mL×2). The combined organic phase was washed with brine (20 mL), dried over sodium sulfate, filtered and concentrated. The residue was purified by flash chromatography (eluted with petroleum ether in ethyl acetate from 30% to 60%) to obtain 4-(4-chloro-5-methoxy-6-(pyridin-4-yl)pyrimidin-2-yl)morpholine (0.2 g, 38.4%) as pale yellow solid. LCMS (ESI) m/z: 307.1 [M+H]⁺.

### Step 2: Synthesis of 4-(5-methoxy-4-(3-(1-methyl-1H-pyrazol-3-yl)phenyl)-6-(pyridin-4-yl)pyrimidin-2-yl)morpholine

A mixture of 4-(4-chloro-5-methoxy-6-(pyridin-4-yl)pyrimidin-2-yl)morpholine (100 mg, 0.33 mmol), 1-methyl-3-(3-(4,4,5,5-tetramethyl-1,3,2-dioxaborolan-2-yl)phenyl)-1H-pyrazole (92.8 mg, 0.33 mmol), 1,1′-bis(diphenylphosphino)ferrocenepalladium (II)dichloride dichloromethane complex (27 mg, 0.033 mmol) and cesium carbonate (0.27 g, 0.82 mmol) in 1,4-dioxane/water (8 mL/1 mL) was stirred at 95° C. for 16 h. It was concentrated, the residue was diluted with water (20 mL) and extracted with ethyl acetate (20 mL×2). The combined organic phase was washed with brine (20 mL), dried over sodium sulfate, filtered and concentrated. The residue was purified by prep-HPLC (base) to obtain 4-(5-methoxy-4-(3-(1-methyl-1H-pyrazol-3-yl)phenyl)-6-(pyridin-4-yl)pyrimidin-2-yl)morpholine (40.8 mg, 28.9%) as yellow solid. ¹H NMR (400 MHz, DMSO-d₆) δ 8.76 (d, J=5.8 Hz, 2H), 8.43 (s, 1H), 8.00 (d, J=6.0 Hz, 2H), 7.92 (dd, J=7.8, 1.6 Hz, 2H), 7.77 (d, J=2.1 Hz, 1H), 7.55 (t, J=7.8 Hz, 1H), 6.74 (d, J=2.2 Hz, 1H), 3.91 (s, 3H), 3.78 (d, J=5.0 Hz, 4H), 3.72 (d, J=4.8 Hz, 4H), 3.28 (s, 3H). LCMS (ESI) m/z: 429.3 [M+H]⁺.

### Example 24. Synthesis of 5-methoxy-6-(3-(1-methyl-1H-pyrazol-3-yl)phenyl)-2-morpholino-N-(pyridazin-3-ylmethyl)pyrimidin-4-amine (Compound 31)

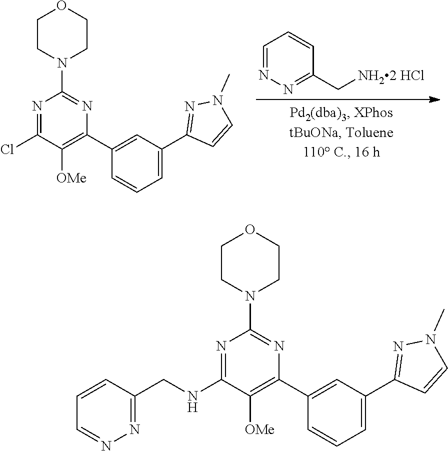

A mixture of 4-(4-chloro-5-methoxy-6-(3-(1-methyl-1H-pyrazol-3-yl)phenyl)pyrimidin-2-yl)morpholine (150 mg, 0.390 mmol), pyridazin-3-ylmethanamine·2HCl (71 mg, 0.390 mmol), tris(dibenzylideneacetone)dipalladium (30 mg, 0.05 mmol), X-Phos (56 mg, 0.06 mmol) and sodium tert-butoxide (75 mg, 0.780 mmol) in toluene (5 mL) was stirred at 110° C. for 16 h under nitrogen atmosphere. The reaction was then quenched with water (15 mL) and extracted with ethyl acetate (20 mL*3). The combined organic layer was washed with brine (30 mL), dried over sodium sulfate, filtered and concentrated. The residue was subjected to prep-HPLC to obtain 5-methoxy-6-(3-(1-methyl-1H-pyrazol-3-yl)phenyl)-2-morpholino-N-(pyridazin-3-ylmethyl)pyrimidin-4-amine (25.3 mg, 14.2%) as white solid. ¹H NMR (400 MHz, DMSO-d₆) δ 9.12 (dd, J=4.7, 1.6 Hz, 1H), 8.37 (s, 1H), 7.88-7.74 (m, 4H), 7.62 (ddd, J=10.1, 8.5, 3.2 Hz, 2H), 7.47 (t, J=7.7 Hz, 1H), 6.69 (d, J=2.2 Hz, 1H), 4.84 (d, J=5.9 Hz, 2H), 3.90 (s, 3H), 3.53 (d, J=4 Hz, 4H), 3.49 (d, J=4 Hz, 4H), 3.45 (s, 3H); LCMS (ESI) m/z: 459.0 [M+H]⁺.

### Example 25. Synthesis of (5-methoxy-63-(1-methyl-1H-pyrazol-3-yl)phenyl)-2-morpholinopyrimidin-4-yl)pyridin-4-yl)methanol (Compound 32)

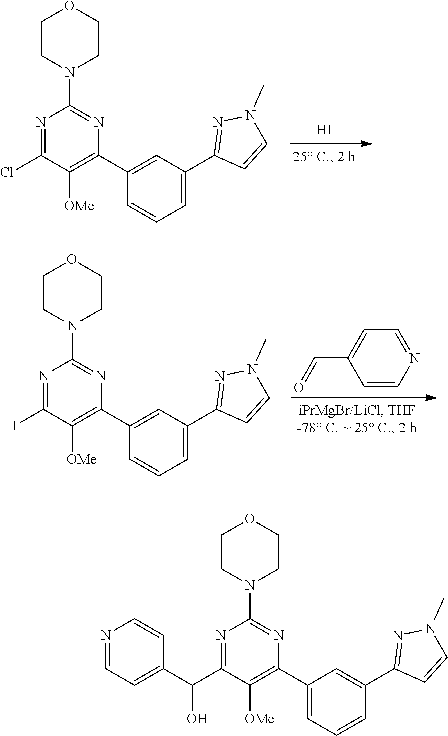

### Step 1: Synthesis of 4-(4-iodo-6-methoxy-6-(3-(1-methyl-1H-pyrazol-3-yl)phenyl)pyrimidin-2-yl)morpholine

A solution of 4-(4-chloro-5-methoxy-6-(3-(1-methyl-1H-pyrazol-3-yl)phenyl)pyrimidin-2-yl)morpholine (0.3 g, 0.78 mmol) in hydrogen iodide (5 mL) was stirred at 25° C. for 2 h under argon atmosphere. The reaction was quenched with water (10 mL) and extracted with ethyl acetate (25 mL*3). The combined organic layer was concentrated and purified by silica gel column chromatography (petroleum ether ethyl acetate=2:1) to obtain 4-(4-iodo-5-methoxy-6-(3-(1-methyl-1H-pyrazol-3-yl)phenyl)-pyrimidin-2-yl)morpholine (0.3 g, 54%) as yellow solid. LCMS (ESI) m/z: 477.1 [M+H]⁺.

### Step 2: Synthesis of (5-methoxy-6-(3-(1-methyl-1H-pyrazol-3-yl)phenyl)-2-morpholinopyrimidin-4-yl)(pyridin-4-yl)methanol

Isopropylmagnesium chloride (1.3M in tetrahydrofuran, 0.65 mL, 0.84 mmol) was added to a solution of 4-(4-iodo-5-methoxy-6-(3-(1-methyl-1H-pyrazol-3-yl)phenyl)pyrimidin-2-yl)morpholine (100 mg, 0.21 mmol) in anhydrous tetrahydrofuran (3 mL) at −78° C. under nitrogen atmosphere. The reaction mixture was stirred at −78° C. for 30 min followed by the addition of isonicotinaldehyde (90 mg, 0.84 mmol) in tetrahydrofuran (1.0 mL) at the same temperature. The reaction mixture was warmed up and stirred at room temperature for 2 h. It was then quenched with aqueous ammonium chloride solution (5 mL) and extracted with ethyl acetate (20 mL*3). The combined organic layer was washed with brine (20 mL), dried over sodium sulphate, filtered and concentrated to give the crude product It was then purified by prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% ammonium bicarbonate aqueous solution) to obtain (5-methoxy-6-(3-(1-methyl-1H-pyrazol-3-yl)phenyl)-2-morpholinopyrimidin-4-yl(pyridin-4-ylmethanol (34.3 mg, 36%) as white solid. ¹H NMR (400 MHz, DMSO-d₆) δ 8.54 (dd, J=4.5, 1.6 Hz, 2H), 8.37 (t, J=1.5 Hz, 1H), 7.93-7.82 (m, 2H), 7.76 (d, J=2.2 Hz, 1H), 7.52 (t, J=7.8 Hz, 1H), 7.49-7.44 (m, 2H), 6.72 (d, J=2.3 Hz, 1H), 6.11 (bs, 1H), 5.97 (s, 1H), 3.89 (d, J=8.1 Hz, 3H), 3.74-3.63 (m, 8H), 3.36 (s, 3H); LCMS (ESI) m/z: 458.5 [M]⁺.

### Example 26. Synthesis of N,N-dimethyl-2-morpholino-4-(3-pyrazol-1-ylphenyl)-6-(4-pyridylamino)pyrimidine-6-carboxamide (Compound 33)

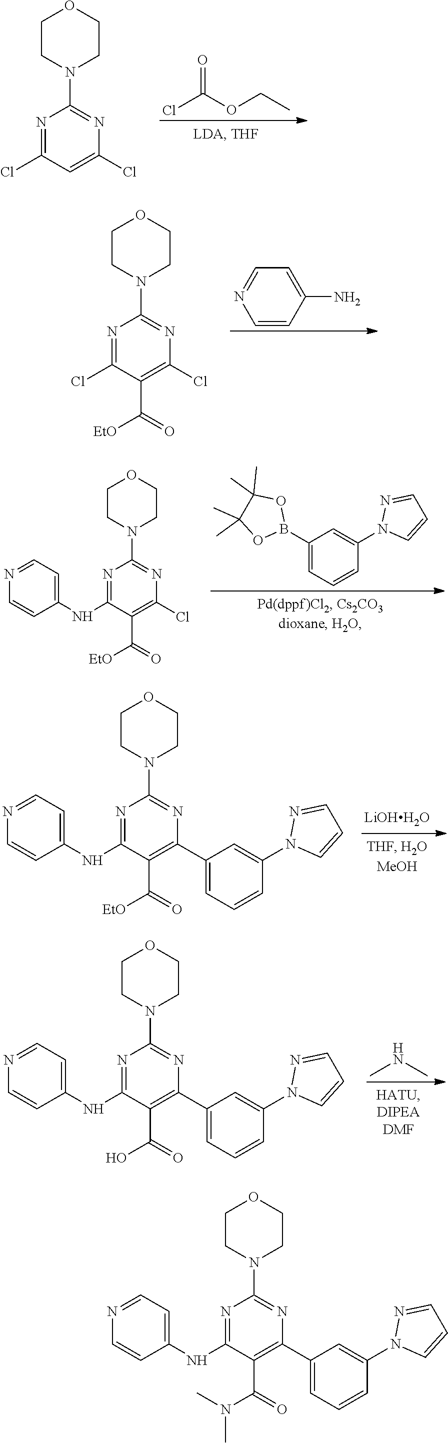

### Step 1: Synthesis of ethyl 4,6-dichloro-2-morpholino-pyrimidine-4-carboxylate

A solution of LDA (2.0M in THF, 1.92 mL) was added dropwise to a solution of 4-(4,6-dichloropyrimidin-2-yl)morpholine (750 mg, 3.20 mmol) in anhydrous THF (10 mL) at −70° C. and the resultant mixture was stirred under nitrogen atmosphere for 1 h. Then ethyl carbonochloridate (670 mg, 6.17 mmol) was added via syringe and the mixture was stirred at −70° C. for another 2 h and at 20° C. for 30 min. The reaction mixture was poured into ice-water (15 mL), the aqueous phase was extracted with ethyl acetate (15 mL*3). The combined organic phase was washed with brine (10 mL), dried with anhydrous Na₂SO₄, filtered and concentrated to obtain the crude product. It was purified by flash column (ISCO 20 g silica, 0-20% ethyl acetate in petroleum ether, gradient over 20 min) to obtain ethyl 4,6-dichloro-2-morpholino-pyrimidine-5-carboxylate (900 mg, 92%) as white solid.

### Step 2: Synthesis of ethyl 4-chloro-2-morpholino-6-(4-pyridylamino)pyrimidine-5-carboxylate

To a solution of pyridin-4-amine (120 mg, 1.28 mmol) in DMSO (10 mL) at 0° C. was added NaH (131 mg, 3.27 mmol). The mixture was warmed up and stirred at 20° C. for 0.5 h and cooled to 0° C. again. A solution of ethyl 4,6-dichloro-2-morpholino-pyrimidine-5-carboxylate (500 mg, 1.63 mmol) in DMSO (5 mL) was added and the mixture was warmed up and stirred at 20° C. for 2 h. The mixture was then poured into aqueous saturated NH₄Cl solution (15 mL) and was extracted with ethyl acetate (15 mL*3). The combined organic phase was washed with brine (10 mL), dried with anhydrous Na₂SO₄, filtered and concentrated to obtain ethyl 4-chloro-2-morpholino-6-(4-pyridylamino)pyrimidine-5-carboxylate (500 mg, 84%) as yellow solid.

### Step 3: Synthesis of ethyl 2-morpholino-4-(3-pyrazol-1-ylphenyl)-6-(4-pyridylamino)pyrimidine-5-carboxylate

To a solution of ethyl 4-chloro-2-morpholino-6-(4-pyridylamino)pyrimidine-5-carboxylate (400 mg, 1.10 mmol) in dioxane (8 mL) and H₂O (1.4 mL) was added 1-[3-(4,4,5,5-tetramethyl-1,3,2-dioxaborolan-2-yl)phenyl]pyrazole (297 mg, 1.10 mmol), Cs₂CO₃ (1.07 g, 3.30 mmol) and Pd(dppf)Cl₂ (80 mg, 110 umol) under nitrogen atmosphere. The resultant mixture was stirred at 100° C. for 2 h, then cooled to 15° C. and poured into ice-water (10 mL). The aqueous phase was extracted with ethyl acetate (15 mL*3), the combined organic phase was washed with brine (10 mL), dried with anhydrous Na₂SO₄, filtered and concentrated to afford the crude product. It was then purified by flash column chromatography (ISCO 40 g silica, 0-80% ethyl acetate in petroleum ether, gradient over 20 min) to obtain ethyl 2-morpholino-4-(3-pyrazol-1-ylphenyl)-6-(4-pyridylamino)pyrimidine-5-carboxylate (300 mg, 58%) as yellow solid.

### Step 4: Synthesis of 2-morpholino-4-(3-pyrazol-1-ylphenyl)-6-(4-pyridylamino)pyrimidine-5-carboxylic acid

To a solution of ethyl 2-morpholino-4-(3-pyrazol-1-ylphenyl)-6-(4-pyridylamino)pyrimidine-5-carboxylate (250 mg, 530 umol) in MeOH (0.5 mL) and THF (1 ml) and H₂O (0.5 mL) was added LiOH·H₂O (67 mg, 1.59 mmol). The mixture was stirred at 20° C. for 12 h and concentrated. It was then diluted with water (1 mL) and the aqueous phase was extracted with ethyl acetate (1 mL*3). The pH of the aqueous phase was then brought to ˜5 with saturated citric acid and the resultant precipitate was collected by filtration and dried to obtain 2-morpholino-4-(3-pyrazol-1-ylphenyl)-6-(4-pyridylamino)pyrimidine-5-carboxylic acid (130 mg, 55%) as pale yellow solid.

### Step 5: Synthesis of N,N-dimethyl-2-morpholino-4-(3-pyrazol-1-ylphenyl)-6-(4-pyridylamino)pyrimidine-5-carboxamide

To a solution of 2-morpholino-4-(3-pyrazol-1-ylphenyl)-6-(4-pyridylamino)pyrimidine-5-carboxylic acid (80 mg, 180 umol) in DMF (1 mL) were added N-methylmethanamine (2.0M, 90 uL) HATU (69 mg, 180 umol) and DIPEA (70 mg, 541 umol). The resultant mixture was stirred at 20° C. for 2 h. The crude product from DMF was isolated by subjecting it to prep-HPLC (Waters Xbridge BEH C18 100*30 mm*10 um column; 10-50% acetonitrile in an a 0.05% ammonia solution and an a 10 mM ammonium bicarbonate solution in water, 8 min gradient) to obtain N,N-dimethyl-2-morpholino-4-(3-pyrazol-1-ylphenyl)-6-(4-pyridylamino)pyrimidine-5-carboxamide (31 mg, 36%) as pale yellow solid. ¹H NMR (400 MHz, CHLOROFORM-d) δ 9.15 (bs, 1H), 8.49 (d, J=6.3 Hz, 2H), 8.11 (s, 1H), 7.99 (d, J=2.4 Hz, 1H), 7.86 (d, J=7.9 Hz, 1H), 7.77 (d, J=1.5 Hz, 1H), 7.66 (bs, 2H), 7.60-7.46 (m, 2H), 6.52 (t, J=1.9 Hz, 1H), 3.94 (s, 4H), 3.84 (t, J=4.8 Hz, 4H), 2.88 (s, 3H), 2.42 (s, 3H). LCMS (ESI for C25H26N8O2) [M+H]⁺: 471.1.

### Example 27. Synthesis of 5-methoxy-6-(3-(1-methyl-H-pyrazol-3-yl)phenyl)-N-(pyridin-3-yl)-2-(pyridin-4-yl)pyrimidin-4-amine (Compound 34)

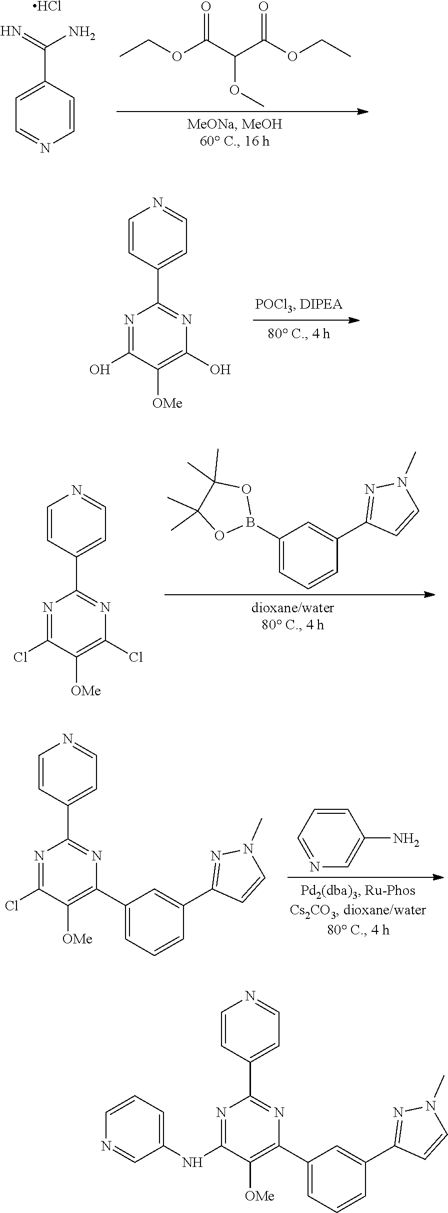

### Step 1: Synthesis of 5-methoxy-2-(pyridin-4-yl)pyrimidine-4,6-diol

A mixture of isonicotinimidamide hydrochloride (2 g, 12.7 mmol), methanol (10 mL) and sodium methoxide in methanol (25% wt, 4.6 ml, 25.4 mmol) was heated to reflux for 30 min followed by the addition of diethyl 2-methoxymalonate (2.4 g, 12.7 mmol). The resultant mixture was refluxed for another 5 h. The reaction mixture was then cooled, poured onto ice/water (˜50 ml) and acidified using 2N HCl to give a precipitate. This was collected by filtration and air-dried to give 5-methoxy-2-(pyridin-4-yl)pyrimidine-4,6-diol (1.5 g, 54%) as yellow solid. LCMS (ESI) m/z: 220.1 [M+H]⁺.

### Step 2: Synthesis of 4,6-dichloro-5-methoxy-2-(pyridin-4-yl)pyrimidine

To a mixture of 5-methoxy-2-(pyridin-4-yl)pyrimidine-4,6-diol (1 g, 4.56 mmol) in phosphorus oxychloride (30 mL) was added N,N-diisopropylethylamine (2.0 mL). The resultant mixture was stirred at 90° C. for 5 h. The volatiles were evaporated and further azeotroped with toluene (2×100 mL). The resultant residue was subjected to flash chromatography (petroleum ether/ethyl acetate=2:1) to obtain 4,6-dichloro-5-methoxy-2-(pyridin-4-yl)pyrimidine (0.2 g, 17%) as yellow solid. LCMS (ESI) m/z: 256.1 [M+H]⁺.

### Step 3: Synthesis of 4-chloro-5-methoxy-6-(3-(1-methyl-1H-pyrazol-3-yl)phenyl)-2-(pyridin-4-yl)pyrimidine

To a solution of 4,6-dichloro-5-methoxy-2-(pyridin-4-yl)pyrimidine (0.2 g, 0.78 mmol) and 1-methyl-3-(3-(4,4,5,5-tetramethyl-1,3,2-dioxaborolan-2-yl)phenyl)-1H-pyrazole (0.2 g, 0.7 mmol) in dioxane/water (5 mL/1.5 mL) were added cesium carbonate (0.51 g, 1.56 mmol) and [1,1′-bis(diphenylphosphino)ferrocene]dichloropalladium(II) (0.058 g, 0.08 mmol) and the resultant mixture was stirred at 90° C. for 2 h. The mixture was then poured into ice-water and extracted with ethyl acetate (15 mL*3). The organic layer was washed with brine, dried and concentrated. The crude product obtained was purified by flash chromatography on silica gel (petroleum ether/ethyl acetate=10:1) to obtain 4-chloro-5-methoxy-6-(3-(1-methyl-1H-pyrazol-3-yl)phenyl)-2-(pyridin-4-yl)pyrimidine (100 mg, 34%) as yellow solid. LCMS (ESI) m/z: 378.1 [M+H]⁺.

### Step 4: Synthesis of 5-methoxy-6-(3-(1-methyl-1H-pyrazol-3-yl)phenyl)-N-(pyridin-3-yl)-2-(pyridin-4-yl)pyrimidin-4-amine formate

A mixture of 4-chloro-5-methoxy-6-(3-(1-methyl-1H-pyrazol-3-yl)phenyl)-2-(pyridin-4-yl)pyrimidine (0.08 g, 0.2 mmol), pyridin-3-amine (0.04 g, 0.42 mmol), cesium carbonate (0.21 g, 0.64 mmol), tris(dibenzylideneacetone)dipalladium(0) (0.02 g, 0.02 mmol), and 2-dicyclohexylphosphino-2′,6′-di-i-propoxy-1,1′-biphenyl (0.015 g, 0.03 mmol) in toluene (4 mL) was stirred at 100° C. for 4 h. It was concentrated; the crude product was dissolved in DMF and subjected to prep-HPLC (SunFire C18, 4.6*50 mm, 3.5 um column. The elution system used was a gradient of 5%-95% over 1.5 min at 2 ml/min and the solvent was acetonitrile/0.01% ammonium bicarbonate aqueous solution) to afford 5-methoxy-6-(3-(1-methyl-1H-pyrazol-3-yl)phenyl)-N-(pyridin-3-yl)-2-(pyridin-4-yl)pyrimidin-4-amine as a yellow solid (38.6 mg, 42%). ¹H NMR (400 MHz, DMSO-d₆) δ 9.63 (s, 1H), 9.11 (d, J=2.4 Hz, 1H), 8.76 (dd, J=4.5, 1.5 Hz, 2H), 8.54 (t, J=1.5 Hz, 1H), 8.36-8.33 (m, 3H), 8.20 (dd, J=4.5, 1.6 Hz, 2H), 8.04-8.02 (m, 1H), 7.95-7.93 (m, 1H), 7.79 (d, J=2.2 Hz, 1H), 7.61 (t, J=7.8 Hz, 1H), 7.49 (dd, J=8.2, 4.7 Hz, 1H), 6.78 (d, J=2.2 Hz, 1H), 3.93 (d, J=6.6 Hz, 3H), 3.63 (s, 3H). LCMS (ESI) m/z: 435.8 [M+H]⁺.

### Example 28. Synthesis of 4-((1-methylpiperidin-3-yl)oxy)-2-morpholino-8-phenyl-6H-pyrazolo[1,5-d]pyrimido[5,4-b][1,4]oxazine (Compound 35)

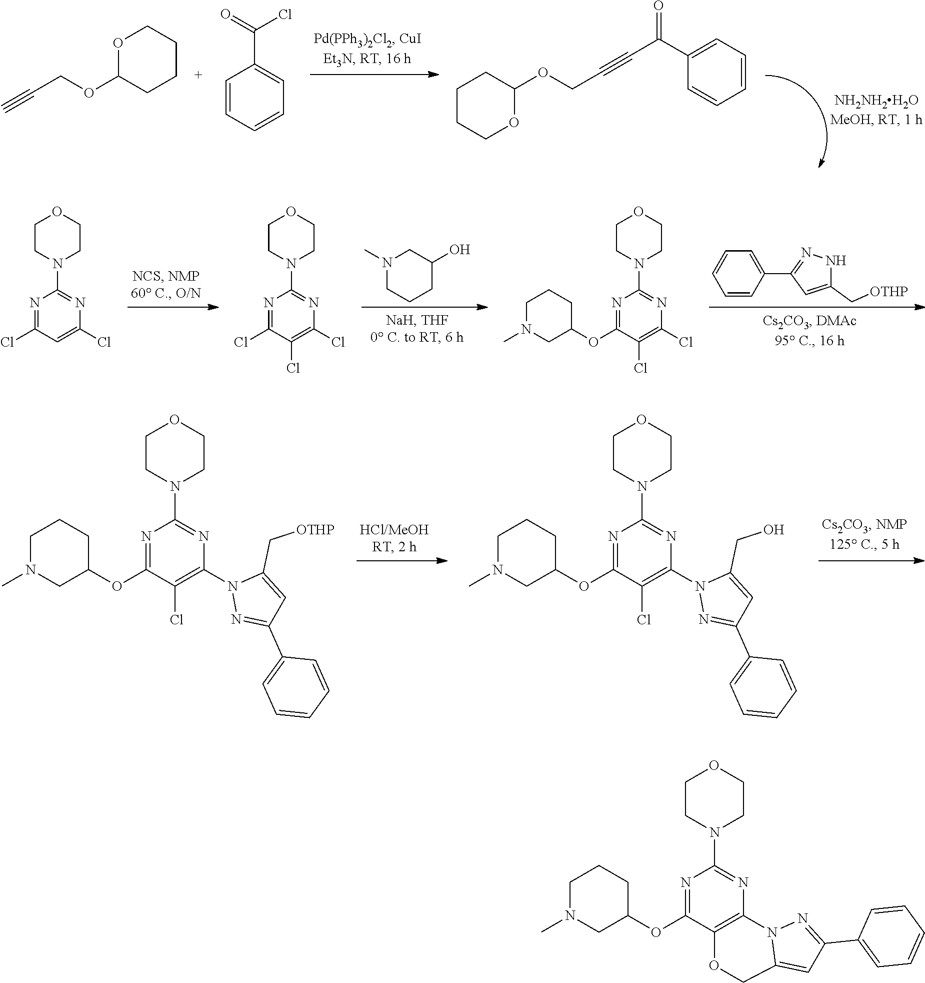

### Step 1a: Synthesis of 1-phenyl-4-((tetrahydro-2H-pyran-2-yl)oxy)but-2-yn-1-one

A mixture of 2-(prop-2-yn-1-yloxy)tetrahydro-2H-pyran (1.0 g, 7.13 mmol), benzoyl chloride (1.0 g, 7.13 mmol), cuprous iodide (67 mg, 0.36 mmol) and bis(triphenylphosphine)palladium(II) chloride (50 mg, 0.07 mmol) in triethylamine (15 mL) was stirred at 25° C. for 16 h. The reaction mixture was filtered and the filtrate was concentrated to obtain the title compound (1.2 g, 69%) as yellow solid. LCMS (ESI) m/z: 245.0 [M+H]⁺.

### Step 1b: Synthesis of 3-phenyl-5-(((tetrahydro-2H-pyran-2-yl)oxy)methyl)-1H-pyrazole

A mixture of 1-phenyl-4-((tetrahydro-2H-pyran-2-yl)oxy)but-2-yn-1-one (1 g, 4.1 mmol) and hydrazine hydrate (1 mL) in methanol (15 mL) was stirred at 25° C. for 2 h. The reaction mixture was concentrated and the resultant crude product was purified by silica gel column chromatography (petroleum ether ethyl acetate=2:1) to obtain the title compound (0.6 g, 57%) as yellow solid. LCMS (ESI) m/z: 259.0 [M+H]⁺.

### Step 1: Synthesis of 4-(4,5,6-trichloropyrimidin-2-yl)morpholine

To a solution of 4-(4,6-dichloropyrimidin-2-yl)morpholine (5 g, 21.3 mmol) in 1-methyl-2-pyrrolidinone (100 mL) was added N-chlorosuccinimide (5.7 g, 42.7 mmol) in portions at room temperature. The resultant mixture was stirred at 60° C. for 16 h. It was cooled down, the mixture was diluted with ethyl acetate/water (20 mL/20 mL), the organic layer was separated and the aqueous layer was extracted with ethyl acetate (20 mL×2). The combined organic phase was washed with brine (30 mL), dried over sodium sulfate, filtered and concentrated. The residue was subjected to flash chromatography (Biotage, 40 g silica gel, eluted with ethyl acetate in petroleum ether from 20% to 30%) to obtain 4-(4,5,6-trichloropyrimidin-2-yl) morpholine (5.1 g, 89.7%) as off-white solid.

### Step 2: Synthesis of 4-(4,5-dichloro-6-((1-methylpiperidin-3-yl)oxy)pyrimidin-2-yl)morpholine

To a solution of 1-methylpiperidin-3-ol (0.45 g, 3.91 mmol) in tetrahydrofuran (20 mL) at 0° C. was added sodium hydride (60%, 0.23 g, 5.87 mmol) in portions. After the addition, the reaction mixture was stirred at 0° C. for 30 min followed by the drop wise addition of a solution of 4-(4,5,6-trichloropyrimidin-2-yl) morpholine (1 g, 3.91 mmol) in tetrahydrofuran (10 mL). The resultant mixture was warmed up and stirred at room temperature for 16 h. It was then diluted with ethyl acetate/water (20 mL/20 mL), the organic layer was separated, and the aqueous layer was extracted with ethyl acetate (20 mL×2). The combined organic phase was washed with brine (30 mL), dried over sodium sulfate, filtered, and concentrated. The crude product thus obtained was purified by flash chromatography (Biotage, 40 g silica gel, eluted with ethyl acetate in petroleum ether from 20% to 40%) to obtain 4-(4,5-dichloro-6-((1-methylpiperidin-3-yl)oxy)pyrimidin-2-ylmorpholine (1.2 g, 88.7%) as white solid. LCMS (ESI) m/z: 347.1 [M+H]⁺.

### Step 3: Synthesis of 4-(5-chloro-4-((1-methylpiperidin-3-yl)oxy)-6-(3-phenyl-6-(((tetrahydro-2H-pyran-2-yl)oxy)methyl)-1H-pyrazol-1-yl)pyrimidin-2-yl)morpholine

A mixture of 4-(4,5-dichloro-6-((1-methylpiperidin-3-yl)oxy)pyrimidin-2-yl)morpholine (0.5 g, 1.45 mmol), 3-phenyl-5-(((tetrahydro-2H-pyran-2-yl)oxy)methyl)-1H-pyrazole (0.37 g, 1.45 mmol) and cesium carbonate (0.94 g, 2.89 mmol) in N,N-dimethylacetamide (25 mL) was stirred at 95° C. for 16 h. The reaction was cooled down, the mixture was diluted with ethyl acetate/water (20 mL/20 mL), the organic layer was separated and the aqueous layer was extracted with ethyl acetate (20 mL×2). The combined organic phase was washed with brine (20 mL), dried over sodium sulfate, filtered, and concentrated. The residue was subjected to flash chromatography (Biotage, 40 g silica gel, eluted with methanol:dichloromethane=1:10 in dichloromethane from 15% to 30%) to obtain 4-(5-chloro-4-((1-methylpiperidin-3-yl)oxy)-6-(3-phenyl-5-(((tetrahydro-2H-pyran-2-yl)oxy)methyl)-1H-pyrazol-1-yl)pyrimidin-2-yl)morpholine (0.4 g, 48.6%) as white solid. LCMS (ESI) m/z: 569.2 [M+H]⁺.

### Step 4: Synthesis of (1-(5-chloro-6-((1-methylpiperidin-3-yl)oxy)-2-morpholinopyrimidin-4-yl)-3-phenyl-1H-pyrazol-6-yl)methanol

To a solution of 4-(5-chloro-4-((1-methylpiperidin-3-yl)oxy)-6-(3-phenyl-5-(((tetrahydro-2H-pyran-2-yloxy)methyl)-1H-pyrazol-1-yl)pyrimidin-2-yl)morpholine (0.4 g, 0.7 mmol) in methanol (20 mL) was added hydrochloric acid (3.0M in methanol, 2 mL) and the resultant mixture was stirred at room temperature for 2 h. The reaction mixture was concentrated, the residue was diluted with dichloromethane (10 mL) and the pH of the medium was increased above 7 using sodium bicarbonate solution. The organic layer was separated and the aqueous layer was extracted with dichloromethane (20 mL×2). The combined organic phase was washed with brine (20 mL), dried over sodium sulfate, filtered and concentrated to obtain (1-(5-chloro-6-((1-methylpiperidin-3-yl)oxy)-2-morpholinopyrimidin-4-yl)-3-phenyl-1H-pyrazol-5-yl)methanol (0.28 g, 82.6%) as white foam. LCMS (ESI) m/z: 485.1 [M+H]⁺.

### Step 5: Synthesis of 4-((1-methylpiperidin-3-yl)oxy)-2-morpholino-8-phenyl-6H-pyrazolo[1,5-d]pyrimido[5,4-b][1,4]oxazine

A mixture of (1-(5-chloro-6-((1-methylpiperidin-3-yl)oxy)-2-morpholinopyrimidin-4-yl)-3-phenyl-1H-pyrazol-5-ylmethanol (0.11 g, 0.23 mmol) and cesium carbonate (0.15 g, 0.46 mmol) in NMP (3 mL) was stirred at 125° C. for 5 h. The resultant reaction mixture was filtered and the filtrate was subjected to prep-HPLC (BOSTON pHlex ODS 10 um 21.2×250 mm 120 A. The mobile phase was acetonitrile/0.1% ammonium bicarbonate) to obtain the target compound (1.1 mg, 1%) as white solid. ¹H NMR (400 MHz, CDCl₃) δ 7.82 (d, J=7.4 Hz, 2H), 7.41 (d, J=7.1 Hz, 2H), 7.34 (d, J=7.2 Hz, 1H), 6.49 (s, 1H), 5.47 (bs, 1H), 5.40 (s, 2H), 3.76 (s, 8H), 3.33 (bs, 1H), 2.94 (bs, 1H), 2.69 (bs, 1H), 2.56 (s, 3H), 2.53-2.44 (m, 2H), 2.29 (bs, 1H), 1.86 (bs, 2H); LCMS (ESI) m/z: 448.8 [M]⁺.

### Example 29. Synthesis of 4-[5-methoxy-6-morpholino-2-(3-pyrazol-1-yl)phenyl)pyrimidin-4-yl]-1-methyl-piperazin-2-one (Compound 36) and 4-[5-methoxy-4-morpholino-6-(3-pyrazol-1-ylphenyl)pyrimidin-2-yl]-1-methyl-piperazin-2-one (Compound 37)

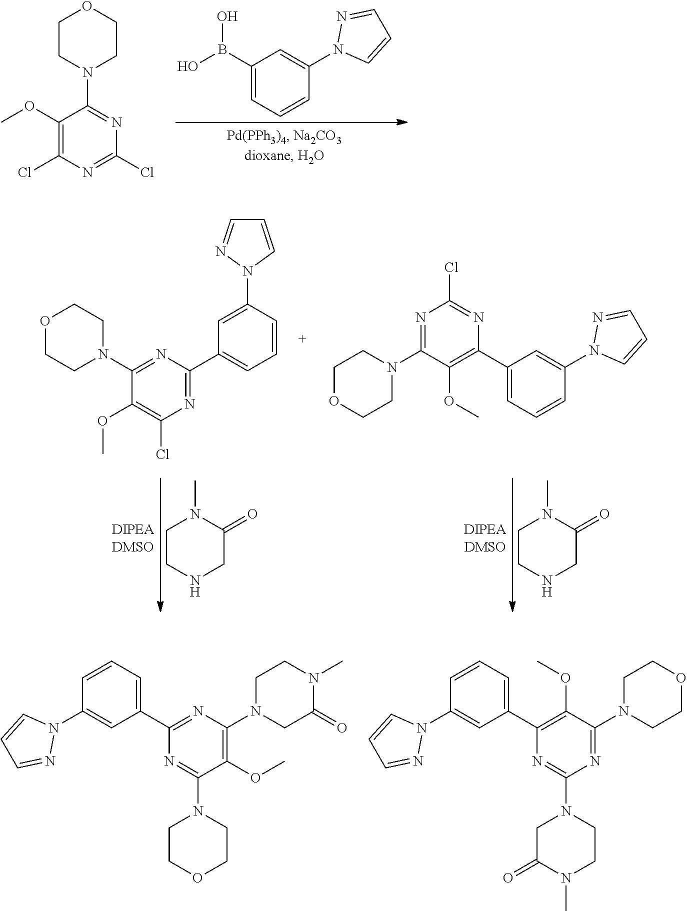

### Step 1: Synthesis of 4-[2-chloro-6-methoxy-6-(3-pyrazol-1-ylphenyl)pyrimidin-4-yl]morpholine and 4-[6-chloro-5-methoxy-2-(3-pyrazol-1-ylphenyl)pyrimidin-4-yl]morpholine

To a solution of 4-(2,6-dichloro-5-methoxy-pyrimidin-4-yl)morpholine (500 mg, 1.89 mmol) in dioxane (1 mL) and H₂O (0.1 mL) were added 1-[3-(4,4,5,5-tetramethyl-1,3,2-dioxaborolan-2-yl)phenyl]pyrazole (511 mg, 1.89 mmol), Na₂CO₃ (2 M, 2.84 mL) and Pd(PPh₃)₄ (219 mg, 189 umol) under nitrogen atmosphere and the resulting mixture was stirred at 100° C. for 24 h. The mixture was then filtered and filtrate was subjected to prep-HPLC (Waters Xbridge Prep OBD C18 150*40 mm*10 um column; 30-70% acetonitrile in an a 0.04% ammonia solution in water, 8 min gradient) to obtain 4-[2-chloro-5-methoxy-6-(3-pyrazol-1-ylphenyl)pyrimidin-4-yl]morpholine (170 mg) and 4-[6-chloro-5-methoxy-2-(3-pyrazol-1-ylphenyl)pyrimidin-4-yl]morpholine (40 mg) as yellow solids. LCMS (ESI) m/z: 372.1 [M+H]⁺ (both isomers).

### Step 2: Synthesis of 4-[5-methoxy-4-morpholino-2-(3-pyrazol-1-ylphenyl)pyrimidin-4-yl]-1-methyl-piperazin-2-one (Compound 37)

To a solution of 4-[6-chloro-5-methoxy-2-(3-pyrazol-1-ylphenyl)pyrimidin-4-yl]morpholine (170 mg, 457 umol) in DMSO (3 mL) were added 1-methylpiperazin-2-one (157 mg, 1.37 mmol) and DIPEA (177 mg, 1.37 mmol). The resulting mixture was stirred at 110° C. for 12 h. Then water (5 mL) was added to the reaction mixture and it was extracted with ethyl acetate (3 mL*2). The combined organic layers were washed with brine (5 mL) and dried over Na₂SO₄. Concentration followed by subjecting the residue to prep-HPLC (Waters Xbridge BEH C18 100*25 mm*5 um column; 40-75% acetonitrile in an a 10 mM ammonium bicarbonate solution in water, 10 min gradient) afforded 4-[5-methoxy-6-morpholino-2-(3-pyrazol-1-ylphenyl)pyrimidin-4-yl]-1-methyl-piperazin-2-one (86 mg, 42%) as light yellow solid. ¹H NMR (400 MHz, CHLOROFORM-d) δ 8.57 (s, 1H), 8.27 (d, J=7.9 Hz, 1H), 8.03 (d, J=2.4 Hz, 1H), 7.82-7.73 (m, 2H), 7.51 (t, J=8 Hz, 1H), 6.51 (t, J=2.1 Hz, 1H), 4.41 (s, 2H), 4.02 (t, J=5.3 Hz, 2H), 3.89-3.72 (m, 8H), 3.65 (s, 3H), 3.52 (t, J=5.4 Hz, 2H), 3.04 (s, 3H). LCMS (ESI) for (C23H27N7O3) [M+H]⁺: 450.1

### Step 3: Synthesis of 4-[5-methoxy-4-morpholino-6-(3-pyrazol-1-ylphenyl)pyrimidin-2-yl]-1-methyl-piperazin-2-one (Compound 38)

The compound 38 was synthesized according to the protocol described for compound 37 and was isolated as white solid. ¹H NMR (400 MHz, CHLOROFORM-d) δ 8.31 (t, J=2.0 Hz, 1H), 8.00 (d, J=2.2 Hz, 1H), 7.92 (d, J=7.8 Hz, 1H), 7.82-7.73 (m, 2H), 7.52 (t, J=8.0 Hz, 1H), 6.49 (t, J=2.1 Hz, 1H), 4.41 (s, 2H), 4.09 (bs, 2H), 3.82 (m, 8H), 3.46 (t, J=5.2 Hz, 2H), 3.42 (s, 3H), 3.05 (s, 3H). LCMS (ESI) for (C23H27N7O3) [M+H]⁺: 450.2.

### Example 30. Synthesis of 9-(1-methyl-1H-pyrazol-3-yl)-2-morpholino-N-(pyridin-4-yl)-6H-pyrido[4′,3′:4,5]pyrano[3,2-d]pyrimidin-4-amine (Compound 38)

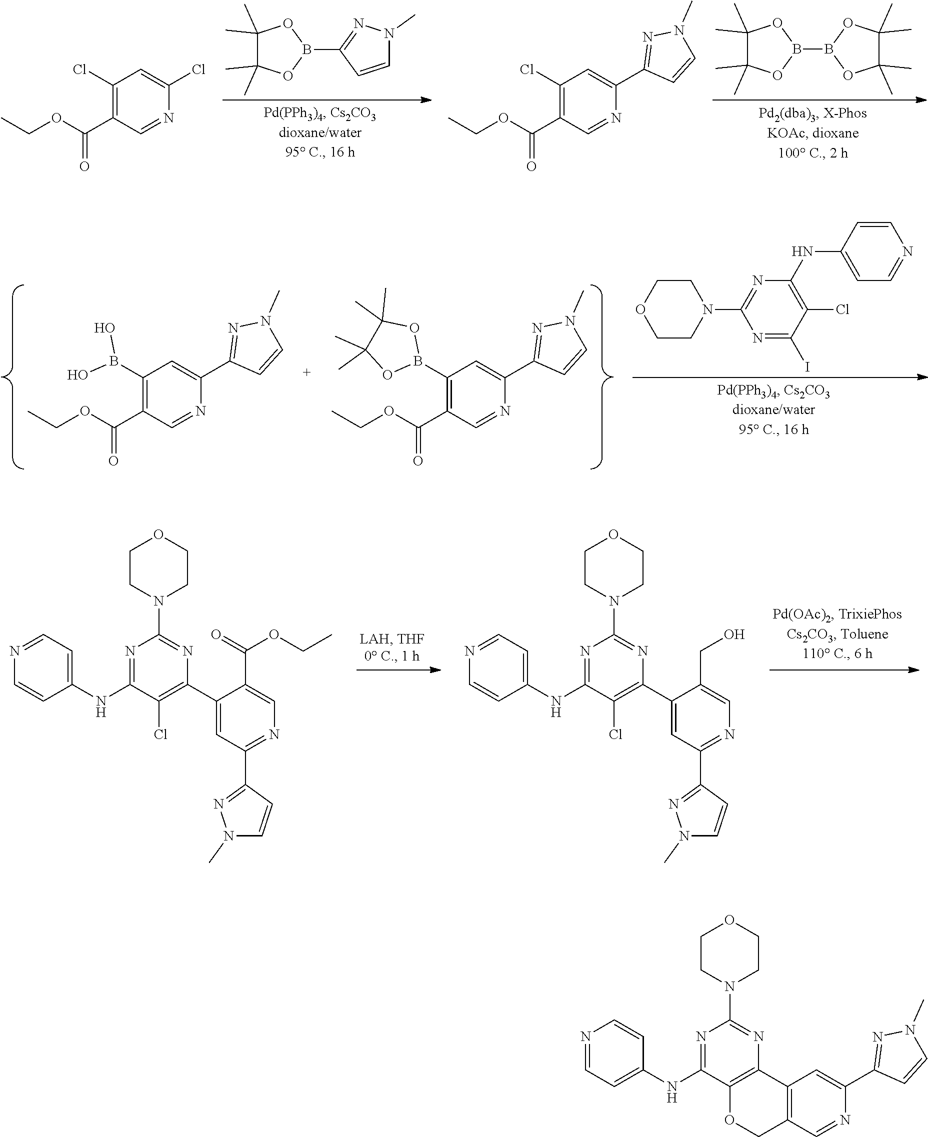

### Step 1: Synthesis of ethyl 4-chloro-6-(1-methyl-1H-pyrazol-3-yl)nicotinate

To a mixture of ethyl 4,6-dichloronicotinate (3 g, 13.63 mmol) in 1,4-dioxane/water (50 mL/10 mL) was added 1-methyl-3-(4,4,5,5-tetramethyl-1,3,2-dioxaborolan-2-yl)-1H-pyrazole (2.84 g, 13.63 mmol), cesium carbonate (8.88 g, 27.27 mmol) and tetrakis(triphenylphosphine)palladium (0.95 g, 0.82 mmol). The reaction mixture was stirred at 100° C. for 4 h under argon atmosphere. It was then filtered, diluted with water and extracted with ethyl acetate (50 mL*3). The organic layers were combined, concentrated, and subjected to silica gel column (petroleum ether:ethyl acetate=2:1) to obtain ethyl 4-chloro-6-(1-methyl-1H-pyrazol-3-ylnicotinate (2 g, 55%). as yellow solid LCMS (ESI) m/z: 266.1 [M+H]⁺.

### Step 2: Synthesis of (5-(ethoxycarbonyl)-2-(1-methyl-1H-pyrazol-3-yl)pyridin-4-yl)boronic acid and ethyl 6-(1-methyl-1H-pyrazol-3-yl)-4-(4,4,5,5-tetramethyl-1,3,2-dioxaborolan-2-yl)nicotinate

A mixture of ethyl 4-chloro-6-(1-methyl-1H-pyrazol-3-ylnicotinate (1.28 g, 4.82 mmol), 4,4,4′,4′,5,5,5′-heptamethyl-2,2′-bi(1,3,2-dioxaborolane) (6.12 g, 24.09 mmol), potassium acetate (1.28 g, 13.01 mmol), tris(dibenzylideneacetone) dipalladium(0) (0.44 g, 0.48 mmol), and 2-(dicyclohexylphosphino)-2′,4′,6′-triisopropylbiphenyl (0.34 g, 0.72 mmol) in 1,4-dioxane (15 mL) was stirred at 100° C. for 4 h. The mixture was filtered, the filtrate was concentrated and the residue was subjected to silica gel column chromatography (dichloromethane:methanol=10:1) to obtain a mixture of (5-(ethoxycarbonyl)-2-(1-methyl-1H-pyrazol-3-yl)pyridin-4-yl)boronic acid and ethyl 6-(1-methyl-1H-pyrazol-3-yl)-4-(4,4,5,5-tetramethyl-1,3,2-dioxaborolan-2-yl)nicotinate (0.9 g, 68%) as yellow solid. LCMS (ESI) m/z: 276.1 [M+H]⁺, 358.1 [M+H]⁺.

### Step 3: Synthesis of ethyl 4-(5-chloro-2-morpholino-6-(pyridin-4-ylamino)pyrimidin-4-yl)-6-(1-methyl-1H-pyrazol-3-yl)nicotinate

To a solution of 5-chloro-6-iodo-2-morpholino-N-(pyridin-4-yl)pyrimidin-4-amine (0.3 g, 0.72 mmol) and the mixture [(5-(ethoxycarbonyl)-2-(1-methyl-1H-pyrazol-3-yl)pyridin-4-yl)boronic acid and ethyl 6-(1-methyl-1H-pyrazol-3-yl)-4-(4,4,5,5-tetramethyl-1,3,2-dioxaborolan-2-yl)nicotinate](0.69 g, 2.51 mmol) from step-2 in N,N-dimethylformamide (10 mL) were added potassium phosphate tribasic (0.56 g, 2.51 mmol) and 1,1′-bis(diphenylphosphino)ferrocene-palladium(II)dichloride dichloromethane complex (0.05 g, 0.07 mmol). The resultant mixture was stirred at 100° C. for 3 h. The reaction mixture was filtered, the filtrate was diluted with water (30 mL) and extracted with ethyl acetate (20 mL*3). The organic layers were concentrated, and the residue was subjected to flash chromatography on silica gel (dichloromethane:methanol=10:1) to obtain ethyl 4-(5-chloro-2-morpholino-6-(pyridin-4-ylamino)pyrimidin-4-yl)-6-(1-methyl-1H-pyrazol-3-ylnicotinate as a yellow solid. (0.13 g, 35%). LCMS (ESI) m/z: 521.3 [M+H]⁺.

### Step 4: Synthesis of (4-(5-chloro-2-morpholino-6-(pyridin-4-ylamino)pyrimidin-4-yl)-6-(1-methyl-1H-pyrazol-3-yl)pyridin-3-yl)methanol

Lithium aluminum hydride (1.0M in THF, 2.56 mL, 2.56 mmol) was added to a solution of ethyl 4-(5-chloro-2-morpholino-6-(pyridin-4-ylamino)pyrimidin-4-yl)-6-(1-methyl-1H-pyrazol-3-yl)nicotinate (130 mg, 0.25 mmol) in anhydrous tetrahydrofuran (5 mL) at 0° C. under nitrogen atmosphere. The mixture was then warmed up and stirred for 1.5 h at 25° C. The reaction was then quenched by the careful addition of sodium sulfate decahydrate with ice-bath cooling. Tetrahydrofuran (50 mL) was added to the reaction mixture, stirred for 15 min and the resultant solids were filtered off. The filtrate was concentrated, and the residue obtained was subjected to flash chromatography on silica gel (dichloromethane:methanol=10:1) to afford (4-(5-chloro-2-morpholino-6-(pyridin-4-ylamino)pyrimidin-4-yl)-6-(1-methyl-1H-pyrazol-3-ylpyridin-3-ylmethanol (50 mg, 80%) as yellow solid. LCMS (ESI) m/z: 479.3 [M+H]⁺.

### Step 5: Synthesis of 9-(1-methyl-1H-pyrazol-3-yl)-2-morpholino-N-(pyridin-4-yl)-6H-pyrido[4′,3′:4,5]pyrano[3,2-d]pyrimidin-4-amine

To a mixture of ((4-(5-chloro-2-morpholino-6-(pyridin-4-ylamino)pyrimidin-4-yl)-6-(1-methyl-1H-pyrazol-3-yl)pyridin-3-yl)methanol (0.06 g, 0.13 mmol) in toluene (2.5 mL) were added cesium carbonate (0.08 g, 0.25 mmol), palladium(II)acetate (0.006 g, 0.03 mmol) and racemic-2-di-t-butylphosphino-1,1′-binaphthyl (0.026 g, 0.06 mmol). The reaction mixture was stirred at 110° C. for 5 h under argon atmosphere. The resultant mixture was filtered to remove the solids, the filtrate was concentrated and the residue was subjected to prep-HPLC (BOSTON pHlex ODS 10 um 21.2×250 mm 120 A, with mobile phase acetonitrile/0.1% ammonium bicarbonate) to obtain 9-(1-methyl-1H-pyrazol-3-yl)-2-morpholino-N-(pyridin-4-yl)-6H-pyrido[4′,3′:4,5]pyrano[3,2-d]pyrimidin-4-amine as an off-white solid (4.0 mg, 7%). ¹H NMR (400 MHz, DMSO-d₆) δ 9.45 (s, 1H), 8.58 (s, 1H), 8.41 (d, J=6.1 Hz, 2H), 8.35 (s, 1H), 7.87 (d, J=6.2 Hz, 2H), 7.80 (d, J=2.2 Hz, 1H), 6.84 (d, J=2.1 Hz, 1H), 5.37 (s, 2H), 3.96 (s, 3H), 3.73 (d, J=8.5 Hz, 8H). LCMS (ESI) m/z: 443.3 [M+H]⁺.

### Example 31. Synthesis of 5-methoxy-6-(2-(1-methyl-1H-pyrazol-3-yl)pyridin-4-yl)-N, 2-di(pyridin-4-yl)pyrimidin-4-amine (Compound 39)

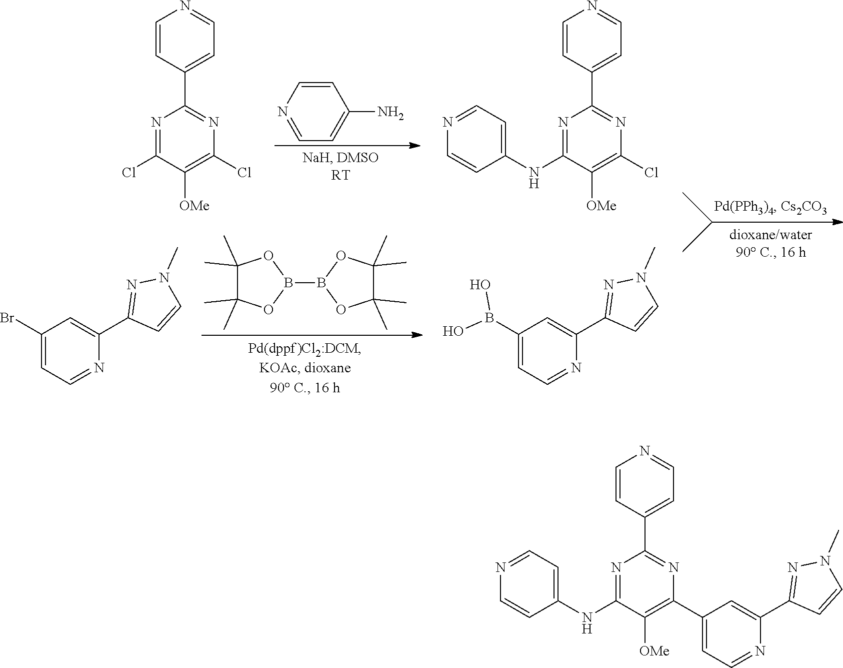

### Step 1: Synthesis of 6-chloro-5-methoxy-N, 2-di(pyridin-4-yl)pyrimidin-4-amine

A mixture of pyridin-4-amine (0.11 g, 1.2 mmol) and sodium hydride (0.06 g, 2.4 mmol) in dimethyl sulfoxide was stirred at 25° C. for 0.5 h. To the mixture was added 4,6-dichloro-5-methoxy-2-(pyridin-4-yl)pyrimidine (0.31 g, 1.2 mmol) and the reaction mixture was stirred at 25° C. for an additional 2 h. The mixture was then poured into ice-water and extracted with ethyl acetate (15 mL*3). The combined organic phase was washed with brine, dried over sodium sulfate and concentrated. The crude product was purified by flash chromatography on silica gel (dichloromethane:methanol=20:1) to obtain 6-chloro-5-methoxy-N, 2-di(pyridin-4-yl)pyrimidin-4-amine (70 mg, 18%) as yellow solid. LCMS (ESI) m/z: 314.1 [M+H]⁺.

### Step 2: Synthesis of (2-(1-methyl-1H-pyrazol-3-yl)pyridin-4-yl)boronic acid

To a solution of 4-bromo-2-(1-methyl-1H-pyrazol-3-yl)pyridine (0.08 g, 0.34 mmol) and 4,4,4′,4′,5,5,5′,5′-octamethyl-2,2′-bi(1,3,2-dioxaborolane) (0.11 g, 0.44 mmol) in 1,4-dioxane (7 mL) were added potassium acetate (0.067 g, 0.68 mmol) and 1,1′-bis(diphenylphosphino)ferrocene-palladium(II)dichloride dichloromethane complex (0.025 g, 0.034 mmol). The resultant mixture was stirred at 90° C. for 16 h and cooled. This reaction mixture was used directly in next step without further purification. LCMS (ESI) m/z: 204.1 [M+H]⁺.

### Step 3: Synthesis of 5-methoxy-6-(2-(1-methyl-1H-pyrazol-3-yl)pyridin-4-yl)-N, 2-di(pyridin-4-yl)pyrimidin-4-amine

To the mixture from step-2 was added 5 mL of 1,4-dioxane/water (4 mL/1 mL) followed by 6-chloro-5-methoxy-N, 2-di(pyridin-4-yl)pyrimidin-4-amine (0.08 g, 0.25 mmol), cesium carbonate (0.16 g, 0.5 mmol) and tetrakis(triphenylphosphine)palladium (0.03 g, 0.025 mmol). The resultant reaction mixture was stirred at 90° C. for 16 h under argon atmosphere and concentrated. The residue was then subjected to prep-HPLC (BOSTON pHlex ODS 10 um 21.2×250 mm 120 A, with mobile phase acetonitrile/0.1% ammonium bicarbonate) to obtain 5-methoxy-6-(2-(1-methyl-1H-pyrazol-3-yl)pyridin-4-yl)-N, 2-di(pyridin-4-yl)pyrimidin-4-amine as an off-white solid (3.7 mg, 3%). ¹H NMR (400 MHz, DMSO-d₆) δ 9.86 (s, 1H), 8.80 (dd, J=8.3, 3.7 Hz, 3H), 8.61 (s, 1H), 8.55 (d, J=6.3 Hz, 2H), 8.26 (dd, J=4.5, 1.5 Hz, 2H), 8.05 (dd, J=4.9, 1.5 Hz, 2H), 7.96 (dd, J=5.1, 1.7 Hz, 1H), 7.83 (d, J=2.2 Hz, 1H), 6.89 (d, J=2.2 Hz, 1H), 3.96 (s, 3H), 3.67 (s, 3H). LCMS (ESI) m/z: 437.3 [M+H]⁺.

### Example 32. Synthesis of 9-(1-methyl-1H-pyrazol-3-yl)-2-morpholino-N-(pyridin-4-yl)-6H-pyrido[3′,2′:4,5] pyrano[3,2-d]pyrimidin-4-amine (Compound 40)

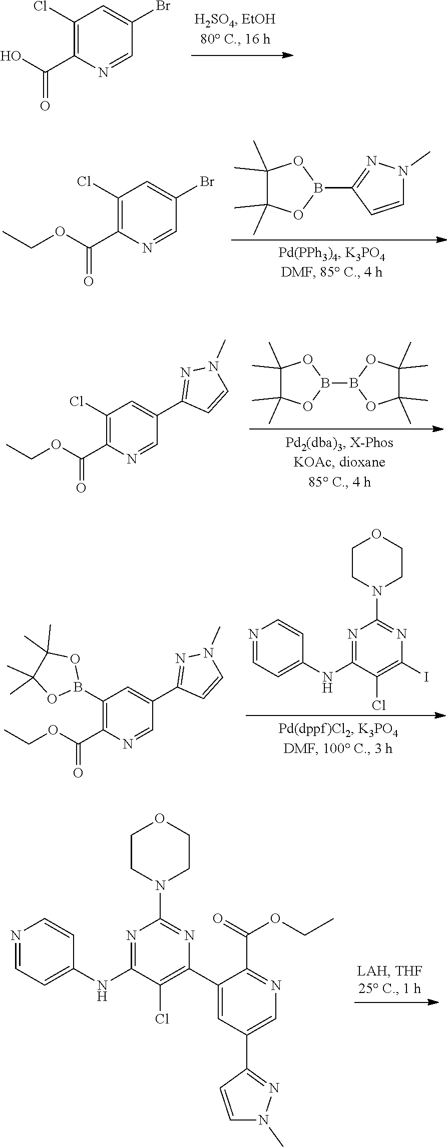

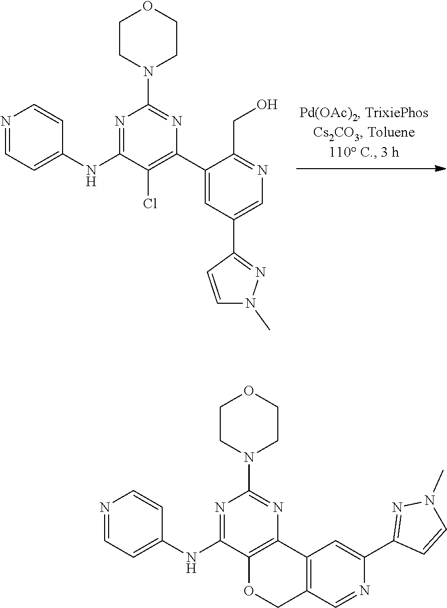 [SMILES: Cn1ccc(-c2cnc(CO)c(-c3nc(N4CCOCC4)nc(Nc4ccncc4)c3Cl)c2)n1 ; Cn1ccc(-c2cc3c(cn2)COc2c(Nc4ccncc4)nc(N4CCOCC4)nc2-3)n1]

### Step 1: Synthesis of ethyl 5-bromo-3-chloropicolinate

To a solution of 5-bromo-3-chloropicolinic acid (5 g, 21.2 mmol) in ethanol (50 mL) was added sulfuric acid (0.1 mL). The reaction mixture was stirred at 80° C. for 16 h and concentrated. The residue was dissolved with ethyl acetate (100 mL), filtered and concentrated to obtain ethyl 5-bromo-3-chloropicolinate as yellow oil (3.5 g, 63%). LCMS (ESI) m/z: 264.1 [M+H]⁺.

### Step 2: Synthesis of ethyl 3-chloro-5-(1-methyl-1H-pyrazol-3-yl)picolinate

To a mixture of ethyl 5-bromo-3-chloropicolinate (1.5 g, 5.67 mmol) in N,N-dimethylformamide (25 mL) were added 1-methyl-3-(4,4,5,5-tetramethyl-1,3,2-dioxaborolan-2-yl)-1H-pyrazole (1.06 g, 5.10 mmol), potassium phosphate tribasic (0.66 g, 0.57 mmol) and tetrakis(triphenylphosphine)palladium (0.66 g, 0.57 mmol). The resultant reaction mixture was stirred at 85° C. for 4 h under nitrogen atmosphere. The reaction mixture was then cooled, filtered, the filtrate was diluted with water and extracted with ethyl acetate (50 mL*3). The organic layers were combined, concentrated and the residue was subjected to silica gel column chromatography (petroleum ether ethyl acetate=2:1) to obtain ethyl 3-chloro-5-(1-methyl-1H-pyrazol-3-yl)picolinate as a yellow solid (1 g, 66%). LCMS (ESI) m/z: 266.1 [M+H]⁺.

### Step 3: Synthesis of ethyl 5-(1-methyl-1H-pyrazol-3-yl)-3-(4,4,5,5-tetramethyl-1,3,2-dioxaborolan-2-yl)picolinate

A mixture of ethyl 3-chloro-5-(1-methyl-1H-pyrazol-3-yl)picolinate (1.28 g, 4.82 mmol), 4,4,4′,4′,5,5,5′-heptamethyl-2,2′-bi(1,3,2-dioxaborolane) (6.12 g, 24.09 mmol), potassium acetate (1.28 g, 13.01 mmol), tris(dibenzylideneacetone) dipalladium(0) (0.44 g, 0.48 mmol), and 2-(dicyclohexylphosphino)-2′,4′,6′-triisopropylbiphenyl (0.34 g, 0.72 mmol) in 1,4-dioxane (15 mL) was stirred at 85° C. for 4 h. The resultant mixture was filtered, the filtrate was concentrated, and the residue was subjected to silica gel column chromatography (dichloromethane:methanol=10:1) to obtain ethyl 5-(1-methyl-1H-pyrazol-3-yl)-3-(4,4,5,5-tetramethyl-1,3,2-dioxaborolan-2-yl)picolinate as a yellow solid (0.9 g, 68%). LCMS (ESI) m/z: 276.1 [M+H]⁺.

### Step 4: Synthesis of ethyl 3-(5-chloro-2-morpholino-6-(pyridin-4-ylamino)pyrimidin-4-yl)-5(1-methyl-1H-pyrazol-3-yl)picolinate

To a solution of 5-choro-6-iodo-2-morpholino-N-(pyridin-4-yl)pyrimidin-4-amine (0.3 g, 0.72 mmol) and ethyl 5-(1-methyl-1H-pyrazol-3-yl)-3-(4,4,5,5-tetramethyl-1,3,2-dioxaborolan-2-yl)picolinate (0.9 g, 2.51 mmol) in N,N-dimethylformamide (10 mL) were added potassium phosphate tribasic (0.53 g, 2.51 mmol) and 1,1′-bis(diphenylphosphino)ferrocene-palladium(II)dichloride dichloromethane complex (0.05 g, 0.07 mmol). The resultant mixture was stirred at 100° C. for 3 h and cooled. It was filtered to remove solids, the filtrate was diluted with water (30 mL) and extracted with ethyl acetate (20 mL*3). The combined organic phase was concentrated, and the residue was subjected to flash chromatography on silica gel (dichloromethane:methanol=10:1) to obtain ethyl 3-(5-chloro-2-morpholino-6-(pyridin-4-ylamino)pyrimidin-4-yl)-5-(1-methyl-1H-pyrazol-3-yl)picolinate as yellow solid. (0.11 g, 29%). LCMS (ESI) m/z: 521.3 [M+H]⁺.

### Step 5: Synthesis of (3-(5-chloro-2-morpholino-6-(pyridin-4-ylamino)pyrimidin-4-yl)-5(1-methyl-1H-pyrazol-3-yl)pyridin-2-yl)methanol

A solution of lithium aluminum hydride (1.0M in THF, 2.56 mL, 2.56 mmol) was added to a solution of ethyl 3-(5-chloro-2-morpholino-6-(pyridin-4-ylamino)pyrimidin-4-yl-5-(1-methyl-1H-pyrazol-3-yl)picolinate (130 mg, 0.25 mmol) in anhydrous tetrahydrofuran (5 mL) at 0° C. and the mixture was stirred under nitrogen atmosphere for 1.5 h at 25° C. The reaction mixture with was then quenched by the careful addition of sodium sulfate decahydrate with ice-bath cooling. It was further diluted with THF (50 mL), stirred for 15 min, filtered and the filtrate was collected. Concentration and followed by subjecting the residue to flash chromatography on silica gel (dichloromethane:methanol=10:1) afforded (3-(5-chloro-2-morpholino-6-(pyridin-4-ylamino)pyrimidin-4-yl-5-(1-methyl-1H-pyrazol-3-ylpyridin-2-ylmethanol (60 mg, 50%) as yellow solid. LCMS (ESI) m/z: 479.3 [M+H]⁺.

### Step 6: Synthesis of 9-(1-methyl-1H-pyrazol-3-yl)-2-morpholino-N-(pyridin-4-yl)-6H-pyrido[3′,2′:4,5]pyrano[3,2-d]pyrimidin-4-amine

To a mixture of (3-(5-chloro-2-morpholino-6-(pyridin-4-ylamino)pyrimidin-4-yl)-5-(1-methyl-1H-pyrazol-3-yl)pyridin-2-ylmethanol (0.045 g, 0.09 mmol) in toluene (2.5 mL) were added cesium carbonate (0.06 g, 0.19 mmol), palladium(II)acetate (0.004 g, 0.02 mmol) and racemic-2-di-t-butylphosphino-1,1′-binaphthyl (0.019 g, 0.05 mmol). The mixture was stirred at 110° C. for 5 h under argon atmosphere. The mixture was then filtered, and filtrate was concentrated. The residue was subjected to prep-HPLC (BOSTON pHlex ODS 10 um 21.2×250 mm 120 A, with mobile phase acetonitrile/0.1% ammonium bicarbonate) to obtain 9-(1-methyl-1H-pyrazol-3-yl)-2-morpholino-N-(pyridin-4-yl)-6H-pyrido[3′,2′:4,5]pyrano[3,2-d]pyrimidin-4-amine as off-white solid (10.0 mg, 24%). ¹H NMR (400 MHz, DMSO-d₆) δ 9.42 (s, 1H), 9.01 (d, J=2.1 Hz, 1H), 8.60 (d, J=2.1 Hz, 1H), 8.40 (d, J=6.2 Hz, 2H), 7.87 (d, J=6.4 Hz, 2H), 7.83 (d, J=2.2 Hz, 1H), 6.93 (d, J=2.3 Hz, 1H), 5.35 (s, 2H), 3.94 (s, 3H), 3.73 (d, J=4.0 Hz, 8H). LCMS (ESI) m/z: 443.3 [M+H]⁺.

### Example 33. Synthesis of 6-(3-(1,4-dimethyl-1H-pyrazol-3-yl)phenyl)-6-methoxy-2-morpholino-N-(pyridin-4-yl)pyrimidin-4-amine (Compound 41)

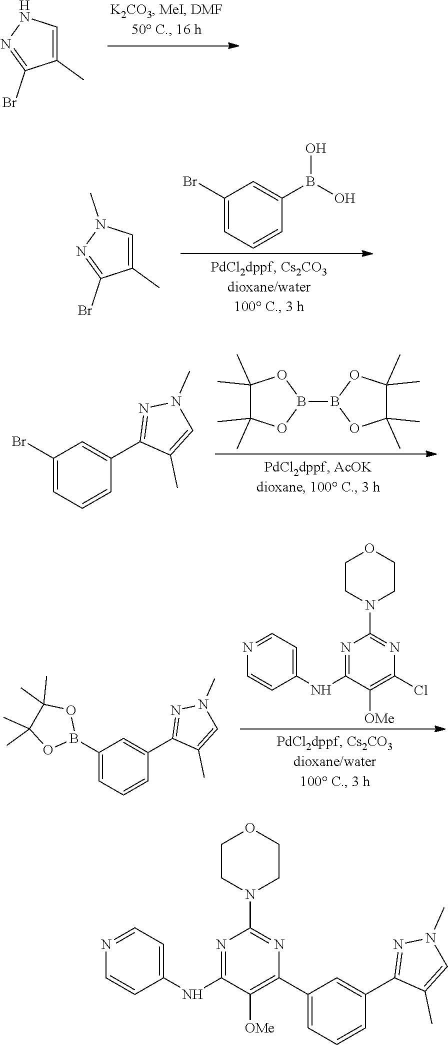

### Step 1: Synthesis of 3-bromo-1,4-dimethyl-1H-pyrazole

To a solution of 3-bromo-4-methyl-1H-pyrazole (800 mg, 5 mmol) and potassium carbonate (1.3 g, mmol) in tetrahydrofuran (25 mL) was added iodomethane (710 mg, 5 mmol) dropwise at 0° C. and the resulting reaction mixture was stirred 0.5 hour at 0° C. It was then filtered, and the filtrate was concentrated. The residue was subjected to silica gel column chromatography (petroleum ether:acetic ester=1:4) to obtain 3-bromo-1,4-dimethyl-1H-pyrazole (700 mg, 80%) as white solid. LCMS (ESI) m/z: 175.1 [M+H]⁺.

### Step 2: Synthesis of 3-(3-bromophenyl)-1,4-dimethyl-1H-pyrazole

A mixture of 3-bromo-1,4-dimethyl-1H-pyrazole (194 mg, 0.82 mmol), (3-bromophenyl)boronic acid (165 mg, 0.82 mmol), [1,1′-bis(diphenylphosphino)ferrocene]dichloropalladium(II) dichloromethane complex (66 mg, 0.082 mmol) and cesium carbonate (668 mg, 2.05 mmol) in 1,4-dioxane/water (5 mL/1 mL) was stirred at 90° C. under argon atmosphere for 16 h. The mixture was concentrated, and the residue was subjected to silica gel column chromatography (petroleum ether-acetic ester=10:1) to afford 3-(3-bromophenyl)-1,4-dimethyl-1H-pyrazole as white solid. (140 mg, 64%). LCMS (ESI) m/z: 251.1 [M+H]⁺.

### Step 3: Synthesis of 1,4-dimethyl-3-(3-(4,4,5,5-tetramethyl-1,3,2-dioxaborolan-2-yl)phenyl)-1H-pyrazole

A mixture of 3-(3-bromophenyl)-1,4-dimethyl-1H-pyrazole (140 mg, 0.52 mmol), 1,1′-bis(diphenylphosphino) ferrocene-palladium(II) dichloride dichloromethane complex (21 mg, 0.026 mmol), potassium acetate (153 mg, 1.56 mmol) and bis(pinacolato)diboron (172 mg, 3.7 mmol) in 1,4-dioxane (5 mL) was stirred at 110° C. for 16 h. The mixture was filtered, the filtrate was concentrated, and the residue was subjected to silica gel column chromatography (petroleum ether:acetic ester=4:1) to obtain 1,4-dimethyl-3-(3-(4,4,5,5-tetramethyl-1,3,2-dioxaborolan-2-yl)phenyl)-1H-pyrazole as off-yellow oil (100 mg, 61%). LCMS (ESI) m/z: 299.3 [M+H]⁺.

### Step 4: Synthesis of 6-(3-(1,4-dimethyl-1H-pyrazol-3-yl)phenyl)-5-methoxy-2-morpholino-N-(pyridin-4-yl)pyrimidin-4-amine

A mixture of 1,4-dimethyl-3-(3-(4,4,5,5-tetramethyl-1,3,2-dioxaborolan-2-yl)phenyl)-1H-pyrazole (100 mg, 0.31 mmol), 6-chloro-5-methoxy-2-morpholino-N-(pyridin-4-yl)pyrimidin-4-amine (100 mg, 0.31 mmol), 1,1′-bis(diphenylphosphino)ferrocene-palladium(II)dichloride dichloromethane complex (25 mg, 0.03 mmol) and cesium carbonate (245 mg, 0.75 mmol) in 1,4-dioxane (5 mL) with water (1 mL) was stirred at 90° C. under argon atmosphere for 16 h. The reaction mixture was filtered, and the filtrate was subjected to prep-HPLC [Welch Xtimate C18 21.2×250 mm, 10 um, with mobile phase acetonitrile/water (10 mM NH₄HCO₃ and NH₃·H₂O)] to obtain 6-(3-(1,4-dimethyl-1H-pyrazol-3-yl)phenyl)-5-methoxy-2-morpholino-N-(pyridin-4-ylpyrimidin-4-amine (20.0 mg, 14%) as yellow solid. ¹H NMR (400 MHz, DMSO-d₆) δ 9.38 (s, 1H), 8.41 (s, 2H), 8.37 (s, 1H), 7.96 (d, J=7.3 Hz, 1H), 7.90 (s, 2H), 7.77 (d, J=7.6 Hz, 1H), 7.57 (s, 1H), 7.54 (s, 1H), 3.84 (s, 3H), 3.70 (s, 8H), 3.48 (s, 3H), 2.23 (s, 3H). LCMS (ESI) m/z: 458.1 [M+H]⁺.

### Example 34. Synthesis of (R)-5-methoxy-6-(3-(1-methyl-1H-pyrazol-3-yl)phenyl)-2-(morpholin-3-ylmethoxy)-N-(pyridin-4-yl)pyrimidin-4-amine (Compound 42)

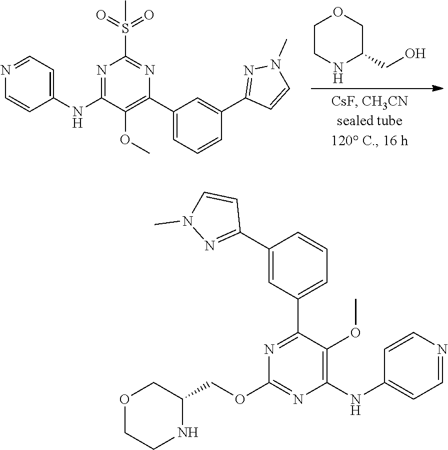

A mixture of 2-chloro-5-methoxy-6-(3-(1-methyl-1H-pyrazol-3-yl)phenyl)-N-(pyridin-4-yl)pyrimidin-4-amine (60 mg, 0.14 mmol), (S)-morpholin-3-ylmethanol (97 mg, 0.82 mmol), cesium fluoride (63 mg, 0.41 mmol) in dry acetonitrile (4 ml) was stirred at 120° C. for 16 hours in a sealed tube. The reaction mixture was filtered, concentrated and purified by pre-HPLC (BOSTON pHlex ODS 10 um 21.2×250 mm 120 A. The mobile phase was acetonitrile/0.1% ammonium bicarbonate) to afford (R)-5-methoxy-6-(3-(1-methyl-1H-pyrazol-3-yl)phenyl)-2-(morpholin-3-ylmethoxy)-N-(pyridin-4-yl)pyrimidin-4-amine (17.2 mg, 0.036 mmol, yield: 26%) as white solid. ¹H NMR (400 MHz, DMSO-d₆) δ 9.60 (s, 1H), 8.52-8.40 (m, 3H), 7.98-7.87 (m, 4H), 7.78 (d, J=2.2 Hz, 1H), 7.56 (t, J=7.8 Hz, 1H), 6.73 (d, J=2.2 Hz, 1H), 4.21 (d, J=5.9 Hz, 2H), 3.91 (s, 3H), 3.84 (dd, J=10.8, 2.8 Hz, 1H), 3.68 (d, J=10.9 Hz, 1H), 3.51 (s, 3H), 3.40 (dd, J=14.0, 6.7 Hz, 1H), 3.26 (d, J=10.2 Hz, 1H), 3.15 (s, 1H), 2.79 (d, J=13.1 Hz, 2H). LCMS (ESI) m/z: 474.1 [M+H]⁺.

### Example 35. Synthesis of R)-(4-(5-methoxy-4-(3-(1-methyl-1H-pyrazol-3-yl)phenyl)-6-(pyridin-4-ylamino) pyrimidin-2-yl)morpholin-3-yl)methanol (Compound 43)

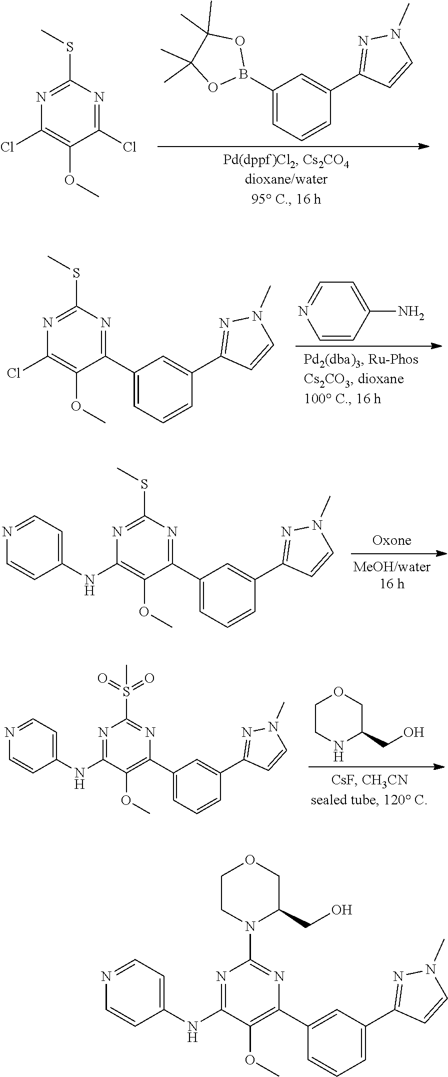

### Step 1: Synthesis of 4-chloro-5-methoxy-6-(3-(1-methyl-H-pyrazol-3-yl)phenyl)-2-(methylthio)pyrimidine

A mixture of 4,6-dichloro-5-methoxy-2-(methylthio)pyrimidine (1 g, 4.46 mmol), 1-methyl-3-(3-(4,4,5,5-tetramethyl-1,3,2-dioxaborolan-2-yl)phenyl)-1H-pyrazole (1.26 g, 4.44 mmol), 1,1′-bis(diphenylphosphino)ferrocene-palladium(II)dichloride dichloromethane complex (0.36 g, 0.44 mmol) and cesium carbonate (3.6 g, 11 mmol) in 1,4-dioxane/water (40 mL/4 mL) was stirred at 95° C. for 16 h under nitrogen atmosphere. The reaction mixture was filtered, and the filtrate was concentrated. The residue was subjected to silica gel chromatography (eluted with ethyl acetate in petroleum ether from 20% to 40%) to obtain 4-chloro-5-methoxy-6-(3-(1-methyl-1H-pyrazol-3-yl)phenyl)-2-(methylthio)pyrimidine (0.75 g, 48.6%) as white solid. LCMS (ESI) m/z: 347.0 [M+H]⁺.

### Step 2: Synthesis of 5-methoxy-6-(3-(1-methyl-1H-pyrazol-3-yl)phenyl)-2-(methylthio)-N-(pyridin-4-yl)pyrimidin-4-amine

A mixture of 4-chloro-5-methoxy-6-(3-(1-methyl-1H-pyrazol-3-yl)phenyl)-2-(methylthio)pyrimidine (0.66 g, 1.91 mmol), pyridin-4-amine (0.18 g, 1.91 mmol), tris(dibenzylideneacetone)dipalladium (0.17 g, 0.19 mmol), 2-dicyclohexylphosphino-2′,6′-diisopropoxybiphenyl (0.18 g, 0.38 mmol) and cesium carbonate (1.55 g, 4.77 mmol) in dry 1,4-dioxane (40 mL) was stirred at 100° C. for 16 h under nitrogen atmosphere. The reaction mixture was filtered, concentrated and the residue was subjected to silica gel chromatography (eluted with 7 N ammonia methanol in dichloromethane from 20% to 40%) to obtain 5-methoxy-6-(3-(1-methyl-1H-pyrazol-3-yl)phenyl)-2-(methylthio)-N-(pyridin-4-yl)pyrimidin-4-amine (0.6 g, 78.2%) as white solid. LCMS (ESI) m/z: 405.1[M+H]⁺.

### Step 3: Synthesis of 5-methoxy-6-(3-(1-methyl-1H-pyrazol-3-yl)phenyl)-2-(methylsulfonyl)-N-(pyridin-4-yl)pyrimidin-4-amine

To a solution of 5-methoxy-6-(3-(1-methyl-1H-pyrazol-3-yl)phenyl)-2-(methylthio)-N-(pyridin-4-yl)pyrimidin-4-amine (0.53 g, 1.31 mmol) in methanol (15 mL) at 0° C., was added a solution of oxone (1.61 g, 2.62 mmol) in water (7.5 mL) drop-wise. After the addition, the reaction mixture was stirred at room temperature for 3 h and the resultant precipitate was collected by filtration. The solid was further dispersed in sodium bicarbonate aqueous solution and stirred at room temperate for 20 min. The resulting solids were collected again by filtration, washed with water and dried in vacuo to afford 5-methoxy-6-(3-(1-methyl-1H-pyrazol-3-ylphenyl-2-(methylsulfonyl)-N-(pyridin-4-yl)pyrimidin-4-amine (0.4 g, 70%) as white solid. LCMS (ESI) m/z: 437.1 [M+H]⁺.

### Step 4: Synthesis of (R)-(4-(5-methoxy-4-(3-(1-methyl-1H-pyrazol-3-yl)phenyl)-6-(pyridin-4-ylamino)pyrimidin-2-yl)morpholin-3-yl)methanol

A mixture of 2-chloro-5-methoxy-6-(3-(1-methyl-1H-pyrazol-3-yl)phenyl))-N-(pyridin-4-yl)pyrimidin-4-amine (50 mg, 0.11 mmol), (R)-morpholin-3-ylmethanol (80 mg, 0.69 mmol), cesium fluoride (52 mg, 0.34 mmol) in dry acetonitrile (2 mL) was stirred at 120° C. for 16 h in a sealed tube. The reaction mixture was filtered to remove solids, the filtrate was concentrated, and the residue was subjected to prep-HPLC (BOSTON pHlex ODS 10 um 21.2×250 mm 120 A. The mobile phase was acetonitrile/0.1% ammonium bicarbonate) to obtain (R)-(4-(5-methoxy-4-(3-(1-methyl-1H-pyrazol-3-yl)phenyl)-6-(pyridin-4-ylamino)pyrimidin-2-yl)morpholin-3-yl)methanol (4 mg, 7.7%) as white solid. ¹H NMR (400 MHz, DMSO-d₆) δ 9.32 (s, 1H), 8.45 (s, 1H), 8.38 (d, J=6.2 Hz, 2H), 7.93-7.81 (m, 4H), 7.77 (d, J=2.1 Hz, 1H), 7.52 (t, J=7.7 Hz, 1H), 6.71 (d, J=2.2 Hz, 1H), 4.91 (t, J=5.1 Hz, 1H), 4.39 (s, 1H), 4.26 (d, J=13.8 Hz, 1H), 4.11 (d, J=11.2 Hz, 1H), 4.00-3.90 (m, 4H), 3.79 (d, J=5.7 Hz, 1H), 3.57-3.40 (m, 6H), 3.16 (t, J=16.3 Hz, 1H). LCMS (ESI) m/z: 474.2 [M+H]⁺.

### Example 36. Synthesis of 1-(5-methoxy-4-(3-(1-methyl-1H-pyrazol-3-yl)phenyl)-6-(pyridin-4-ylamino)pyrimidin-2-yl)pyrrolidin-2-one (Compound 44)

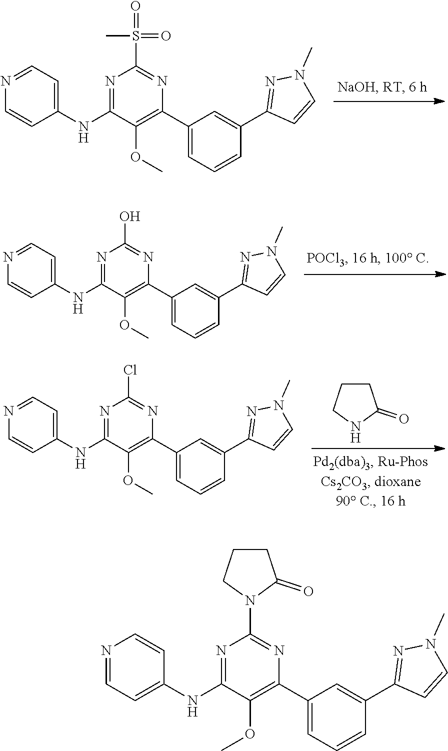

### Step 1: Synthesis of 5-methoxy-4-(3-(1-methyl-1H-pyrazol-3-yl)phenyl)-6-(pyridin-4-ylamino)pyrimidin-2-od

A solution of 5-methoxy-6-(3-(1-methyl-1H-pyrazol-3-yl)phenyl-2-(methylsulfonyl)-N-(pyridin-4-yl)pyrimidin-4-amine (180 mg, 0.413 mmol) in 2.0 M sodium hydroxide (5 mL) was stirred at room temperature for 6 h under nitrogen atmosphere. The mixture was triturated with water and the resultant precipitate was collected by filtration and dried in vacuo to obtain 5-methoxy-4-(3-(1-methyl-1H-pyrazol-3-yl)phenyl)-6-(pyridin-4-ylamino)pyrimidin-2-ol (152 mg, 88.64%) as white solid. LCMS (ESI) m/z: 375.0 [M+H]⁺.

### Step 2: Synthesis of 2-chloro-5-methoxy-6-(3-(1-methyl-1H-pyrazol-3-yl)phenyl)-N-(pyridin-4-yl)pyrimidin-4-amine

A solution of 5-methoxy-4-(3-(1-methyl-1H-pyrazol-3-yl)phenyl)-6-(pyridin-4-ylamino)pyrimidin-2-ol (132 mg, 0.353 mmol) in phosphorus oxychloride (4 ml) was stirred at 100° C. for 16 h under nitrogen atmosphere. The mixture was cooled down and neutralized by the addition of saturated aqueous sodium bicarbonate. The phases were separated, and the aqueous phase was extracted with 20 mL of dichloromethane. The combined organic phases were dried over sodium sulfate, filtered and concentrated. The residue was subjected to silica gel chromatography eluting with a linear gradient of 0% to 63% ethyl acetate in petroleum ether to afford 2-chloro-5-methoxy-6-(3-(1-methyl-1H-pyrazol-3-yl)phenyl)-N-(pyridin-4-yl)pyrimidin-4-amine (40 mg, 25.95%) as white solid. LCMS (ESI) m/z: 393.0 [M+H]⁺.

### Step 3: Synthesis of 1-(5-methoxy-4-(3-(1-methyl-1H-pyrazol-3-yl)phenyl)-6-(pyridin-4-ylamino)pyrimidin-2-yl)pyrrolidin-2-one

A mixture of 2-chloro-5-methoxy-6-(3-(1-methyl-1H-pyrazol-3-yl)phenyl)-N-(pyridin-4-yl)pyrimidin-4-amine (40 mg, 0.102 mmol), pyrrolidin-2-one (13 mg, 0.153 mmol), tris(dibenzylideneacetone)dipalladium(9 mg, 0.01 mmol), 2-dicyclohexylphosphino-2′,6′-diisopropoxybiphenyl (9 mg, 0.02 mmol) and potassium carbonate (84 mg, 0.255 mmol) in dry 1,4-dioxane (6 ml) was stirred at 90° C. for 16 h under nitrogen atmosphere. The mixture was filtered to remove the solids, the filtrate was concentrated, and the residue was subjected to prep-HPLC (BOSTON pHlex ODS 10 um 21.2×250 mm 120 A). The mobile phase was acetonitrile/0.1% ammonium bicarbonate) to obtain 1-(5-methoxy-4-(3-(1-methyl-1H-pyrazol-3-yl)phenyl)-6-(pyridin-4-ylamino)pyrimidin-2-yl)pyrrolidin-2-one (8.1 mg, 18.04%) as white solid. ¹H NMR (400 MHz, DMSO-d₆) δ 10.17 (s, 1H), 8.64 (s, 2H), 8.54 (d, J=6.4 Hz, 2H), 8.51 (s, 1H), 7.94 (dd, J=15.3, 7.8 Hz, 2H), 7.78 (d, J=2.2 Hz, 1H), 7.58 (t, J=7.8 Hz, 1H), 6.74 (d, J=2.2 Hz, 1H), 4.08 (t, J=7.0 Hz, 2H), 3.91 (s, 3H), 3.56 (s, 3H), 2.61 (t, J=8.0 Hz, 2H), 2.10-2.04 (m, 2H). LCMS (ESI) m/z: 442.0 [M+H]⁺.

### Example 37. Synthesis of dimethyl(4-(3-(1-methyl-1H-pyrazol-3-yl)phenyl)-2-morpholino-6-(pyridin-3-ylamino)pyrimidin-6-yl)phosphine oxide (Compound 45)

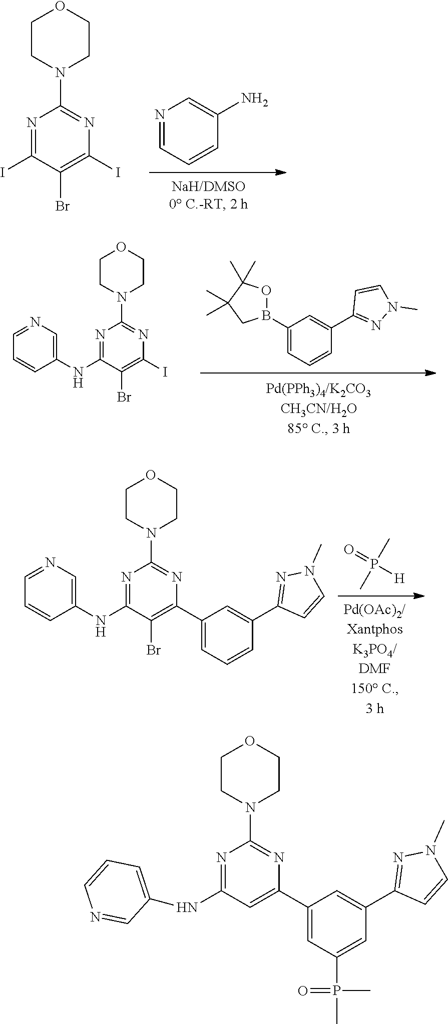

### Step 1: Synthesis of 5-bromo-6-iodo-2-morpholino-N-(pyridin-3-yl)pyrimidin-4-amine

To a mixture of pyridin-3-amine (376 mg, 4.0 mmol) in dimethyl sulfoxide (15 mL) was added sodium hydride (320 mg, 8.0 mmol) at 0° C. The mixture was warmed up and stirred at 28° C. for 20 min followed by the addition of 4-(5-bromo-4,6-diiodopyrimidin-2-yl)morpholine (2.0 g, 4.0 mmol). Stirring was continued at 28° C. for another 30 min and then the mixture was poured into dilute hydrochloric acid (cooled with crushed ice). The formed precipitate was collected by filtration and dried under vacuum to afford 5-bromo-6-iodo-2-morpholino-N-(pyridin-3-yl)pyrimidin-4-amine (500 mg, 24.2%) as grey solid. LCMS (ESI) m/z: 461.9/464.9 [M+H]⁺.

### Step 2: Synthesis of 5-bromo-6-(3-(1-methyl-H-pyrazol-3-yl)phenyl)-2-morpholino-N-(pyridin-3-yl)pyrimidin-4-amine

To a mixture of 1-methyl-3-(3-(4,4,5,5-tetramethyl-1,3,2-dioxaborolan-2-yl)phenyl)-1H-pyrazole (0.28 g, 0.97 mmol) in acetonitrile/water (15 mL/3 mL) were added 5-bromo-6-iodo-2-morpholino-N-(pyridin-3-yl)pyrimidin-4-amine (0.5 g, 1.1 mmol), potassium carbonate (0.37 g, 2.71 mmol) and tetrakis(triphenylphosphine)palladium (0.13 g, 0.11 mmol). The resultant reaction mixture was stirred at 85° C. for 3 h under argon atmosphere and cooled. It was filtered to remove the solids, the filtrate was concentrated, and the residue was subjected to silica gel column chromatography (dichloromethane:methanol=15:1) to obtain 5-bromo-6-(3-(1-methyl-1H-pyrazol-3-yl)phenyl)-2-morpholino-N-(pyridin-3-yl)pyrimidin-4-amine as a yellow solid (0.12 g, 23%). LCMS (ESI) m/z: 492.1 [M+H]⁺.

### Step 3: Synthesis of dimethyl(4-(3-(1-methyl-1H-pyrazol-3-yl)phenyl)-2-morpholino-6-(pyridin-3-ylamino)pyrimidin-6-yl)phosphine oxide

To a mixture of dimethylphosphine oxide (0.023 g, 0.29 mmol) in N,N-dimethylformamide (5 mL) were added 5-bromo-6-(3-(1-methyl-1H-pyrazol-3-yl)phenyl)-2-morpholino-N-(pyridin-3-yl)pyrimidin-4-amine (0.12 g, 0.24 mmol), potassium phosphate tribasic (0.062 g, 0.29 mmol), palladium(II)acetate (0.006 g, 0.024 mmol) and 4,5-bis(diphenylphosphino)-9,9-dimethylxanthene (0.028 g, 0.048 mmol). The resultant mixture was stirred at 150° C. for 3 h under argon atmosphere and cooled. The mixture was filtered to remove the solids, the filtrate was concentrated, and the residue was subjected to prep-HPLC (BOSTON pHlex ODS 10 um 21.2×250 mm 120 A, with mobile phase acetonitrile/0.1% ammonium bicarbonate) to obtain dimethyl(4-(3-(1-methyl-1H-pyrazol-3-yl)phenyl)-2-morpholino-6-(pyridin-3-ylamino)pyrimidin-5-yl)phosphine oxide (2.6 mg, 2%). as off-white solid ¹H NMR (400 MHz, DMSO-d₆) δ 11.97 (s, 1H), 8.71 (d, J=2.4 Hz, 1H), 8.24 (d, J=4.7 Hz, 1H), 7.99 (d, J=8.5 Hz, 1H), 7.90 (d, J=7.9 Hz, 1H), 7.75 (d, J=1.9 Hz, 2H), 7.48 (t, J=7.7 Hz, 1H), 7.39 (dd, J=8.2, 4.7 Hz, 1H), 7.28 (d, J=7.6 Hz, 1H), 6.77 (d, J=2.2 Hz, 1H), 3.88 (s, 3H), 3.70 (d, J=4.7 Hz, 4H), 3.64 (s, 4H), 1.36 (s, 3H), 1.33 (s, 3H). LCMS (ESI) m/z: 490.2 [M+H]⁺.

### Example 38. Synthesis of 1-(5-methoxy-4-(3-(1-methyl-1H-pyrazol-3-yl)phenyl)-6-(pyridin-4-ylamino)pyrimidin-2-yl)piperidin-4-ol (Compound 46)

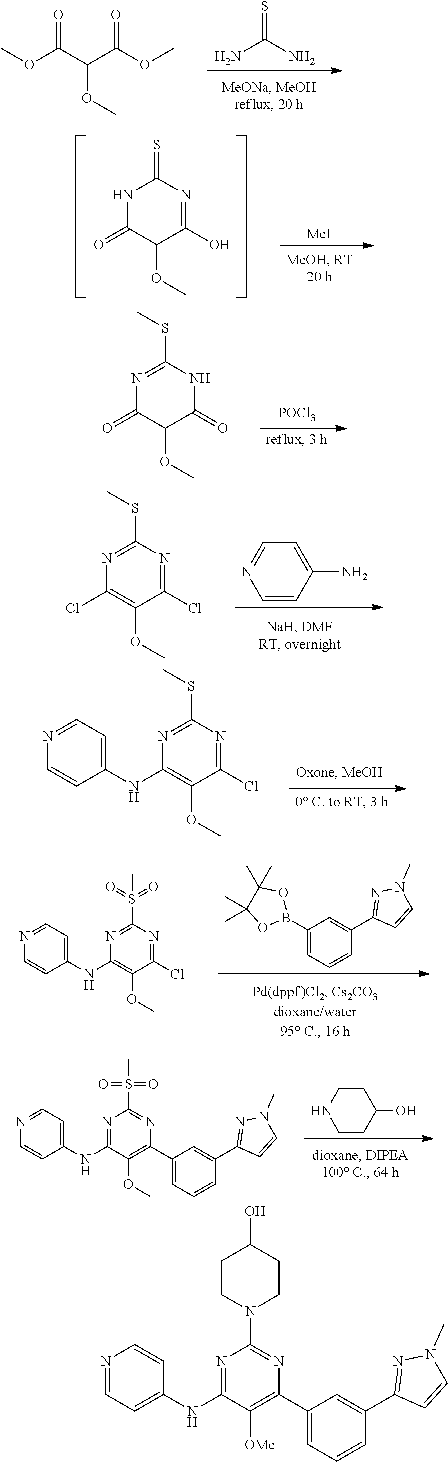

### Step 1: Synthesis of 6-hydroxy-6-methoxy-2-thioxo-2,5-dihydropyrimidin-4(3H)-one

To a solution of thiourea (4.7 g, 61.7 mmol) and dimethyl 2-methoxymalonate (10 g, 61.7 mmol) in methanol (50 mL) was added sodium methoxide solution (30% solution in methanol, 11.6 mL, 61.7 mmol). After the addition, the reaction mixture was stirred at 80° C. for 20 h. It was cooled down and used directly in next step without further purification. LCMS (ESI) 174.7 [M+H]⁺.

### Step 2: Synthesis of 5-methoxy-2-(methylthio)pyrimidine-4,6(1H,5H)-dione

To the reaction mixture from above step was added iodomethane (10.5 g, 74 mmol) dropwise. After the addition, the reaction mixture was stirred at room temperature for 20 h and concentrated. The residue was triturated with water (30 mL), the precipitate was collected by filtration, the solids were washed with water (30 mL) and dried in vacuo to afford 5-methoxy-2-(methylthio)pyrimidine-4,6(1H,5H)-dione (7.5 g, 64.7% for two steps) as white solid. LCMS (ESI) 188.7 [M+H]⁺.

### Step 3: Synthesis of 4,6-dichloro-6-methoxy-2-(methylthio)pyrimidine

A mixture of 5-methoxy-2-(methylthio)pyrimidine-4,6(1H,5H)-dione (7 g, 37.2 mmol) and phosphorus oxychloride (50 mL) was stirred at 110° C. for 3 h and concentrated. The residue was poured slowly into warm water (˜40° C.) with vigorous stirring. After the addition, the mixture was extracted with ethyl acetate (50 mL) twice. The combined organic phase was washed with brine (50 mL), dried over sodium sulfate, filtered and concentrated. The residue was subjected to column chromatography on silica gel (eluted with ethyl acetate in petroleum ether from 20% to 40%) to obtain 4,6-dichloro-5-methoxy-2-(methylthio)pyrimidine (7 g, 84%) as white solid. LCMS (ESI) m/z: 224.9 [M+H]⁺.

### Step 4: Synthesis of 6-chloro-5-methoxy-2-(methylthio)-N-(pyridin-4-yl)pyrimidin-4-amine

To a solution of pyridin-4-amine (0.28 g, 3 mmol) in N,N-dimethylformamide (10 mL), at 0° C., was added sodium hydride (60%, 0.18 g, 4.5 mmol) in portions. After the addition, the mixture was stirred at room temperature for 30 mins and cooled to 0° C. again. Then a solution of 4,6-dichloro-5-methoxy-2-(methylthio)pyrimidine (0.7 g, 3.13 mmol) in N,N-dimethylformamide (5 mL) was added dropwise. The reaction mixture was warmed up to room temperature and stirred for additional 16 h. It was diluted with ethyl acetate and water (20 mL/20 mL), organic layer separated, and aqueous layer was extracted with ethyl acetate (20 mL) twice. The combined organic phase was dried over sodium sulfate, filtered and the filtrate was concentrated. The residue was subjected to column chromatography on silica gel (eluted with 7N ammonia methanol in dichloromethane from 5% to 15%) to obtain 6-chloro-5-methoxy-2-(methylthio)-N-(pyridin-4-yl)pyrimidin-4-amine (0.4 g, 47.3%) as brown solid. LCMS (ESI) m/z: 282.8 [M+H]⁺.

### Step 5: Synthesis of 6-chloro-5-methoxy-2-(methylsulfonyl)-N-(pyridin-4-yl)pyrimidin-4-amine

To a solution of 6-chloro-5-methoxy-2-(methylthio)-N-(pyridin-4-yl)pyrimidin-4-amine (0.4 g, 1.42 mmol) in methanol (15 mL) was added a solution of oxone (1.74 g, 2.8 mmol) in water (8 mL) dropwise at 0° C. After the addition, the reaction mixture was stirred at room temperature for 3 h. It was then neutralized to pH=7-8 with aqueous sodium bicarbonate solution and the resulting solids were collected by filtration, washed with water and dried in vacuo to afford 6-chloro-5-methoxy-2-(methylsulfonyl)-N-(pyridin-4-ylpyrimidin-4-amine (0.25 g, 56.3%) as brown solid. LCMS (ESI) m/z: 314.7 [M+H]⁺.

### Step 6: Synthesis of 5-methoxy-6-(3-(1-methyl-1H-pyrazol-3-yl)phenyl)-2-(methylsulfonyl)-N-(pyridin-4-yl)pyrimidin-4-amine

A mixture of 6-chloro-5-methoxy-2-(methylsulfonyl)-N-(pyridin-4-yl)pyrimidin-4-amine (0.25 g, 0.8 mmol), 1-methyl-3-(3-(4,4,5,5-tetramethyl-1,3,2-dioxaborolan-2-yl)phenyl)-1H-pyrazole (0.23 g, 0.8 mmol), 1,1′-bis(diphenylphosphino)ferrocene palladium (II)dichloride dichloromethane complex (65 mg, 0.08 mmol) and cesium carbonate (0.65 g, 2 mmol) in 1,4-dioxane/water (25 mL/4 mL) was stirred at 95° C. for 16 h under nitrogen atmosphere. The resultant reaction mixture was concentrated, diluted with water (20 mL) and extracted with ethyl acetate (20 mL×2). The combined organic phase was washed with brine (20 mL), dried over sodium sulfate, filtered and concentrated. The residue was subjected to column chromatography on silica gel (eluted with 7N ammonia methanol in dichloromethane from 5% to 15%) to afford 5-methoxy-6-(3-(1-methyl-1H-pyrazol-3-yl)phenyl)-2-(methylsulfonyl)-N-(pyridin-4-yl)pyrimidin-4-amine (0.18 g, 51.6%) as yellow solid. LCMS (ESI) m/z: 437.1 [M+H]⁺.

### Step 7: Synthesis of 1-(5-methoxy-4-(3-(1-methyl-1H-pyrazol-3-yl)phenyl)-6-(pyridin-4-ylamino)pyrimidin-2-yl)piperidin-4-ol

A mixture of 5-methoxy-6-(3-(1-methyl-1H-pyrazol-3-yl)phenyl)-2-(methylsulfonyl)-N-(pyridin-4-ylpyrimidin-4-amine (80 mg, 0.18 mmol), piperidin-4-ol (37 mg, 0.36 mmol), and N,N-diisopropylethylamine (71 mg, 0.55 mmol) in 1,4-dioxane (5 mL) was stirred at 100° C. for 64 h. The reaction mixture was concentrated, and the residue was subjected to prep-HPLC (base) to obtain 1-(5-methoxy-4-(3-(1-methyl-1H-pyrazol-3-ylphenyl)-6-(pyridin-4-ylamino)pyrimidin-2-ylpiperidin-4-ol (6.5 mg, 8%) as white solid. ¹H NMR (400 MHz, DMSO-d₆) δ 9.29 (s, 1H), 8.47-8.35 (m, 3H), 7.87 (dt, J=7.0, 5.0 Hz, 4H), 7.77 (d, J=2.2 Hz, 1H), 7.52 (t, J=7.8 Hz, 1H), 6.70 (d, J=2.2 Hz, 1H), 4.72 (d, J=4.3 Hz, 1H), 4.28 (d, J=13.6 Hz, 2H), 3.90 (s, 3H), 3.73 (s, 1H), 3.44 (s, 3H), 3.26 (t, J=10.2 Hz, 2H), 1.82 (s, 2H), 1.39 (d, J=9.5 Hz, 2H). LCMS (ESI) m/z: 458.3 [M+H]⁺.

### Example 39. Synthesis of 5-methoxy-6-(3-(4-methoxy-1-methyl-1H-pyrazol-3-yl)phenyl)-2-morpholino-N-(pyridin-4-yl)pyrimidin-4-amine (Compound 47)

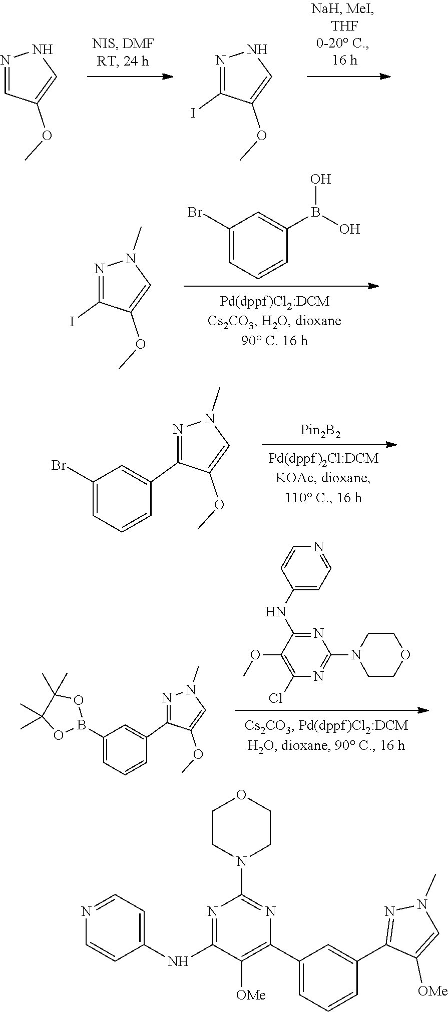

### Step 1: Synthesis of 3-iodo-4-methoxy-1H-pyrazole

A mixture of 4-methoxy-1H-pyrazole (600 mg, 6.0 mmol), N-iodosuccinimide (1.08 g, 4.8 mmol) in N,N-dimethylformamide (50 mL) was stirred at 25° C. under nitrogen atmosphere for 24 h. The reaction mixture was filtered, the filtrate was concentrated, and the residue was subjected to silica gel column chromatography (petroleum ether:ethyl acetate=12:78) to obtain 3-iodo-4-methoxy-1H-pyrazole (800 mg, 60%) as white solid. LCMS (ESI) m/z: 225.1 [M+H]⁺.

### Step 2: Synthesis of 3-iodo-4-methoxy-1-methyl-1H-pyrazole

To a solution of 3-iodo-4-methoxy-1H-pyrazole (800 mg, 3.57 mmol) in tetrahydrofuran (25 mL) at 0° C., was added sodium hydride (60%, 171.36 mg, 4.28 mmol) in portions. After the addition, the mixture was stirred for another 30 min. It was then filtered, the filtrate was concentrated, and residue was subjected to silica gel column chromatography (petroleum ether:ethyl acetate=1:4) to obtain 3-iodo-4-methoxy-1-methyl-1H-pyrazole (194 mg, 19%) as white solid. LCMS (ESI) m/z: 239.1 [M+H]⁺.

### Step 3: Synthesis of 3-(3-bromophenyl)-4-methoxy-1-methyl-1H-pyrazole

A mixture of 3-iodo-4-methoxy-1-methyl-1H-pyrazole (194 mg, 0.82 mmol), (3-bromophenyl)boronic acid (165 mg, 0.82 mmol), [1,1′-bis(diphenylphosphino)ferrocene]dichloropalladium(II) dichloromethane complex (66 mg, 0.082 mmol) and cesium carbonate (668 mg, 2.05 mmol) in 1,4-dioxane/water (5 mL/1 mL) was stirred at 90° C. under argon atmosphere for 16 h. The mixture was concentrated, and the residue was subjected to silica gel column chromatography (petroleum ether:ethyl acetate=10:1) to obtain 3-(3-bromophenyl)-4-methoxy-1-methyl-1H-pyrazole (140 mg, 64%) as white solid. LCMS (ESI) m/z: 269.0 [M+H]⁺.

### Step 4: Synthesis of 4-methoxy-1-methyl-3-(3-(4,4,5,5-tetramethyl-1,3,2-dioxaborolan-2-yl)phenyl)-1H-pyrazole

A mixture of 3-(3-bromophenyl)-4-methoxy-1-methyl-1H-pyrazole (140 mg, 0.52 mmol), 1,1′-bis(diphenylphosphino) ferrocene-palladium(II) dichloride dichloromethane complex (21 mg, 0.026 mmol), potassium acetate (153 mg, 1.56 mmol) and bis(pinacolato)diboron (172 mg, 3.7 mmol) in 1,4-dioxane (5 mL) was stirred at 110° C. for 16 h. The mixture was filtered, the filtrate was concentrated, and the residue was subjected to silica gel column chromatography (petroleum ether:ethyl acetate=4:1) to obtain 4-methoxy-1-methyl-3-(3-(4,4,5,5-tetramethyl-1,3,2-dioxaborolan-2-yl)phenyl)-1H-pyrazole as pale-yellow oil (100 mg, 61%). LCMS (ESI) m/z: 315.3 [M+H]⁺.

### Step 5: Synthesis of 5-methoxy-6-(3-(4-methoxy-1-methyl-1H-pyrazol-3-yl)phenyl)-2-morpholino-N-(pyridin-4-yl)pyrimidin-4-amine

A mixture of 4-methoxy-1-methyl-3-(3-(4,4,5,5-tetramethyl-1,3,2-dioxaborolan-2-yl)phenyl)-1H-pyrazole (100 mg, 0.31 mmol), 6-chloro-5-methoxy-2-morpholino-N-(pyridin-4-yl)pyrimidin-4-amine (100 mg, 0.31 mmol), 1,1′-Bis(diphenylphosphino) ferrocene-palladium (II)dichloride dichloromethane complex (25 mg, 0.03 mmol) and cesium carbonate (245 mg, 0.75 mmol) in 1,4-dioxane (5 mL) and water (1 mL) was stirred at 90° C. under argon atmosphere for 16 h. The reaction mixture was filtered, the filtrate was subjected to prep-HPLC [Welch Xtimate C18 21.2×250 mm, 10 um, with mobile phase acetonitrile/water (10 mM NH₄HCO₃ and NH₃·H₂O)] to obtain 5-methoxy-6-(3-(4-methoxy-1-methyl-1H-pyrazol-3-yl)phenyl)-2-morpholino-N-(pyridin-4-yl)pyrimidin-4-amine (58.8 mg, 40%) as yellow solid. ¹H NMR (500 MHz, DMSO-d₆) δ 9.35 (s, 1H), 8.63 (s, 1H), 8.41 (d, J=6.2 Hz, 2H), 7.91 (t, J=7.6 Hz, 2H), 7.88 (d, J=6.3 Hz, 2H), 7.63 (s, 1H), 7.50 (t, J=7.8 Hz, 1H), 3.82 (s, 3H), 3.78 (s, 3H), 3.71 (d, J=1.8 Hz, 8H), 3.47 (s, 3H). LCMS (ESI) m/z: 474.2 [M+H]⁺.

### Example 40. Synthesis of 1-(3-(5-methoxy-2-morpholino-6-(pyridin-4-ylamino)pyrimidin-4-yl)phenyl)piperidin-4-ol (Compound 48)

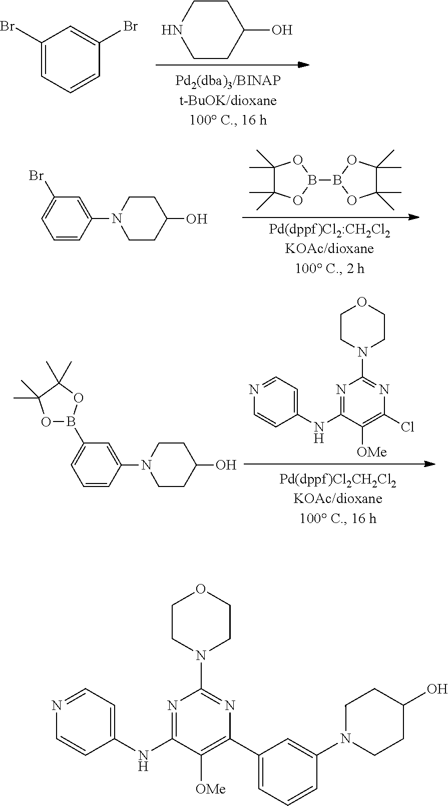

### Step 1: Synthesis of 1-(3-bromophenyl)piperidin-4-ol

A mixture of 1,3-dibromobenzene (2.34 g, 10.0 mmol), piperidin-4-ol (870 mg, 10.0 mmol), tris(dibenzylideneacetone)dipalladium(0) (458 mg, 0.5 mmol), 1.1′-binaphthyl-2.2′-diphemyl phosphine (622 mg, 1.0 mmol) and potassium tert-butoxide (2.24 g, 20.0 mmol) in 1,4-dioxane (40 mL) was stirred at 100° C. under nitrogen atmosphere for 16 h. The mixture was poured into water and extracted with dichloromethane (200 mL*2). The combined organic phase was concentrated, and the residue was subjected to silica gel column chromatography (10% of methanol in dichloromethane) to afford 1-(3-bromophenyl)piperidin-4-ol (300 mg, 12%) as red oil. LCMS (ESI) m/z: 255.9/257.9 [M+H]⁺.

### Step 2: Synthesis of 1-(3-(4,4,5,5-tetramethyl-1,3,2-dioxaborolan-2-yl)phenyl)piperidin-4-ol

A mixture of 1-(3-bromophenyl)piperidin-4-ol (275 mg, 1.1 mmol), 4,4,4′,4′,5,5,5′,5′-octamethyl-2,2′-bi(1,3,2-dioxaborolane) (559 mg, 2.2 mmol), 1,1′-bis(diphenylphosphino)ferrocene-palladium(II)dichloride dichloromethane complex (90 mg, 0.11 mmol) and potassium acetate (216 mg, 2.2 mmol) in dioxane (10 mL) was stirred at 100° C. under nitrogen atmosphere for 4 h. The mixture was poured into water and extracted with ethyl acetate (150 mL*2). The combined organic phase was concentrated, and the residue was subjected to silica gel column chromatography (5% methanol in dichloromethane) to obtain 1-(3-(4,4,5,5-tetramethyl-1,3,2-dioxaborolan-2-yl)phenyl)piperidin-4-ol (250 mg, 74.5%) as brown oil. LCMS (ESI) m/z: 304.2 [M+H]⁺.

### Step 3: Synthesis of 1-(3-(5-methoxy-2-morpholino-6-(pyridin-4-ylamino)pyrimidin-4-yl)phenyl)piperidin-4-ol

A mixture of 6-chloro-5-methoxy-2-morpholino-N-(pyridin-3-yl)pyrimidin-4-amine (160 mg, 0.5 mmol), 1-(3-(4,4,5,5-tetramethyl-1,3,2-dioxaborolan-2-yl)phenyl) piperidin-4-ol (227 mg, 0.75 mmol), 1,1′-bis(diphenylphosphino)ferrocene-palladium(II)dichloride dichloromethane complex (41 mg, 0.05 mmol) and cesium carbonate (325 mg, 1.0 mmol) in dioxane (6 mL) and water (0.6 mL) was stirred at 100° C. under nitrogen atmosphere for 2 h. The mixture was poured into water and extracted with dichloromethane (100 mL*2). The combined organic phase was concentrated, and the residue was subjected to silica gel column chromatography (15% methanol in dichloromethane) and prep-HPLC [Welch Xtimate C18 21.2×250 mm, 10 um, with mobile phase acetonitrile/water (10 mM NH₄HCO₃ and NH₃·H₂O)] to obtain 1-(3-(5-methoxy-2-morpholino-6-(pyridin-4-ylamino)pyrimidin-4-yl)phenyl)piperidin-4-ol (45.5 mg, 19.6%) as light-yellow solid. ¹H NMR (500 MHz, DMSO-d₆) δ 9.29 (s, 1H), 8.40 (d, J=6.3 Hz, 2H), 7.87 (d, J=6.4 Hz, 2H), 7.58 (s, 1H), 7.39 (d, J=7.7 Hz, 1H), 7.31 (t, J=7.9 Hz, 1H), 7.06 (dd, J=8.2, 2.0 Hz, 1H), 4.69 (d, J=4.3 Hz, 1H), 3.70-3.67 (m, 9H), 3.58-3.53 (m, 2H), 3.44 (s, 3H), 2.93-2.86 (m, 2H), 1.86-1.81 (m, 2H), 1.53-1.44 (m, 2H); LCMS (ESI) m/z: 463.2 [M+H]⁺.

### Example 41. Synthesis of 9-(1-methyl-1H-pyrazol-3-yl)-2-morpholino-N-(pyridin-4-yl)-6H-isochromeno[4,3-d]pyrimidin-4-amine (Compound 49)

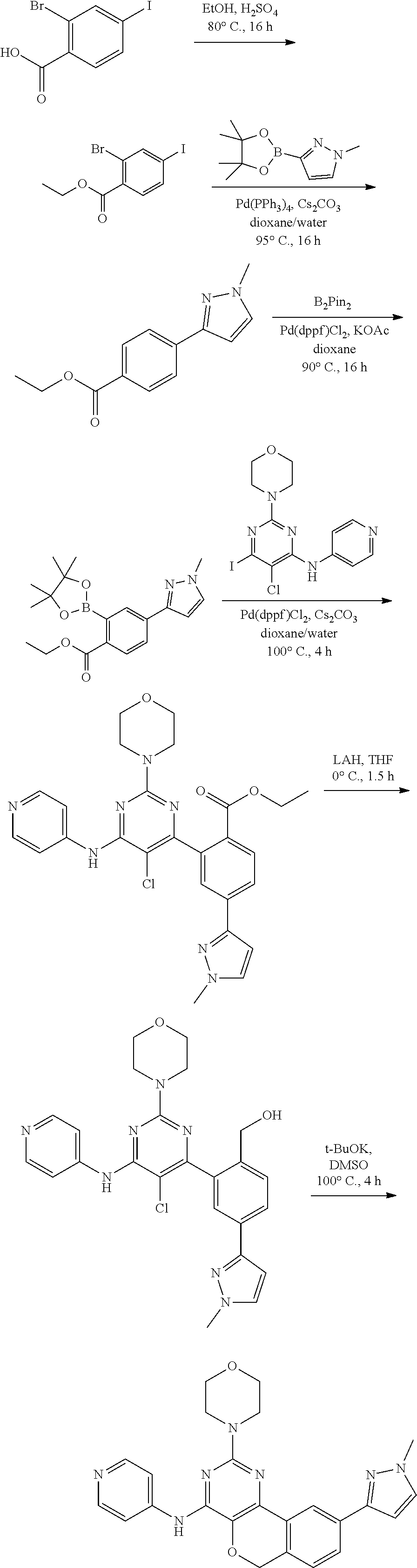

### Step 1: Synthesis of ethyl 2-bromo-4-iodobenzoate

To a solution of 2-bromo-4-iodobenzoic acid (5 g, 15.3 mmol) in ethanol (100 mL) was added sulfuric acid (5 mL) and the reaction mixture was stirred at 80° C. for 16 h. The mixture was concentrated, and the residue was subjected to column chromatography on silica gel (5% to 10% ethyl acetate in petroleum ether) to obtain ethyl 2-bromo-4-iodobenzoate (5.3 g, 97.8%) as yellow oil. LCMS (ESI) m/z: 355.0, 357.0 [M+H]⁺.

### Step 2: Synthesis of ethyl 2-bromo-4-(l-methyl-1H-pyrazol-3-yl)benzoate

A mixture of ethyl 2-bromo-4-iodobenzoate (2 g, 5.63 mmol), 1-methyl-3-(4,4,5,5-tetramethyl-1,3,2-dioxaborolan-2-yl)-1H-pyrazole (1 g, 4.8 mmol), 1,1′-bis(diphenylphosphino)ferrocene-palladium(II)dichloride dichloromethane complex (0.46 g, 0.56 mmol), and cesium carbonate (4.6 g, 14.1 mmol) in 1,4-dioxane/water (50 mL/5 mL) was stirred at 85° C. for 16 h under argon atmosphere. It was concentrated, the residue was diluted with water (20 mL) and extracted with dichloromethane (20 mL×2). The combined organic phase was washed with brine (20 mL), dried over sodium sulfate, filtered and concentrated. The residue was subjected to flash chromatography (eluted with ethyl acetate in petroleum ether from 30% to 60%) to obtain ethyl 2-bromo-4-(1-methyl-1H-pyrazol-3-yl)benzoate (1.45 g, 83.6%) as white solid. LCMS (ESI) m/z: 310.6 [M+H]⁺.

### Step 3: Synthesis of ethyl 4-(1-methyl-1H-pyrazol-3-yl)-2-(4,4,5,5-tetramethyl-1,3,2-dioxaborolan-2-yl)benzoate

A mixture of ethyl 2-bromo-4-(1-methyl-1H-pyrazol-3-yl)benzoate (1.1 g, 3.57 mmol), bis(pinacolato)diboron (1.36 g, 5.36 mmol), 1,1′-bis(diphenylphosphino)ferrocene-palladium(II)dichloride dichloromethane complex (0.29 g, 0.36 mmol) and potassium acetate (0.87 g, 8.92 mmol) in 1,4-dioxane (25 mL) was stirred at 90° C. for 16 h. The mixture was then concentrated, and the residue was subjected to flash chromatography (eluted with ethyl acetate in petroleum ether from 30% to 60%) to obtain ethyl 4-(1-methyl-1H-pyrazol-3-yl-2-(4,4,5,5-tetramethyl-1,3,2-dioxaborolan-2-yl)benzoate (1 g, 78.7%) as white solid. LCMS (ESI) m/z: 357.1[M+H]⁺.

### Step 4: Synthesis of ethyl 2-(5-chloro-2-morpholino-6-(pyridin-4-ylamino)pyrimidin-4-yl)-4-(1-methyl-1H-pyrazol-3-yl)benzoate

To a solution of 5-chloro-6-iodo-2-morpholino-N-(pyridin-4-yl)pyrimidin-4-amine (0.26 g, 0.62 mmol) and ethyl 4-(1-methyl-1H-pyrazol-3-yl)-2-(4,4,5,5-tetramethyl-1,3,2-dioxaborolan-2-yl)benzoate (0.27 g, 0.75 mmol) in 1,4-dioxane/water (7 mL/2 mL) were added cesium carbonate (0.41 g, 1.25 mmol) and [1,1′-bis(diphenylphosphino)ferrocene]dichloropalladium(II) (0.044 g, 0.06 mmol). The mixture was stirred at 90° C. for 2 h under argon atmosphere and cooled. It was poured into ice-water and the mixture was extracted with ethyl acetate (15 mL*3). The combined organic layer was washed with brine, dried over sodium sulfate and concentrated. The residue was subjected to flash chromatography on silica gel (dichloromethane:methanol=10:1) to obtain ethyl 2-(5-chloro-2-morpholino-6-(pyridin-4-ylamino)pyrimidin-4-yl)-4-(1-methyl-1H-pyrazol-3-yl)benzoate (0.19 g, 59%) as yellow solid. LCMS (ESI) m/z: 520.3 [M+H]⁺.

### Step 5: Synthesis of (2-(5-chloro-2-morpholino-6-(pyridin-4-ylamino)pyrimidin-4-yl)-4-(I-methyl-1H-pyrazol-3-yl)phenyl)methanol

A solution of lithium aluminum hydride (1.0M in THF, 2.56 mL, 2.56 mmol) was added to a solution of ethyl 2-(5-chloro-2-morpholino-6-(pyridin-4-ylamino)pyrimidin-4-yl)-4-(1-methyl-1H-pyrazol-3-yl)benzoate (190 mg, 0.37 mmol) in anhydrous tetrahydrofuran (8 mL) at 0° C. and the resulting mixture was warmed up and stirred under nitrogen for 1.5 h at 25° C. The reaction was then quenched by the careful addition of sodium sulfate decahydrate with ice-bath cooling. Tetrahydrofuran (50 mL) was added to the reaction mixture, stirred for 15 minutes, filtered and the solids were washed with tetrahydrofuran (50 mL) and the filtrates were collected. The combined filtrates were concentrated to obtain (2-(5-chloro-2-morpholino-6-(pyridin-4-ylamino)pyrimidin-4-yl-4-(1-methyl-1H-pyrazol-3-yl)phenyl)methanol (140 mg, 80%) as yellow solid. LCMS (ESI) m/z: 478.3 [M+H]⁺.

### Step 6: Synthesis of 9-(1-methyl-H-pyrazol-3-yl)-2-morpholino-N-(pyridin-4-yl)-6H-isochromeno[4,3-d]pyrimidin-4-amine

A mixture of (2-(5-chloro-2-morpholino-6-(pyridin-4-ylamino)pyrimidin-4-yl)-4-(1-methyl-1H-pyrazol-3-yl)phenyl)methanol (0.11 g, 0.23 mmol) and potassium tert-butoxide (0.92 mL, 0.92 mmol, in tetrahydrofuran) in dimethyl sulfoxide (6 mL) was stirred at 100° C. for 4 h. The mixture was then filtered, the filtrate was concentrated, and the residue was subjected to prep-HPLC (BOSTON pHlex ODS 10 um 21.2×250 mm 120 A, with mobile phase acetonitrile/0.1% ammonium bicarbonate) to obtain 9-(1-methyl-1H-pyrazol-3-yl-2-morpholino-N-(pyridin-4-yl-6H-isochromeno[4,3-d]pyrimidin-4-amine (12.7 mg, 13%) as yellow solid. ¹H NMR (400 MHz, DMSO-d₆) δ 9.32 (s, 1H), 8.39 (d, J=6.1 Hz, 3H), 7.88 (dd, J=9.1, 3.2 Hz, 3H), 7.77 (d, J=2.1 Hz, 1H), 7.38 (d, J=7.9 Hz, 1H), 6.78 (d, J=2.2 Hz, 1H), 5.29 (s, 2H), 3.92 (s, 3H), 3.75 (d, J=4.6 Hz, 4H), 3.72 (d, J=4.7 Hz, 4H). LCMS (ESI) m/z: 442.3 [M+H]⁺.

### Example 42. Synthesis of 6-(3-(1H-pyrazol-1-yl)phenyl)-5-chloro-2-morpholino-N-(pyridin-4-yl)pyrimidin-4-amine (Compound 50)

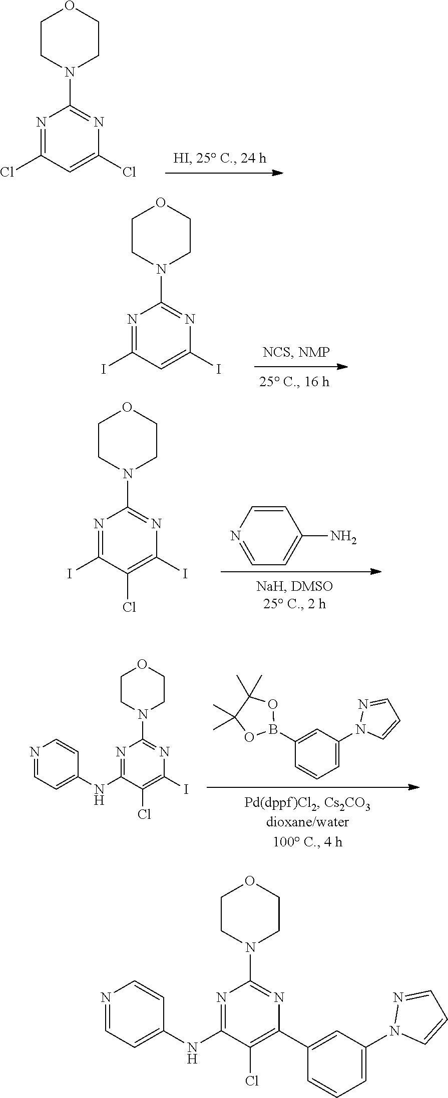

### Step 1: Synthesis of 4-(4,6-diiodopyrimidin-2-yl)morpholine

A mixture of 4-(4,6-dichloropyrimidin-2-yl)morpholine (3 g, 12.8 mmol) and hydriodic acid (30 mL) was stirred at 25° C. for 24 h. The reaction mixture was filtered, the solids were dissolved in dichloromethane and ethyl acetate, washed with water and concentrated. The residue was subjected to silica gel column chromatography (eluted with dichloromethane:methanol=10:1) to obtain 4-(4,6-diiodopyrimidin-2-yl)morpholine as a yellow solid (2.5 g, 47%). LCMS (ESI) m/z: 418.1 [M+H]⁺.

### Step 2: Synthesis of 4-(5-chloro-4,6-diiodopyrimidin-2-yl)morpholine

To a solution of 4-(4,6-diiodopyrimidin-2-yl)morpholine (2.5 g, 6 mmol) in 1-methyl-2-pyrrolidinone (40 mL) was added N-chlorosuccinimide (1.6 g, 12 mmol) in portions. After the addition the reaction mixture was stirred at 25° C. for 16 h. It was diluted with ethyl acetate/water (20 mL/20 mL), the organic layer was separated, and the aqueous layer was extracted with ethyl acetate (20 mL) twice. The combined organic phase was washed with brine (30 mL), dried over sodium sulfate, filtered and concentrated. The residue was subjected to column chromatography (Biotage, 40 g silica gel, eluted with ethyl acetate in petroleum ether from 20% to 30%) to obtain 4-(5-chloro-4,6-diiodopyrimidin-2-yl)morpholine (1.8 g, 67%) as off-white solid. LCMS (ESI) m/z: 452.1 [M+H]⁺.

### Step 3: Synthesis of 5-chloro-6-iodo-2-morpholino-N-(pyridin-4-yl)pyrimidin-4-amine

A mixture of pyridin-4-amine (0.21 g, 2.22 mmol) and sodium hydride (0.177 g, 4.43 mmol) in dimethyl sulfoxide (5 mL) was stirred at 25° C. for 0.5 h. To the resultant mixture was added 4-(5-chloro-4,6-diiodopyrimidin-2-yl)morpholine (1 g, 2.22 mmol) and stirring was continued for another 2 h. The mixture was then poured into ice-water, extracted with ethyl acetate (15 mL*3), the combined organic layer was washed with brine, dried over sodium sulfate and concentrated. The residue was subjected to chromatography on silica gel (dichloromethane:methanol=20:1) to obtain 5-chloro-6-iodo-2-morpholino-N-(pyridin-4-yl)pyrimidin-4-amine (400 mg, 43%) as yellow solid. LCMS (ESI) m/z: 418.1 [M+H]⁺.

### Step 4: Synthesis of 6-(3-(1H-pyrazol-1-yl)phenyl)-5-chloro-2-morpholino-N-(pyridin-4-yl)pyrimidin-4-amine

To a solution of 5-chloro-6-iodo-2-morpholino-N-(pyridin-4-yl)pyrimidin-4-amine (0.05 g, 0.12 mmol) and 1-(3-(4,4,5,5-tetramethyl-1,3,2-dioxaborolan-2-yl)phenyl)-1H-pyrazole (0.035 g, 0.13 mmol) in 1,4-dioxane/water (1.5 mL/0.4 mL) were added cesium carbonate (0.078 g, 0.24 mmol) and 1,1′-bis(diphenylphosphino)ferrocene-palladium(II)dichloride dichloromethane complex (0.007 g, 0.01 mmol) at room temperature. The resultant mixture was heated and stirred at 90° C. for 2 h. It was concentrated and the residue was subjected to prep-HPLC (BOSTON pHlex ODS 10 um 21.2×250 mm 120 A, with mobile phase acetonitrile/0.1% ammonium bicarbonate) to obtain 6-(3-(1H-pyrazol-1-yl)phenyl)-5-chloro-2-morpholino-N-(pyridin-4-ylpyrimidin-4-amine as a yellow solid (28.3 mg, 54%). ¹H NMR (400 MHz, DMSO-d₆) δ 9.25 (s, 1H), 8.57 (d, J=2.5 Hz, 1H), 8.46 (d, J=6.3 Hz, 2H), 8.13 (s, 1H), 7.96 (dt, J=7.3, 2.1 Hz, 1H), 7.83-7.79 (m, 2H), 7.78 (d, J=1.6 Hz, 1H), 7.64-7.59 (m, 2H), 6.59-6.56 (m, 1H), 3.68 (s, 8H). LCMS (ESI) m/z: 434.3 [M+H]⁺.

### Example 43. Synthesis of 6-(5,6-dimethoxypyridin-2-yl)-5-methoxy-2-morpholino-N-(pyridin-4-yl)pyrimidin-4-amine (Compound 51)

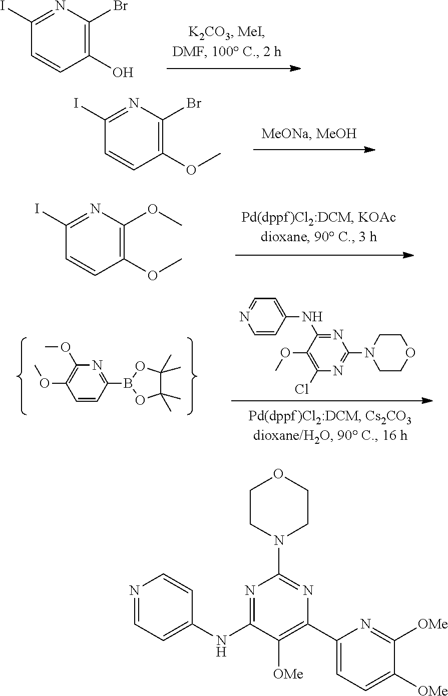

### Step 1: Synthesis of 2-bromo-6-iodo-3-methoxypyridine

A solution of 2-bromo-6-iodopyridin-3-ol (1 g, 3.36 mmol), iodomethane (955 mg, 6.72 mmol), potassium carbonate (484 mg, 3.36 mmol) in DMF (40 mL) was stirred at 100° C. for 2 h under nitrogen atmosphere. The mixture was cooled to room temperature, treated with water (30 mL) and stirred for 0.5 h. The resulting precipitate was isolated by filtration and dried to obtain 2-bromo-6-iodo-3-methoxypyridine (0.92 g, 87.5%) as brown solid. LCMS (ESI) m/z: 313.9 [M+H]⁺.

### Step 2: Synthesis of 6-iodo-2,3-dimethoxypyridine

A solution of 2-bromo-6-iodo-3-methoxypyridine (0.92 g, 2.94 mmol), sodium methanolate (320 mg, 5.9 mmol) in dry methanol (30 mL) was stirred at 100° C. for 2 h under nitrogen atmosphere. The mixture was cooled to room temperature and then partitioned between aqueous saturated sodium bicarbonate and dichloromethane. The organic phase was washed with water, dried over sodium sulfate, filtered and concentrated. The residue was subjected to silica gel column chromatography eluting with a linear gradient of 0% to 10% ethyl acetate in petroleum ether to obtain 6-iodo-2,3-dimethoxypyridine (664 mg, 85.1%) as brown solid. LCMS (ESI) m/z: 268.0 [M+H]⁺.

### Step 3: Synthesis of 3-dimethoxy-6-(4,4,5,5-tetramethyl-1,3,2-dioxaborolan-2-yl)pyridine

A solution of 6-iodo-2,3-dimethoxypyridine (200 mg, 0.75 mmol), 4,4,4′,4′,5,5,5′,5′-octamethyl-2,2′-bi(1,3,2-dioxaborolane) (230 mg, 0.9 mmol), 1,1′-bis(diphenylphosphino) ferrocene-palladium(II)dichloride dichloromethane complex (61 mg, 0.075 mmol) and potassium acetate (180 mg, 1.5 mmol) in dry 1,4-dioxane (10 mL) was stirred at 90° C. for 16 h under nitrogen atmosphere. The reaction mixture was directly used in the next step without further purification. LCMS (ESI) m/z: 266.0 [M+H]⁺.

### Step 4: Synthesis of 6-(5,6-dimethoxypyridin-2-yl)-5-methoxy-2-morpholino-N-(pyridin-4-yl)pyrimidin-4-amine

To the reaction mixture from above step was added 6-chloro-5-methoxy-2-morpholino-N-(pyridin-4-ylpyrimidin-4-amine (50 mg, 0.156 mmol), 1,1′-bis(diphenylphosphino) ferrocene-palladium(II)dichloride: dichloromethane complex (12 mg, 0.015 mmol), potassium carbonate (146 mg, 0.45 mmol) and water (1 mL). The resulting mixture was stirred at 90° C. for 16 h under nitrogen atmosphere. It was then filtered to remove the solids; the filtrate was concentrated, and residue was subjected to prep-HPLC (BOSTON pHlex ODS 10 um 21.2×250 mm 120 A. The mobile phase was acetonitrile/0.1% ammonium bicarbonate) to obtain 6-(5,6-dimethoxypyridin-2-yl)-5-methoxy-2-morpholino-N-(pyridin-4-ylpyrimidin-4-amine (20.6 mg, 31.2%) as yellow solid. ¹H NMR (400 MHz, DMSO-d₆) δ 9.31 (s, 1H), 8.40 (s, 2H), 7.88 (d, J=4.6 Hz, 2H), 7.70 (d, J=8.1 Hz, 1H), 7.41 (d, J=8.2 Hz, 1H), 3.95 (s, 3H), 3.85 (s, 3H), 3.69 (d, J=6.4 Hz, 8H), 3.65 (s, 3H). LCMS (ESI) m/z: 425.2 [M+H]⁺.

### Example 44. Synthesis of 4-(5-methoxy-4-(3-(1-methyl-1H-pyrazol-3-yl)phenyl)-6-(1-methyl-1H-pyrazol-4-yl)pyrimidin-2-yl)morpholine (Compound 52)

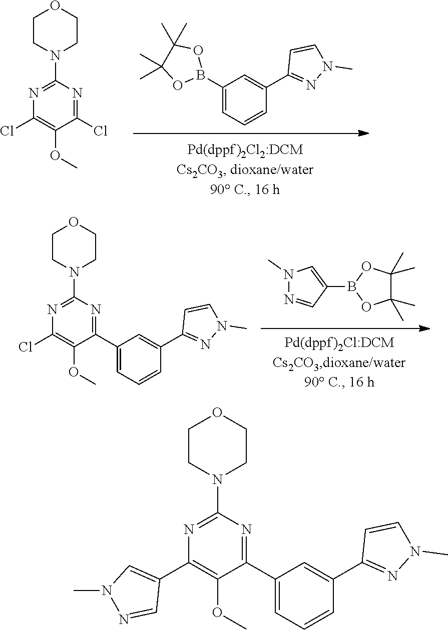

### Step 1: Synthesis of 4-(4-chloro-5-methoxy-6-(3-(1-methyl-1H-pyrazol-3-yl)phenyl)pyrimidin-2-yl)morpholine

A mixture of 4-(4,6-dichloro-5-methoxypyrimidin-2-yl)morpholine (90 mg, 0.341 mmol), 1-methyl-3-(3-(4,4,5,5-tetramethyl-1,3,2-dioxaborolan-2-yl)phenyl)-1H-pyrazole (88 mg, 0.31 mmol), 1,1′-bis(diphenylphosphino)ferrocene-palladium(II)dichloride dichloromethane complex (25 mg, 0.03 mmol) and cesium carbonate (245 mg, 0.75 mmol) in 1,4-dioxane/water (5 mL/1 mL) was stirred at 90° C. under argon atmosphere for 16 h. The mixture was filtered, the filtrate was concentrated, and the residue was subjected to silica gel column chromatography (petroleum ether-acetic ester=3:1) to obtain 4-(4-chloro-5-methoxy-6-(3-(1-methyl-1H-pyrazol-3-yl)phenyl)pyrimidin-2-yl)morpholine (80 mg, 61%) as white solid. LCMS (ESI) m/z: 386.2 [M+H]⁺.

### Step 2: Synthesis of 4-(5-methoxy-4-(3-(1-methyl-1H-pyrazol-3-yl)phenyl)-6-(1-methyl-1H-pyrazol-4-yl)pyrimidin-2-yl)morpholine

A mixture of 4-(4-chloro-5-methoxy-6-(3-(1-methyl-1H-pyrazol-3-yl)phenyl)pyrimidin-2-yl)morpholine (118 mg, 0.3 mmol), 1-methyl-4-(4,4,5,5-tetramethyl-1,3,2-dioxaborolan-2-yl)-1H-pyrazole (62 mg, 0.3 mmol), 1,1′-bis(diphenylphosphino) ferrocene-palladium(II)dichloride dichloromethane complex (25 mg, 0.03 mmol) and cesium carbonate (245 mg, 0.75 mmol) in 1,4-dioxane/water (5 mL/1 mL) was stirred at 90° C. under argon atmosphere for 16 h. The mixture was filtered, the filtrate was concentrated, and the residue was subjected to prep-HPLC [Welch Xtimate C18 21.2×250 mm, 10 um, with mobile phase acetonitrile/water (10 mM NH₄HCO₃ and NH₃·H₂O)] to afford 4-(5-methoxy-4-(3-(1-methyl-1H-pyrazol-3-yl)phenyl-6-(1-methyl-1H-pyrazol-4-yl)pyrimidin-2-yl)morpholine (15.3 mg, 12%) as pale-yellow solid. ¹H NMR (400 MHz, DMSO-d₆) δ 8.48 (s, 1H), 8.42 (s, 1H), 8.15 (s, 1H), 7.89 (d, J=7.8 Hz, 2H), 7.77 (d, J=2.2 Hz, 1H), 7.53 (t, J=7.8 Hz, 1H), 6.74 (d, J=2.2 Hz, 1H), 3.94 (s, 3H), 3.91 (s, 3H), 3.75 (d, J=4.8 Hz, 4H), 3.71 (d, J=4.7 Hz, 4H), 3.40 (s, 3H). LCMS (ESI) m/z: 432.2 [M+H]⁺.

### Example 45. Synthesis of 5-methoxy-6-(3-(1-methyl-1H-pyrazol-3-yl)phenyl)-N, 2-di(pyridin-4-yl)pyrimidin-4-amine (Compound 53)

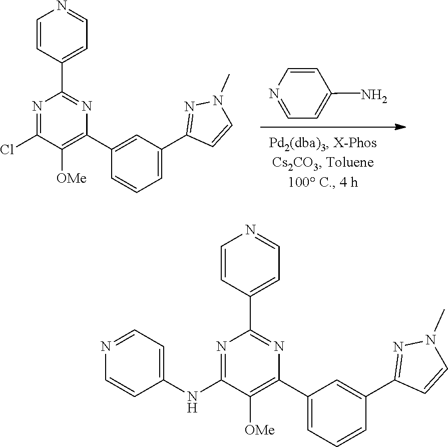

A mixture of 4-chloro-5-methoxy-6-(3-(1-methyl-1H-pyrazol-3-yl)phenyl)-2-(pyridin-4-yl)pyrimidine (0.08 g, 0.2 mmol), pyridin-4-amine (0.04 g, 0.42 mmol), cesium carbonate (0.21 g, 0.64 mmol), tris(dibenzylideneacetone)dipalladium(0) (0.02 g, 0.02 mmol) and 2-dicyclohexylphosphino-2′,6′-di-i-propoxy-1,1′-biphenyl (0.015 g, 0.03 mmol) in toluene (4 mL) was stirred at 100° C. for 4 h. The reaction mixture was concentrated, and the residue was subjected to prep-HPLC (BOSTON pHlex ODS 10 um 21.2×250 mm 120 A, with mobile phase acetonitrile/0.1% ammonium bicarbonate) to obtain 5-methoxy-6-(3-(1-methyl-1H-pyrazol-3-yl)phenyl)-N, 2-di(pyridin-4-yl)pyrimidin-4-amine as yellow solid (8.8 mg, 10%). ¹H NMR (400 MHz, DMSO-d₆) δ 8.79 (dd, J=4.5, 1.5 Hz, 2H), 8.54 (t, J=1.9 Hz, 2H), 8.53 (d, J=1.4 Hz, 1H), 8.27 (dd, J=4.5, 1.6 Hz, 2H), 8.05 (dd, J=4.8, 1.5 Hz, 2H), 8.03 (d, J=7.9 Hz, 1H), 7.95 (d, J=7.8 Hz, 1H), 7.79 (d, J=2.2 Hz, 1H), 7.62 (t, J=7.8 Hz, 1H), 6.78 (d, J=2.2 Hz, 1H), 6.07 (s, 1H), 3.92 (s, 3H), 3.62 (s, 3H). LCMS (ESI) m/z: 435.8 [M+H]⁺.

### Example 46. Synthesis of 5-methoxy-2-morpholino-6-(3-(oxazol-2-yl)phenyl)-N-(pyridin-4-yl)pyrimidin-4-amine (Compound 54)

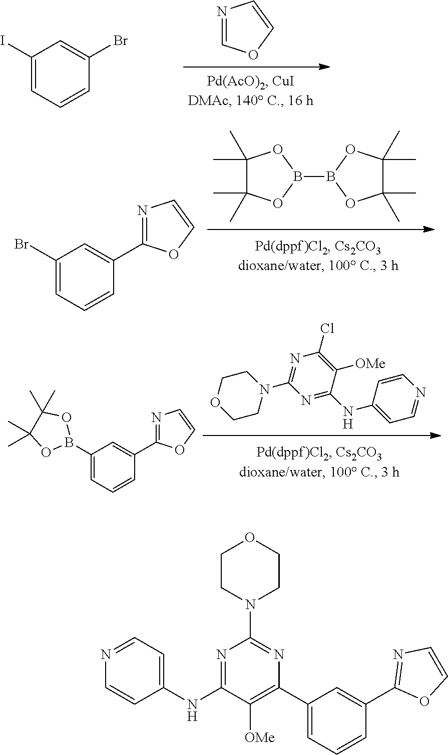

### Step 1: Synthesis of 2-(3-bromophenyl)oxazole

A mixture of 1-bromo-3-iodobenzene (0.5 g, 1.77 mmol), oxazole (0.12 g, 1.77 mmol), palladium acetate (0.04 g, 0.18 mmol) copper(I) iodide (0.068 g, 0.36 mmol) in N,N-dimethylacetamide (25 mL) was stirred at 140° C. for 16 h under argon atmosphere. The reaction mixture was concentrated, the residue was diluted with water (20 mL) and extracted with dichloromethane (20 mL×2). The combined organic phase was washed with brine (20 mL), dried over sodium sulfate, filtered and concentrated. The residue was purified by flash chromatography (eluted with petroleum ether in ethyl acetate from 30% to 60%) to obtain 2-(3-bromophenyl)oxazole (0.3 g, 76.7%) as oil. LCMS (ESI) m/z: 223.8 [M+H]⁺.

### Step 2: Synthesis of 2-(3-(4,4,5,5-tetramethyl-1,3,2-dioxaborolan-2-yl)phenyl) oxazole

A mixture of 2-(3-bromophenyl)oxazole (250 mg, 1.12 mmol), 4,4,4′,4′,5,5,5′,5′-octamethyl-2,2′-bi(1,3,2-dioxaborolane) (320 mg, 1.12 mmol), 1,1′-bis(diphenylphosphino)ferrocenepalladium (II)dichloride dichloromethane complex (92 mg, 0.112 mmol) and cesium carbonate (1.08 g, 3.36 mmol) in 1,4-dioxane/water (10 mL 2 mL) was stirred at 100° C. for 3 h under argon atmosphere. The mixture was concentrated, the residue was diluted with water (20 mL) and extracted with ethyl acetate (20 mL×2). The combined organic phase was washed with brine (20 mL), dried over sodium sulfate, filtered and concentrated. The residue was purified prep-HPLC (base) to obtain 2-(3-(4,4,5,5-tetramethyl-1,3,2-dioxaborolan-2-yl)phenyl)oxazole (180 mg, 59%) as white solid. LCMS (ESI) m/z: 271.9 [M+H]⁺.

### Step 3: Synthesis of 5-methoxy-2-morpholino-6-(3-(oxazol-2-yl)phenyl)-N-(pyridin-4-yl)pyrimidin-4-amine

A mixture of 6-chloro-5-methoxy-2-morpholino-N-(pyridin-4-yl)pyrimidin-4-amine (100 mg, 0.31 mmol), 2-(3-(4,4,5,5-tetramethyl-1,3,2-dioxaborolan-2-yl)phenyl)oxazole (84 mg, 0.31 mmol), 1,1′-bis(diphenylphosphino)ferrocene-palladium(II)dichloride dichloromethane complex (25 mg, 0.031 mmol) and cesium carbonate (0.3 g, 0.93 mmol) in 1,4-dioxane/water (15 mL/3 mL) was stirred at 100° C. for 3 h under argon atmosphere. The mixture was concentrated, the residue was diluted with water (20 mL) and extracted with ethyl acetate (20 mL×2). The combined organic phase was washed with brine (20 mL), dried over sodium sulfate, filtered and concentrated. The residue was subjected to prep-HPLC (base) to afford 5-methoxy-2-morpholino-6-(3-(oxazol-2-ylphenyl)-N-(pyridin-4-ylpyrimidin-4-amine (23.4 mg, 17%) as white solid. ¹H NMR (400 MHz, DMSO-d₆) δ 9.41 (s, 1H), 8.67 (s, 1H), 8.42 (s, 2H), 8.27 (s, 1H), 8.17 (d, J=7.7 Hz, 1H), 8.09 (d, J=7.6 Hz, 1H), 7.89 (s, 2H), 7.69 (t, J=7.7 Hz, 1H), 7.43 (s, 1H), 3.71 (s, 8H), 3.48 (s, 3H). LCMS (ESI) m/z: 430.8 [M+H]⁺.

### Example 47. Synthesis of 1-(3-(5-methoxy-2-morpholino-6-(pyridin-3-ylamino)pyrimidin-4-yl)phenyl)piperidin-4-ol (Compound 55)

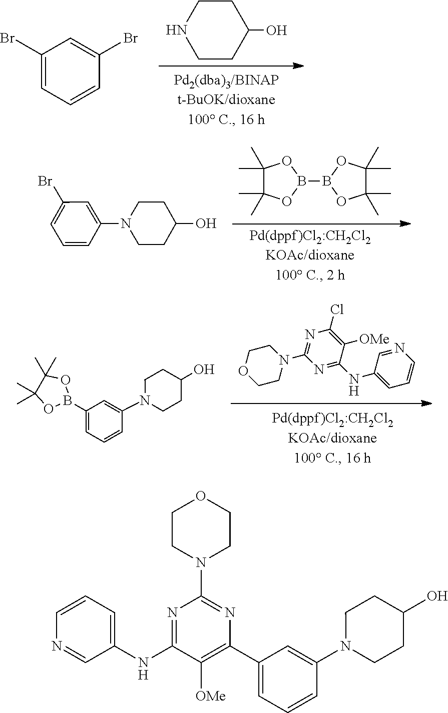

### Step 1: Synthesis of 1-(3-bromophenyl)piperidin-4-ol

A mixture of 1,3-dibromobenzene (2.34 g, 10.0 mmol), piperidin-4-ol (870 mg, 10.0 mmol), tris(dibenzylideneacetone)dipalladium(0) (458 mg, 0.5 mmol), 1.1′-binaphthyl-2.2′-diphemyl phosphine (622 mg, 1.0 mmol) and potassium tert-butoxide (2.24 g, 20.0 mmol) in 1,4-dioxane (40 mL) was stirred at 100° C. under nitrogen atmosphere for 16 h. The mixture was then poured into water and extracted with dichloromethane (200 mL*2). The combined organic phase was concentrated, and the residue was subjected to silica gel column chromatography (10% of methanol in dichloromethane) to obtain 1-(3-bromophenyl)piperidin-4-ol (300 mg, 12%) as red oil. LCMS (ESI) m/z: 255.9/257.9 [M+H]⁺.

### Step 2: Synthesis of 1-(3-(4,4,5,5-tetramethyl-1,3,2-dioxaborolan-2-yl)phenyl)piperidin-4-ol

A mixture of 1-(3-bromophenyl)piperidin-4-ol (275 mg, 1.1 mmol), 4,4,4′,4′,5,5,5′,5′-octamethyl-2,2′-bi(1,3,2-dioxaborolane) (559 mg, 2.2 mmol), 1,1′-bis(diphenylphosphino)ferrocene-palladium(II)dichloride dichloromethane complex (90 mg, 0.11 mmol) and potassium acetate (216 mg, 2.2 mmol) in 1,4-dioxane (10 mL) was stirred at 100° C. under nitrogen atmosphere for 4 h. The mixture was then poured into water and extracted with ethyl acetate (150 mL*2). The combined organic phase was concentrated, and the residue was subjected to silica gel column chromatography (5% methanol in dichloromethane) to obtain 1-(3-(4,4,5,5-tetramethyl-1,3,2-dioxaborolan-2-yl)phenyl)piperidin-4-ol (250 mg, 74.5%) as brown oil. LCMS (ESI) m/z: 304.2 [M+H]⁺.

### Step 3: Synthesis of 1-(3-(5-methoxy-2-morpholino-6-(pyridin-3-ylamino)pyrimidin-4-yl)phenyl)piperidin-4-ol

A mixture of 6-chloro-5-methoxy-2-morpholino-N-(pyridin-3-yl)pyrimidin-4-amine (100 mg, 0.38 mmol), 1-(3-(4,4,5,5-tetramethyl-1,3,2-dioxaborolan-2-yl)phenyl)piperidin-4-ol (136 mg, 0.47 mmol), 1,1′-bis(diphenylphosphino)ferrocene-palladium(II)dichloride dichloromethane complex (25 mg, 0.03 mmol) and cesium carbonate (201 mg, 0.62 mmol) in 1,4-dioxane/water (5 mL/0.5 mL) was stirred at 100° C. under nitrogen atmosphere for 16 h. The mixture was then poured into water and extracted with dichloromethane (100 mL*2). The combined organic phase was concentrated and the residue was subjected to silica gel column chromatography (15% methanol in dichloromethane) to obtain an impure product which was further subjected to prep-HPLC [Welch Xtimate C18 21.2×250 mm, 10 um, with mobile phase acetonitrile/water (10 mM NH₄HCO₃ and NH₃·H₂O)] to obtain 1-(3-(5-methoxy-2-morpholino-6-(pyridin-3-ylamino)pyrimidin-4-yl)phenyl)piperidin-4-ol (23.5 mg, 16%) as an off-white solid. ¹H NMR (400 MHz, DMSO-d₆) δ 9.12 (s, 1H), 8.99 (s, 1H), 8.34-8.16 (m, 2H), 7.58 (s, 1H), 7.35 (m, 3H), 7.05 (d, J=7.9 Hz, 1H), 4.71 (d, J=4.1 Hz, 1H), 3.71-3.37 (m, 14H), 2.90 (t, J=11.2 Hz, 2H), 1.84 (d, J=11.2 Hz, 2H), 1.50 (d, J=9.5 Hz, 2H); LCMS (ESI) m/z: 463.2 [M+H]⁺.

### Example 48. Synthesis of 5-methoxy-2′-(I-methyl-1H-pyrazol-3-yl)-2-morpholino-N-(pyridin-4-yl)-4,4′-bipyrimidin-4-amine (Compound 56)

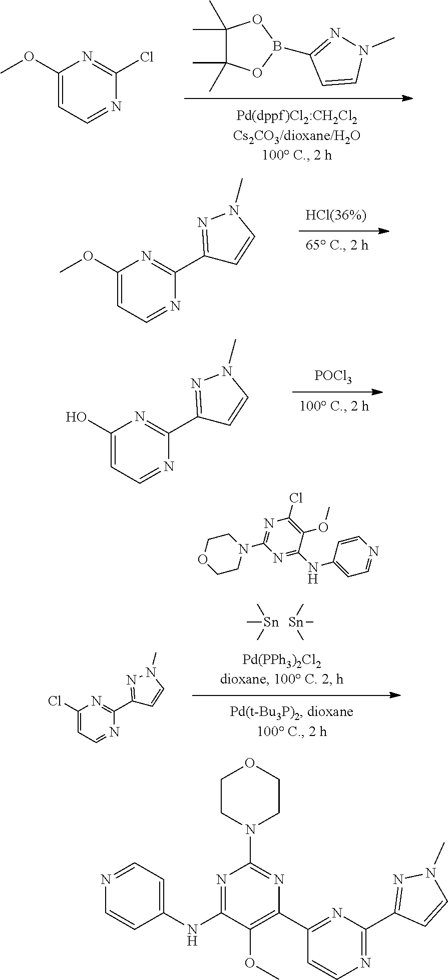

### Step 1: Synthesis of 4-methoxy-2-(1-methyl-1H-pyrazol-3-yl)pyrimidine

A mixture of 2-chloro-4-methoxypyrimidine (1.44 g, 10.0 mmol, 1-methyl-3-(4,4,5,5-tetramethyl-1,3,2-dioxaborolan-2-yl)-1H-pyrazole (2.08 g, 10.0 mmol), 1,1′-bis(diphenylphosphino)ferrocene-palladium(II)dichloride dichloromethane complex (163 mg, 0.2 mmol) and cesium carbonate (6.5 g, 20.0 mmol) in dioxane/water (50 mL/6.0 mL) was stirred at 100° C. under nitrogen atmosphere for 2 h. The resultant mixture was poured into water and extracted with ethyl acetate (200 mL*2). The combined organic phase was concentrated, and the residue was subjected to silica gel column chromatography (50% ethyl acetate in petroleum ether) to obtain 4-methoxy-2-(1-methyl-1H-pyrazol-3-yl)pyrimidine (1.2 g, 63%) as viscous liquid. LCMS (ESI) m/z: 191.1 [M+H]⁺.

### Step 2: Synthesis of 2-(1-methyl-1H-pyrazol-3-yl)pyrimidin-4-ol

A mixture of 4-methoxy-2-(1-methyl-1H-pyrazol-3-yl)pyrimidine (1 g, 5.2 mmol) and concentrated hydrochloric acid (10.0 mL) was stirred at 65° C. for 2 h. The mixture was poured into crushed ice, basified with solid sodium bicarbonate and extracted with dichloromethane (150 mL*5). The combined organic phase was concentrated, and the residue was subjected to silica gel column chromatography (20% methanol in dichloromethane) to obtain 2-(1-methyl-1H-pyrazol-3-yl)pyrimidin-4-ol (650 mg, 54.7%) as grey solid. LCMS (ESI) m/z: 177.0 [M+H]⁺.

### Step 3: Synthesis of 4-chloro-2-(1-methyl-1H-pyrazol-3-yl)pyrimidine

A mixture of 2-(1-methyl-1H-pyrazol-3-yl)pyrimidin-4-ol (600 mg, 3.4 mmol) and phosphorus oxychloride (10.0 mL) was stirred at 100° C. for 2 h. The mixture was concentrated, the residue was poured into crushed ice and extracted with dichloromethane (200 mL*3). The combined organic phase was concentrated, and the residue was subjected to silica gel column chromatography (5% methanol in dichloromethane) to obtain 4-chloro-2-(1-methyl-1H-pyrazol-3-yl)pyrimidine (450 mg, 58.1%) as grey solid. LCMS (ESI) m/z: 194.9/196.9 [M+H]⁺.

### Step 4: Synthesis of 5-methoxy-2′-(1-methyl-1H-pyrazol-3-yl)-2-morpholino-N-(pyridin-4-yl)-4,4′-bipyrimidin-4-amine

A mixture of 6-chloro-5-methoxy-2-morpholino-N-(pyridin-4-yl)pyrimidin-4-amine (150 mg, 0.46 mmol), 1,1,1,2,2,2-hexamethyldistannane (229 mg, 0.7 mmol) and bis(triphenylphosphine)palladium(II) chloride (28 mg, 0.04 mmol) in dioxane (6 mL) was stirred at 100° C. under nitrogen atmosphere for 2 h. To the resultant mixture were added, 4-chloro-2-(1-methyl-1H-pyrazol-3-yl)pyrimidine (90 mg, 0.46 mmol) and bis(tri-tert-butylphosphine)palladium (20 mg, 0.04 mmol) and stirring was continued at 100° C. for another 2 h. It was concentrated and the residue was subjected to silica gel column chromatography (10% methanol in dichloromethane) and then to prep-HPLC [Weich Xtimate C18 21.2×250 mm, 10 um, with mobile phase acetonitrile/water (10 mM NH₄HCO₃ and NH₃·H₂O)] to obtain 5-methoxy-2′-(1-methyl-1H-pyrazol-3-yl-2-morpholino-N-(pyridin-4-yl)-4,4′-bipyrimidin-6-amine (9.5 mg, 4.5%) as yellow solid. ¹H NMR (400 MHz, DMSO-d₆) δ 9.46 (s, 1H), 8.97 (d, J=5.1 Hz, 1H), 8.44 (d, J=6.3 Hz, 2H), 7.92-7.88 (m, 2H), 7.83 (m, 2H), 6.95 (d, J=2.2 Hz, 1H), 3.96 (s, 3H), 3.80 (s, 3H), 3.69 (s, 8H); LCMS (ESI) m/z: 445.8 [M+H]⁺.

### Example 49. Synthesis of 5-methoxy-2-morpholino-65-(pyridazin-3-yl)pyridin-3-yl)-N-(pyridin-4-yl)pyrimidin-4-amine (Compound 57)

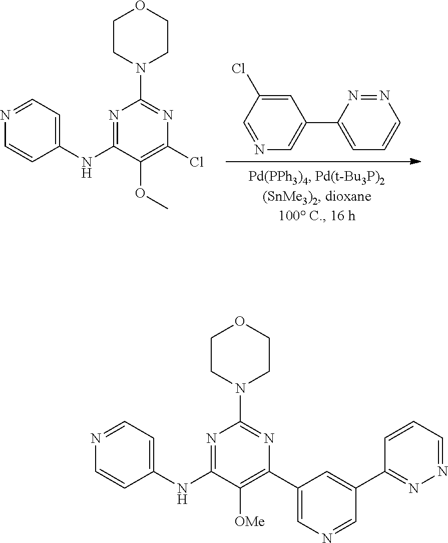 [SMILES: COc1c(Cl)nc(N2CCOCC2)nc1Nc1ccncc1 ; Clc1cncc(-c2cccnn2)c1 ; COc1c(Nc2ccncc2)nc(N2CCOCC2)nc1-c1cncc(-c2cccnn2)c1]

A solution of 6-chloro-5-methoxy-2-morpholino-N-(pyridin-4-yl)pyrimidin-4-amine (100 mg, 0.31 mmol), 3-(5-chloropyridin-3-yl)pyridazine (70 mg, 0.36 mmol), tetrakis(triphenylphosphine)palladium(34 mg, 0.06 mmol), bis(tri-tert-butylphosphine) palladium(0) (30 mg, 0.03 mmol) and hexamethyldistannane (153 mg, 0.46 mmol) in dry 1,4-dioxane (10 mL) was stirred at 100° C. for 16 hours under argon atmosphere. The resultant mixture was filtered, the filtrate was concentrated, and the residue was subjected to prep-HPLC (BOSTON pHlex ODS 10 um 21.2×250 mm 120 A). The mobile phase was acetonitrile/0.1% ammonium bicarbonate) to obtain 5-methoxy-2-morpholino-6-(5-(pyridazin-3-yl)pyridin-3-yl)-N-(pyridin-4-yl)pyrimidin-4-amine (12.7 mg, 9.26%) as yellow solid. ¹H NMR (400 MHz, DMSO-d₆) δ 9.45 (s, 1H), 9.39 (d, J=2.2 Hz, 1H), 9.32 (dd, J=4.0, 1.8 Hz, 2H), 9.09 (t, J=2.1 Hz, 1H), 8.42 (dd, J=10.3, 3.2 Hz, 3H), 7.89 (dd, J=7.7, 5.8 Hz, 3H), 3.72 (s, 8H), 3.53 (s, 3H). LCMS (ESI) m/z: 443.1 [M+H]⁺.

### Example 50. Synthesis of 4-(5-methoxy-4-(3-(1-methyl-1H-pyrazol-3-yl)phenyl)-6-(quinolin-6-yl)pyrimidin-2-yl)morpholine (Compound 58)

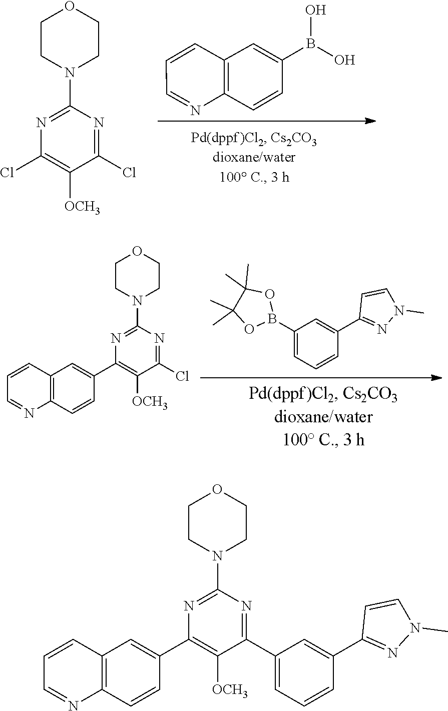

### Step 1: Synthesis of 4-(4-chloro-5-methoxy-6-(quinolin-6-yl)pyrimidin-2-yl)morpholine

A mixture of 4-(4,6-dichloro-5-methoxypyrimidin-2-yl)morpholine (0.4 g, 1.70 mmol), quinolin-6-ylboronic acid (0.29 g, 1.70 mmol), 1,1′-bis(diphenylphosphino)ferrocene-palladium(II)dichloride dichloromethane complex (0.14 g, 0.17 mmol), and cesium carbonate (1.39 g, 4.26 mmol) in 1,4-dioxane/water (20 mL/3 mL) was stirred at 100° C. for 2 h. The reaction mixture was concentrated, and the residue was diluted with water (20 mL) and extracted with dichloromethane (20 mL×2). The combined organic phase was washed with brine (20 mL), dried over sodium sulfate, filtered and concentrated. The residue was subjected to flash chromatography (eluted with petroleum ether in ethyl acetate from 30% to 60%) to obtain 4-(4-chloro-5-methoxy-6-(quinolin-6-yl)pyrimidin-2-yl)morpholine (0.2 g, 33.1%) as pale-yellow solid. LCMS (ESI) m/z: 356.8 [M+H]⁺.

### Step 2: Synthesis of 4-(5-methoxy-4-(3-(1-methyl-1H-pyrazol-3-yl)phenyl)-6-(quinolin-6-yl)pyrimidin-2-yl)morpholine

A mixture of 4-(4-chloro-5-methoxy-6-(quinolin-6-yl)pyrimidin-2-yl)morpholine (200 mg, 0.56 mmol), 1-methyl-3-(3-(4,4,5,5-tetramethyl-1,3,2-dioxaborolan-2-yl)phenyl)-1H-pyrazole (160 mg, 0.56 mmol), 1,1′-bis(diphenylphosphino)ferrocenepalladium (II)dichloride dichloromethane complex (46 mg, 0.056 mmol) and cesium carbonate (0.54 g, 1.68 mmol) in 1,4-dioxane/water (10 mL/2 mL) was stirred at 100° C. for 32 h. The mixture was concentrated, the residue was diluted with water (20 mL) and extracted with ethyl acetate (20 mL×2). The combined organic phase was washed with brine (20 mL), dried over sodium sulfate, filtered and concentrated. The resulting residue was subjected to prep-HPLC (base) to obtain 4-(5-methoxy-4-(3-(1-methyl-1H-pyrazol-3-yl)phenyl)-6-(quinolin-6-yl)pyrimidin-2-yl)morpholine (59.6 mg, 22.3%) as yellow solid. ¹H NMR (400 MHz, DMSO-d₆) δ 8.99 (d, J=4.1 Hz, 1H), 8.76 (s, 1H), 8.57 (d, J=8.0 Hz, 1H), 8.48 (s, 1H), 8.44 (m, 1H), 8.15 (d, J=8.9 Hz, 1H), 7.98-7.91 (m, 2H), 7.78 (d, J=2.0 Hz, 1H), 7.56 (t, J=7.7 Hz, 1H), 6.76 (d, J=2.1 Hz, 1H), 3.92 (s, 3H), 3.81 (d, J=4.1 Hz, 4H), 3.75 (d, J=4.0 Hz, 4H), 3.27 (s, 3H). LCMS (ESI) m/z: 478.6 [M+H]⁺.

### Example 51. Synthesis of 6-(3-(1-methyl-1H-pyrazol-3-yl)phenyl)-6-(methylsulfinyl)-2-morpholino-N-(pyridin-3-yl)pyrimidin-4-amine (Compound 59)

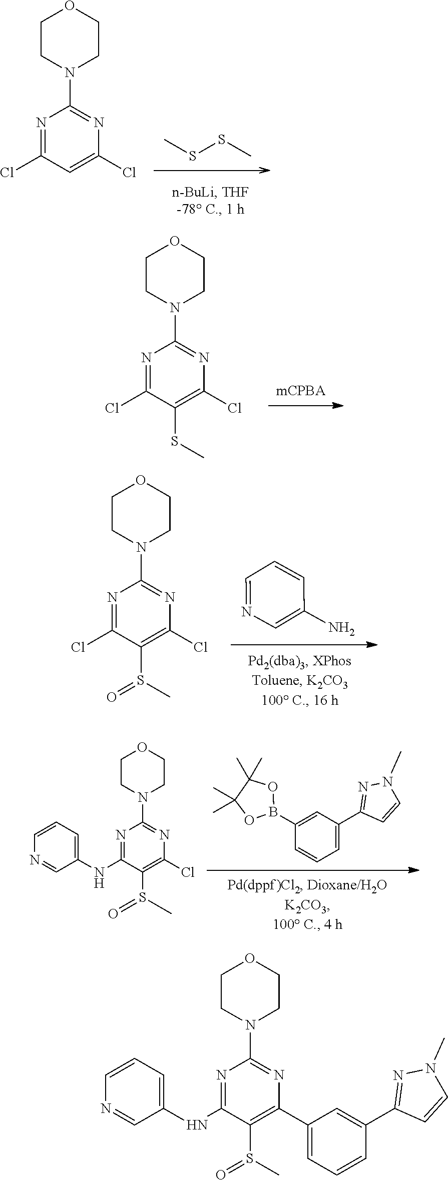

### Step 1: Synthesis of 4-(4,6-dichloro-5-(methylthio)pyrimidin-2-yl)morpholine

To a solution of 4-(4,6-dichloropyrimidin-2-yl)morpholine (2.4 g, 10.2 mmol) in tetrahydrofuran (20 mL) was added n-butyllithium (8 mL, 20.4 mmol) at −78° C. The resultant mixture was stirred at −78° C. for 1 h followed by the addition of 1,2-dimethyldisulfane (964 mg, 10.2 mmol) and it was stirred at −78° C. for an additional 1 h. Then the reaction was quenched with water (15 mL) and the mixture was extracted with ethyl acetate (20 mL*3). The organic layers were combined, washed with brine (30 mL), dried over sodium sulfate, filtered and concentrated. The residue was subjected to flash chromatography on silica gel (petroleum ether:ethyl acetate=75:25) to obtain 4-(4,6-dichloro-5-(methylthio)pyrimidin-2-yl)morpholine as yellow oil (1.2 g, 42.5%). LCMS (ESI) m/z: 279.9[M+H]⁺.

### Step 2: Synthesis of 4-(4,6-dichloro-5-(methylsulfinyl)pyrimidin-2-yl)morpholine

To a solution of 4-(4,6-dichloro-5-(methylthio)pyrimidin-2-yl)morpholine (250 mg, 0.896 mmol) in dichloromethane (10 mL) was added 3-chlorobenzoperoxoic acid (154 mg, 0.89 mmol) and the resulting mixture was stirred at room temperature for 8 h. Then the reaction was quenched with water (15 mL) and the mixture was extracted with ethyl acetate (20 mL*3). The organic layers were combined, washed with brine (30 mL), dried over sodium sulfate, filtered and concentrated. The residue was subjected to flash chromatography on silica gel (petroleum ether:ethyl acetate=75:25) to obtain 4-(4,6-dichloro-5-(methylsulfinyl)pyrimidin-2-yl)morpholine as yellow solid. (215 mg, 81.1%). LCMS (ESI) m/z: 296.0[M+H]⁺.

### Step 3: Synthesis of 6-chloro-5-(methylsulfinyl)-2-morpholino-N-(pyridin-3-yl)pyrimidin-4-amine

A mixture of 4-(4,6-dichloro-5-(methylsulfinyl)pyrimidin-2-yl)morpholine (215 mg, 0.726 mmol), pyridin-3-amine (68 mg, 0.726 mmol), tris(dibenzylideneacetone) dipalladium (30 mg, 0.05 mmol), 2-(dicyclohexylphosphino)-2′,4′,6′-triisopropylbiphenyl (56 mg, 0.06 mmol) and potassium carbonate (201 mg, 1.45 mmol) in toluene (10 mL) was stirred at 85° C. for 16 h. Then the reaction was quenched with water (15 mL) and extracted with ethyl acetate (20 mL*3). The organic layers were combined, washed with brine (30 mL), dried over sodium sulfate, filtered and concentrated. The residue was subjected to flash chromatography on silica gel (petroleum ether:ethyl acetate=75:25) to obtain 6-chloro-5-(methylsulfinyl)-2-morpholino-N-(pyridin-3-ylpyrimidin-4-amine as yellow solid (180 mg, 70.3%). LCMS (ESI) m/z: 354.0[M+H]⁺.

### Step 4: Synthesis of 6-(3-(1-methyl-1H-pyrazol-3-yl)phenyl)-5-(methylsulfinyl)-2-morpholino-N-(pyridin-3-yl)pyrimidin-4-amine

To a solution of 6-chloro-5-(methylsulfinyl)-2-morpholino-N-(pyridin-3-yl)pyrimidin-4-amine (180 mg, 0.508 mmol) in 1,4-dioxane (5 mL) and water (5 mL) were added 1-methyl-3-(3-(4,4,5,5-tetramethyl-1,3,2-dioxaborolan-2-yl)phenyl)-1H-pyrazole (224 mg, 0.792 mmol), potassium carbonate (147 mg, 1.05 mmol) and dichloro[1.1′-bis(diphenylphosphino)ferrocene]palladium(II) dichloromethane adduct (50 mg, 0.196 mmol). The resulting mixture was stirred at 90° C. for 2 h. The resultant mixture was filtered, diluted with water (10 mL) and extracted with ethyl acetate (10 mL×3). The organic layers were combined, washed with brine (30 mL), dried over sodium sulfate, filtered and concentrated. The residue was subjected to prep-HPLC (0.05% ammonium bicarbonate:acetonitrile=5%˜95%) to obtain 6-(3-(1-methyl-1H-pyrazol-3-yl)phenyl)-5-(methylsulfinyl)-2-morpholino-N-(pyridin-3-ylpyrimidin-4-amine (26.6 mg, 28.5%) as white solid. ¹H NMR (400 MHz, DMSO-d₆) δ 10.32 (s, 1H), 8.78 (d, J=2.4 Hz, 1H), 8.31 (dd, J=4.6, 1.2 Hz, 1H), 8.04 (ddd, J=8.3, 2.6, 1.5 Hz, 1H), 7.92-7.81 (m, 2H), 7.76 (d, J=2.2 Hz, 1H), 7.52-7.33 (m, 3H), 6.76 (d, J=2.3 Hz, 1H), 3.90 (s, 3H), 3.74 (s, 4H), 3.67 (s, 4H), 2.98 (s, 3H). LCMS (ESI) m/z: 475.6[M+H]⁺.

### Example 52. Synthesis of 4-(5-methoxy-6-(3-(1-methyl-1H-pyrazol-3-yl)phenyl)-2-morpholinopyrimidin-4-yl)-1-methylpiperazin-2-one (Compound 60)

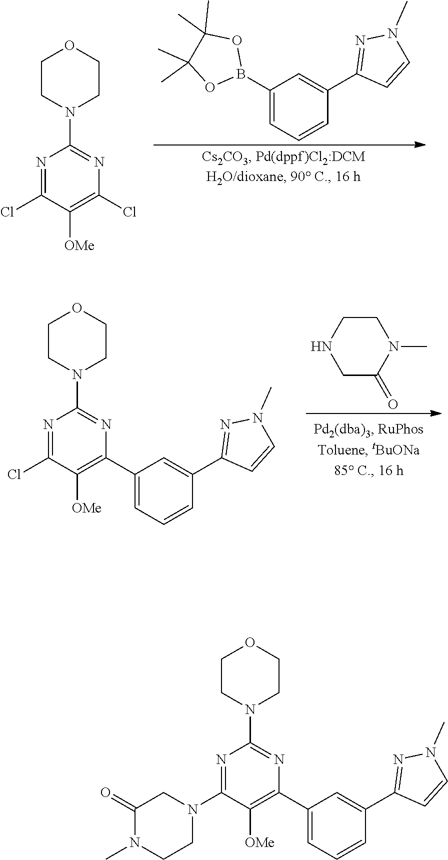

### Step 1: Synthesis of 4-(4-chloro-5-methoxy-6-(3-(1-methyl-1H-pyrazol-3-yl)phenyl)pyrimidin-2-yl)morpholine

A mixture of 4-(4,6-dichloro-5-methoxypyrimidin-2-yl)morpholine (782 mg, 3 mmol), 1-methyl-3-(3-(4,4,5,5-tetramethyl-1,3,2-dioxaborolan-2-yl)phenyl)-1H-pyrazole (852 mg, 3 mmol), 1,1′-bis(diphenylphosphino)ferrocene-palladium(II)dichloride dichloromethane complex (245 mg, 0.3 mmol) and cesium carbonate (2445 mg, 7.5 mmol) in 1,4-dioxane/water (20 mL/4 mL) was stirred at 90° C. under argon atmosphere for 16 h. The reaction mixture was filtered to remove the solids, the filtrate was concentrated, and the residue was subjected to silica gel column chromatography (petroleum ether:acetic ester=3:1) to obtain 4-(4-chloro-5-methoxy-6-(3-(1-methyl-1H-pyrazol-3-yl)phenyl)pyrimidin-2-yl)morpholine (523 mg, 45%) as white solid. LCMS (ESI) m/z: 386.2 [M+H]⁺.

### Step 2: Synthesis of 4-(5-methoxy-6-(3-(1-methyl-1H-pyrazol-3-yl)phenyl)-2-morpholinopyrimidin-4-yl)-1-methylpiperazin-2-one

A mixture of 4-(4-chloro-5-methoxy-6-(3-(1-methyl-1H-pyrazol-3-yl)phenyl)pyrimidin-2-yl)morpholine (153.5 mg, 0.4 mmol, 1-methylpiperazin-2-one (45 mg, 0.4 mmol), tris(dibenzylideneacetone)dipalladium (37 mg, 0.04 mmol), 2-dicyclohexylphosphino-2′,6′-di-i-propoxy-1,1′-biphenyl (37 mg, 0.08 mmol) and sodium tert-butoxide (115 mg, 4.14 mmol) in toluene (10 mL) was stirred at 85° C. under argon atmosphere for 16 h. The reaction mixture was filtered to remove solids, the filtrate was concentrated, and the residue was subjected to prep-HPLC [Welch Xtimate C18 21.2×250 mm, 10 um, with mobile phase acetonitrile/water (10 mM NH₄HCO₃ and NH₃·H₂O)] to obtain 4-(5-methoxy-6-(3-(1-methyl-1H-pyrazol-3-yl)phenyl)-2-morpholinopyrimidin-4-yl-1-methylpiperazin-2-one (64 mg, 34.5%) as white solid. ¹H NMR (400 MHz, DMSO-d₆) δ 8.29 (s, 1H), 7.84-7.74 (m, 3H), 7.47 (t, J=7.7 Hz, 1H), 6.70 (d, J=2.1 Hz, 1H), 4.26 (s, 2H), 4.00 (t, J=5.3 Hz, 2H), 3.90 (s, 3H), 3.65 (d, J=2.3 Hz, 8H), 3.46 (t, J=5.3 Hz, 2H), 3.31 (s, 3H), 2.89 (s, 3H). LCMS (ESI) m/z: 464.4 [M+H]⁺.

### Example 53. Synthesis of 4-(4-(3-(1H-pyrazol-1-yl)phenyl)-6-methoxy-6-(4-phenyl-1H-imidazol-2-yl)pyrimidin-2-yl)morpholine (Compound 61)

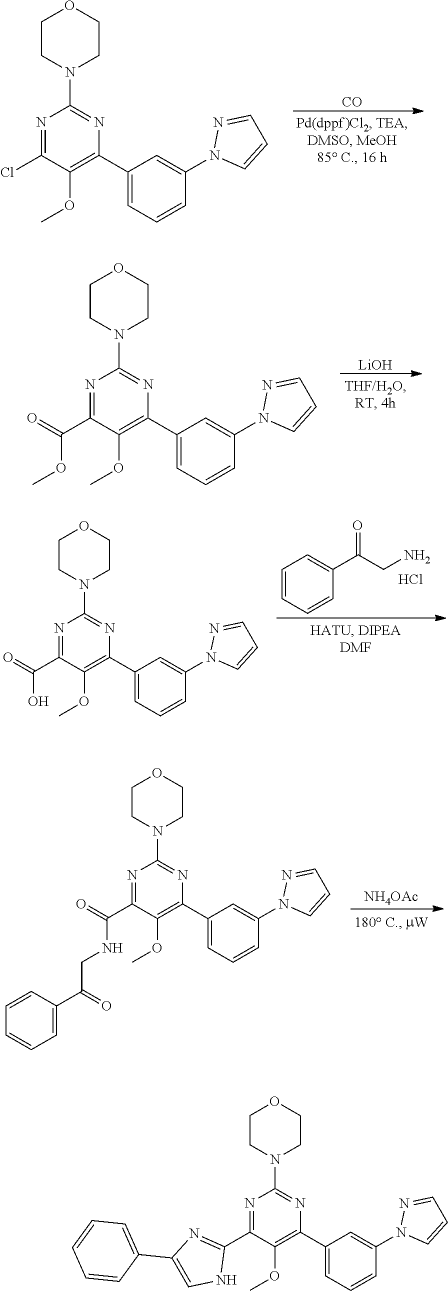

### Step 1: Synthesis of methyl 6-(3-(1H-pyrazol-1-yl)phenyl)-6-methoxy-2-morpholinopyrimidine-4-carboxylate

To a solution of 4-(4-(3-(1H-pyrazol-1-yl)phenyl)-6-chloro-5-methoxypyrimidin-2-yl)morpholine (400 mg, 1.08 mmol) in dimethyl sulfoxide (8 mL) and methanol (10 mL) were added palladium (II) acetate (20 mg, 0.088 mmol), 1,1′-bis(diphenylphosphino)ferrocene (244 mg, 0.44 mmol) and triethylamine (266 mg, 2.64 mmol) and the reaction mixture was stirred at 85° C. for 16 h under carbon monoxide atmosphere. The mixture was extracted with dichloromethane (20 mL*2) and washed with water (10 mL*2). The organic layer was dried over sodium sulfate, filtered and filtrate was concentrated. The residue was then subjected to silica gel column chromatography (3% methanol in dichloromethane) to obtain methyl 6-(3-(1H-pyrazol-1-yl)phenyl)-5-methoxy-2-morpholino-pyrimidine-4-carboxylate (130 mg, 30%). LCMS (ESI) m/z: 395.9 [M+H]⁺.

### Step 2: Synthesis of 6-(3-(1H-pyrazol-1-yl)phenyl)-5-methoxy-2-morpholinopyrimidine-4-carboxylic acid

To a solution of methyl 6-(3-(1H-pyrazol-1-yl)phenyl)-5-methoxy-2-morpholinopyrimidine-4-carboxylate (130 mg, 0.3 mmol) in tetrahydrofuran (5 mL) and water (1 mL) was added lithium hydroxide monohydrate (25 mg, 0.6 mmol) at 0° C. and the reaction mixture was stirred at room temperature for 1 h. Then the mixture was quenched by the addition with water (10 mL) and then the mixture was filtered to remove the solids. The filtrate was extracted with dichloromethane (10 mL*3), the combined organic phase was dried over sodium sulphate, filtered and concentrated. The residue was purified by flash chromatography on silica gel (methanol:dichloromethane=1:8) to obtain 6-(3-(1H-pyrazol-1-yl)phenyl)-5-methoxy-2-morpholinopyrimidine-4-carboxylic acid (110 mg, 90%) as yellow solid. LCMS (ESI) m/z: 381.8 [M+H]⁺.

### Step 3: Synthesis of 6-(3-(1H-pyrazol-1-yl)phenyl)-5-methoxy-2-morpholino-N-(2-oxo-2-phenylethyl)pyrimidine-4-carboxamide

To a stirred solution of 6-(3-(1H-pyrazol-1-yl)phenyl)-5-methoxy-2-morpholinopyrimidine-4-carboxylic acid (110 mg, 0.27 mmol) and 2-amino-1-phenylethan-1-one (40 mg, 0.30 mmol) in N,N-dimethylformamide (10 mL) were added O-(7-azabenzotriazol-1-yl)-N,N,N′,N′-tetraethyluroniumhexafluorophosphate (150 mg, 0.4 mmol) and N,N-diisopropylethylamine (130 mg, 1 mmol). The resultant mixture was stirred at 20° C. for 16 h and concentrated. The residue was subjected to flash chromatography (dichloromethane:methanol=50:1) to obtain 6-(3-(1H-pyrazol-1-yl)phenyl)-5-methoxy-2-morpholino-N-(2-oxo-2-phenylethyl)pyrimidine-4-carboxamide (125 mg, 90%) as white solid. LCMS (ESI) m/z: 498.7 [M+H]⁺.

### Step 4: Synthesis of 4-(4-(3-(1H-pyrazol-1-yl)phenyl)-5-methoxy-6-(4-phenyl-1H-imidazol-2-yl)pyrimidin-2-yl)morpholine

A solution of 6-(3-(1H-pyrazol-1-ylphenyl)-5-methoxy-2-morpholino-N-(2-oxo-2-phenylethyl)pyrimidine-4-carboxamide (125 mg, 0.24 mmol) and ammonium acetate (500 mg, 5.5 mmol) was stirred at 180° C. for 2 h under argon atmosphere. It was cooled and the mixture was diluted with ethyl acetate (25 mL) and washed with water (25 mL). The organic layer was dried over sodium sulphate, filtered and concentrated under the reduced pressure. The residue was subjected to flash chromatography on silica gel (petroleum ether ethyl acetate=1:1) to obtain 4-(4-(3-(1H-pyrazol-1-yl)phenyl)-5-methoxy-6-(4-phenyl-1H-imidazol-2-yl)pyrimidin-2-yl)morpholine (4.3 mg, 4%) as yellow solid. ¹H NMR (400 MHz, DMSO-d₆) δ 12.88 (s, 1H), 8.59 (d, J=2.4 Hz, 1H), 8.52 (s, 1H), 8.00 (s, 1H), 7.98 (s, 1H), 7.94 (d, J=8.0 Hz, 3H), 7.80 (s, 1H), 7.65 (t, J=7.9 Hz, 1H), 7.41 (t, J=7.6 Hz, 2H), 7.26 (s, 1H), 6.59 (s, 1H), 3.86 (s, 4H), 3.74 (d, J=4.6 Hz, 4H), 3.70 (s, 3H). LCMS (ESI) m/z: 479.7 [M+H]⁺.

### Example 53. Synthesis of 5-methoxy-6-(3-(1-methyl-1H-pyrazol-3-yl)phenyl)-N-(pyridin-3-ylmethyl)-2-(pyridin-4-yl)pyrimidin-4-amine (Compound 62)

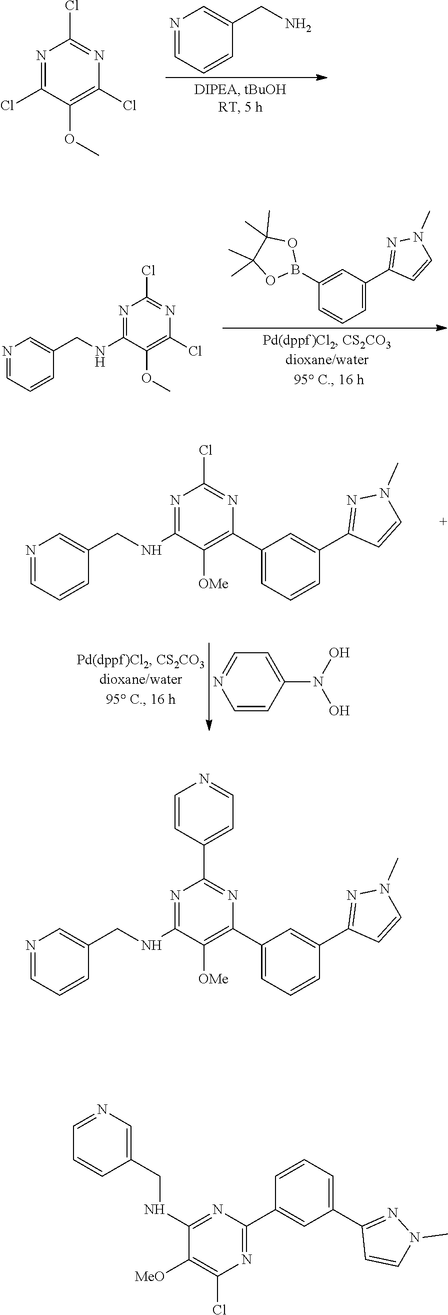

### Step 1: Synthesis of 2,6-dichloro-5-methoxy-N-(pyridin-3-ylmethyl)pyrimidin-4-amine

A mixture of 2,4,6-trichloro-5-methoxypyrimidine (0.45 g, 2.11 mmol), pyridin-3-ylmethanamine (0.23 g, 2.11 mmol) and N,N-diisopropylethylamine (0.55 g, 4.22 mmol) in tert-butanol (15 mL) was stirred at room temperature for 5 h. It was concentrated and the residue was subjected to column chromatography (Biotage, 40 g silica gel, eluted with 7N ammonia methanol in dichloromethane from 5% to 15%) to obtain 2,6dichloro-5-methoxy-N-(pyridin-3-ylmethyl)pyrimidin-4-amine (0.39 g, 65%) as brown solid. LCMS (ESI) m/z: 284.8 [M+H]⁺.

### Step 2: Synthesis of 2-chloro-5-methoxy-6-(3-(1-methyl-H-pyrazol-3-yl)phenyl)-N-(pyridin-3-ylmethyl)pyrimidin-4-amine and 6-chloro-6-methoxy-2-(3-(1-methyl-1H-pyrazol-3-yl)phenyl)-N-(pyridin-3-ylmethyl)pyrimidin-4-amine

A mixture of 2,6-dichloro-5-methoxy-N-(pyridin-3-ylmethyl)pyrimidin-4-amine (0.35 g, 1.23 mmol), 1-methyl-3-(3-(4,4,5,5-tetramethyl-1,3,2-dioxaborolan-2-yl)phenyl)-1H-pyrazole (314 mg, 1.1 mmol), 1,1′-bis(diphenylphosphino)ferrocenepalladium (II)dichloride dichloromethane complex (100 mg, 0.12 mmol) and cesium carbonate (1 g, 3.07 mmol) in 1,4-dioxane/water (25 mL/4 mL) was stirred at 95° C. for 16 h. It was concentrated, the residue was diluted with water (20 mL) and extracted with ethyl acetate (20 mL×2). The combined organic phase was washed with brine (20 mL), dried over sodium sulfate, filtered and concentrated. The residue was subjected to prep-HPLC (base) to obtain 2-chloro-5-methoxy-6-(3-(1-methyl-1H-pyrazol-3-yl)phenyl)-N-(pyridin-3-ylmethyl)pyrimidin-4-amine (50 mg, 11.2%) and 6-chloro-5-methoxy-2-(3-(1-methyl-1H-pyrazol-3-yl)phenyl)-N-(pyridin-3-ylmethyl)pyrimidin-4-amine (100 mg, 22.4%) as white solids. LCMS (ESI) m/z: 407.1 [M+H]⁺.

### Step 3: Synthesis of 5-methoxy-4-(3-(1-methyl-1H-pyrazol-3-yl)phenyl)-N-(pyridin-3-ylmethyl)-2-(pyridin-4-yl)pyrimidin-4-amine

A mixture of 2-chloro-5-methoxy-6-(3-(1-methyl-1H-pyrazol-3-yl)phenyl))-N-(pyridin-3-ylmethyl)pyrimidin-4-amine (40 mg, 0.098 mmol), pyridin-4-ylboronic acid (15 mg, 0.12 mmol), 1,1′-bis(diphenylphosphino)ferrocenepalladium (II)dichloride dichloromethane complex (10 mg, 0.01 mmol) and cesium carbonate (0.08 g, 0.25 mmol) in 1,4-dioxane/water (5 mL/1 mL) was stirred at 95° C. for 16 h. The mixture was concentrated, and the residue was subjected to prep-HPLC (base) to obtain 5-methoxy-6-(3-(1-methyl-1H-pyrazol-3-yl)phenyl)-N-(pyridin-3-ylmethyl)-2-(pyridin-4-yl)pyrimidin-4-amine (5.9 mg, 13.4%) as white solid. ¹H NMR (400 MHz, CDCl₃) δ 8.77-8.66 (m, 3H), 8.62-8.54 (m, 1H), 8.48 (t, J=1.6 Hz, 1H), 8.28 (dd, J=4.5, 1.6 Hz, 2H), 8.04-7.99 (m, 1H), 7.93 (dd, J=7.8, 1.4 Hz, 1H), 7.78 (d, J=7.8, 1 Hz, 1H), 7.53 (t, J=7.8 Hz, 1H), 7.42 (d, J=2.2 Hz, 1H), 7.31 (dd, J=7.8, 4.8 Hz, 1H), 6.63 (d, J=2.3 Hz, 1H), 5.95 (t, J=6.0 Hz, 1H), 4.89 (d, J=6.0 Hz, 2H), 3.98 (s, 3H), 3.57 (s, 3H). LCMS (ESI) m/z: 450.1 [M+H]⁺.

### Example 54. Synthesis of 5-methoxy-2-(3-(1-methyl-1H-pyrazol-3-yl)phenyl)-N-(pyridin-3-ylmethyl)-6-(pyridin-4-yl)pyrimidin-4-amine (Compound 63)

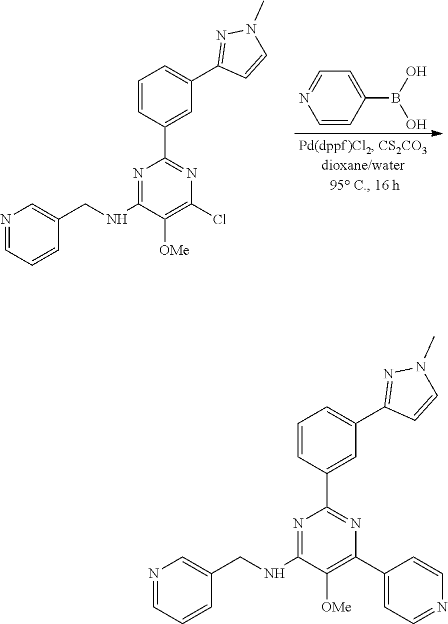 [SMILES: COc1c(Cl)nc(-c2cccc(-c3ccn(C)n3)c2)nc1NCc1cccnc1 ; OB(O)c1ccncc1 ; COc1c(NCc2cccnc2)nc(-c2cccc(-c3ccn(C)n3)c2)nc1-c1ccncc1]

A mixture of 6-chloro-5-methoxy-2-(3-(1-methyl-1H-pyrazol-3-yl)phenyl))-N-(pyridin-3-ylmethyl)pyrimidin-4-amine (70 mg, 0.17 mmol), pyridin-4-ylboronic acid (25 mg, 0.21 mmol), 1,1′-bis(diphenylphosphino)ferrocenepalladium (II)dichloride dichloromethane complex (14 mg, 0.02 mmol) and cesium carbonate (0.14 g, 0.43 mmol) in 1,4-dioxane/water (8 mL/1 mL) was stirred at 95° C. for 16 h. The mixture was concentrated, and the residue was subjected to prep-HPLC (base) to obtain 5-methoxy-2-(3-(1-methyl-1H-pyrazol-3-yl)phenyl)-N-(pyridin-3-ylmethyl)-6-(pyridin-4-yl)pyrimidin-4-amine (22.9 mg, 30%) as white solid. ¹H NMR (400 MHz, DMSO-d₆) δ 8.80-8.73 (m, 3H), 8.70 (s, 1H), 8.44 (dd, J=4.8, 1.5 Hz, 1H), 8.33 (t, J=6.1 Hz, 1H), 8.23 (d, J=7.8 Hz, 1H), 8.04 (dd, J=4.6, 1.5 Hz, 2H), 7.90 (d, J=7.9, 1H), 7.85 (d, J=7.8 Hz, 1H), 7.78 (d, J=2.2 Hz, 1H), 7.48 (t, J=7.7 Hz, 1H), 7.37 (dd, J=7.8, 4.8 Hz, 1H), 6.73 (d, J=2.2 Hz, 1H), 4.77 (d, J=6.0 Hz, 2H), 3.92 (s, 3H), 3.60 (s, 3H). LCMS (ESI) m/z: 450.1 [M+H]⁺.

### Example 55. Synthesis of 2-morpholino-9-pyrazol-1-yl-4-(4-pyridylamino)-6H-pyrimido[5,4-c]quinolin-5-one (Compound 64) and 6-methyl-2-morpholino-9-pyrazol-1-yl-4-(4-pyridylamino)pyrimido[5,4-c]quinolin-5-one (Compound 65)

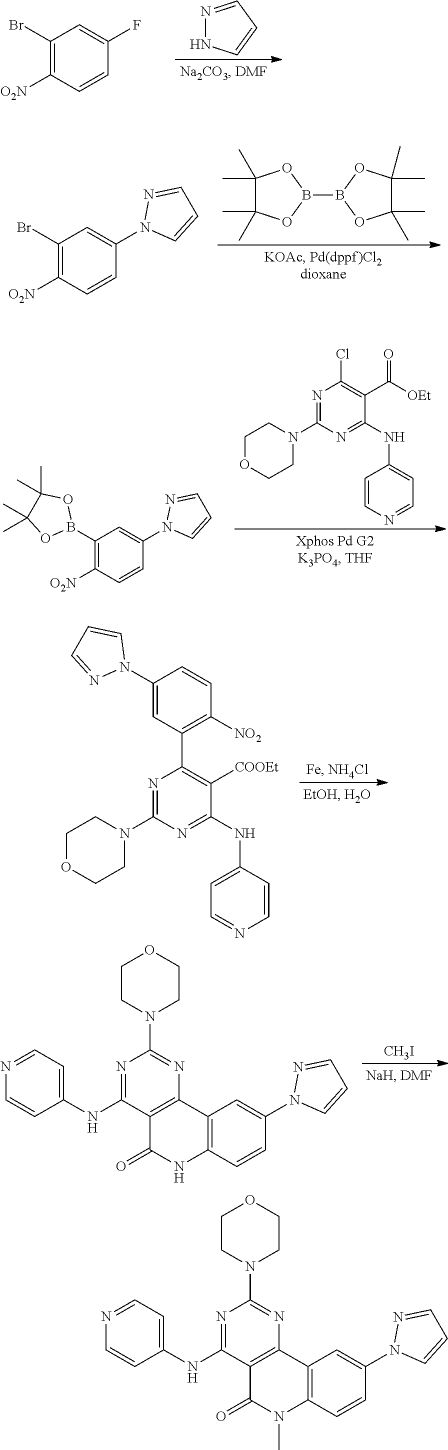

### Step 1: Synthesis of 1-(3-bromo-4-nitro-phenyl)pyrazole

To a solution of 2-bromo-4-fluoro-1-nitrobenzene (2 g, 9.09 mmol) in DMF (20 mL) were added 1H-pyrazole (619 mg, 9.09 mmol) and Na₂CO₃ (2.41 g, 22.73 mmol). The resulting mixture was stirred at 100° C. for 12 h, then cooled to 15° C. and poured into ice-water (50 mL). The aqueous phase was extracted with ethyl acetate (50 mL*3), the combined organic phase was washed with brine (10 mL), dried over anhydrous Na₂SO₄, filtered and concentrated. The residue was subjected to flash column chromatography (ISCO 40 g silica, 0-30% ethyl acetate in petroleum ether, gradient over 20 min) to obtain 1-(3-bromo-4-nitro-phenyl)pyrazole (1.3 g, 53%) as yellow solid.

### Step 2: Synthesis of 1-[4-nitro-3-(4,4,5,5-tetramethyl-1,3,2-dioxaborolan-2-yl)phenyl]pyrazole

To a solution of 1-(3-bromo-4-nitro-phenyl)pyrazole (1.15 g, 4.29 mmol) in dioxane (10 mL) were added 4,4,5,5-tetramethyl-2-(4,4,5,5-tetramethyl-1,3,2-dioxaborolan-2-yl)-1,3,2-dioxaborolane (1.31 g, 5.15 mmol), Pd(dppf)Cl₂ (314 mg, 429 umol) and KOAc (1.26 g, 12.87 mmol). The resultant mixture was stirred at 80° C. for 12 h. It was then filtered to remove the solids and the filtrate was concentrated to afford the crude product. It was purified by flash column chromatography (ISCO 40 g silica, 0-50% ethyl acetate in petroleum ether, gradient over 20 min) to obtain 1-[4-nitro-3-(4,4,5,5-tetramethyl-1,3,2-dioxaborolan-2-yl)phenyl]pyrazole (1 g, 74%) as yellow solid. ¹H NMR (400 MHz, CHLOROFORM-d) δ 8.29 (d, J=8.8 Hz, 1H), 8.05 (d, J=2.6 Hz, 1H), 7.91-7.76 (m, 3H), 6.59-6.52 (m, 1H), 1.28 (s, 12H)

### Step 3: Synthesis of ethyl 2-morpholino-4-(2-nitro-5-pyrazol-1-yl-phenyl)-64-pyridylamino)pyrimidine-5-carboxylate

To a solution of ethyl 4-chloro-2-morpholino-6-(4-pyridylamino)pyrimidine-5-carboxylate (200 mg, 550 umol) in THF (5 mL) were added 1-[4-nitro-3-(4,4,5,5-tetramethyl-1,3,2-dioxaborolan-2-yl)phenyl]pyrazole (208 mg, 660 umol), K₃PO₄ (350 mg, 1.65 mmol) and [2-(2-aminophenyl)phenyl]-chloro-palladium; dicyclohexyl-[2-(2,6-dimethoxyphenyl)phenyl]phosphane (43 mg, 55 umol) under nitrogen atmosphere. The mixture was stirred at 80° C. for 4 h, then cooled to 15° C. and then poured into ice-water (15 mL). The aqueous phase was extracted with ethyl acetate (15 mL*3), the combined organic phase was washed with brine (10 mL), dried over anhydrous Na₂SO₄, filtered and concentrated. The residue was subjected to flash column chromatography (ISCO 10 g silica, 0-100% ethyl acetate in petroleum ether, gradient over 20 min) to obtain ethyl 2-morpholino-4-(2-nitro-5-pyrazol-1-yl-phenyl)-6-(4-pyridylamino)pyrimidine-5-carboxylate (340 mg, 60%) as yellow solid.

### Step 4: Synthesis of 2-morpholino-9-pyrazol-1-yl-4-(4-pyridylamino)-6H-pyrimido[5,4-c]quinolin-6-one

To a solution of ethyl 2-morpholino-4-(2-nitro-5-pyrazol-1-yl-phenyl)-6-(4-pyridylamino)pyrimidine-5-carboxylate (230 mg, 445 umol) in EtOH (3 mL) and H₂O (1 mL) were added Fe (249 mg, 4.45 mmol) and NH₄Cl (238 mg, 4.45 mmol). The mixture was stirred at 80° C. for 8 h and the iron powder was removed by filtration. The filtrate was then concentrated to obtain 2-morpholino-9-pyrazol-1-yl-4-(4-pyridylamino)-6H-pyrimido[5,4-c]quinolin-5-one (150 mg crude) as black solid. The crude product (30 mg) was purified by prep-HPLC [Welch Xtimate C18 21.2×250 mm, 10 um, with mobile phase acetonitrile/water (10 mM NH₄HCO₃ and NH₃·H₂O)] to obtain 2-morpholino-9-pyrazol-1-yl-4-(4-pyridylamino)-6H-pyrimido[5,4-c]quinolin-5-one (2 mg, 4.75 umol) as white solid. ¹H NMR (400 MHz, DMSO-d₆) δ 12.34 (s, 1H), 8.75 (d, J=2.4 Hz, 1H), 8.58 (d, J=2.4 Hz, 1H), 8.50 (d, J=5.4 Hz, 2H), 8.13 (dd, J=8.9, 2.6 Hz, 1H), 7.81-7.66 (m, 3H), 7.48 (d, J=8.7 Hz, 1H), 6.59 (s, 1H), 3.80-3.79 (m, 4H), 3.78 (d, J=4.4 Hz, 4H). LCMS (ESI for C23H20N8O2) [M+H]⁺: 441.1.

### Step 5: Synthesis of 6-methyl-2-morpholino-9-pyrazol-1-yl-4-(4-pyridylamino)pyrimido[5,4-c]quinolin-one

To a solution of 2-morpholino-9-pyrazol-1-yl-4-(4-pyridylamino)-6H-pyrimido[5,4-c]quinolin-5-one (100 mg, 227 umol) in DMF (1 mL) was added NaH (18 mg, 454 umol, 60% suspension in oil) at 0° C. The mixture was warmed up and stirred at 25° C. for 0.5 h. Then iodomethane (32 mg, 227 umol) was added to the mixture and it was stirred at 25° C. for another 2 h. The mixture was then poured into ice-water (2 mL) at 0° C., filtered and the filtrate was concentrated to afford crude product. It was then dissolved in DMF (1 mL), let it stand for one day, the solid formed was then removed by filtration, washed with H₂O (1 mL*2) and the combined filtrates were concentrated to afford 6-methyl-2-morpholino-9-pyrazol-1-yl-4-(4-pyridylamino)pyrimido[5,4-c]quinolin-5-one (9 mg, 8%) was obtained as a white solid. ¹H NMR (400 MHz, DMSO-d₆) δ 12.21 (bs, 1H), 8.68 (bs, 1H), 8.56-8.33 (m, 3H), 8.09-7.95 (m, 1H), 7.80 (s, 1H), 7.61 (bs, 2H), 7.48 (bs, 1H), 6.58 (bs, 1H), 4.08-3.38 (m, 11H). LCMS (ESI for C24H22N8O2) [M+H]⁺: 455.1.

### Example 56. Synthesis of N,N-dimethyl-4-[3-(1-methylpyrazol-3-yl)phenyl]-2-morpholino-(4-pyridylamino)pyrimidine-6-carboxamide (Compound 66)

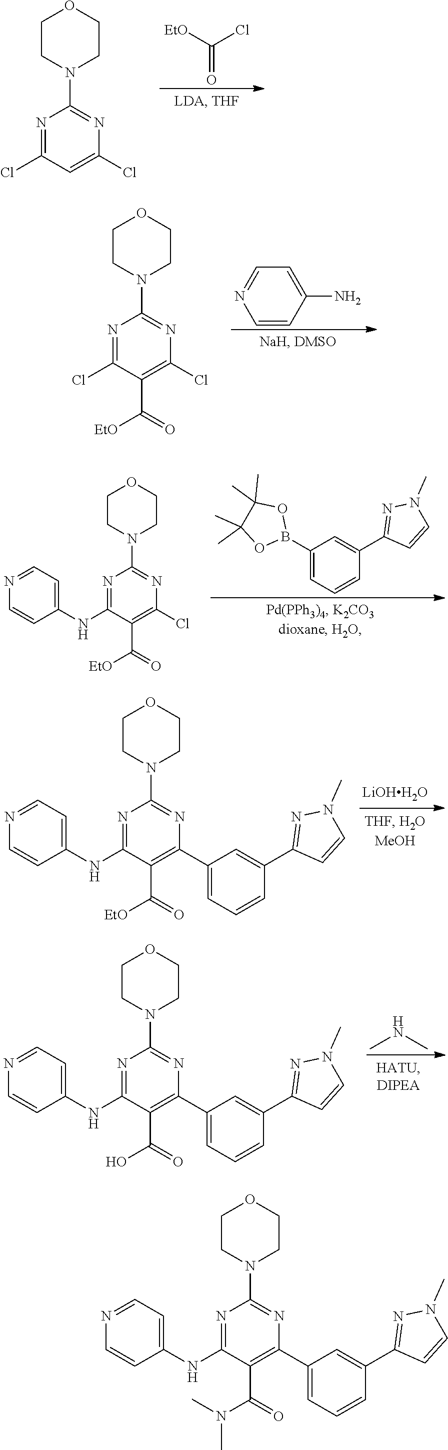

### Step 1: Synthesis of ethyl 4,6-dichloro-2-morpholino-pyrimidine-6-carboxylate

A solution of LDA (2M in THF, 17.94 mL) was added dropwise to a solution of 4-(4,6-dichloropyrimidin-2-yl)morpholine (7 g, 29.90 mmol) in anhydrous THF (70 mL) at −70-−60° C. under nitrogen atmosphere and the mixture was stirred for 1 h. Ethyl carbonochloridate (8.68 g, 79.98 mmol) was then added via syringe and the mixture was stirred at −70-−60° C. for an additional 2 h. The contents were allowed to warm up to 25° C. and stirred for another 0.5 h. It was then poured into ice-water (100 mL) and the aqueous phase was extracted with ethyl acetate (100 mL*3). The combined organic phase was washed with brine (50 mL), dried with anhydrous Na₂SO₄, filtered and concentrated. The residue was subjected to flash column chromatography (ISCO 40 g silica, 0-20% ethyl acetate in petroleum ether, gradient over 20 min) to obtain ethyl 4,6-dichloro-2-morpholino-pyrimidine-5-carboxylate (8.4 g, 92%) as white solid.

### Step 2: Synthesis of ethyl 4-chloro-2-morpholino-6(4-pyridylamino)pyrimidine-6-carboxylate

To a solution of pyridin-4-amine (0.96 g, 10.20 mmol) in DMSO (15 mL) was added NaH (392 mg, 9.80 mmol, 60% suspension) at 0° C. The mixture was stirred at 20° C. for 0.5 h, cooled again to 0° C., followed by the addition of ethyl 4,6-dichloro-2-morpholino-pyrimidine-5-carboxylate (1.5 g, 4.90 mmol). The mixture was further stirred at 20° C. for an additional 2 h and then poured into ice-water (30 mL). The aqueous phase was extracted with ethyl acetate (30 mL*3), the combined organic phase was washed with brine (10 mL), dried over anhydrous Na₂SO₄, filtered and concentrated. The residue was subjected to flash column chromatography (ISCO 20 g silica, 0-100% ethyl acetate in petroleum ether, gradient over 20 min) to obtain ethyl 4-chloro-2-morpholino-6-(4-pyridylamino)pyrimidine-5-carboxylate (850 mg) as pale-yellow solid. LCMS (ESI) m/z: 364.0 [M+H]⁺

### Step 3: Synthesis of ethyl 4-[3-(1-methylpyrazol-3-yl)phenyl]-2-morpholino-6(4-pyridylamino)pyrimidine-6-carboxylate

To a solution of ethyl 4-chloro-2-morpholino-6-(4-pyridylamino)pyrimidine-5-carboxylate (500 mg, 1.37 mmol) in dioxane (7 mL) and H₂O (0.7 mL) were added 1-methyl-3-[3-(4,4,5,5-tetramethyl-1,3,2-dioxaborolan-2-yl)phenyl]pyrazole (586 mg, 2.06 mmol), K₂CO_{M}(570 mg, 4.12 mmol) and Pd(PPh₃)₄ (159 mg, 137 umol). The resultant mixture was stirred at 100° C. for 3 h under nitrogen atmosphere. The reaction mixture was filtered, and the filtrate was concentrated in vacuo. To the residue was added 10 mL of water and the aqueous mixture was extracted with ethyl acetate (10 mL*2). The combined organic layers were washed with brine (10 mL) and dried over Na₂SO₄ and concentrated. The residue was subjected to flash column chromatography (ISCO 20 g silica, 0-87% ethyl acetate in petroleum ether, gradient over 20 min) to obtain ethyl 4-[3-(1-methylpyrazol-3-yl)phenyl]-2-morpholino-6-(4-pyridylamino)pyrimidine-5-carboxylate (520 mg, 78%) as pale-yellow solid. ¹H NMR (400 MHz, CHLOROFORM-d) δ 8.51 (d, J=6.3 Hz, 2H), 7.93-7.80 (m, 2H), 7.69-7.61 (m, 2H), 7.49-7.33 (m, 3H), 6.56 (d, J=2.3 Hz, 1H), 4.04-3.86 (m, 9H), 3.80 (bs, 4H), 0.76 (t, J=7.2 Hz, 3H); LCMS (ESI) m/z: 486.3 [M+H]⁺.

### Step 4: Synthesis of 4-[3-(1-methylpyrazol-3-yl)phenyl]-2-morpholino-6-(4-pyridylamino)pyrimidine-5-carboxylic acid

To a solution of ethyl 4-[3-(1-methylpyrazol-3-yl)phenyl]-2-morpholino-6-(4-pyridylamino)pyrimidine-5-carboxylate (500 mg, 1.03 mmol) in THF (4 mL), MeOH (2 mL) and H₂O (2 mL) was added LIOH·H₂O (129 mg, 3.09 mmol). The mixture was stirred at 25° C. for 12 h and concentrated. The residue was diluted with 5 mL H₂O and saturated aqueous citric acid was added to the mixture at 0° C. until PH=3-4. The resultant precipitate was collected by filtration, washed with water and dried in vacuo to obtain 4-[3-(1-methylpyrazol-3-yl)phenyl]-2-morpholino-6-(4-pyridylamino)pyrimidine-5-carboxylic acid (450 mg, 96%) as yellow solid. LCMS (ESI) m/z: 458.2 [M+H]⁺

### Step 5: Synthesis of N,N-dimethyl-4-[3-(1-methylpyrazol-3-yl)phenyl]-2-morpholino-6(4-pyridylamino)pyrimidine-5-carboxamide

To a mixture of 4-[3-(1-methylpyrazol-3-yl)phenyl]-2-morpholino-6-(4-pyridylamino) pyrimidine-5-carboxylic acid (120 mg, 262 umol) and N,N-dimethylamine (2M in THF, 1 mL) were added HATU (120 mg, 315 umol) and DIPEA (102 mg, 787 umol). The mixture was stirred at 20° C. for 2 h followed by the addition of 1 mL of water. The mixture was extracted with ethyl acetate (3 mL*2), the combined organic layers were washed with brine (1 mL) and dried over Na₂SO₄ and concentrated. The residue was subjected to prep-HPLC [Welch Xtimate C18 21.2×250 mm, 10 um, with mobile phase acetonitrile/water (10 mM NH₄HCO₃ and NH₃·H₂O)] to obtain N,N-dimethyl-4-[3-(1-methylpyrazol-3-yl)phenyl]-2-morpholino-6-(4-pyridylamino)pyrimidine-5-carboxamide (51 mg, 40%) as white solid. ¹H NMR (400 MHz, CHLOROFORM-d) δ 8.47 (d, J=5.8 Hz, 2H), 8.14-8.09 (m, 1H), 7.97 (d, J=7.8 Hz, 1H), 7.77-7.66 (m, 2H), 7.61 (d, J=7.8 Hz, 1H), 7.51-7.44 (m, 1H), 7.42 (d, J=2.1 Hz, 1H), 6.59 (d, J=2.3 Hz, 1H), 4.06-3.89 (m, 7H), 3.88-3.79 (m, 4H), 2.85 (s, 3H), 2.37 (s, 3H). LCMS (ESI) for (C26H28N8O2) [M+H]⁺: 485.3.

### Example 57. Synthesis of 4-(3-cyanophenyl)-N,N-dimethyl-2-morpholino-6-(4-pyridylamino)pyrimidine-6-carboxamide (Compound 67)

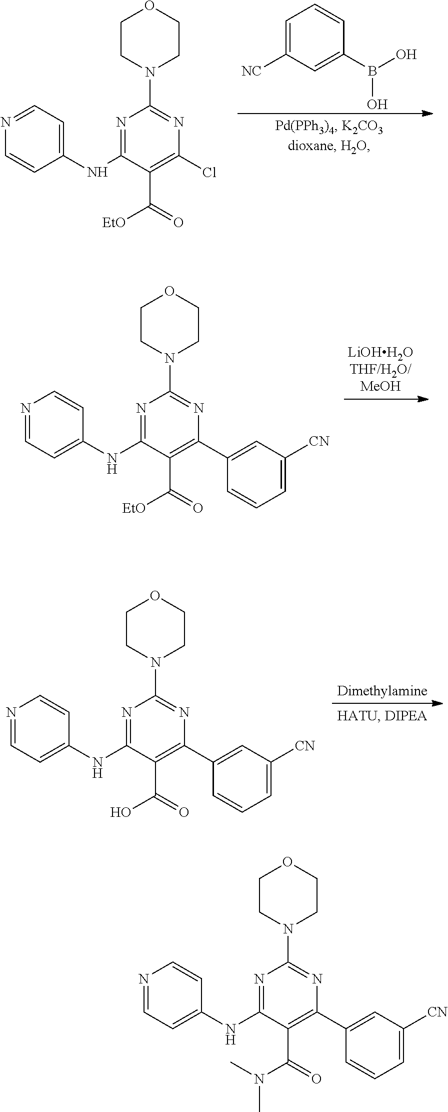 [SMILES: CCOC(=O)c1c(Cl)nc(N2CCOCC2)nc1Nc1ccncc1 ; N#Cc1cccc(B(O)O)c1 ; CCOC(=O)c1c(Nc2ccncc2)nc(N2CCOCC2)nc1-c1cccc(C#N)c1 ; N#Cc1cccc(-c2nc(N3CCOCC3)nc(Nc3ccncc3)c2C(=O)O)c1 ; CN(C)C(=O)c1c(Nc2ccncc2)nc(N2CCOCC2)nc1-c1cccc(C#N)c1]

### Step 1: Synthesis of ethyl 4-(3-cyanophenyl)-2-morpholino-6(4-pyridylamino)pyrimidine-6-carboxylate

To a solution of ethyl 4-chloro-2-morpholino-6-(4-pyridylamino)pyrimidine-5-carboxylate (400 mg, 1.10 mmol) in dioxane (5 mL) and H₂O (0.5 mL) were added (3-cyanophenyl)boronic acid (242 mg, 1.65 mmol), K₂CO (456 mg, 3.30 mmol) and Pd(PPh₃)₄ (127 mg, 110 umol). The mixture was stirred at 100° C. for 3 h under nitrogen atmosphere. It was then filtered; the filtrate was concentrated, and the residue was diluted with 5 mL of water. The aqueous phase was extracted with ethyl acetate (5 mL*2), the combined organic layers were washed with brine (5 mL), dried over Na₂SO₄ and concentrated. The residue was subjected to flash column chromatography (ISCO 20 g silica, 0-80% ethyl acetate in petroleum ether, gradient over 20 min) to obtain ethyl 4-(3-cyanophenyl)-2-morpholino-6-(4-pyridylamino)pyrimidine-5-carboxylate (250 mg, 53%) as yellow solid. LCMS (ESI) m/z: 431.1 [M+H]⁺

### Step 2: Synthesis of 4-(3-cyanophenyl)-2-morpholino-6(4-pyridylamino)pyrimidine-6-carboxylic acid

To a solution of ethyl 4-(3-cyanophenyl)-2-morpholino-6-(4-pyridylamino)pyrimidine-5-carboxylate (240 mg, 558 umol) in THF (2 mL), MeOH (1 mL) and H₂O (1 mL) was added LiOH·H₂O (70 mg, 1.67 mmol). The mixture was stirred at 25° C. for 12 h and concentrated. To the residue was added 3 mL water and saturated aqueous citric acid at 0° C. until pH=4. The resulting precipitate was collected by filtration, washed with water and dried in vacuo to obtain 4-(3-cyanophenyl)-2-morpholino-6-(4-pyridylamino)pyrimidine-5-carboxylic acid (220 mg, 98%) as yellow solid. LCMS (ESI) m/z: 403.1 [M+H]⁺

### Step 3: Synthesis of 4-(3-cyanophenyl)-N,N-dimethyl-2-morpholino-6(4-pyridylamino)pyrimidine-5-carboxamide

To a mixture of 4-(3-cyanophenyl)-2-morpholino-6-(4-pyridylamino)pyrimidine-5-carboxylic acid (110 mg, 273 umol) and N,N-dimethylamine (2M in THF, 1 mL) were added HATU (125 mg, 328 umol) and DIPEA (106 mg, 820 umol). The mixture was stirred at 25° C. for 2 h and concentrated. The residue was subjected to prep-HPLC (Phenomenex C18 75*30 mm*3 um column; 10-55% acetonitrile in an a 10 mM ammonium hydroxide solution in water, 8 min gradient) to obtain 4-(3-cyanophenyl)-N,N-dimethyl-2-morpholino-6-(4-pyridylamino)pyrimidine-5-carboxamide (8 mg, 7%) as white solid. ¹H NMR (400 MHz, CHLOROFORM-d) δ 8.77 (s, 1H), 8.50 (d, J=5.8 Hz, 2H), 8.11 (t, J=1.5 Hz, 1H), 7.87 (td, J=8.2, 1.4 Hz, 1H), 7.77 (td, J=7.8, 1.4 Hz, 1H), 7.57-7.51 (m, 3H), 3.93 (bs, 4H), 3.86-3.81 (m, 4H), 2.89 (s, 3H), 2.38 (s, 3H). LCMS (ESI) for (C23H23N7O2) [M+H]⁺: 430.3.

### Example 58. Synthesis of azetidin-1-yl[4-[3-(1-methylpyrazol-3-yl)phenyl]-2-morpholino-6-(4-pyridylamino)pyrimidin-6-yl]methanone (Compound 68)

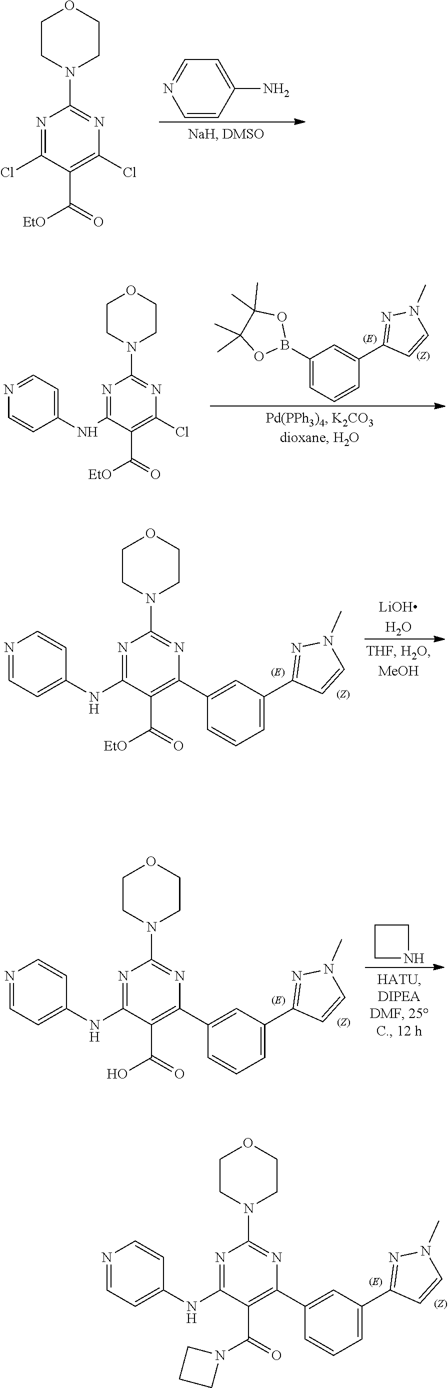

### Step 1: Synthesis of ethyl 4-chloro-2-morpholino-64-pyridylamino)pyrimidine-6-carboxylate

To a solution of pyridin-4-amine (154 mg, 1.63 mmol) in DMSO (30 mL) was added NaH (131 mg, 3.27 mmol, 60% suspension) in portions at 0° C. Then the mixture was stirred at 25° C. for 0.5 h and then ethyl 4,6-dichloro-2-morpholino-pyrimidine-5-carboxylate (500 mg, 1.63 mmol) was added to the above mixture at 0° C. The resultant mixture was stirred at 25° C. for 2 h and then poured into aqueous saturated NH₄Cl solution (30 mL) at 0° C. and the aqueous phase was extracted with ethyl acetate (30 mL*3). The combined extracts were washed with brine (20 mL), dried with anhydrous Na₂SO₄ and concentrated. The residue was subjected to flash chromatography (ISCO 40 g silica, 0-56% ethyl acetate in petroleum ether, gradient over 20 min) to obtain ethyl 4-chloro-2-morpholino-6-(4-pyridylamino)pyrimidine-5-carboxylate (1.66 g, 28%) as yellow solid. ¹H NMR (400 MHz, CHLOROFORM-d) δ 10.82 (s, 1H), 8.46 (d, J=6.1 Hz, 2H), 7.49 (d, J=6.2 Hz, 2H), 4.36 (q, J=7.1 Hz, 2H), 3.86-3.72 (m, 8H), 1.39 (t, J=7.2 Hz, 3H)

### Step 2: Synthesis of ethyl 4-[3-(1-methylpyrazol-3-yl)phenyl)]-2-morpholino-6(4-pyridylamino)pyrimidine-6-carboxylate

To a solution of ethyl 4-chloro-2-morpholino-6-(4-pyridylamino)pyrimidine-5-carboxylate (1 g, 2.06 mmol) in dioxane (15 mL) and H₂O (1.5 mL) were added 1-methyl-3-[3-(4,4,5,5-tetramethyl-1,3,2-dioxaborolan-2-yl)phenyl]pyrazole (879 mg, 2.47 mmol), K₂CO₃ (855 mg, 6.18 mmol) and Pd(PPh₃)₄ (238 mg, 206 umol). The resultant mixture was stirred at 100° C. for 3 h under nitrogen atmosphere. The reaction mixture was then diluted with water and extracted with ethyl acetate (20 mL*2). The combined organic layers were washed with brine (20 mL), dried over Na₂SO₄ and concentrated. The residue was subjected to flash chromatography (ISCO 20 g silica, 0-77% ethyl acetate in petroleum ether, gradient over 20 min) to obtain ethyl 4-[3-(1-methylpyrazol-3-yl)phenyl]-2-morpholino-6-(4-pyridylamino)pyrimidine-5-carboxylate (600 mg, 60%) as pale yellow solid. LCMS (ESI) m/z: 486.3 [M+H]⁺

### Step 3: Synthesis of 4-[3-(1-methylpyrazol-3-yl)phenyl]-2-morpholino-6-(4-pyridylamino)pyrimidine-6-carboxylic acid

To a solution of ethyl 4-[3-(1-methylpyrazol-3-yl)phenyl]-2-morpholino-6-(4-pyridylamino)pyrimidine-5-carboxylate (200 mg, umol) in THF (4 mL), MeOH (2 mL) and H₂O (2 mL) was added LiOH·H₂O (52 mg, 1.24 mmol). The mixture was stirred at 25° C. for 12 h and concentrated. The residue was diluted with 5 mL H₂O and saturated aqueous citric acid solution was added to the mixture at 0° C. until pH=5-6. The resultant precipitate was filtered and dried in vacuum to obtain the crude product (300 mg). 150 mg of this product was purified by prep-HPLC (Waters Xbridge Prep OBD C18 150*40 mm*10 um; 5-45% acetonitrile in an a 10 mM ammonium bicarbonate solution in water, 8 min gradient) to obtain compound 4-[3-(1-methylpyrazol-3-yl)phenyl]-2-morpholino-6-(4-pyridylamino)pyrimidine-5-carboxylic acid (78 mg, 41%) as white solid. ¹H NMR (400 MHz, DMSO-d₆) δ 11.00-10.64 (m, 1H), 8.50-8.42 (m, 2H), 7.84 (s, 1H), 7.81 (td, J=1.5, 7.4 Hz, 1H), 7.74 (d, J=2.1 Hz, 1H), 7.72-7.67 (m, 2H), 7.45-7.35 (m, 2H), 6.71 (d, J=2.3 Hz, 1H), 3.88 (s, 3H), 3.85-3.78 (m, 4H), 3.71 (d, J=4.1 Hz, 4H). LCMS (ESI) for (C24H23N7O3) [M+H]⁺: 458.1

### Step 4: Synthesis of azetidin-1-yl(4-(3-(1-methyl-1H-pyrazol-3-yl)phenyl)-2-morpholino-6-(pyridin-4-ylamino)pyrimidin-a-yl)methanone

To a solution of 4-[3-(1-methylpyrazol-3-yl)phenyl]-2-morpholino-6-(4-pyridylamino)pyrimidine-5-carboxylic acid (150 mg, 327.88 umol) in DMF (2 mL) were added azetidine (423 mg, 7.41 mmol), HATU (150 mg, 393 umol) and DIPEA (127 mg, 984 umol). The mixture was stirred at 25° C. for 2 h and the entire mixture was subjected to prep-HPLC (Waters Xbridge Prep OBD C18 150*40 mm*10 um column; 25-55% acetonitrile in an a 10 mM ammonium hydroxide solution in water, 8 min gradient) to obtain azetidin-1-yl(4-(3-(1-methyl-1H-pyrazol-3-ylphenyl)-2-morpholino-6-(pyridin-4-ylamino)pyrimidin-5-ylmethanone (78 mg, 48%) as white solid. ¹H NMR (400 MHz, CHLOROFORM-d) δ 9.79 (s, 1H), 8.48 (d, J=6.3 Hz, 2H), 8.18 (t, J=1.6 Hz, 1H), 7.99 (td, J=7.8, 1.3 Hz, 1H), 7.73-7.60 (m, 3H), 7.52-7.45 (m, 1H), 7.42 (d, J=2.3 Hz, 1H), 6.61 (d, J=2.3 Hz, 1H), 4.04-3.93 (m, 8H), 3.90-3.73 (m, 5H), 3.42-3.27 (m, 2H), 2.07-1.99 (m, 2H). LCMS (ESI) for (C27H28N8O2) [M+H]⁺: 497.3

### Example 59. Synthesis of N,N-dimethyl-2-morpholino-4-(3-pyrazol-1-ylphenyl)-6-(3-pyridylamino)pyrimidine-6-carboxamide (Compound 69)

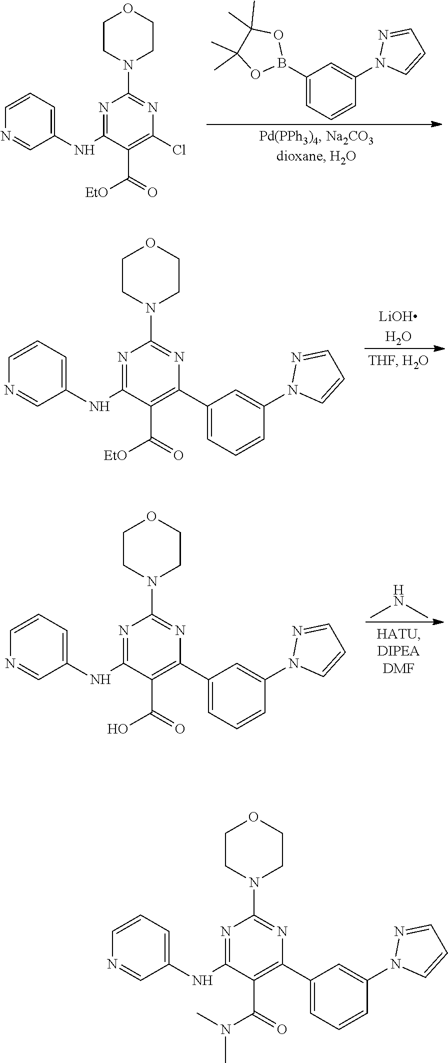

### Step 1: Synthesis of ethyl 2-morpholino-4-(3-pyrazol-1-ylphenyl)-6-(3-pyridylamino)pyrimidine-6-carboxylate

To a solution of ethyl 4-chloro-2-morpholino-6-(3-pyridylamino)pyrimidine-5-carboxylate (500 mg, 1.37 mmol) in dioxane (5 mL) and H₂O (0.5 mL) were added 1-[3-(4,4,5,5-tetramethyl-1,3,2-dioxaborolan-2-yl)phenyl]pyrazole (371 mg, 1.37 mmol), Pd(PPh₃)₄ (159 mg, 137 umol) and K₂CO₃ (570 mg, 4.12 mmol). The mixture was stirred at 100° C. for 3 h under nitrogen atmosphere, then diluted with 10 mL of water and the aqueous mixture was extracted with ethyl acetate (5 mL*3). The combined organic layers were dried over Na₂SO₄ and concentrated. The residue was subjected to flash column chromatography (ISCO 12 g silica, 0-60% ethyl acetate in petroleum ether, gradient over 30 min) to obtain ethyl 2-morpholino-4-(3-pyrazol-1-ylphenyl)-6-(3-pyridylamino)pyrimidine-5-carboxylate (500 mg, 46%) as yellow oil. LCMS (ESI) for [M+H]⁺: 472.2

### Step 2: Synthesis of 2-morpholino-4-(3-pyrazol-1-ylphenyl)-6-(3-pyridylamino)pyrimidine-6-carboxylic acid

To a solution of ethyl 2-morpholino-4-(3-pyrazol-1-yl)phenyl)-6-(3-pyridylamino)pyrimidine-5-carboxylate (500 mg, 530 umol) in THF (4 mL) and MeOH (2 mL) was added a solution of LiOH·H₂O (67 mg, 1.59 mmol) in H₂O (2 mL) at 0° C. The mixture was then warmed up and stirred at 20° C. for 3 h and concentrated. It was then diluted with 8 mL of water and the mixture was extracted with ethyl acetate (5 mL*3). The organic phase was discarded, and the aqueous phase was treated with saturated aqueous citric acid solution until pH=5 under ice-bath conditions. The solid formed was collected by filtration and dried under reduced pressure to obtain 2-morpholino-4-(3-pyrazol-1-ylphenyl)-6-(3-pyridylamino)pyrimidine-5-carboxylic acid (150 mg, 64%) as yellow solid. LCMS (ESI) for [M+H]⁺: 444.1

### Step 3: Synthesis of N,N-dimethyl-2-morpholino-4-(3-pyrazol-1-ylphenyl)-6-(3-pyridylamino)pyrimidine-6-carboxamide

To a solution of 2-morpholino-4-(3-pyrazol-1-yl)phenyl)-6-(3-pyridylamino)pyrimidine-5-carboxylic acid (120 mg, 271 umol) in DMF (2 mL) were added N-methylmethanamine (2M, 406 uL), HATU (123 mg, 325 umol) and DIPEA (105 mg, 812 umol). The mixture was stirred at 20° C. for 14 h and concentrated. The residue was subjected to prep-HPLC [Welch Xtimate C18 21.2×250 mm, 10 um, with mobile phase acetonitrile/water (10 mM NH₄HCO₃ and NH₃·H₂O)] to obtain N,N-dimethyl-2-morpholino-4-(3-pyrazol-1-ylphenyl)-6-(3-pyridylamino)pyrimidine-5-carboxamide (82 mg, 65%) as white solid. ¹H NMR (400 MHz, CHLOROFORM-d) δ 8.95 (d, J=2.4 Hz, 1H), 8.56 (s, 1H), 8.32 (d, 1H, J=3.6 Hz), 8.09 (s, 1H), 7.99 (s, 1H), 7.84 (q, J=1.4 Hz, 2H), 7.58 (s, 1H), 7.53-7.52 (m, 1H), 7.52-7.51 (m, 1H), 7.29-7.28 (m, 1H), 6.51 (s, 1H), 3.89 (s, 4H), 3.80-3.78 (m, 4H), 2.88 (s, 3H), 2.45 (s, 3H). LCMS (ESI for C25H26N8O2) [M+H]⁺: 471.2

### Example 60. Synthesis of [2-morpholino-4-(3-pyrazol-1-ylphenyl)-6-(3-pyridylamino)pyrimidin-6-yl]methanol (Compound 70)

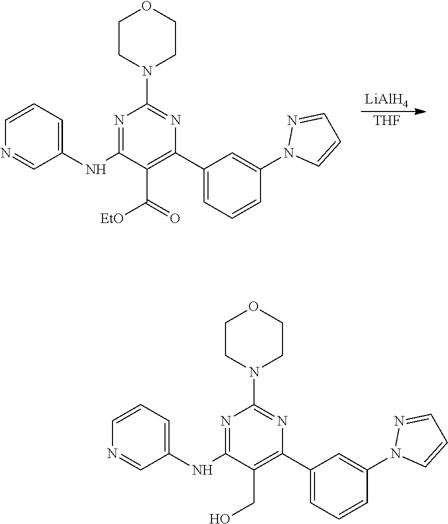 [SMILES: CCOC(=O)c1c(Nc2cccnc2)nc(N2CCOCC2)nc1-c1cccc(-n2cccn2)c1 ; "LiAlH4" ; "THF" ; OCc1c(Nc2cccnc2)nc(N2CCOCC2)nc1-c1cccc(-n2cccn2)c1]

To a solution of ethyl 2-morpholino-4-(3-pyrazol-1-yl)phenyl)-6-(3-pyridylamino)pyrimidine-5-carboxylate (210 mg, 445 umol) in THF (3 mL) was added LiAlH₄ (34 mg, 891 umol) in portions at 0° C. The resultant mixture was stirred at 20° C. for 12 h. Then the reaction was quenched with Na₂SO₄·10H₂O (2 eq) at 0° C. and the mixture was warmed up and stirred at 20° C. for 30 min. The resulting heterogeneous mixture was filtered to remove the solids and the filtrate was concentrated. The residue was then subjected to prep-HPLC (Phenomenex luna C18 80*40 mm*3 um column; 8-28% acetonitrile in an a 0.04% hydrochloric acid solution in water, 7 min gradient) and then again to an additional prep-HPLC condition (Phenomenex luna C18 80*40 mm*3 um column; 18-33% acetonitrile in an a 0.04% hydrochloric acid solution in water, 6 min gradient) to obtain [2-morpholino-4-(3-pyrazol-1-ylphenyl)-6-(3-pyridylamino)pyrimidin-5-yl]methanol (17 mg, 8%) as pale-yellow viscous liquid. ¹H NMR (400 MHz, METHANOL-d) δ=9.37 (d, J=1.6 Hz, 1H), 8.80 (d, J=8.4 Hz, 1H), 8.71 (d, J=5.6 Hz, 1H), 8.38 (s, 1H), 8.11-8.05 (m, 3H), 7.82 (d, J=1.6 Hz, 1H), 7.76-7.74 (m, 1H), 7.62-7.60 (m, 1H), 6.62 (t, J=2.0 Hz, 1H), 4.62 (s, 2H), 3.83-3.79 (m, 8H). LCMS (ESI for C23H23N7O2) [M+H]J: 430.0.

### Example 61. Synthesis of 6-methyl-9-(1-methylpyrazol-3-yl)-2-morpholino-4-(4-pyridylamino)pyrimido[5,4-c]quinolin-one (Compound 71)

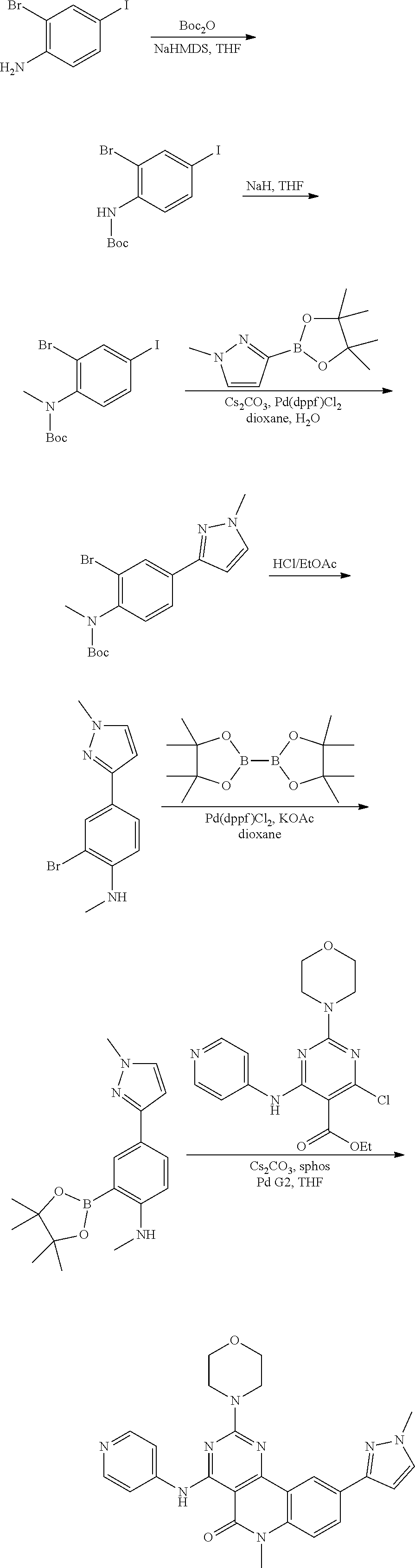

### Step 1: Synthesis of tert-butyl N-(2-bromo-4-iodo-phenyl)carbamate

To a solution of 2-bromo-4-iodo-aniline (4 g, 13.43 mmol) in THF (40 mL) was added NaHMDS (1M, 20.14 mL) at 0° C. The mixture was stirred at 0° C. for 30 min, and then a solution of Boc₂O (3.22 g, 14.77 mmol) in THF (10 mL) was added dropwise at 0° C. The mixture was stirred at 25° C. for 2 h and a saturated aqueous solution of NH₄Cl (80 mL) was added to the flask carefully. The resultant mixture was extracted with ethyl acetate (20 mL*3), the combined organic layers were washed with brine (15 mL), dried over Na₂SO₄ and concentrated. The residue was subjected to flash column chromatography (ISCO 20 g silica, 0-3% ethyl acetate in petroleum ether, gradient over 10 min) to obtain tert-butyl N-(2-bromo-4-iodo-phenyl)carbamate (3 g, 56%) as yellow solid. LCMS (ESI) m/z: 397.1 [M+H]⁺.

### Step 2: Synthesis of tert-butyl) N-(2-bromo-4-iodo-phenyl)-N-methylcarbamate

To a solution of tert-butyl N-(2-bromo-4-iodo-phenyl)carbamate (3 g, 7.54 mmol) in THF (40 mL) was added NaH (361 mg, 9.04 mmol, 60% suspension in oil) in portions at 0° C. The resultant heterogeneous mixture was stirred at 0° C. for 30 min followed by the addition of CH₃I (2.14 g, 15.07 mmol). The mixture was warmed up and stirred at 25° C. for 2 h followed by the addition of 50 mL of aqueous saturated NH₄Cl solution. The resultant mixture was then extracted with ethyl acetate (30 mL*2), the combined organic layers were washed with brine (3 mL), dried over Na₂SO₄ and concentrated to obtain tert-butyl N-(2-bromo-4-iodo-phenyl))-N-methylcarbamate (3 g, 96%) as yellow oil. ¹H NMR (400 MHz, CHLOROFORM-d) δ 7.95 (s, 1H), 7.62 (d, J=8.0 Hz, 1H), 6.95 (d, J=8.3 Hz, 1H), 3.13 (s, 3H), 1.36 (s, 9H).

### Step 3: Synthesis of tert-butyl) N-[2-bromo-4-(1-methylpyrazol-3-yl)phenyl]-N-methylcarbamate

To a solution of tert-butyl N-(2-bromo-4-iodo-phenyl)-N-methylcarbamate (1.7 g, 4.13 mmol) in dioxane (20 mL) and H₂O (2 mL) were added 1-methyl-3-(4,4,5,5-tetramethyl-1,3,2-dioxaborolan-2-yl)pyrazole (858 mg, 4.13 mmol), Cs₂CO₃ (2.69 g, 8.25 mmol) and Pd(dppf)Cl₂ (301 mg, 412 umol). The resultant mixture was stirred at 80° C. for 5 h under argon atmosphere. It was then diluted with 40 mL of water and the mixture was extracted with ethyl acetate (20 mL*3). The combined organic layers were washed with brine (15 mL), dried over Na₂SO₄ and concentrated. The residue was subjected to flash column chromatography (ISCO 20 g silica, 0-30% ethyl acetate in petroleum ether, gradient over 20 min) to obtain tert-butyl N-[2-bromo-4-(1-methylpyrazol-3-yl)phenyl]-N-methylcarbamate (900 mg, 59%) as yellow oil. ¹H NMR (400 MHz, CHLOROFORM-d) δ 8.05 (s, 1H), 7.70 (dd, J=8.1, 1.6 Hz, 1H), 7.38-7.26 (m, 1H), 7.22 (d, J=8.1 Hz, 1H), 6.53 (d, J=2.0 Hz, 1H), 3.96 (s, 3H), 3.17 (s, 3H), 1.35 (s, 9H).

### Step 4: Synthesis of 2-bromo-N-methyl-4-(1-methylpyrazol-3-yl)aniline

A solution of tert-butyl N-[2-bromo-4-(1-methylpyrazol-3-yl)phenyl]-N-methylcarbamate (850 mg, 2.32 mmol) in HCl/EtOAc (4M, 580 uL) was stirred at 25° C. for 2 h and concentrated. The residue was dissolved in water (5 mL) and then basified with saturated aqueous NaHCO₃ at 0° C. The resulting mixture was extracted with ethyl acetate (10 mL*2), the combined organic layers were washed with brine (5 mL), dried over Na₂SO₄ and concentrated to obtain 2-bromo-N-methyl-4-(1-methylpyrazol-3-yl) aniline (600 mg, 97%) as yellow oil. LCMS (ESI) m/z: 266.2 [M+H]⁺

### Step 5: Synthesis of N-methyl-4-(1-methylpyrazol-3-yl)-2-(4,4,5,5-tetramethyl-1,3,2-dioxaborolan-2-yl)aniline

To a solution of 4,4,4′,4′,5,5,5′,5′-octamethyl-2,2′-bi(1,3,2-dioxaborolane) (687 mg, 2.71 mmol) in dioxane (10 mL) were added 2-bromo-N-methyl-4-(1-methylpyrazol-3-yl)aniline (600 mg, 2.25 mmol), Pd(dppf)Cl₂ (165 mg, 225 umol) and AcOK (443 mg, 4.51 mmol). The resultant mixture was stirred at 80° C. for 5 h under argon atmosphere and then diluted with 20 mL of water. It was then extracted with ethyl acetate (10 mL*3), the combined organic layers were dried over Na₂SO₄ and concentrated. The residue was then subjected to flash column chromatography (ISCO 20 g silica, 30-100% ethyl acetate in petroleum ether, gradient over 30 min) to obtain N-methyl-4-(1-methylpyrazol-3-yl)-2-(4,4,5,5-tetramethyl-1,3,2-dioxaborolan-2-yl)aniline (400 mg, 57%) as yellow oil. LCMS (ESI) m/z: 314.2 [M+H]⁺

### Step 6: Synthesis of 6-methyl-9-(1-methylpyrazol-3-yl)-2-morpholino-4-(4-pyridylamino)pyrimido[5,4-c]quinolin-one

To a solution of ethyl 4-chloro-2-morpholino-6-(4-pyridylamino)pyrimidine-5-carboxylate (140 mg, 384 umol) and N-methyl-4-(1-methylpyrazol-3-yl-2-(4,4,5,5-tetramethyl-1,3,2-dioxaborolan-2-yl)aniline (132 mg, 423 umol) in THF (3 mL) were added [2-(2-aminophenyl)phenyl]-chloro-palladium; dicyclohexyl-[2-(2,6-dimethoxyphenyl)phenyl]phosphane (27 mg, 38 umol) and K₃PO₄ (245 mg, 1.15 mmol). The resultant reaction mixture was stirred at 80° C. for 4 h under argon atmosphere and concentrated. The residue was then subjected to prep-HPLC (Phenomenex Luna C18 75*30 mm*3 um column; 10%-50% acetonitrile in 0.05% formic acid solution in water, 8 min gradient) to obtain 6-methyl-9-(1-methylpyrazol-3-yl)-2-morpholino-4-(4-pyridylamino)pyrimido[5,4-c]quinolin-5-one (3 mg, 1.40%) as pale-yellow solid. ¹H NMR (400 MHz, METHANOL-d4) δ 8.96 (s, 1H), 8.40 (d, J=6.1 Hz, 2H), 8.02 (d, J=8.9 Hz, 1H), 7.78 (d, J=5.8 Hz, 2H), 7.67 (s, 1H), 7.48 (d, J=8.5 Hz, 1H), 6.68 (d, J=2.3 Hz, 1H), 4.03-4.01 (m, 6H), 3.85-3.83 (m, 5H), 3.68 (s, 3H). LC-MS: (ESI for C25H24N8O2) m/z: 469.1 [M+H]⁺.

### Example 62. Synthesis of N-[2-(4-oxo-1-phenyl-1,3,8-triazaspiro[4.5]decan-8-yl)ethyl]naphthalene-2-carboxamide (Compound 72)

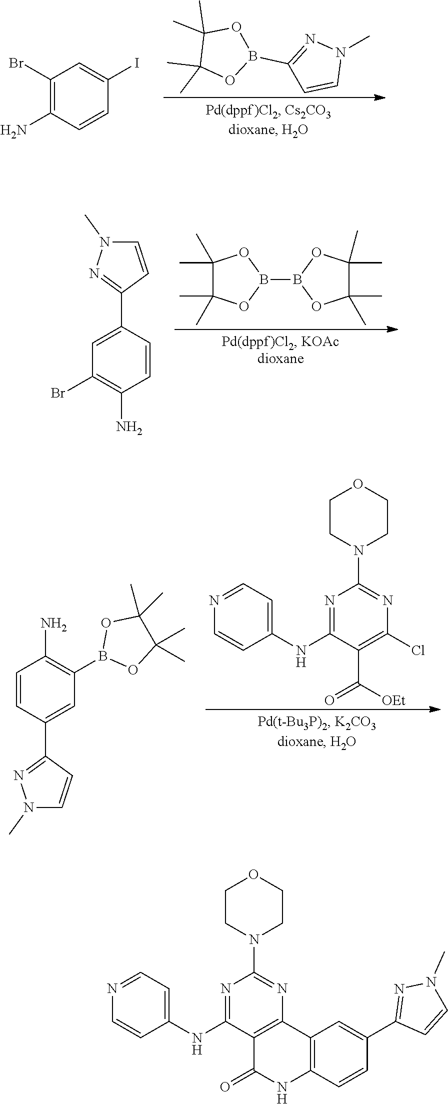

### Step 1: Synthesis of 2-bromo-4-(1-methylpyrazol-3-yl)aniline

To a solution of 2-bromo-4-iodo-aniline (1.2 g, 4.03 mmol) in dioxane (10 mL) and H₂O (1 mL) were added 1-methyl-3-(4,4,5,5-tetramethyl-1,3,2-dioxaborolan-2-yl)pyrazole (838 mg, 4.03 mmol), Cs₂CO₃ (2.62 g, 8.06 mmol) and Pd(dppf)Cl₂ (295 mg, 403 umol). The mixture was stirred at 80° C. for 5 h under argon atmosphere. It was then diluted with 20 mL of water and extracted with ethyl acetate (10 mL*3). The combined organic layers were washed with brine (10 mL), dried over Na₂SO₄ and concentrated. The residue was subjected to flash column chromatography (ISCO 20 g silica, 0-30% ethyl acetate in petroleum ether, gradient over 20 min) to obtain 2-bromo-4-(1-methylpyrazol-3-yl)aniline (620 mg, 61%) as yellow oil. ¹H NMR (400 MHz, CHLOROFORM-d) δ 7.87 (d, J=1.8 Hz, 1H), 7.53 (dd, J=8.3, 1.8 Hz, 1H), 7.34 (d, J=2.1 Hz, 1H), 6.79 (d, J=8.3 Hz, 1H), 6.41 (d, J=2.1 Hz, 1H), 4.14 (s, 2H), 3.94 (s, 3H); LCMS (ESI) m/z: 253.9 [M+H]⁺.

### Step 2: Synthesis of 4-(1-methylpyrazol-3-yl)-2-(4,4,5,5-tetramethyl-1,3,2-dioxaborolan-2-yl)aniline

To a solution of 4,4,4′,4′,5,5,5′,5′-octamethyl-2,2′-bi(1,3,2-dioxaborolane) (725 mg, 2.86 mmol) in dioxane (10 mL) were added 2-bromo-4-(1-methylpyrazol-3-yl)aniline (600 mg, 2.38 mmol), Pd(dppf)Cl₂ (174 mg, 238 umol) and KOAc (584 mg, 5.95 mmol). The mixture was stirred at 90° C. for 15 h under argon atmosphere and then diluted with 20 mL of water. It was extracted with ethyl acetate (20 mL*3), the combined organic phase was washed with brine, dried over Na₂SO₄ and concentrated. The residue was subjected to flash column chromatography (ISCO 20 g silica, 0-100% ethyl acetate in petroleum ether, gradient over 20 min) to obtain 4-(1-methylpyrazol-3-yl)-2-(4,4,5,5-tetramethyl-1,3,2-dioxaborolan-2-yl)aniline (400 mg, 56%) as yellow oil. LCMS (ESI) m/z: 300.2 [M+H]⁺

### Step 3: Synthesis of 9-(1-methylpyrazol-3-yl)-2-morpholino-4-(4-pyridylamino)-6H-pyrimido[5,4-c]quinolin-one

To a solution of ethyl 4-chloro-2-morpholino-6-(4-pyridylamino)pyrimidine-5-carboxylate (200 mg, 549 umol) and 4-(1-methylpyrazol-3-yl-2-(4,4,5,5-tetramethyl-1,3,2-dioxaborolan-2-yl)aniline (181 mg, 605 umol) in THF (2 mL) were added [2-(2-aminophenyl)phenyl]-chloro-palladium; dicyclohexyl-[2-(2,6-dimethoxyphenyl)phenyl]phosphane (40 mg, 55 umol) and K₃PO₄ (350 mg, 1.65 mmol). The reaction mixture was stirred at 80° C. for 4 h under argon atmosphere and concentrated. The residue was subjected to prep-HPLC (Waters Xbridge Prep OBD C18 150*40 mm*10 um column; 25%-65% acetonitrile in 0.05% ammonium hydroxide and 10 mM sodium bicarbonate solution in water, 8 min gradient) to obtain 9-(1-methylpyrazol-3-yl-2-morpholino-4-(4-pyridylamino)-6H-pyrimido[5,4-c]quinolin-5-one (7 mg, 3%) as pale-yellow solid. ¹H NMR (400 MHz, DMSO-d6) δ 12.36 (s, 1H), 11.83 (s, 1H), 8.75 (d, J=2.0 Hz, 1H), 8.48 (d, J=6.0 Hz, 2H), 8.04 (dd, J=8.6, 1.9 Hz, 1H), 7.80-7.74 (m, 3H), 7.38 (d, J=8.5 Hz, 1H), 6.78 (s, 1H), 4.00-3.76 (m, 7H), 3.79 (t, J=4.4 Hz, 4H). LC-MS: (ESI for C24H22N8O2) m/z: 455.1 [M+H]⁺.

### Example 63. Synthesis of 6-[3-(1-methylpyrazol-3-yl)phenyl]-N-(1-methylpyridin-1-ium-4-yl)-2-morpholino-1-oxazol-2-yl-pyrimidin-4-amine (Compound 73) and 6-[3-(1-methylpyrazol-3-yl)phenyl]-2-morpholino-5-oxazol-2-yl-N-(4-pyridyl)pyrimidin-4-amine (Compound 74)

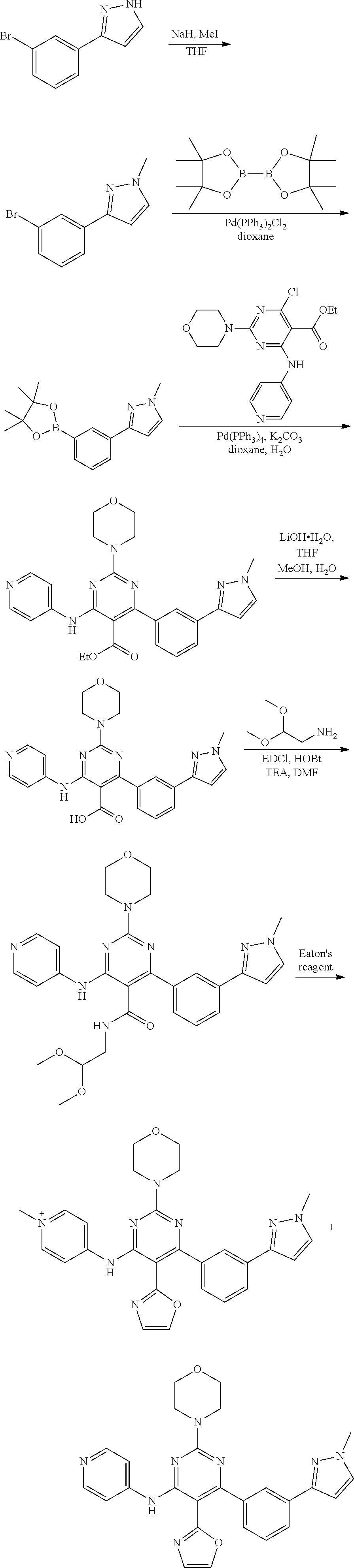

### Step 1: Synthesis of 3-(3-bromophenyl)-1-methyl-pyrazole

To a solution of 3-(3-bromophenyl)-1H-pyrazole (5 g, 19.50 mmol) in DMF (35 mL) was added NaH (1.17 g, 29.25 mmol, 60% suspension in oil) in portions at 0° C. and the mixture was stirred at 0° C. for 0.5 h. Then under argon atmosphere Mel (4.15 g, 29.25 mmol) was added dropwise at 0° C. to the mixture and stirring was continued at 25° C. for 12 h. The reaction was quenched with H₂O (10 mL) at 0° C. and the mixture was extracted with ethyl acetate (30 mL*2). The combined organic layers were washed with brine (30 mL), dried over Na₂SO₄ and concentrated. The residue was subjected to flash column chromatography (ISCO 20 g silica, 0-17% ethyl acetate in petroleum ether, gradient over 20 min) to obtain 3-(3-bromophenyl)-1-methyl-pyrazole (1.6 g, 34%) as a white solid. ¹H NMR (400 MHz, CHLOROFORM-d) δ 7.99 (t, J=1.6 Hz, 1H), 7.74 (d, J=7.8 Hz, 1H), 7.44 (dd, J=7.9, 0.9 Hz, 1H), 7.40 (d, J=2.2 Hz, 1H), 7.32-7.24 (m, 1H), 6.55 (d, J=2.2 Hz, 1H), 3.97 (s, 3H); LCMS (ESI) m/z: 237.0 [M+H]⁺.

### Step 2: Synthesis 1-methyl-3-[3-(4,4,5,5-tetramethyl-1,3,2-dioxaborolan-2-yl)phenyl]pyrazole

To a solution of 3-(3-bromophenyl)-1-methyl-pyrazole (1.6 g, 6.75 mmol) in dioxane (30 mL) were added 4,4,5,5-tetramethyl-2-(4,4,5,5-tetramethyl-1,3,2-dioxaborolan-2-yl)-1,3,2-dioxaborolane (2.57 g, 10.12 mmol), KOAc (1.99 g, 20.25 mmol) and Pd(PPh₃)₂Cl₂ (474 mg, 675 umol). The mixture was stirred at 80° C. for 12 h under argon atmosphere. The mixture was then filtered to remove the solids and the filtrate was concentrated. The residue was diluted with 30 mL of water, and it was extracted with ethyl acetate (30 mL*2). The combined organic layers were washed with brine (20 mL), dried over Na₂SO₄ and concentrated. The residue was subjected to flash column chromatography (ISCO 20 g silica, 0-27% ethyl acetate in petroleum ether, gradient over 20 min) to obtain 1-methyl-3-[3-(4,4,5,5-tetramethyl-1,3,2-dioxaborolan-2-yl)phenyl]pyrazole (730 mg, 30%) as a colorless oil. ¹H NMR (400 MHz, CHLOROFORM-d) δ 8.12 (s, 1H), 7.85 (td, J=7.8, 1.5 Hz, 1H), 7.66 (td, J=7.4, 1.1 Hz, 1H), 7.32 (t, J=7.5 Hz, 1H), 7.29 (d, J=2.1 Hz, 1H), 6.56-6.46 (m, 1H), 3.87 (d, J=0.9 Hz, 3H), 1.28 (s, 12H); LCMS (ESI) m/z: 285.1 [M+H]⁺.

### Step 3: Synthesis of ethyl 4-[3-(1-methylpyrazol-3-yl)phenyl]-2-morpholino-6-(4-pyridylamino)pyrimidine-5-carboxylate

To a solution of ethyl 4-chloro-2-morpholino-6-(4-pyridylamino)pyrimidine-5-carboxylate (500 mg, 1.37 mmol) in dioxane (7 mL) and H₂O (0.7 mL) were added 1-methyl-3-[3-(4,4,5,5-tetramethyl-1,3,2-dioxaborolan-2-yl)phenyl]pyrazole (586 mg, 2.06 mmol), K₂CO_{M}(570 mg, 4.12 mmol) and Pd(PPh₃)₄ (159 mg, 137 umol). The mixture was stirred at 100° C. for 3 h under nitrogen atmosphere and concentrated. The residue was diluted with 10 mL of water and the mixture was extracted with ethyl acetate (10 mL*2). The combined organic layers were washed with brine (10 mL), dried over Na₂SO₄ and concentrated. The resultant residue was subjected to flash column chromatography (ISCO 20 g silica, 0-87% ethyl acetate in petroleum ether, gradient over 20 min) to obtain ethyl 4-[3-(1-methylpyrazol-3-yl)phenyl]-2-morpholino-6-(4-pyridylamino)pyrimidine-5-carboxylate (520 mg, 78%) as a pale solid. ¹H NMR (400 MHz, CHLOROFORM-d) δ 8.51 (d, J=6.3 Hz, 2H), 7.93-7.80 (m, 2H), 7.69-7.61 (m, 2H), 7.49-7.33 (m, 3H), 6.56 (d, J=2.3 Hz, 1H), 4.04-3.86 (m, 9H), 3.80 (bs, 4H), 0.76 (t, J=7.2 Hz, 3H); LCMS (ESI) m/z: 486.3 [M+H]⁺.

### Step 4: Synthesis of 4-[3-(1-methylpyrazol-3-yl)phenyl]-2-morpholino-6-(4-pyridylamino)pyrimidine-6-carboxylic acid

To a solution of ethyl 4-[3-(1-methylpyrazol-3-yl)phenyl]-2-morpholino-6-(4-pyridylamino)pyrimidine-5-carboxylate (500 mg, 1.03 mmol) in THF (4 mL), MeOH (2 mL) and H₂O (2 mL) was added LIOH·H₂O (130 mg, 3.09 mmol). The mixture was stirred at 25° C. for 12 h and concentrated. To the residue was added, 5 mL H₂O and saturated aqueous citric acid solution at 0° C. until pH=3-4 was reached. The resultant precipitate was collected by filtration, rinsed with water and vacuum dried to obtain 4-[3-(1-methylpyrazol-3-yl)phenyl]-2-morpholino-6-(4-pyridylamino)pyrimidine-5-carboxylic acid (450 mg) as a yellow solid. LCMS (ESI) m/z: 458.2 [M+H]⁺

### Step 5: Synthesis of N-(2,2-dimethoxyethyl)-4-[3-(1-methylpyrazol-3-yl)phenyl]-2-morpholino-6-(4-pyridylamino)pyrimidine-5-carboxamide

To a solution of 4-[3-(1-methylpyrazol-3-yl)phenyl]-2-morpholino-6-(4-pyridylamino)pyrimidine-5-carboxylic acid (200 mg, 437 umol) in DMF (2 mL) were added 2,2-dimethoxyethanamine (46 mg, 437 umol), EDCl (109 mg, 568 umol), HOBt (77 mg, 568 umol) and TEA (133 mg, 1.31 mmol). The resultant mixture was stirred at 25° C. for 12 h and then diluted with 3 mL of water. It was then extracted with (dichloromethane/methanol: 10/1) (5 mL*3), the combined organic layers were washed with brine (3 mL), dried over Na₂SO₄ and concentrated. The crude product was subjected to flash column chromatography (ISCO 10 g silica, 0-15% methanol in dichloromethane, gradient over 20 min) to obtain N-(2,2-dimethoxyethyl)-4-[3-(1-methylpyrazol-3-yl)phenyl]-2-morpholino-6-(4-pyridylamino)pyrimidine-5-carboxamide (80 mg, 34%) as pale-yellow solid. LCMS (ESI) m/z: 545.2 [M+H]⁺

### Step 6: Syntheses of 6-[3-(1-methylpyrazol-3-yl)phenyl]-N-(1-methylpyridin-1-ium-4-yl)-2-morpholino-5-oxazol-2-yl-pyrimidin-4-amine and 6-[3-(1-methylpyrazol-3-yl)phenyl]-2-morpholino-5-oxazol-2-yl-N-(4-pyridyl)pyrimidin-4-amine

A mixture of N-(2,2-dimethoxyethyl)-4-[3-(1-methylpyrazol-3-yl)phenyl]-2-morpholino-6-(4-pyridylamino)pyrimidine-5-carboxamide (60 mg, 110 umol) and Eaton's reagent (4.56 g, 19.16 mmol) was stirred at 80° C. for 4 h under nitrogen atmosphere. The reaction mixture was then treated with 25 mL of aqueous saturated NaHCO₃ solution at 0° C. followed by the addition of 15 mL of water. The resultant mixture was extracted with (dichloromethane/methanol: 10/1) (20 mL*2), the combined organic layers were washed with brine (15 mL), dried over Na₂SO₄ and concentrated. The residue was subjected to prep-HPLC (Waters Xbridge BEH C18 100*30 mm*10 um column; 45-65% acetonitrile in an a 10 mM ammonium bicarbonate solution in water, 8 min gradient) to obtain 6-[3-(1-methylpyrazol-3-yl)phenyl]-N-(1-methylpyridin-1-ium-4-yl-2-morpholino-5-oxazol-2-yl-pyrimidin-4-amine (17 mg, 30%) as white solid. ¹H NMR (400 MHz, CHLOROFORM-d) δ 7.84-7.78 (m, 1H), 7.74 (s, 1H), 7.54 (s, 1H), 7.33 (d, J=2.3 Hz, 1H), 7.31 (dd, J=3.3, 1.6 Hz, 1H), 7.29 (s, 1H), 7.09 (s, 1H), 7.06 (d, J=7.3 Hz, 2H), 6.81 (d, J=7.1 Hz, 2H), 6.43 (d, J=2.3 Hz, 1H), 3.98-3.86 (m, 7H), 3.81-3.75 (m, 4H), 3.58 (s, 3H). LCMS (ESI) for (C27H27N8O2) [M+H]+: 495.3. This product was confirmed by 2D NMR experiments.

However, during the workup when methanol was avoided, the compound 6-[3-(1-methylpyrazol-3-yl)phenyl]-2-morpholino-5-oxazol-2-yl-N-(4-pyridyl)pyrimidin-4-amine (11 mg, 13%) was obtained as brown solid. ¹H NMR (400 MHz, CDCl3) δ 11.23 (s, 1H), 8.60-8.23 (m, 1H), 7.93-7.77 (m, 4H), 7.65-7.55 (m, 2H), 7.40 (t, J=8.0 Hz, 1H), 7.35-7.27 (m, 2H), 6.57 (d, J=2.3 Hz, 1H), 4.02-3.90 (m, 7H), 3.85-3.76 (m, 4H). LCMS (ESI) for (C26H24N8O2) [M+H]+: 481.3.

### Example 64. Synthesis of (R)-4-methoxy-6-(3-(1-methyl-1H-pyrazol-3-yl)phenyl)-2-(2-methylmorpholino)-N-(pyridin-4-yl)pyrimidin-4-amine (Compound 75)

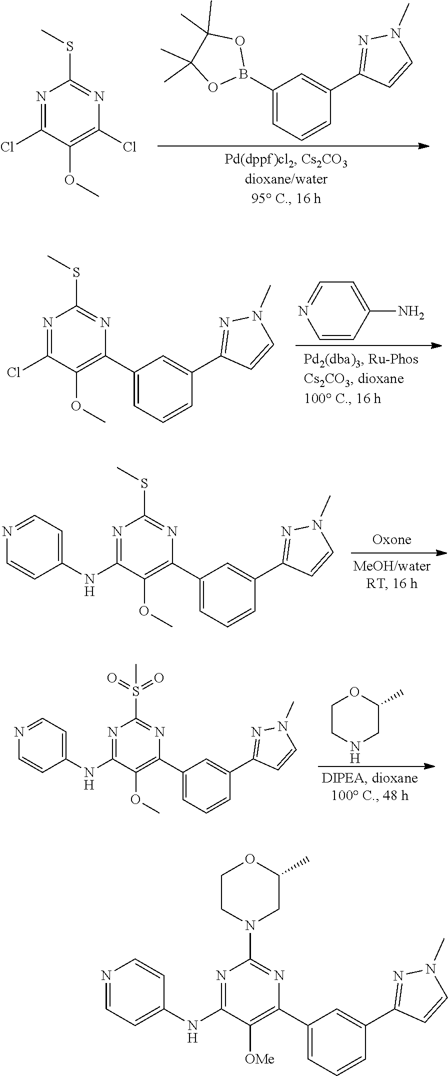

### Step 1: Synthesis of 4-chloro-5-methoxy-6-(3-(1-methyl-1H-pyrazol-3-yl)phenyl)-2-(methylthio)pyrimidine

A mixture of 4,6-dichloro-5-methoxy-2-(methylthio)pyrimidine (1.8 g, 8.04 mmol), 1-methyl-3-(3-(4,4,5,5-tetramethyl-1,3,2-dioxaborolan-2-yl)phenyl)-1H-pyrazole (2.28 g, 8.04 mmol), 1,1′-bis(diphenylphosphino)ferrocene-palladium(II)dichloride dichloromethane complex (0.33 g, 0.40 mmol) and cesium carbonate (6.5 g, 20.1 mmol) in 1,4-dioxane/water (60 mL/8 mL) was stirred at 95° C. for 16 h under nitrogen atmosphere. The reaction mixture was filtered, and the filtrate was concentrated. The residue was subjected to silica gel chromatography (eluted with ethyl acetate in petroleum ether from 20% to 40%) to obtain 4-chloro-5-methoxy-6-(3-(1-methyl-1H-pyrazol-3-ylphenyl)-2-(methylthio)pyrimidine (1.8 g, 64.7%) as white solid. LCMS (ESI) m/z: 347.0 [M+H]⁺.

### Step 2: Synthesis of 5-methoxy-6-(3-(1-methyl-1H-pyrazol-3-yl)phenyl)-2-(methylthio)-N-(pyridin-4-yl)pyrimidin-4-amine

A mixture of 4-chloro-5-methoxy-6-(3-(1-methyl-1H-pyrazol-3-yl)phenyl)-2-(methylthio)pyrimidine (1.7 g, 4.91 mmol), pyridin-4-amine (0.46 g, 4.91 mmol), tris(dibenzylideneacetone)dipalladium (0.45 g, 0.49 mmol), 2-dicyclohexylphosphino-2′,6′-diisopropoxybiphenyl (0.46 g, 0.98 mmol) and cesium carbonate (4 g, 12.3 mmol) in dry 1,4-dioxane (75 mL) was stirred at 100° C. for 16 h under nitrogen atmosphere. The mixture was filtered and the filtrate was concentrated. The residue was subjected to silica gel chromatography (eluted with methanol, containing 0.5% 7 N ammonia methanol, in dichloromethane from 5% to 15%) to afford 5-methoxy-6-(3-(1-methyl-1H-pyrazol-3-yl)phenyl)-2-(methylthio)-N-(pyridin-4-ylpyrimidin-4-amine (1.5 g, 75.6%) as white solid. LCMS (ESI) m/z: 405.1[M+H]⁺.

### Step 3: Synthesis of 5-methoxy-6-(3-(1-methyl-1H-pyrazol-3-yl)phenyl)-2-(methylsulfonyl)-N-(pyridin-4-yl)pyrimidin-4-amine

To a solution of 5-methoxy-6-(3-(1-methyl-1H-pyrazol-3-yl)phenyl)-2-(methylthio)-N-(pyridin-4-ylpyrimidin-4-amine (1.3 g, 3.22 mmol) in methanol (40 mL) at 0° C., was added a solution of oxone (3.95 g, 6.43 mmol) in water (20 mL) drop-wise. After the addition, the reaction mixture was stirred at room temperature for 3 h. The resultant precipitate was filtered, the obtained solid was dispersed in sodium bicarbonate aqueous solution and stirred at room temperate for 20 min. The resulting solids were collected by filtration, washed with water and dried in vacuo to afford 5-methoxy-6-(3-(1-methyl-1H-pyrazol-3-yl)phenyl)-2-(methylsulfonyl)-N-(pyridin-4-yl)pyrimidin-4-amine (0.93 g, 66.2%) as white solid. LCMS (ESI) m/z: 437.1 [M+H]⁺.

### Step 4: Synthesis of (R)-4-methoxy-6-(3-(1-methyl-H-pyrazol-3-yl)phenyl)-2-(2-methylmorpholino)-N-(pyridin-4-yl)pyrimidin-4-amine

A mixture of 5-methoxy-6-(3-(1-methyl-1H-pyrazol-3-yl)phenyl)-2-(methylsulfonyl)-N-(pyridin-4-ylpyrimidin-4-amine (150 mg, 0.34 mmol), (R)-2-methylmorpholine (69.6 mg, 0.69 mmol), N,N-diisopropylethylamine (0.13 g, 1.03 mmol) in dry 1,4-dioxane (10 mL) was stirred at 100° C. for 48 h under nitrogen atmosphere. The reaction mixture was filtered, the filtrate was concentrated and the residue was subjected to prep-HPLC (BOSTON pHlex ODS 10 um 21.2×250 mm 120 A. The mobile phase was acetonitrile/0.1% ammonium bicarbonate) to obtain (R)-5-methoxy-6-(3-(1-methyl-1H-pyrazol-3-yl)phenyl)-2-(2-methylmorpholino)-N-(pyridin-4-yl)pyrimidin-4-amine (12.7 mg, 8.2%) as white solid. ¹H NMR (400 MHz, DMSO-d₆) δ 9.37 (s, 1H), 8.47-8.38 (m, 3H), 7.91-7.73 (m, 4H), 7.77 (d, J=2.1 Hz, 1H), 7.53 (t, J=7.7 Hz, 1H), 6.71 (d, J=2.2 Hz, 1H), 4.48-4.30 (m, 2H), 3.98-3.87 (m, 4H), 3.55 (t, J=10.3 Hz, 2H), 3.45 (s, 3H), 2.99 (t, J=10.7 Hz, 1H), 2.69-2.62 (m, 1H), 1.18 (d, J=6.2 Hz, 3H). LCMS (ESI) m/z: 458.2 [M+H]⁺

The following compounds were synthesized according to the protocol described above:

| Compound No. | | |
|---|---|---|
| Name | Structure | NMR, MS |
| 76 (S)-5-methoxy-6-(3-(1- methyl-1H-pyrazol-3- yl)phenyl)-2-(2- methylmorpholino)-N- (pyridin-4-yl)pyrimidin-4- amine | 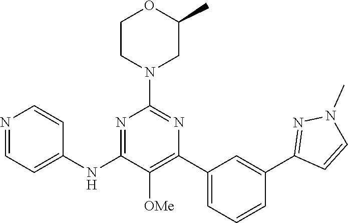 [SMILES: COc1c(Nc2ccncc2)nc(N2CCO[C@@H](C)C2)nc1-c1cccc(-c2ccn(C)n2)c1] | ¹H NMR (400 MHz, DMSO-d₆) δ 9.34 (s, 1H), 8.48-8.37 (m, 3H), 7.91-7.73 (m, 4H), 7.77 (d, J = 2.1 Hz, 1H), 7.52 (t, J = 7.7 Hz, 1H), 6.71 (d, J = 2.1 Hz, 1H), 4.46-4.30 (m, 2H), 3.98-3.84 (m, 4H), 3.54 (t, J = 10.1 Hz, 2H), 3.45 (s, 3H), 2.98 (t, J = 10.8 Hz, 1H), 2.71-2.60 (m, 1H), 1.18 (d, J = 6.2 Hz, 3H). LCMS (ESI) m/z: 458.2 [M + H]⁺. |
| 77 (S)-1-(5-methoxy-4-(3-(1- methyl-1H-pyrazol-3- yl)phenyl)-6-(pyridin-4- ylamino)pyrimidin-2- yl)piperidin-3-ol | 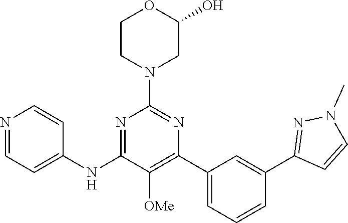 [SMILES: COc1c(Nc2ccncc2)nc(N2CCO[C@H](O)C2)nc1-c1cccc(-c2ccn(C)n2)c1] | ¹H NMR (400 MHz, DMSO-d₆) δ 9.35 (s, 1H), 8.45-8.40 (m, 3H), 7.93-7.84 (m, 4H), 7.77 (d, J = 2.3 Hz, 1H), 7.52 (t, J = 7.8 Hz, 1H), 6.70 (d, J = 2.1 Hz, 1H), 4.92 (d, J = 3.9 Hz, 1H), 4.48-4.43 (m, 1H), 4.32-4.27 (m, 1H), 3.90 (s, 3H), 3.51-3.47 (m, 1H), 3.43 (s, 3H), 3.07-3.01 (m, 1H), 2.92-2.84 (m, 1H), 1.94-1.89 (m, 1H), 1.78-1.72 (m, 1H), 1.48-1.35 (m, 2H). LCMS (ESI) m/z: 458.1 [M + H]⁺. |
| 78 (R)-1-(5-methoxy-4-(3-(1- methyl-1H-pyrazol-3- yl)phenyl)-6-(pyridin-4- ylamino) pyrimidin-2- yl)piperidin-3-ol | 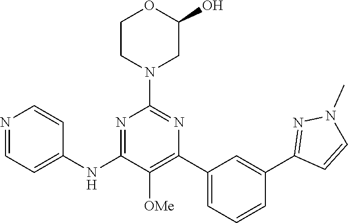 [SMILES: COc1c(Nc2ccncc2)nc(N2CCO[C@@H](O)C2)nc1-c1cccc(-c2ccn(C)n2)c1] | ¹H NMR (400 MHz, DMSO-d₆) δ 9.37 (s, 1H), 8.46-8.40 (m, 3H), 7.94-7.82 (m, 4H), 7.77 (d, J = 2.1 Hz, 1H), 7.52 (t, J = 7.7 Hz, 1H), 6.70 (d, J = 2.2 Hz, 1H), 4.92 (d, J = 4.3 Hz, 1H), 4.48-4.42 (m, 1H), 4.32-4.27 (m, 1H), 3.90 (s, 3H), 3.53-3.46 (m, 1H), 3.43 (s, 3H), 3.08-3.01 (m, 1H), 2.93-2.84 (m, 1H), 1.94-1.88 (m, 1H), 1.79-1.73 (m, 1H), 1.43-1.36 (m, 2H). LCMS (ESI) m/z: 458.1 [M + H]⁺. |

### Example 65. PIKfyve Inhibitory Activity

PIKfyve Biochemical Assay. The biochemical PIKFyve inhibition assays were run by Cama Biosciences according to proprietary methodology based on the Promega ADP-Glo™ Kinase assay. A full-length human PIKFYVE [1-2098(end) amino acids and S696N, L932S, Q995L, T998S, S1033A and Q1183K of the protein having the sequence set forth in NCBI Reference Sequence No. NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega). Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADP-Glo™ reagent was then added for 40 minutes, followed by kinase detection reagent for an additional 40 minutes. The kinase activity was evaluated by detecting relative light units on a luminescence plate reader. Samples were run in duplicate from 10 μM to 3 nM. Data was analyzed by setting the control wells (+ PIKfyve, no compound) to 0% inhibition and the readout value of background (no PIKfyve) set to 100% inhibition, then the % inhibition of each test solution calculated. IC₅₀ values were calculated from concentration versus percent inhibition curves by fitting to a four-parameter logistic curve.

PIKfyve EEA1 Assay. Genetic or pharmacological disruption of PIKfyve activity results in enlargement of endosomal vesicles. This enlargement was utilized as a surrogate readout of PIKFyve inhibition for routine triage of PIKfyve inhibitors. U2OS cells grown in 96-well assay plates were treated with compound diluted in DMEM media containing 10% fetal bovine serum. After 3 hours of treatment, cells were fixed with paraformaldehyde, permeabilized with 0.2% Triton-X in phosphate buffered saline and stained against EEA1. During the secondary antibody staining, cells were also stained with CellMask™ Deep Red and Hoechst to detect cytoplasms and nuclei respectively. Endosomal structures were visualized using a high content imager at 40× magnification. Images were analyzed using a linear classifier algorithm integrating EEA1 spot size, intensity and texture trained on images of cells treated with the potent reference compound APY0201. Compound activity was calculated by subtracting the DMSO signal and calculating percentage activity relative to maximal APY0201 activity. IC₅₀ values were then calculated from concentration versus percent inhibition data by logistic regression.

The results of the PIKfyve inhibition assays are summarized in Table 3 below.

TABLE 3
PIKfyve inhibition

| Compound | PIKfyve Biochemistry | PIKfyve EEA1 |
|---|---|---|
| No. | IC5₀ (μM)* | IC₅₀ (μM)* |
| 1 | +++ | + |
| 2 | +++ | + |
| 3 | nt | +++ |
| 4 | nt | + |
| 5 | nt | + |
| 6 | +++ | + |
| 7 | ++++ | +++ |
| 8 | ++++ | ++ |
| 9 | ++++ | +++ |
| 10 | +++ | ++ |
| 11 | +++ | + |
| 12 | ++++ | ++ |
| 13 | ++++ | ++ |
| 14 | ++++ | +++ |
| 15 | +++ | ++ |
| 16 | ++++ | +++ |
| 17 | ++++ | +++ |
| 18 | nt | + |
| 19 | +++ | ++ |
| 20 | nt | ++ |
| 21 | nt | + |
| 22 | nt | + |
| 23 | nt | + |
| 24 | nt | + |
| 25 | nt | ++ |
| 26 | nt | + |
| 27 | nt | + |
| 28 | nt | + |
| 29 | nt | + |
| 30 | +++ | + |
| 31 | nt | + |
| 32 | ++ | + |
| 33 | +++ | ++ |
| 34 | nt | +++ |
| 35 | nt | + |
| 36 | nt | + |
| 37 | nt | + |
| 38 | +++ | ++ |
| 39 | ++++ | +++ |
| 40 | +++ | +++ |
| 41 | +++ | ++ |
| 42 | nt | + |
| 43 | nt | + |
| 44 | + | − |
| 45 | nt | + |
| 46 | ++ | − |
| 47 | +++ | ++ |
| 48 | nt | ++ |
| 49 | +++ | ++ |
| 50 | nt | ++ |
| 51 | nt | ++ |
| 52 | nt | + |
| 53 | nt | +++ |
| 54 | nt | +++ |
| 55 | nt | + |
| 56 | nt | ++ |
| 57 | ++++ | ++ |
| 58 | nt | ++ |
| 59 | nt | + |
| 60 | nt | + |
| 61 | nt | + |
| 62 | ++++ | ++ |
| 63 | nt | + |
| 64 | nt | + |
| 65 | +++ | + |
| 66 | ++++ | ++ |
| 67 | nt | + |
| 68 | ++++ | +++ |
| 69 | nt | + |
| 70 | nt | + |
| 71 | nt | ++ |
| 72 | nt | +++ |
| 73 | nt | + |
| 74 | +++ | ++ |

++++ stands for <10 nM;
+++ stands for 10-100 nM;
++ stands for 100-1000 nM;
+ stands for 1-10 μM;
- stands for >10 μM;
nt stands for not tested

### Example 66. Viability Assay to Assess TDP-43 Toxicity in FAB1 TDP-43 and PIKfyve TDP-43 Yeast Cells

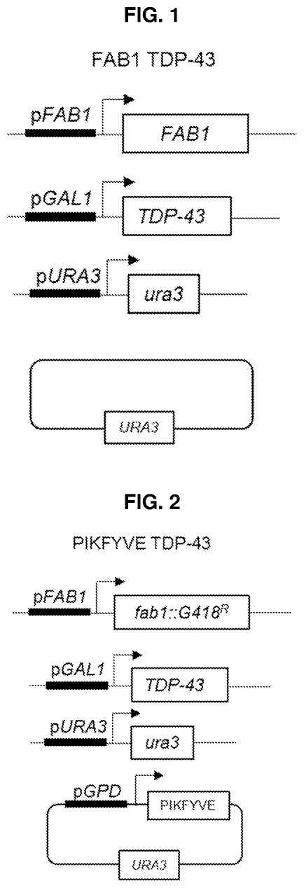

Generation of TDP-43 yeast model expressing human PIKfyve. Human PIKFYVE (“entry clone”) was cloned into pAG416GPDccdB (“destination vector”) according to standard Gateway cloning protocols (Invitrogen, Life Technologies). The resulting pAG416GPD-PIKFYVE plasmids were amplified in E. coli and plasmid identity confirmed by restriction digest and Sanger sequencing. Lithium acetate/polyethylene glycol-based transformation was used to introduce the above PIKFYVE plasmid into a BY4741 yeast strain auxotrophic for the ura3 gene and deleted for two transcription factors that regulate the xenobiotic efflux pumps, a major efflux pump, and FAB1, the yeast ortholog of PIKFYVE (MATa, snq2::KILeu2; pdr3::Klura3; pdr1::NATMX; fab1::G418^{R}, his3; leu2; ura3; met15; LYS2+) (FIG. 2). Transformed yeast were plated on solid agar plates with complete synthetic media lacking uracil (CSM-ura) and containing 2% glucose. Individual colonies harboring the control or PIKFYVE TDP-43 plasmids were recovered. A plasmid containing wild-type TDP-43 under the transcriptional control of the GAL1 promoter and containing the hygromycin-resistance gene as a selectable marker was transformed into the fab1::G418^{R} pAG416GPD-PIKFYVE yeast strain (FIG. 1). Transformed yeast were plated on CSM-ura containing 2% glucose and 200 □g/mL G418 after overnight recovery in media lacking antibiotic. Multiple independent isolates were further evaluated for cytotoxicity and TDP-43 expression levels.

Viability Assay. A control yeast strain with the wild-type yeast FAB1 gene and TDP-43 (“FAB1 TDP-43”, carries empty pAG416 plasmid), and the “PIKFYVE TDP-43” yeast strain, were assessed for toxicity using a propidium iodide viability assay. Both yeast strains were transferred from solid CSM-ura/2% glucose agar plates into 3 mL of liquid CSM-ura/2% glucose media for 6-8 hours at 30° C. with aeration. Yeast cultures were then diluted to an optical density at 600 nm wavelength (OD₆₀₀) of 0.005 in 3 mL of CSM-ura/2% raffinose and grown overnight at 30° C. with aeration to an OD₆₀₀ of 0.3-0.8. Log-phase overnight cultures were diluted to OD₆₀₀ of 0.005 in CSM-ura containing either 2% raffinose or galactose and 150 □L dispensed into each well of a flat bottom 96-well plates. Compounds formulated in 100% dimethyl sulfoxide (DMSO) were serially diluted in DMSO and 1.5 □L diluted compound transferred to the 96-well plates using a multichannel pipet. Wells containing DMSO alone were also evaluated as controls for compound effects. Tested concentrations ranged from 15 □M to 0.11 □M. Cultures were immediately mixed to ensure compound distribution and covered plates incubated at 30° C. for 24 hours in a stationary, humified incubator.

Upon the completion of incubation, cultures were assayed for viability using propidium iodide (PI) to stain for dead/dying cells. A working solution of PI was made where, for each plate, 1 □L of 10 mM PI was added to 10 mL of CSM-ura (raffinose or galactose). The final PI solution (50 □L/well) was dispensed into each well of a new round bottom 96-well plate. The overnight 96-well assay plate was then mixed with a multichannel pipet and 50 □L transferred to the PI-containing plate. This plate was then incubated for 30 minutes at 30° C. in the dark. A benchtop flow cytometer (Miltenyi MACSquant) was then used to assess red fluorescence (B2 channel), forward scatter, and side scatter (with following settings: gentle mix, high flow rate, fast measurement, 10,000 events). Intensity histograms were then gated for “P-positive” or “PI-negative” using the raffinose and galactose cultures treated with DMSO as controls. The DMSO controls for raffinose or galactose-containing cultures were used to determine the window of increased cell death and this difference set to 100. All compounds were similarity gated and then compared to this maximal window to establish the percent reduction in PI-positive cells. IC50 values were then calculated for compounds that demonstrated a concentration-dependent enhancement of viability by fitting a logistic regression curve.

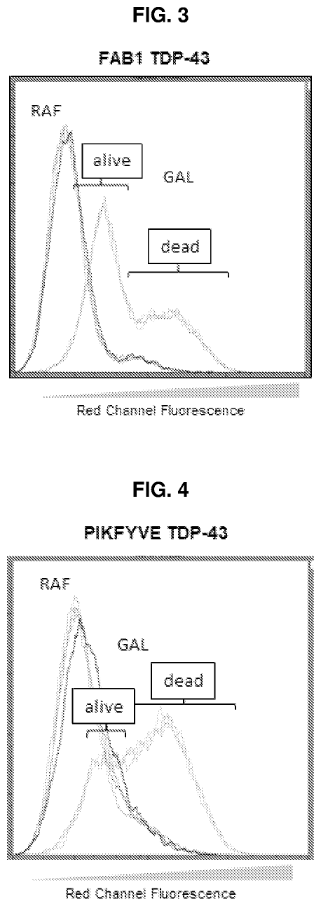

Upon induction of TDP-43 in both strains, there was a marked increase in inviable cells (rightmost population) with both FAB1 TDP-43 and PIKFYVE TDP-43, with a more pronounced effect in PIKFYVE TDP-43 (FIGS. 3 and 4).

PIKfyve Inhibition Suppresses Toxicity in PIKfyve TDP-43 Model. The biochemical PIKFyve inhibition assays were run by Cama Biosciences according to proprietary methodology based on the Promega ADP-Glo™ Kinase assay. A full-length human PIKFYVE [1-2098(end) amino acids and S696N, L932S, Q995L, T998S, S1033A and Q1183K of accession number NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega).

Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADP-Glo™ reagent was then added for 40 minutes, followed by kinase detection reagent for an additional 40 minutes. The kinase activity was evaluated by detecting relative light units on a luminescence plate reader. Samples were run in duplicate from 10 uM to 3 nM. Data was analyzed by setting the control wells (+ PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

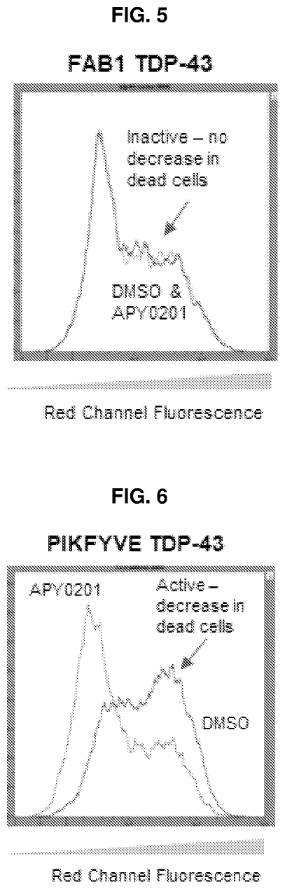

Activity of APY0201, a known PIKFYVE inhibitor, in FAB1 TDP-43 (FIG. 5) and PIKFYVE TDP-43 (FIG. 6). There was no increase in viable cells in FAB1 TDP-43 across a range of compound concentrations as evidenced by a lack in reduction of the right most population of propidium iodide-positive cells (only 0.23 μM is shown). In the PIKFYVE TDP-43 model, 0.23 μM reduced the population of propidium iodide-positive dead cells, indicating PIKFYVE inhibition ameliorated TDP-43 toxicity. Concentrations ranging from 0.5 mM to less than 100 nM afforded increased viability.

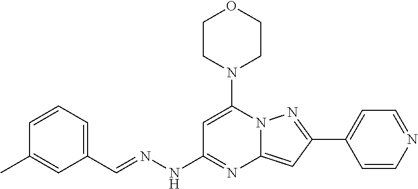 [SMILES: Cc1cccc(/C=N/Nc2cc(N3CCOCC3)n3nc(-c4ccncc4)cc3n2)c1]

A panel of compounds was tested in a biochemical PIKFYVE assay (ADP-Glo™ with full-length PIKfyve) and IC50's determined (nM) (see the Table below). The same compounds were also tested in both FAB1 and PIKFYVE TDP-43 yeast models. Their activity is reported here as “active” or “inactive.” Compounds with low nanomolar potency in the biochemical assay were active in the PIKFYVE TDP-43 yeast model. Compounds that were less potent or inactive in the biochemical assay were inactive in the PIKFYVE TDP-43 model. Compounds that were inactive in the biochemical or PIKFYVE TDP-43 assays were plotted with the highest concentrations tested in that assay.

| | PIKfyve IC₅₀ | FAB1 TDP-43 | PIKfyve TDP-43 |
|---|---|---|---|
| Structure | (nM) | (active/inactive) | (active/inactive) |
| 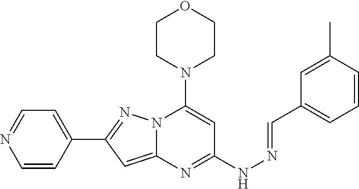 [SMILES: Cc1cccc(/C=N/Nc2cc(N3CCOCC3)n3nc(-c4ccncc4)cc3n2)c1] | 7.5 | Inactive | Active |
| 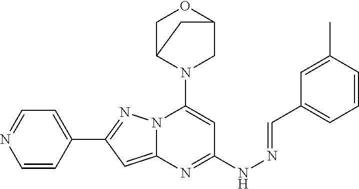 [SMILES: Cc1cccc(/C=N/Nc2cc(N3CC4CC3CO4)n3nc(-c4ccncc4)cc3n2)c1] | 12 | Inactive | Active |
| 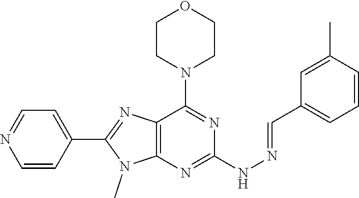 [SMILES: Cc1cccc(/C=N/Nc2nc(N3CCOCC3)c3nc(-c4ccncc4)n(C)c3n2)c1] | 4.9 | Inactive | Active |
| 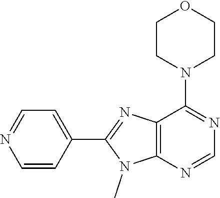 [SMILES: Cn1c(-c2ccncc2)nc2c(N3CCOCC3)ncnc21] | 640 | Inactive | Inactive |
| 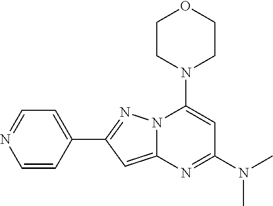 [SMILES: CN(C)c1cc(N2CCOCC2)n2nc(-c3ccncc3)cc2n1] | 2007 | Inactive | Inactive |
| 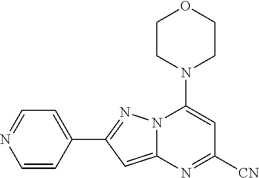 [SMILES: N#Cc1cc(N2CCOCC2)n2nc(-c3ccncc3)cc2n1] | >10000 | Inactive | Inactive |

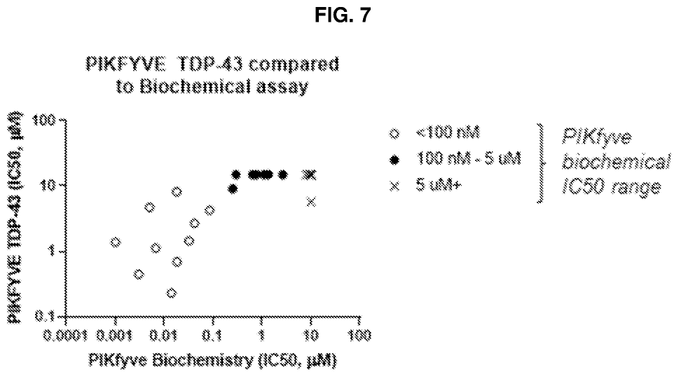

Biochemical and Efficacy Assays. A larger set of PIKfyve inhibitors were evaluated in both a PIKfyve kinase domain binding assay (nanobret) and in the PIKFYVE TDP-43 yeast strain. IC50 values (μM) were plotted. Data points are formatted based on binned potency from the nanobret assay as indicated in the legend (FIG. 7). Below is a table of compounds and their biochemical and PIKFYVE TDP-43 IC50 values plotted in FIG. 7.

| | PIKFYVE Biochemistry | PIKFYVE TDP-43 |
|---|---|---|
| Structure | (IC50, μM) | (IC50, μM) |
| 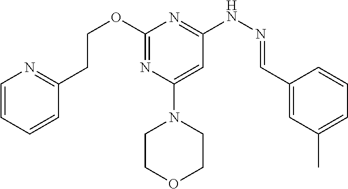 [SMILES: Cc1cccc(/C=N/Nc2cc(N3CCOCC3)nc(OCCc3ccccn3)n2)c1] | 0.003 | 0.450 |
| 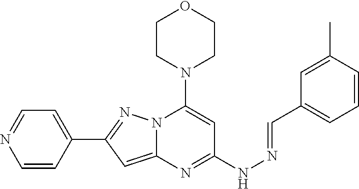 [SMILES: Cc1cccc(/C=N/Nc2cc(N3CCOCC3)n3nc(-c4ccncc4)cc3n2)c1] | 0.001 | 1.390 |
| 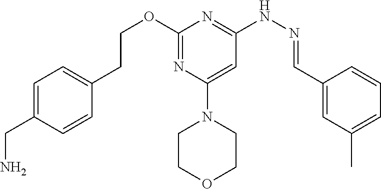 [SMILES: Cc1cccc(/C=N/Nc2cc(N3CCOCC3)nc(OCCc3ccc(CN)cc3)n2)c1] | 0.007 | 1.120 |
| 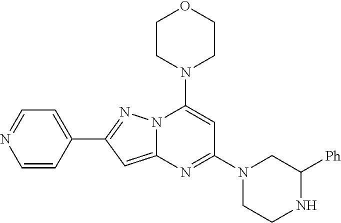 [SMILES: c1ccc(C2CN(c3cc(N4CCOCC4)n4nc(-c5ccncc5)cc4n3)CCN2)cc1] | 2.660 | >15 |
| 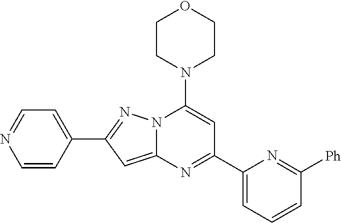 [SMILES: c1ccc(-c2cccc(-c3cc(N4CCOCC4)n4nc(-c5ccncc5)cc4n3)n2)cc1] | 0.014 | 0.230 |
| 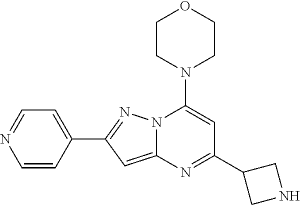 [SMILES: c1cc(-c2cc3nc(C4CNC4)cc(N4CCOCC4)n3n2)ccn1] | 8.020 | >15 |
| 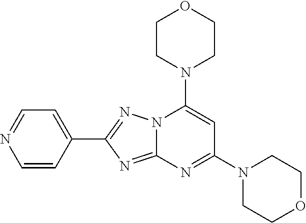 [SMILES: c1cc(-c2nc3nc(N4CCOCC4)cc(N4CCOCC4)n3n2)ccn1] | 9.200 | >15 |
| 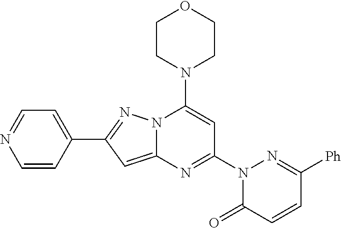 [SMILES: O=c1ccc(-c2ccccc2)nn1-c1cc(N2CCOCC2)n2nc(-c3ccncc3)cc2n1] | 0.295 | >15 |
| 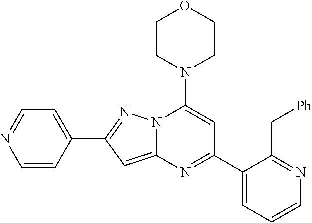 [SMILES: c1ccc(Cc2ncccc2-c2cc(N3CCOCC3)n3nc(-c4ccncc4)cc3n2)cc1] | 1.090 | >15 |
| 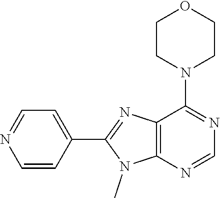 [SMILES: Cn1c(-c2ccncc2)nc2c(N3CCOCC3)ncnc21] | 0.640 | >15 |
| 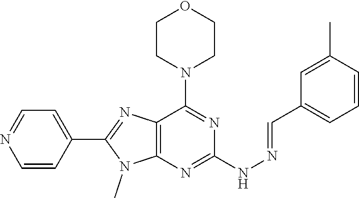 [SMILES: Cc1cccc(/C=N/Nc2nc(N3CCOCC3)c3nc(-c4ccncc4)n(C)c3n2)c1] | 0.005 | 4.720 |
| 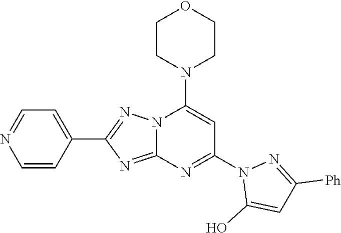 [SMILES: Oc1cc(-c2ccccc2)nn1-c1cc(N2CCOCC2)n2nc(-c3ccncc3)nc2n1] | 0.018 | 0.693 |
| 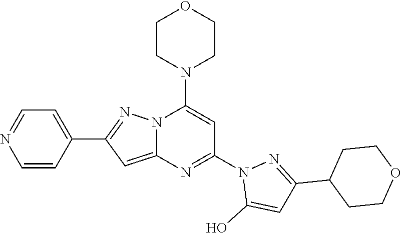 [SMILES: Oc1cc(C2CCOCC2)nn1-c1cc(N2CCOCC2)n2nc(-c3ccncc3)cc2n1] | 0.253 | 9.105 |
| 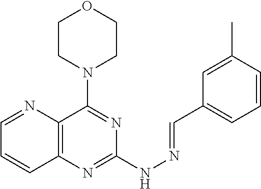 [SMILES: Cc1cccc(/C=N/Nc2nc(N3CCOCC3)c3ncccc3n2)c1] | 0.018 | 8.214 |
| 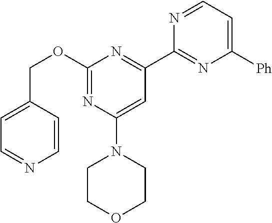 [SMILES: c1ccc(-c2ccnc(-c3cc(N4CCOCC4)nc(OCc4ccncc4)n3)n2)cc1] | 0.032 | 1.447 |
| 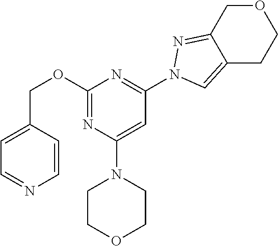 [SMILES: c1cc(COc2nc(N3CCOCC3)cc(-n3cc4c(n3)COCC4)n2)ccn1] | 1.343 | >15 |
| 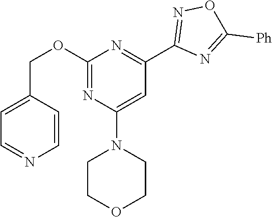 [SMILES: c1ccc(-c2nc(-c3cc(N4CCOCC4)nc(OCc4ccncc4)n3)no2)cc1] | >10 | >15 |
| 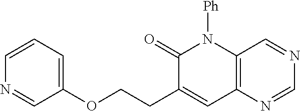 [SMILES: O=c1c(CCOc2cccnc2)cc2ncncc2n1-c1ccccc1] | >10 | >15 |
| 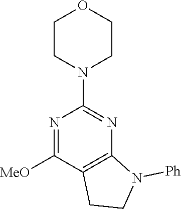 [SMILES: COc1nc(N2CCOCC2)nc2c1CCN2c1ccccc1] | 0.085 | 4.273 |
| 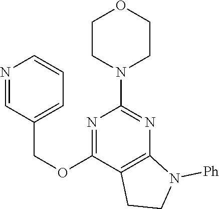 [SMILES: c1ccc(N2CCc3c(OCc4cccnc4)nc(N4CCOCC4)nc32)cc1] | 0.042 | 2.685 |
| 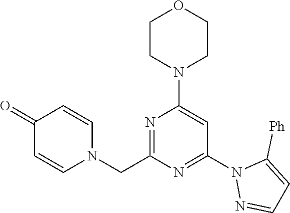 [SMILES: O=c1ccn(Cc2nc(N3CCOCC3)cc(-n3nccc3-c3ccccc3)n2)cc1] | >10 | >15 |
| 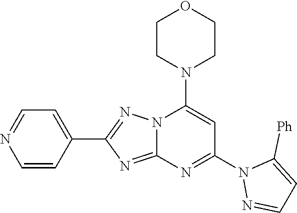 [SMILES: c1ccc(-c2ccnn2-c2cc(N3CCOCC3)n3nc(-c4ccncc4)nc3n2)cc1] | 0.767 | >15 |
| 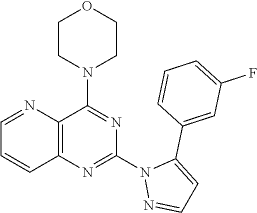 [SMILES: Fc1cccc(-c2ccnn2-c2nc(N3CCOCC3)c3ncccc3n2)c1] | >10 | 5.754 |

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A compound of Formula I: 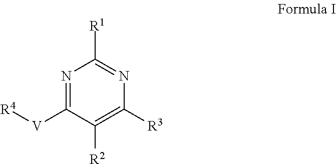
or a pharmaceutically acceptable salt thereof,
wherein
V is —NH—, —NR⁵—, —CH₂NH—, —CH₂NR⁵—, —O—, —CO—, or —CHOH—;
R¹ is optionally substituted morpholin-4-yl, pyridin-4-yl, pyridin-3-yl, optionally substituted 2-oxo-pyrrolidin-1-yl, optionally substituted piperidin-1-yl, or optionally substituted pyridazin-4-yl;
R² is halogen, —(CH₂)ₙOH, optionally substituted C₁₋₆ alkoxy, optionally substituted C₂-C₉ heteroaryl, optionally substituted 2-oxo-pyrrolidin-1-yl, —(CO)NR^{7a}R^{7b}, —P(O)R^{7c}R^{7d}, or —S(O)ₖR^{7e}; and R³ is optionally substituted pyridin-2-yl, optionally substituted pyridin-3-yl, optionally substituted pyridin-4-yl, optionally substituted pyrazol-1-yl, optionally substituted pyrazol-3-yl, optionally substituted pyridazin-3-yl, optionally substituted pyrimidin-4-yl, or optionally substituted C₆-C₁₀ aryl; or R² and R³, together with the ring to which they are attached, combine to form optionally substituted C₄-C₁₂ heteroaryl;
n is 1, 2, 3, 4, 5, or 6;
k is 0, 1, or 2;
R⁴ is optionally substituted pyridin-4-yl, optionally substituted 1-methylpyridin-1-ium-4-yl, optionally substituted pyridin-3-yl, optionally substituted 1-methylpiperidin-3-yl, optionally substituted pyridazine-3-yl, or —NHR⁸;
R⁵ is optionally substituted C₁-C₆ alkyl;
R⁶ is H or optionally substituted C₁-C₆ alkyl;
each of R^{7a} and R^{7b} is, independently, H or optionally substituted C₁-C₆ alkyl; or R^{7a} and R^{7b}, together with the nitrogen atom to which they are attached, combine to form optionally substituted C₂-C₉ heterocyclyl;
each of R^{7c}, R^{7d}, and R^{7e}, is independently optionally substituted C₁₋₆ alkyl, optionally substituted C₁₋₆ alkoxy, or hydroxyl; and
R⁸ is optionally substituted phenyl or optionally substituted C₃-C₆ cycloalkyl.

2-4. (canceled)

5. The compound of claim 1, wherein R¹ is

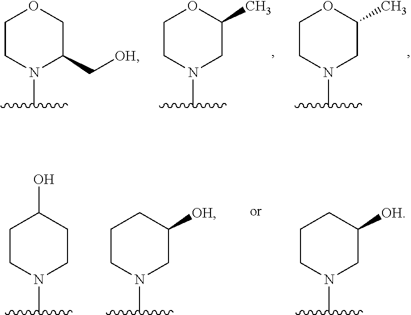

6-18. (canceled)

19. The compound of claim 1, wherein R⁸ is cyclopropyl or phenyl.

20-24. (canceled)

25. The compound of claim 1, wherein R⁴ is pyridin-4-yl or pyridin-3-yl.

26-27. (canceled)

28. The compound of claim 1, wherein the compound is of the following structure:

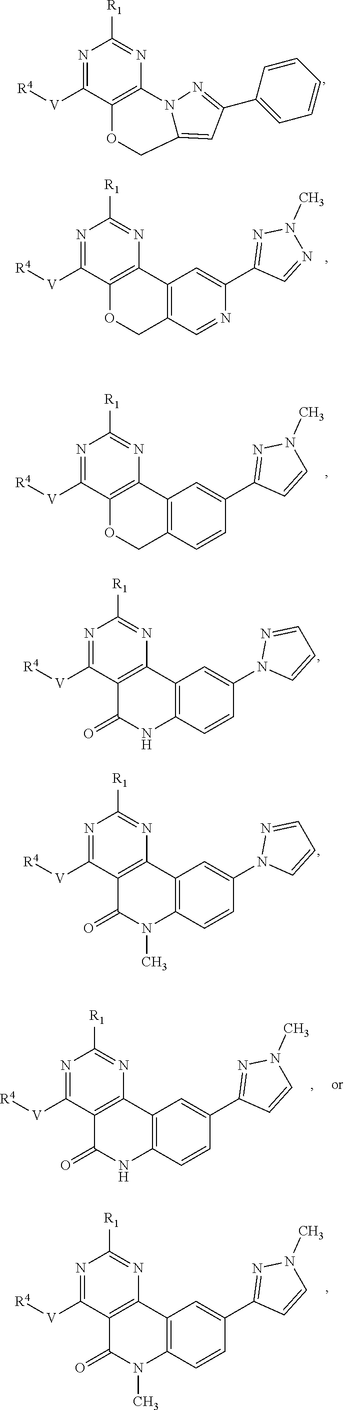

29-31. (canceled)

32. The compound of claim 1, wherein R² is —OCH₃, —(CO)N(CH₃)₂, 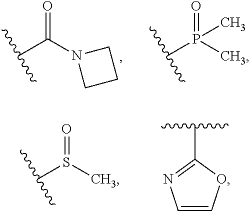
Cl, or —(CH₂)OH.

33-48. (canceled)

49. The compound of claim 1, wherein R³ is

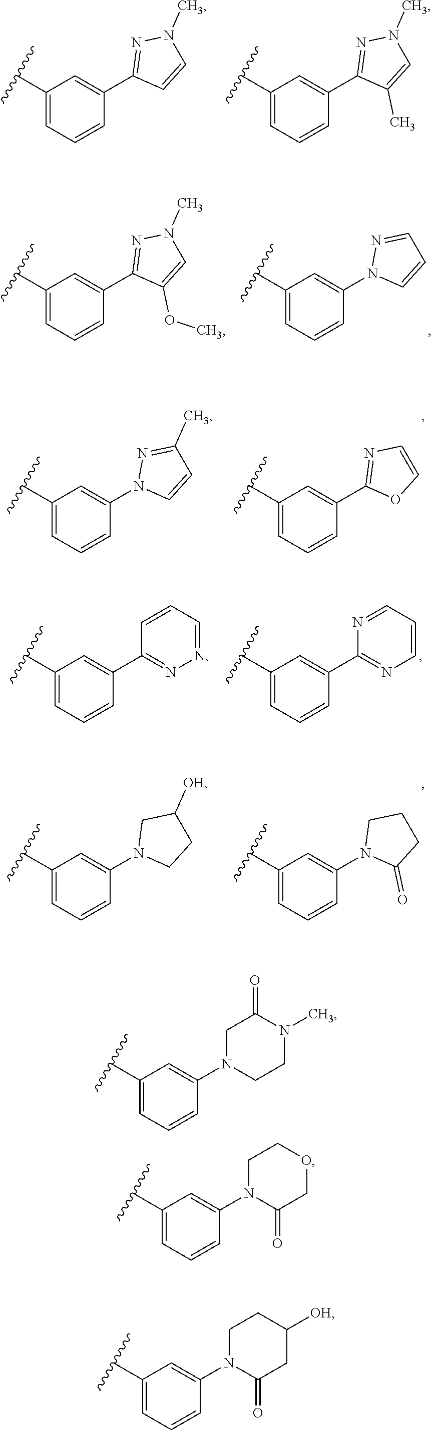

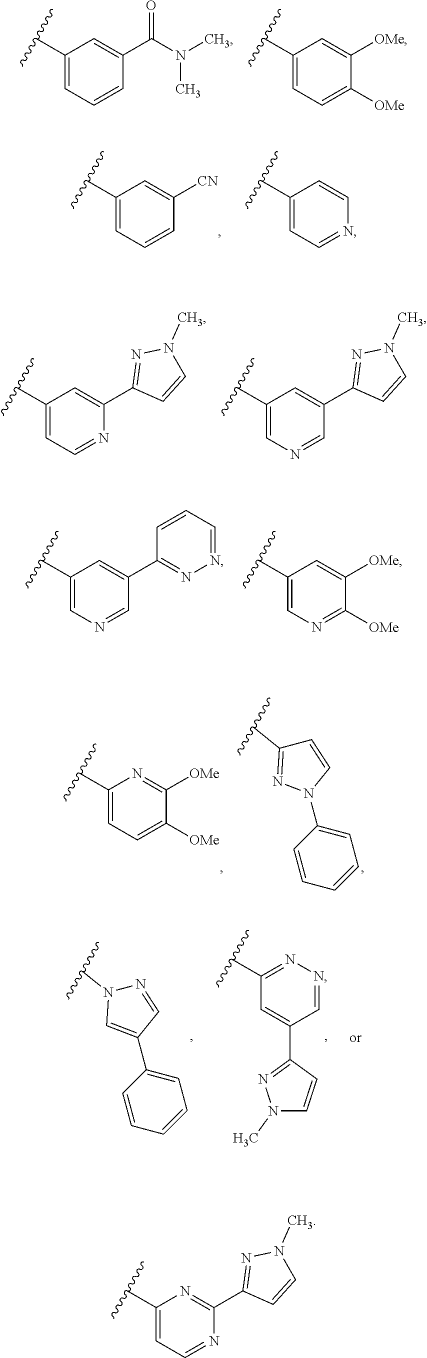

50-53. (canceled)

54. A compound of Formula II: 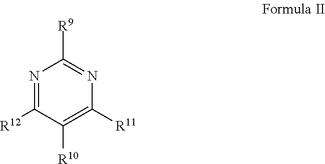
or a pharmaceutically acceptable salt thereof,
wherein
R⁹ is optionally substituted morpholin-4-yl, optionally substituted morpholin-3-ylalkoxy, optionally substituted 2-(pyridin-2-yl)alkoxy, optionally substituted 1-methylpiperazin-2-yl, or optionally substituted C₆-C₁₀ aryl;
R¹⁰ is optionally substituted C₁₋₄ alkoxy, —(CO)NR^{7a}R^{7b}, —P(O)R^{7c}R^{7d}, or —S(O)ₖR^{7e};
each of R^{7a} and R^{7b} is, independently, H or optionally substituted C₁-C₆ alkyl; or R^{7a} and R^{7b}, together with the nitrogen atom to which they are attached, combine to form optionally substituted C₂-C₉ heterocyclyl;
each of R^{7c}, R^{7d}, and R^{7e} is independently optionally substituted C₁₋₆ alkyl, optionally substituted C₁₋₆ alkoxy, or hydroxyl;
k is 0, 1, or 2;
R¹¹ is optionally substituted morpholin-4-yl, optionally substituted pyridin-4-yl, optionally substituted pyrazol-4-yl, optionally substituted 1H-imidazol-2-yl, optionally substituted quinoline-6-yl, or optionally substituted C₆-C₁₀ aryl; and
R¹² is optionally substituted 1-methylpiperazin-2-onyl, optionally substituted 2-(pyridin-2-yl)alkoxy, optionally substituted N-(pyridin-3-ylmethyl)amine, optionally substituted N-(pyridin-4-yl)amine, or optionally substituted C₆-C₁₀ aryl.

55-56. (canceled)

57. The compound of claim 54, wherein R⁹ is

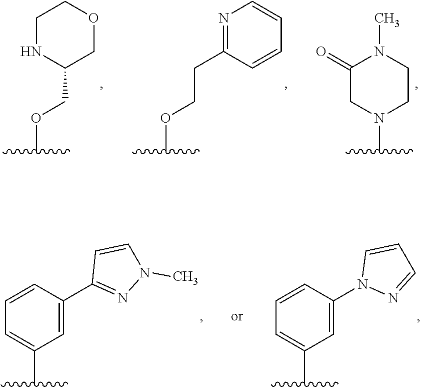

58-65. (canceled)

66. The compound of claim 54, wherein R¹¹ is pyridin-4-yl.

67. (canceled)

68. The compound of claim 54, wherein R¹¹ is

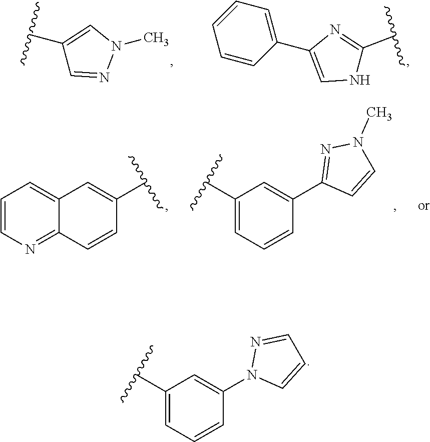

69-75. (canceled)

76. The compound of claim 54, wherein R¹² is

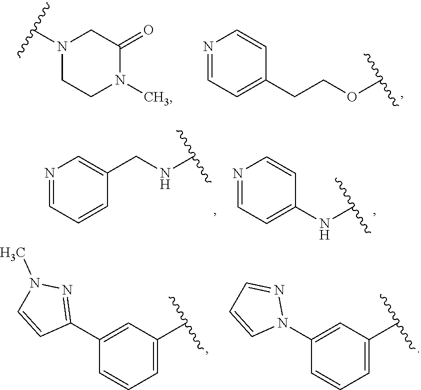

77-85. (canceled)

86. A compound selected from any one of the following: 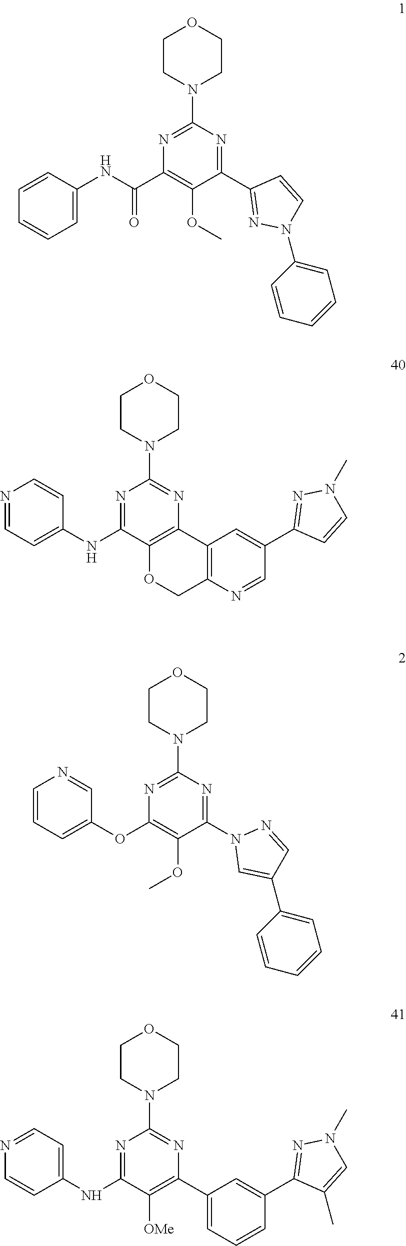 [SMILES: COc1c(C(=O)Nc2ccccc2)nc(N2CCOCC2)nc1-c1ccn(-c2ccccc2)n1 ; Cn1ccc(-c2cnc3c(c2)-c2nc(N4CCOCC4)nc(Nc4ccncc4)c2OC3)n1 ; COc1c(Oc2cccnc2)nc(N2CCOCC2)nc1-n1cc(-c2ccccc2)cn1 ; COc1c(Nc2ccncc2)nc(N2CCOCC2)nc1-c1cccc(-c2nn(C)cc2C)c1] 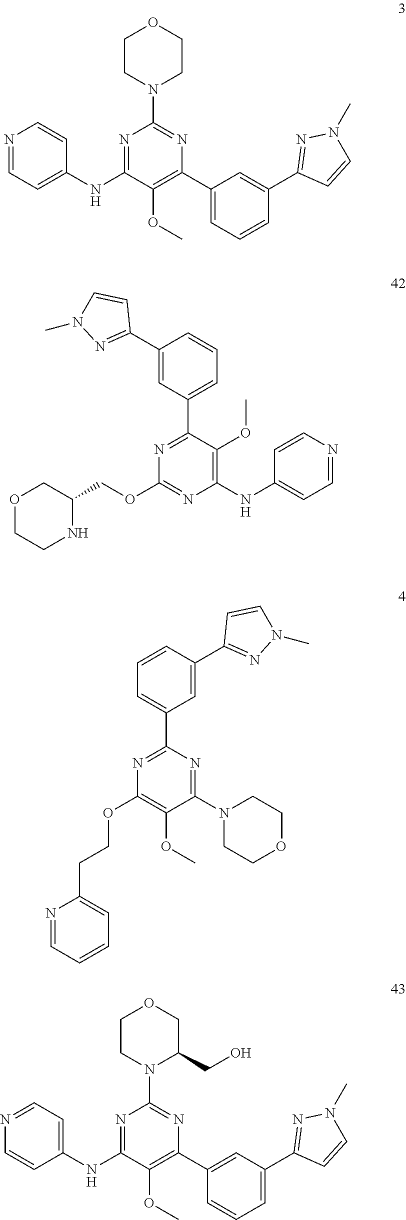 [SMILES: COc1c(Nc2ccncc2)nc(N2CCOCC2)nc1-c1cccc(-c2ccn(C)n2)c1 ; COc1c(Nc2ccncc2)nc(OC[C@H]2COCCN2)nc1-c1cccc(-c2ccn(C)n2)c1 ; COc1c(OCCc2ccccn2)nc(-c2cccc(-c3ccn(C)n3)c2)nc1N1CCOCC1 ; COc1c(Nc2ccncc2)nc(N2CCOC[C@H]2CO)nc1-c1cccc(-c2ccn(C)n2)c1] 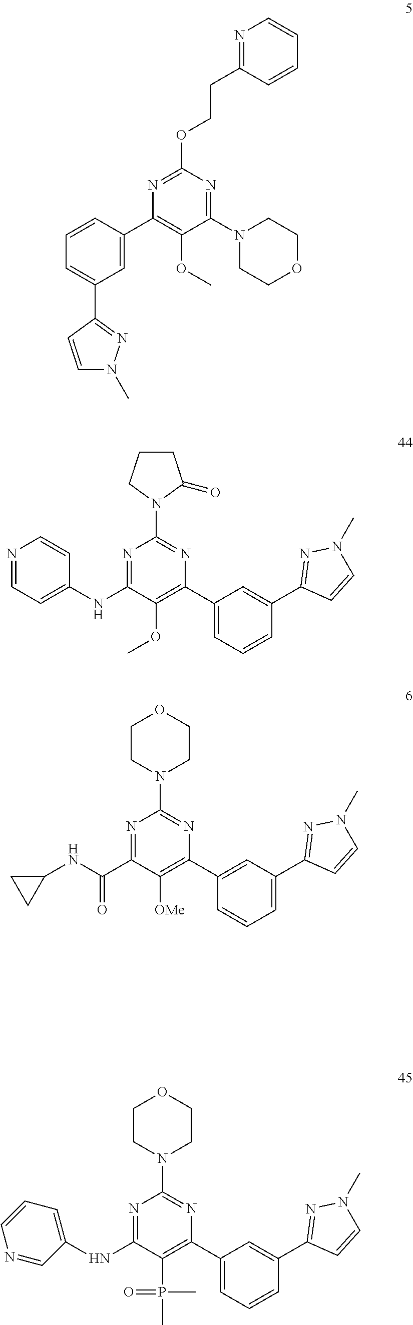 [SMILES: COc1c(-c2cccc(-c3ccn(C)n3)c2)nc(OCCc2ccccn2)nc1N1CCOCC1 ; COc1c(Nc2ccncc2)nc(N2CCCC2=O)nc1-c1cccc(-c2ccn(C)n2)c1 ; COc1c(C(=O)NC2CC2)nc(N2CCOCC2)nc1-c1cccc(-c2ccn(C)n2)c1 ; Cn1ccc(-c2cccc(-c3nc(N4CCOCC4)nc(Nc4cccnc4)c3P(C)(C)=O)c2)n1] 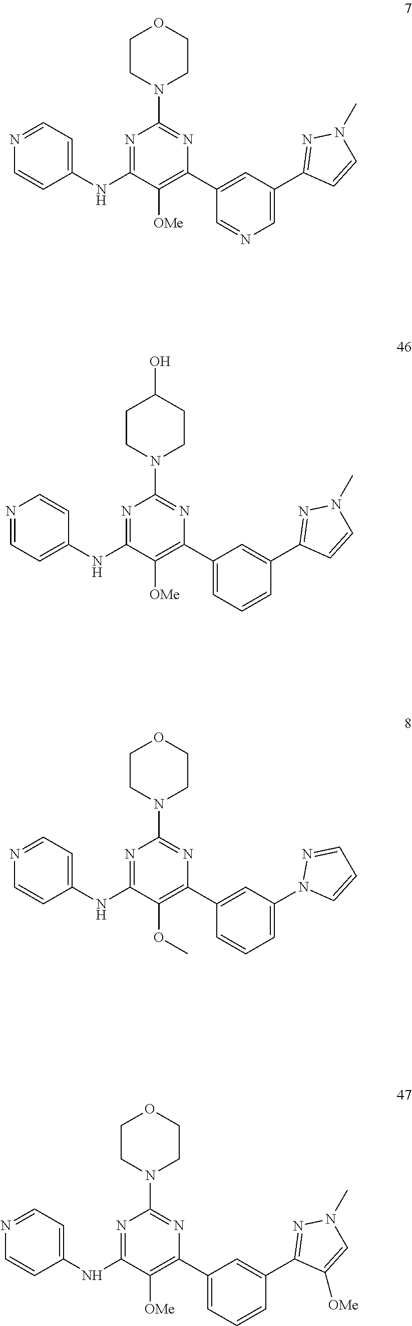 [SMILES: COc1c(Nc2ccncc2)nc(N2CCOCC2)nc1-c1cncc(-c2ccn(C)n2)c1 ; COc1c(Nc2ccncc2)nc(N2CCC(O)CC2)nc1-c1cccc(-c2ccn(C)n2)c1 ; COc1c(Nc2ccncc2)nc(N2CCOCC2)nc1-c1cccc(-n2cccn2)c1 ; COc1cn(C)nc1-c1cccc(-c2nc(N3CCOCC3)nc(Nc3ccncc3)c2OC)c1] 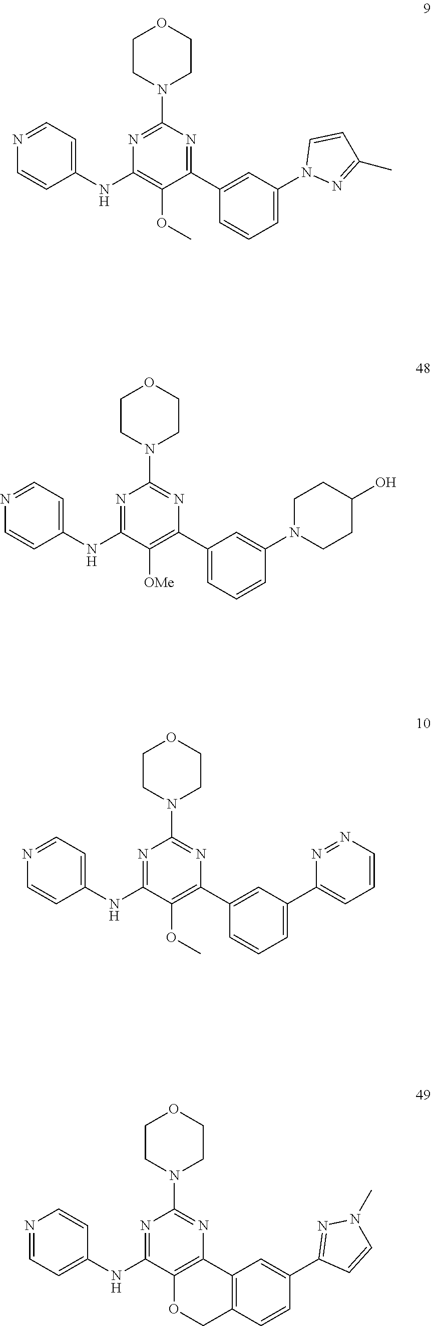 [SMILES: COc1c(Nc2ccncc2)nc(N2CCOCC2)nc1-c1cccc(-n2ccc(C)n2)c1 ; COc1c(Nc2ccncc2)nc(N2CCOCC2)nc1-c1cccc(N2CCC(O)CC2)c1 ; COc1c(Nc2ccncc2)nc(N2CCOCC2)nc1-c1cccc(-c2cccnn2)c1 ; Cn1ccc(-c2ccc3c(c2)-c2nc(N4CCOCC4)nc(Nc4ccncc4)c2OC3)n1] 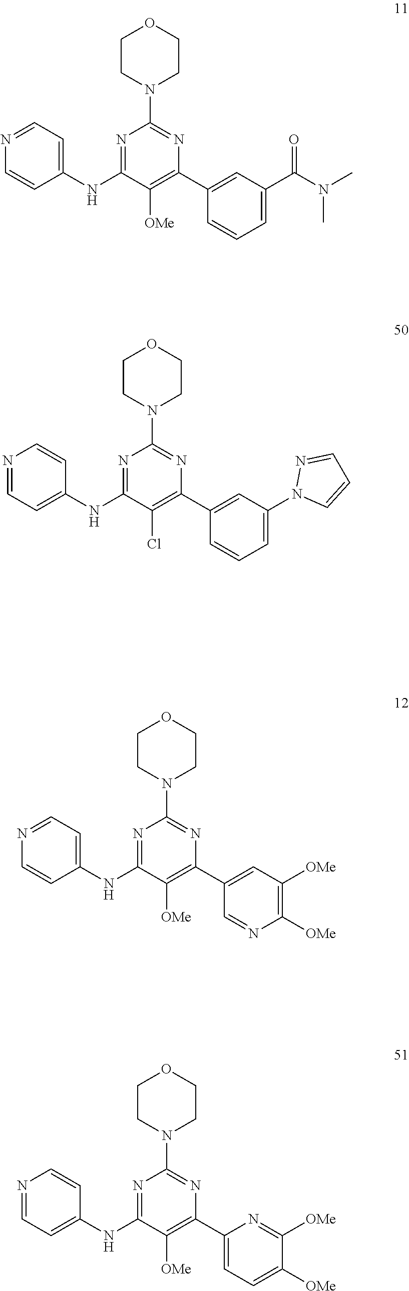 [SMILES: COc1c(Nc2ccncc2)nc(N2CCOCC2)nc1-c1cccc(C(=O)N(C)C)c1 ; Clc1c(Nc2ccncc2)nc(N2CCOCC2)nc1-c1cccc(-n2cccn2)c1 ; COc1cc(-c2nc(N3CCOCC3)nc(Nc3ccncc3)c2OC)cnc1OC ; COc1ccc(-c2nc(N3CCOCC3)nc(Nc3ccncc3)c2OC)nc1OC] 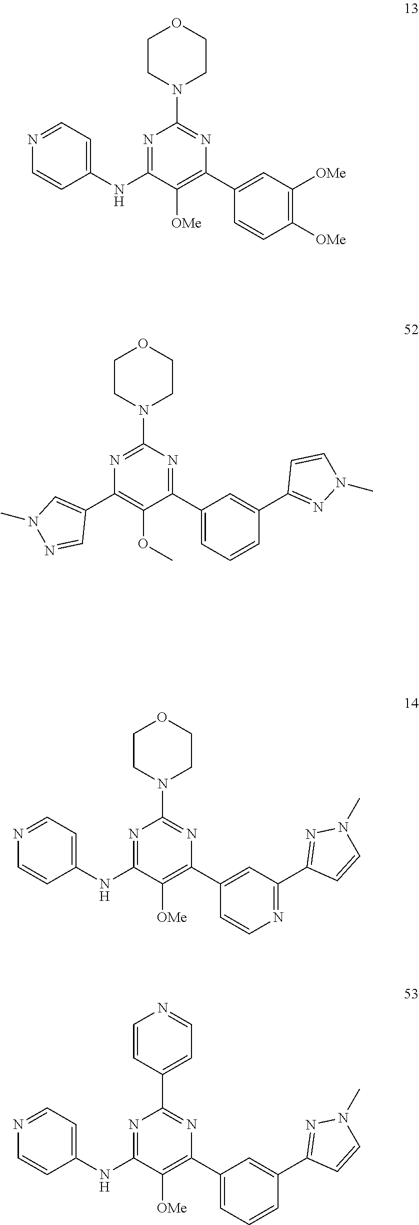 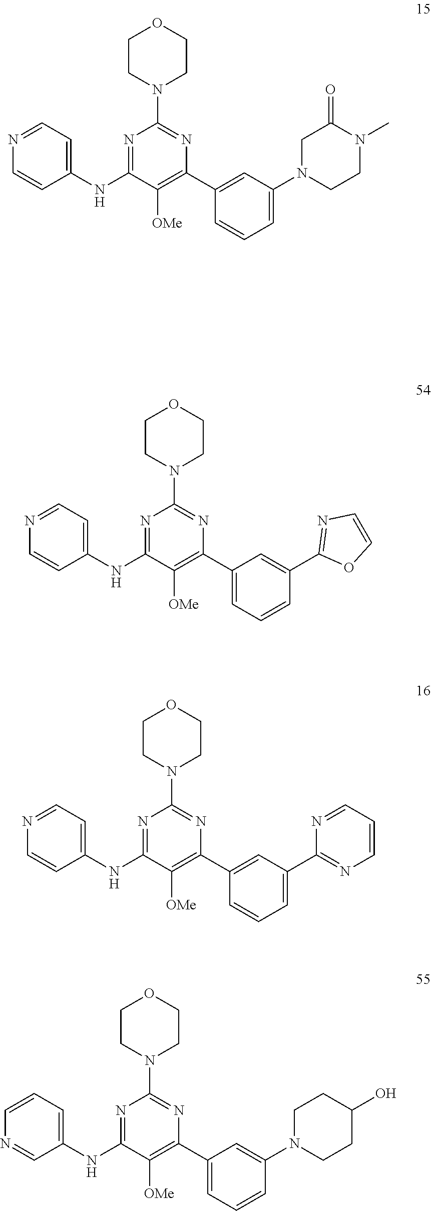 [SMILES: COc1c(Nc2ccncc2)nc(N2CCOCC2)nc1-c1cccc(N2CCN(C)C(=O)C2)c1 ; COc1c(Nc2ccncc2)nc(N2CCOCC2)nc1-c1cccc(-c2ncco2)c1 ; COc1c(Nc2ccncc2)nc(N2CCOCC2)nc1-c1cccc(-c2ncccn2)c1 ; COc1c(Nc2cccnc2)nc(N2CCOCC2)nc1-c1cccc(N2CCC(O)CC2)c1] 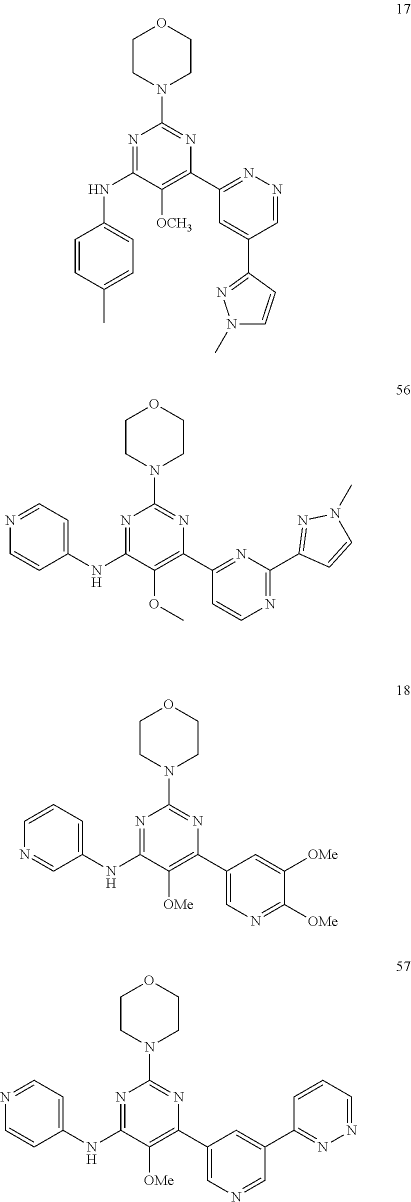 [SMILES: COc1c(Nc2ccc(C)cc2)nc(N2CCOCC2)nc1-c1cc(-c2ccn(C)n2)cnn1 ; COc1c(Nc2ccncc2)nc(N2CCOCC2)nc1-c1ccnc(-c2ccn(C)n2)n1 ; COc1cc(-c2nc(N3CCOCC3)nc(Nc3cccnc3)c2OC)cnc1OC ; COc1c(Nc2ccncc2)nc(N2CCOCC2)nc1-c1cncc(-c2cccnn2)c1] 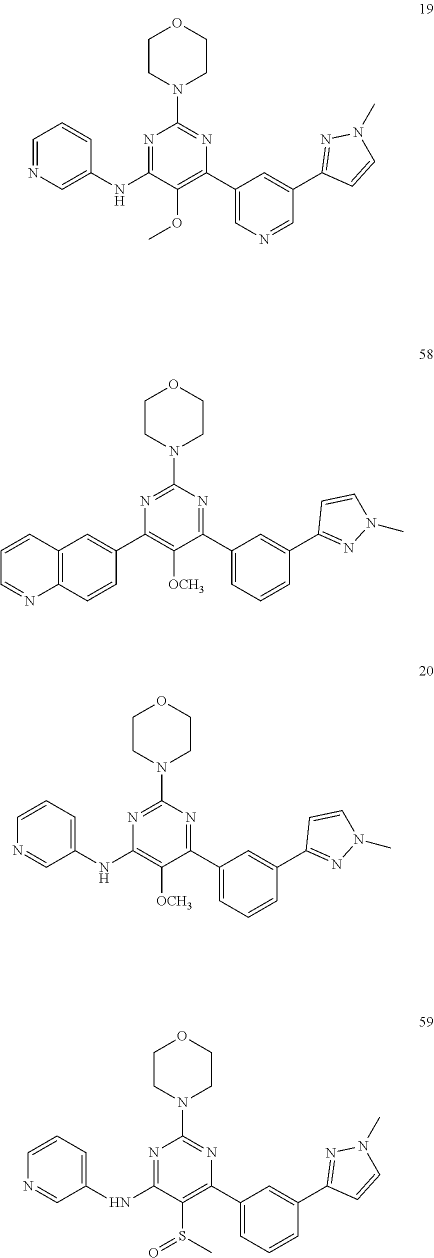 [SMILES: COc1c(Nc2cccnc2)nc(N2CCOCC2)nc1-c1cncc(-c2ccn(C)n2)c1 ; COc1c(-c2cccc(-c3ccn(C)n3)c2)nc(N2CCOCC2)nc1-c1ccc2ncccc2c1 ; COc1c(Nc2cccnc2)nc(N2CCOCC2)nc1-c1cccc(-c2ccn(C)n2)c1 ; Cn1ccc(-c2cccc(-c3nc(N4CCOCC4)nc(Nc4cccnc4)c3S(C)=O)c2)n1] 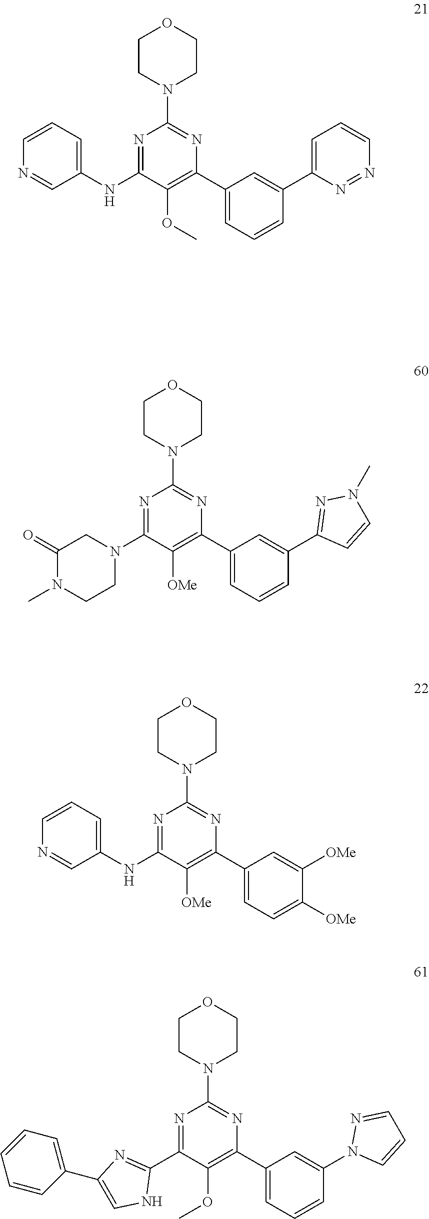 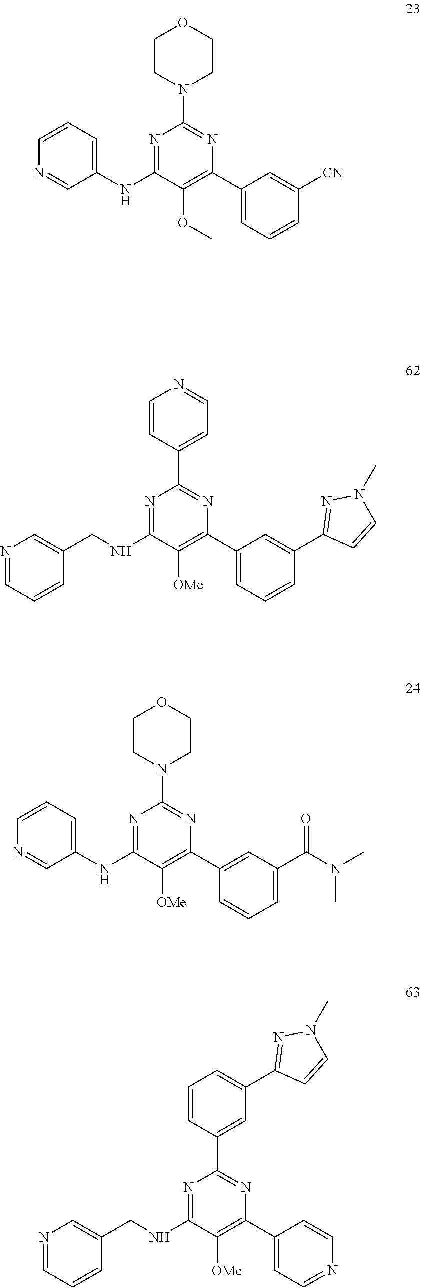 [SMILES: COc1c(Nc2cccnc2)nc(N2CCOCC2)nc1-c1cccc(C#N)c1 ; COc1c(NCc2cccnc2)nc(-c2ccncc2)nc1-c1cccc(-c2ccn(C)n2)c1 ; COc1c(Nc2cccnc2)nc(N2CCOCC2)nc1-c1cccc(C(=O)N(C)C)c1 ; COc1c(NCc2cccnc2)nc(-c2cccc(-c3ccn(C)n3)c2)nc1-c1ccncc1] 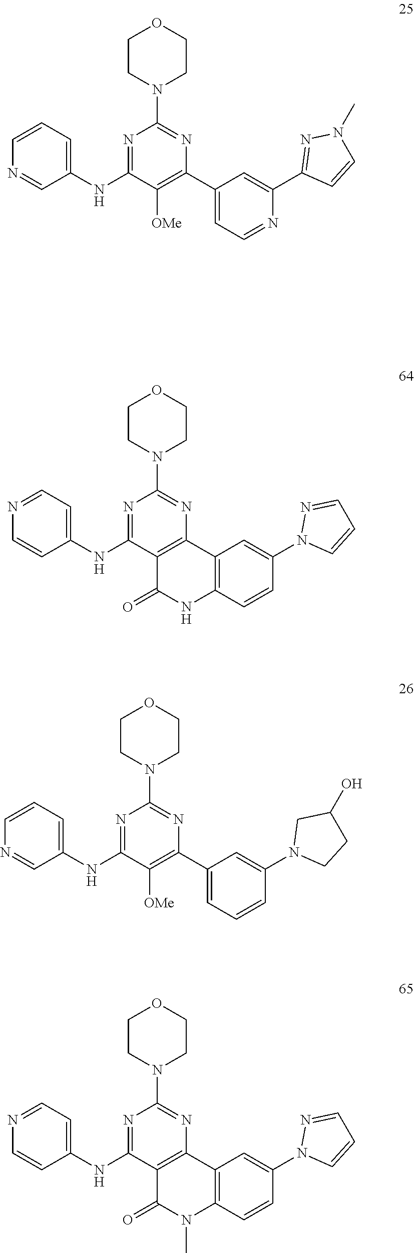 [SMILES: COc1c(Nc2cccnc2)nc(N2CCOCC2)nc1-c1ccnc(-c2ccn(C)n2)c1 ; O=c1[nH]c2ccc(-n3cccn3)cc2c2nc(N3CCOCC3)nc(Nc3ccncc3)c12 ; COc1c(Nc2cccnc2)nc(N2CCOCC2)nc1-c1cccc(N2CCC(O)C2)c1 ; Cn1c(=O)c2c(Nc3ccncc3)nc(N3CCOCC3)nc2c2cc(-n3cccn3)ccc21] 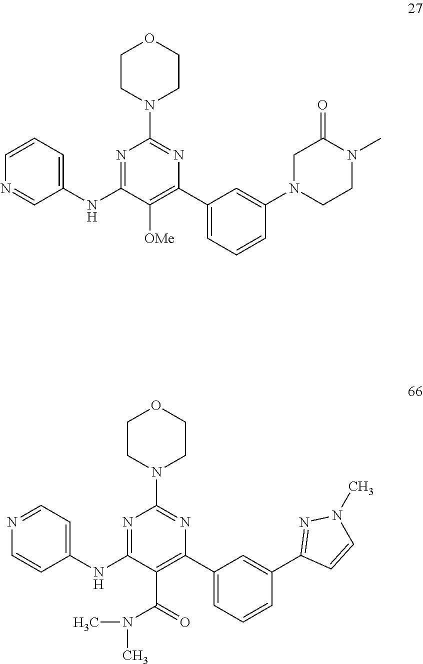 [SMILES: COc1c(Nc2cccnc2)nc(N2CCOCC2)nc1-c1cccc(N2CCN(C)C(=O)C2)c1 ; CN(C)C(=O)c1c(Nc2ccncc2)nc(N2CCOCC2)nc1-c1cccc(-c2ccn(C)n2)c1] 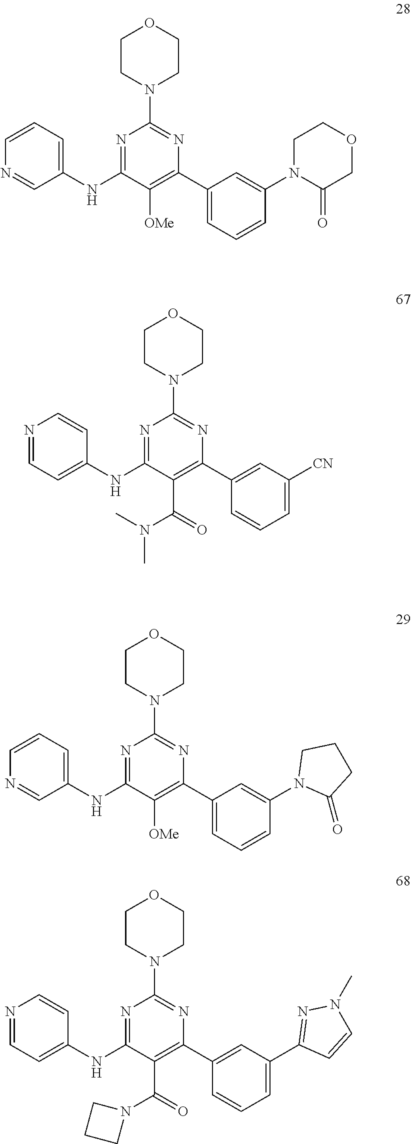 [SMILES: COc1c(Nc2cccnc2)nc(N2CCOCC2)nc1-c1cccc(N2CCOCC2=O)c1 ; CN(C)C(=O)c1c(Nc2ccncc2)nc(N2CCOCC2)nc1-c1cccc(C#N)c1 ; COc1c(Nc2cccnc2)nc(N2CCOCC2)nc1-c1cccc(N2CCCC2=O)c1 ; Cn1ccc(-c2cccc(-c3nc(N4CCOCC4)nc(Nc4ccncc4)c3C(=O)N3CCC3)c2)n1] 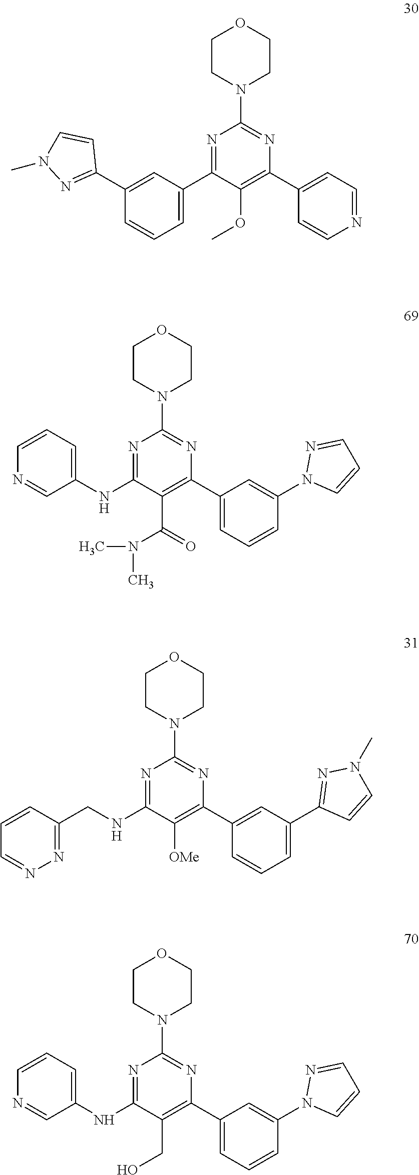 [SMILES: COc1c(-c2ccncc2)nc(N2CCOCC2)nc1-c1cccc(-c2ccn(C)n2)c1 ; CN(C)C(=O)c1c(Nc2cccnc2)nc(N2CCOCC2)nc1-c1cccc(-n2cccn2)c1 ; COc1c(NCc2cccnn2)nc(N2CCOCC2)nc1-c1cccc(-c2ccn(C)n2)c1 ; OCc1c(Nc2cccnc2)nc(N2CCOCC2)nc1-c1cccc(-n2cccn2)c1] 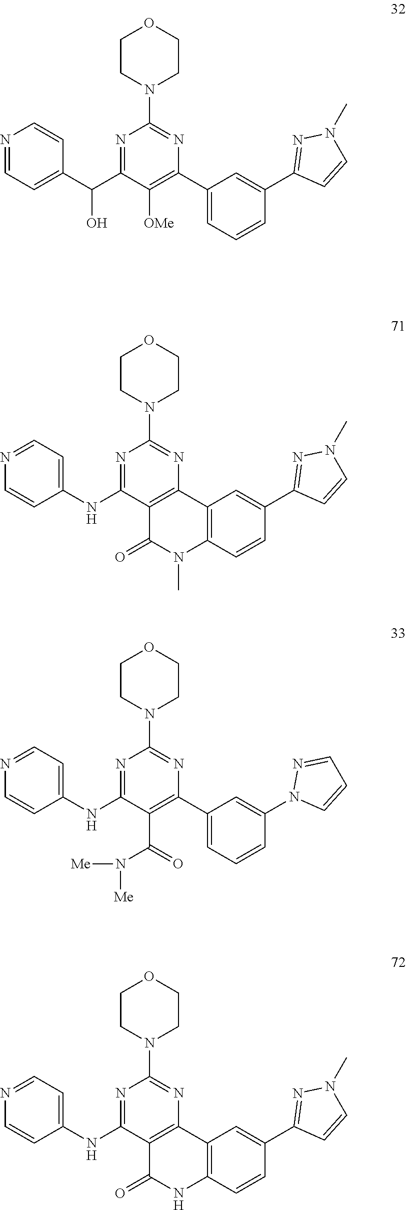 [SMILES: COc1c(-c2cccc(-c3ccn(C)n3)c2)nc(N2CCOCC2)nc1C(O)c1ccncc1 ; Cn1ccc(-c2ccc3c(c2)c2nc(N4CCOCC4)nc(Nc4ccncc4)c2c(=O)n3C)n1 ; CN(C)C(=O)c1c(Nc2ccncc2)nc(N2CCOCC2)nc1-c1cccc(-n2cccn2)c1 ; Cn1ccc(-c2ccc3[nH]c(=O)c4c(Nc5ccncc5)nc(N5CCOCC5)nc4c3c2)n1] 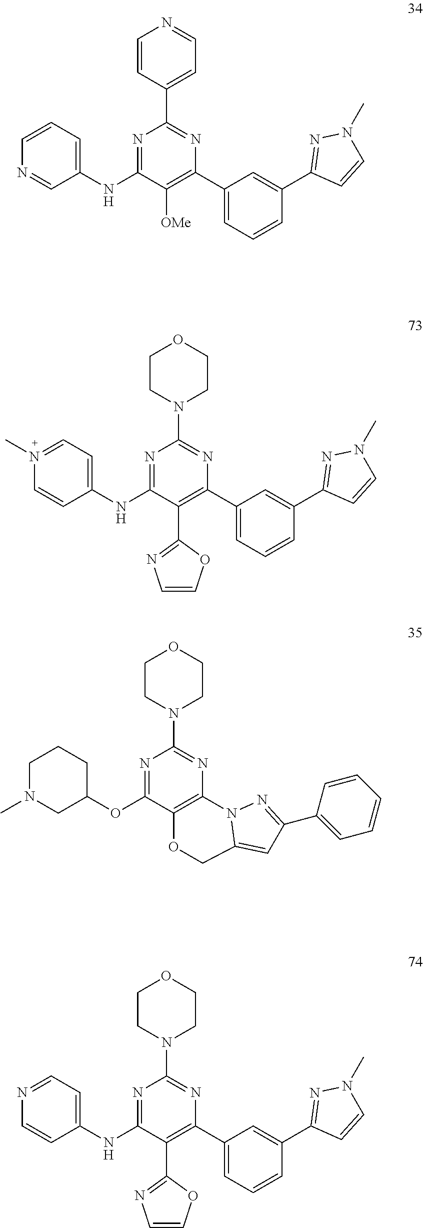 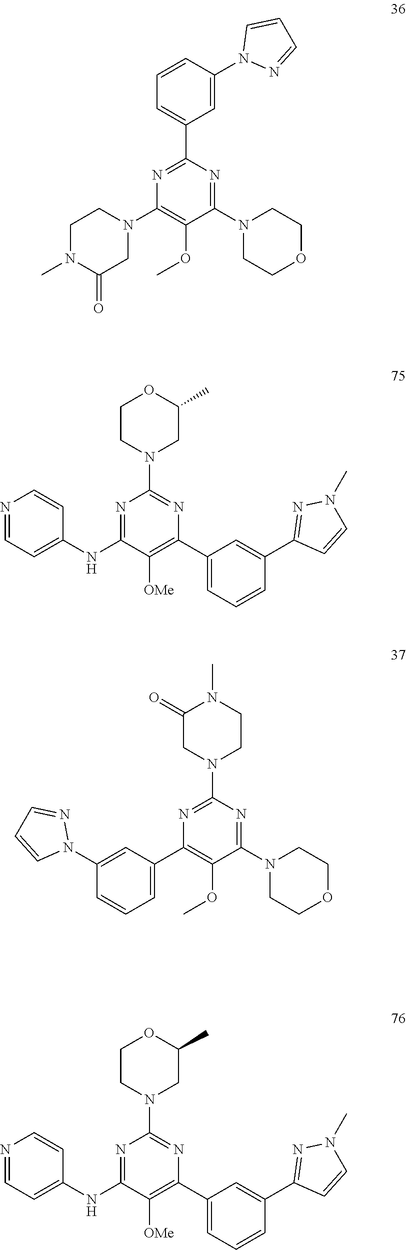 [SMILES: COc1c(N2CCOCC2)nc(-c2cccc(-n3cccn3)c2)nc1N1CCN(C)C(=O)C1 ; COc1c(Nc2ccncc2)nc(N2CCO[C@H](C)C2)nc1-c1cccc(-c2ccn(C)n2)c1 ; COc1c(-c2cccc(-n3cccn3)c2)nc(N2CCN(C)C(=O)C2)nc1N1CCOCC1 ; COc1c(Nc2ccncc2)nc(N2CCO[C@@H](C)C2)nc1-c1cccc(-c2ccn(C)n2)c1] 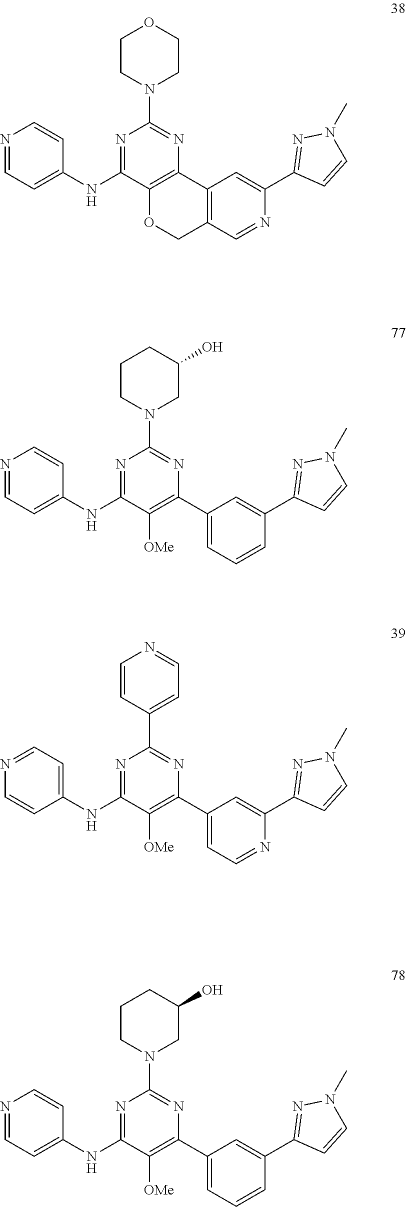 [SMILES: Cn1ccc(-c2cc3c(cn2)COc2c(Nc4ccncc4)nc(N4CCOCC4)nc2-3)n1 ; COc1c(Nc2ccncc2)nc(N2CCC[C@H](O)C2)nc1-c1cccc(-c2ccn(C)n2)c1 ; COc1c(Nc2ccncc2)nc(-c2ccncc2)nc1-c1ccnc(-c2ccn(C)n2)c1 ; COc1c(Nc2ccncc2)nc(N2CCC[C@@H](O)C2)nc1-c1cccc(-c2ccn(C)n2)c1]
or a pharmaceutically acceptable salt of the foregoing.

87-88. (canceled)

89. A pharmaceutical composition comprising the compound of claim 1, or a pharmaceutically acceptable salt thereof, and a pharmaceutically acceptable excipient.

90. A method of treating a neurological disorder in a subject in need thereof, the method comprising administering to the subject an effective amount of the compound of claim 1, or a pharmaceutically acceptable salt thereof.

91. (canceled)

92. The method of claim 90, wherein the neurological disorder is ALS.

93. A method of inhibiting toxicity in a cell related to a protein, the method comprising contacting the cell with the compound of claim 1 or a pharmaceutically acceptable salt thereof.

94. The method of claim 93, wherein the toxicity is TDP-43-related toxicity, or 9orf72-related toxicity.

95. (canceled)

96. A method of inhibiting PIKfyve in a cell expressing PIKfyve protein, the method comprising contacting the cell with the compound of claim 1 or a pharmaceutically acceptable salt thereof.

97-99. (canceled)
